(19) United States
(12) Patent Application Publication
(10) Pub. No.: US 2013/0096104 A1
(43) Pub. Date: Apr. 18, 2013

(54) IMIDAZOPYRIDINE COMPOUNDS, COMPOSITIONS AND METHODS OF USE

(71) Applicants: Yingjie Lai, Cupertino, CA (US); Jun Liang, Palo Alto, CA (US); Steven R. Magnuson, Dublin, CA (US); Kirk D. Robarge, San Francisco, CA (US); Vickie H. Tsui, Burlingame, CA (US); Birong Zhang, Union City, CA (US); Aihe Zhou, San Jose, CA (US)

(72) Inventors: Yingjie Lai, Cupertino, CA (US); Jun Liang, Palo Alto, CA (US); Steven R. Magnuson, Dublin, CA (US); Kirk D. Robarge, San Francisco, CA (US); Vickie H. Tsui, Burlingame, CA (US); Birong Zhang, Union City, CA (US); Aihe Zhou, San Jose, CA (US)

(73) Assignee: GENENTECH, INC., South San Francisco, CA (US)

(21) Appl. No.: 13/635,369

(22) PCT Filed: Mar. 15, 2011
(86) PCT No.: PCT/EP11/53826

§ 371 Date: Dec. 19, 2012

Related U.S. Application Data
(60) Provisional application No. 61/314,932, filed on Mar. 17, 2010.

(51) Int. Cl. C07D 471/04; C07D 473/00

(52) U.S. Cl. CPC C07D 471/04; C07D 473/00
USPC 514/210.21; 546/118; 514/303; 544/264; 514/263.1; 544/295; 514/252.18; 544/328; 514/256; 514/234.2; 544/122; 544/405; 514/255.05; 544/277; 514/263.21; 514/263.4; 514/252.04; 544/238

(57) ABSTRACT

The invention provides compounds of Formulas Ia-Ib, stereoisomers or pharmaceutically acceptable salts thereof, wherein A, X, R¹, R², R⁴, R⁵ and R¹⁶ are defined herein, a pharmaceutical composition that includes a compound of Formulas Ia-Ib and a pharmaceutically acceptable carrier, adjuvant or vehicle, and methods of using the compound or composition in therapy, as an inhibitor of TYK2 kinase and conditions related, such as inflammatory illnesses, inflammatory bowel disease or psoriasis.

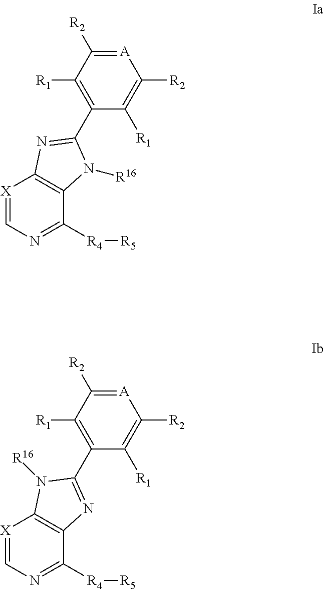

### FIELD OF THE INVENTION

The present invention relates to organic compounds useful for therapy and/or prophylaxis in a patient, and in particular to inhibitors of TYK2 kinase useful for treating diseases mediated by TYK2 kinase.

### BACKGROUND OF INVENTION

Cytokine pathways mediate a broad range of biological functions, including many aspects of inflammation and immunity. Janus kinases (JAK), including JAK1, JAK2, JAK3 and TYK2 are cytoplasmic protein kinases that associate with type I and type II cytokine receptors and regulate cytokine signal transduction. Cytokine engagement with cognate receptors triggers activation of receptor associated JAKs and this leads to JAK-mediated tyrosine phosphorylation of signal transducer and activator of transcription (STAT) proteins and ultimately transcriptional activation of specific gene sets. JAK1, JAK2 and TYK2 exhibit broad patterns of gene expression, while JAK3 expression is limited to leukocytes. Cytokine receptors are typically functional as heterodimers, and as a result, more than one type of JAK kinase is usually associated with cytokine receptor complexes. The specific JAKs associated with different cytokine receptor complexes have been determined in many cases through genetic studies and corroborated by other experimental evidence.

JAK1 is functionally and physically associated with the type I interferon (e.g., IFNalpha), type II interferon (e.g., IFNgamma), IL-2 and IL-6 cytokine receptor complexes. JAK1 knockout mice die perinatally due to defects in LIF receptor signaling. Characterization of tissues derived from JAK1 knockout mice demonstrated critical roles for this kinase in the IFN, IL-10, IL-2/IL-4, and IL-6 pathways. A humanized monoclonal antibody targeting the IL-6 pathway (Tocilizumab) was recently approved by the European Commission for the treatment of moderate-to-severe rheumatoid arthritis.

Biochemical and genetic studies have shown an association between JAK2 and single-chain (e.g., EPO), IL-3 and interferon gamma cytokine receptor families. Consistent with this, JAK2 knockout mice die of anemia. Kinase activating mutations in JAK2 (e.g., JAK2 V617F) are associated with myeloproliferative disorders (MPDs) in humans.

JAK3 associates exclusively with the gamma common cytokine receptor chain, which is present in the IL-2, IL-4, IL-7, IL-9, IL-15 and IL-21 cytokine receptor complexes. JAK3 is critical for lymphoid cell development and proliferation and mutations in JAK3 result in severe combined immunodeficiency (SCID). Based on its role in regulating lymphocytes, JAK3 and JAK3-mediated pathways have been targeted for immunosuppressive indications (e.g., transplantation rejection and rheumatoid arthritis).

TYK2 associates with the type I interferon (e.g., IFNalpha), IL-6, IL-10, IL-12 and IL-23 cytokine receptor complexes. Consistent with this, primary cells derived from a TYK2 deficient human are defective in type I interferon, IL-6, IL-10, IL-12 and IL-23 signaling. A fully human monoclonal antibody targeting the shared p40 subunit of the IL-12 and 11-23 cytokines (Ustekinumab) was recently approved by the European Commission for the treatment of moderate-to-severe plaque psoriasis. In addition, an antibody targeting the IL-12 and IL-23 pathways underwent clinical trials for treating Crohn's Disease.

### SUMMARY OF INVENTION

One embodiment includes a compound of Formulas Ia-Ib:

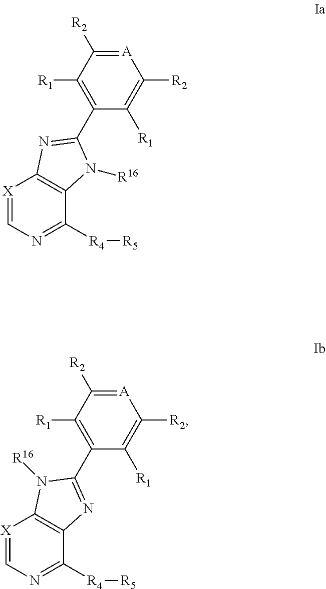

stereoisomers, tautomers or pharmaceutically acceptable salts thereof, wherein A, X, R¹, R², R⁴, R⁵ and R¹⁶ are defined herein.

Another embodiment includes a pharmaceutical composition that includes a compound of Formulas Ia-Ib, stereoisomers, tautomers or pharmaceutically acceptable salts thereof, and a pharmaceutically acceptable carrier, adjuvant or vehicle.

Another embodiment includes a method of inhibiting TYK2 kinase activity in a cell, comprising introducing into said cell an amount effective to inhibit said kinase of a compound of Formulas Ia-Ib, stereoisomers, tautomers or pharmaceutically acceptable salts thereof.

Another embodiment includes a method of treating or lessening the severity of a disease or condition responsive to the inhibition of TYK2 kinase activity in a patient. The method includes administering to the patient a therapeutically effective amount of a compound of Formulas Ia-Ib, stereoisomers, tautomers or pharmaceutically acceptable salts thereof.

Another embodiment includes use of a compound of Formulas Ia-Ib, stereoisomers, tautomers or pharmaceutically acceptable salts thereof, in therapy.

Another embodiment includes use of a compound of Formulas Ia-Ib, stereoisomers, tautomers or pharmaceutically acceptable salts thereof, in manufacturing a medicament for treating a disease responsive to the inhibition of TYK2 kinase.

Another embodiment includes methods of preparing a compound of Formulas Ia-Ib, stereoisomers, tautomers or pharmaceutically acceptable salts thereof.

Another embodiment includes a kit for treating a disease or disorder responsive to the inhibition of TYK2 kinase. The kit includes a first pharmaceutical composition comprising a compound of Formulas Ia-Ib and instructions for use

### DETAILED DESCRIPTION OF THE INVENTION

Reference will now be made in detail to certain embodiments, examples of which are illustrated in the accompanying structures and formulas. While the invention will be described in conjunction with the enumerated embodiments, the invention is intended to cover all alternatives, modifications, and equivalents, which may be included within the scope of the present invention as defined by the claims. One skilled in the art will recognize methods and materials similar or equivalent to those described herein, which could be used in the practice of the present invention.

### DEFINITIONS

The term “alkyl” refers to a saturated linear or branched-chain monovalent hydrocarbon radical, wherein the alkyl radical may be optionally substituted independently with one or more substituents described herein. In one example, the alkyl radical is one to eighteen carbon atoms (C₁-C₁₈). In other examples, the alkyl radical is C₀-C₆, C₀-C₅, C₀-C₃, C₁-C₁₂, C₁-C₁₀, C₁-C₈, C₁-C₆, C₁-C₅, C₁-C₄, or C₁-C₃. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂)_{,} 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl.

The term “alkenyl” refers to linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical is two to eighteen carbon atoms (C₂-C₁₈). In other examples, the alkenyl radical is C₂-C₁₂, C₂-C₁₀, C₂-C₈, C₂-C₆ or C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The term “alkynyl” refers to a linear or branched monovalent hydrocarbon radical with at least one site of unsaturation, i.e., a carbon-carbon, triple bond, wherein the alkynyl radical may be optionally substituted independently with one or more substituents described herein. In one example, the alkynyl radical is two to eighteen carbon atoms (C₂-C₁₈). In other examples, the alkynyl radical is C₂-C₁₂, C₂-C₁₀, C₂-C₈, C₂-C₆ or C₂-C₃. Examples include, but are not limited to, ethynyl (—C≡CH), prop-1-ynyl (—C≡CCH₃), prop-2-ynyl (propargyl, —CH₂C≡CH), but-1-ynyl, but-2-ynyl and but-3-ynyl.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 12 carbon atoms (C₃-C₁₂). In other examples, cycloalkyl is C₃-C₈, C₃-C₁₀ or C₅-C₁₀. In other examples, the cycloalkyl group, as a monocycle, is C₃-C₄, C₃-C₆ or C₅-C₆. In another example, the cycloalkyl group, as a bicycle, is C₇-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane and bicyclo[3.2.2]nonane.

“Aryl” refers to a cyclic aromatic hydrocarbon group optionally substituted independently with one or more substituents described herein. In one example, the aryl group is 6-20 carbon atoms (C₆-C₂₀). In another example, the aryl group is C₆-C₉. In another example, the aryl group is a C₆ aryl group. Aryl includes bicyclic groups comprising an aromatic ring with a fused non-aromatic or partially saturated ring. Example aryl groups include, but are not limited to, phenyl, naphthalenyl, anthracenyl, indenyl, indanyl, 1,2-dihydronapthalenyl and 1,2,3,4-tetrahydronapthyl. In one example, aryl includes phenyl. Substituted phenyl or substituted aryl means a phenyl group or aryl group substituted with one, two, three, four or five, for example 1-2, 1-3 or 1-4 substituents chosen from groups specified herein. In one example, optional substituents on aryl are selected from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulfonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted with a similar group as those denoted above. Examples of the term “substituted phenyl” include a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl, a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

“Halo” or “halogen” refer to F, Cl, Br or I.

The terms “heterocycle,” “heterocyclyl” and “heterocyclic ring” are used interchangeably herein and refer to: (i) a saturated or partially unsaturated cyclic group (i.e., having one or more double and/or triple bonds within the ring) (“heterocycloalkyl”), or (ii) an aromatic cyclic group (“heteroaryl”), and in each case, which at least one ring atom is a heteroatom independently selected from nitrogen, oxygen, phosphorus and sulfur, the remaining ring atoms being carbon. The heterocyclyl group may be optionally substituted with one or more substituents described below. In one embodiment, heterocyclyl includes monocycles or bicycles having 1 to 9 carbon ring members (C₁-C₉) with the remaining ring atoms being heteroatoms selected from N, O, S and P. In other examples, heterocyclyl includes monocycles or bicycles having C₁-C₅, C₃-C₅ or C₄-C₅, with the remaining ring atoms being heteroatoms selected from N, O, S and P. In certain embodiments, heterocyclyl includes 3-10-membered rings, 3-7-membered rings or 3-6 membered rings, containing one or more heteroatoms independently selected from N, O, S and P. In other examples, heterocyclyl includes monocyclic 3-, 4-, 5-, 6- or 7-membered rings, containing one or more heteroatoms independently selected from N, O, S and P. In another embodiment, heterocyclyl includes bi- or poly-cyclic, spiro or bridged 4-, 5-, 6-, 7-, 8-, 9- or 10-membered ring systems, containing one or more heteroatoms independently selected from N, O, S and P. Examples of bicycle systems include, but are not limited to, [3,5], [4,5], [5,5], [3,6], [4,6], [5,6], or [6,6] systems. Examples of bridged ring systems include, but are not limited to [2.2.1], [2.2.2], [3.2.2] and [4.1.0] arrangements, and having 1 to 3 heteroatoms selected from N, O, S and P. In another embodiment, heterocyclyl includes spiro groups having 1 to 4 heteroatoms selected from N, O, S and P. The heterocyclyl group may be a carbon-linked group or heteroatom-linked group. “Heterocyclyl” includes a heterocyclyl group fused to a cycloalkyl group.

Exemplary heterocyclyl groups include, but are not limited to, oxiranyl, aziridinyl, thiiranyl, azetidinyl, oxetanyl, thietanyl, 1,2-dithietanyl, 1,3-dithietanyl, pyrrolidinyl, piperidinyl, morpholinyl, thiomorpholinyl, thioxanyl, piperazinyl, homopiperazinyl, homopiperidinyl, oxepanyl, thiepanyl, oxazepinyl, oxazepanyl, diazepanyl, 1,4-diazepanyl, diazepinyl, thiazepinyl, thiazepanyl, dihydrothienyl, dihydropyranyl, dihydrofuranyl, tetrahydropyranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, dioxanyl, 1,3-dioxolanyl, pyrazolinyl, pyrazolidinyl, dithianyl, dithiolanyl, pyrazolidinylimidazolinyl, imidazolidinyl, 3-azabicyco[3.1.0] hexanyl, 3,6-diazabicyclo[3.1.1]heptanyl, 6-azabicyclo[3.1.1]heptanyl, 3-azabicyclo[3.1.1] heptanyl, 3-azabicyclo[4.1.0] heptanyl, 2-oxa-6-azaspiro[3.3] heptanyl and azabicyclo[2.2.2]hexanyl. Examples of a heterocyclyl group wherein a ring atom is substituted with oxo (═O) are pyrimidinonyl and 1,1-dioxo-thiomorpholinyl. The heterocyclyl groups herein are optionally substituted independently with one or more substituents described herein. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

The term “heteroaryl” refers to an aromatic carbocyclic radical in which at least one ring atom is a heteroatom independently selected from nitrogen, oxygen and sulfur, the remaining ring atoms being carbon. Heteroaryl groups may be optionally substituted with one or more substituents described herein. In one example, the heteroaryl group contains 1 to 9 carbon ring atoms (C₁-C₉). In other examples, the heteroaryl group is C₁-C₅, C₃-C₅ or C₄-C₅. In one embodiment, exemplary heteroaryl groups include 5-10-membered rings or 5-6-membered rings, or monocyclic aromatic 5-, 6- and 7-membered rings containing one or more heteroatoms independently selected from nitrogen, oxygen, and sulfur. In another embodiment, exemplary heteroaryl groups include fused ring systems of up to 10, or in another example 9, carbon atoms wherein at least one aromatic ring contains one or more heteroatoms independently selected from nitrogen, oxygen, and sulfur. “Heteroaryl” includes heteroaryl groups fused with an aryl, cycloalkyl or other heterocyclyl group. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, furazanyl, benzofurazanyl, benzothiophenyl, benzothiazolyl, benzoxazolyl, quinazolinyl, quinoxalinyl, naphthyridinyl, pyrrolopyrimidinyl, pyrazolopyrimidinyl and furopyridinyl.

In certain embodiments, the heterocyclyl or heteroaryl group is C-attached. By way of example and not limitation, carbon bonded heterocyclyls include bonding arrangements at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. (2-pyridyl, 3-pyridyl, 4-pyridyl, 5-pyridyl, 6-pyridyl).

In certain embodiments, the heterocyclyl or heteroaryl group is N-attached. By way of example and not limitation, the nitrogen bonded heterocyclyl or heteroaryl group include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

“Treat” and “treatment” includes both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, remission (whether partial or total), whether detectable or undetectable, sustaining remission and suppressing reoccurrence. “Treatment” can also mean prolonging survival as compared to expected survival if not receiving treatment. Those in need of treatment include those already with the condition or disorder as well as those prone to have the condition or disorder, (for example, through a genetic mutation) or those in which the condition or disorder is to be prevented.

The phrase “therapeutically effective amount” means an amount of a compound of the present invention that (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR). In the case of immunological disorders, the therapeutic effective amount is an amount sufficient to decrease or alleviate an allergic disorder, the symptoms of an autoimmune and/or inflammatory disease, or the symptoms of an acute inflammatory reaction (e.g. asthma). In some embodiments, a therapeutically effective amount is an amount of a chemical entity described herein sufficient to significantly decrease the activity or number of B-cells.

“Inflammatory disorder” as used herein can refer to any disease, disorder, or syndrome in which an excessive or unregulated inflammatory response leads to excessive inflammatory symptoms, host tissue damage, or loss of tissue function. “Inflammatory disorder” also refers to a pathological state mediated by influx of leukocytes and/or neutrophil chemotaxis.

“Inflammation” as used herein refers to a localized, protective response elicited by injury or destruction of tissues, which serves to destroy, dilute, or wall off (sequester) both the injurious agent and the injured tissue. Inflammation is notably associated with influx of leukocytes and/or neutrophil chemotaxis. Inflammation can result from infection with pathogenic organisms and viruses and from noninfectious means such as trauma or reperfusion following myocardial infarction or stroke, immune response to foreign antigen, and autoimmune responses. Accordingly, inflammatory disorders amenable to treatment with Formulas Ia-Ib compounds encompass disorders associated with reactions of the specific defense system as well as with reactions of the nonspecific defense system.

“Specific defense system” refers to the component of the immune system that reacts to the presence of specific antigens. Examples of inflammation resulting from a response of the specific defense system include the classical response to foreign antigens, autoimmune diseases, and delayed type hypersensitivity response mediated by T-cells. Chronic inflammatory diseases, the rejection of solid transplanted tissue and organs, e.g., kidney and bone marrow transplants, and graft versus host disease (GVHD), are further examples of inflammatory reactions of the specific defense system.

The term “nonspecific defense system” as used herein refers to inflammatory disorders that are mediated by leukocytes that are incapable of immunological memory (e.g., granulocytes, and macrophages). Examples of inflammation that result, at least in part, from a reaction of the nonspecific defense system include inflammation associated with conditions such as adult (acute) respiratory distress syndrome (ARDS) or multiple organ injury syndromes; reperfusion injury; acute glomerulonephritis; reactive arthritis; dermatoses with acute inflammatory components; acute purulent meningitis or other central nervous system inflammatory disorders such as stroke; thermal injury; inflammatory bowel disease; granulocyte transfusion associated syndromes; and cytokine-induced toxicity.

“Autoimmune disease” as used herein refers to any group of disorders in which tissue injury is associated with humoral or cell-mediated responses to the body's own constituents.

“Allergic disease” as used herein refers to any symptoms, tissue damage, or loss of tissue function resulting from allergy. “Arthritic disease” as used herein refers to any disease that is characterized by inflammatory lesions of the joints attributable to a variety of etiologies. “Dermatitis” as used herein refers to any of a large family of diseases of the skin that are characterized by inflammation of the skin attributable to a variety of etiologies. “Transplant rejection” as used herein refers to any immune reaction directed against grafted tissue, such as organs or cells (e.g., bone marrow), characterized by a loss of function of the grafted and surrounding tissues, pain, swelling, leukocytosis, and thrombocytopenia. The therapeutic methods of the present invention include methods for the treatment of disorders associated with inflammatory cell activation.

“Inflammatory cell activation” refers to the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes (i.e., polymorphonuclear leukocytes such as neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory disorder.

The term “NSAID” is an acronym for “non-steroidal anti-inflammatory drug” and is a therapeutic agent with analgesic, antipyretic (lowering an elevated body temperature and relieving pain without impairing consciousness) and, in higher doses, with anti-inflammatory effects (reducing inflammation). The term “non-steroidal” is used to distinguish these drugs from steroids, which (among a broad range of other effects) have a similar eicosanoid-depressing, anti-inflammatory action. As analgesics, NSAIDs are unusual in that they are non-narcotic. NSAIDs include aspirin, ibuprofen, and naproxen. NSAIDs are usually indicated for the treatment of acute or chronic conditions where pain and inflammation are present. NSAIDs are generally indicated for the symptomatic relief of the following conditions: rheumatoid arthritis, osteoarthritis, inflammatory arthropathies (e.g. ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic. Most NSAIDs act as non-selective inhibitors of the enzyme cyclooxygenase, inhibiting both the cyclooxygenase-1 (COX-1) and cyclooxygenase-2 (COX-2) isoenzymes. Cyclooxygenase catalyzes the formation of prostaglandins and thromboxane from arachidonic acid (itself derived from the cellular phospholipid bilayer by phospholipase A₂). Prostaglandins act (among other things) as messenger molecules in the process of inflammation. COX-2 inhibitors include celecoxib, etoricoxib, lumiracoxib, parecoxib, rofecoxib, rofecoxib, and valdecoxib.

The terms “cancer” and “cancerous” refer to or describe the physiological condition in patients that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is an agent useful in the treatment of a given disorder, for example, cancer or inflammatory disorders. Examples of chemotherapeutic agents include NSAIDs; hormones such as glucocorticoids; corticosteroids such as hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydro cortisone-17-butyrate, hydrocortisone-17-valerate, aclometas one dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomide, methotrexate (MTX), minocycline, sulfasalazine, cyclophosphamide, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), monoclonal antibodies against B cells such as rituximab (RITUXAN®), T cell costimulation blockers such as abatacept (Orencia), Interleukin 6 (IL-6) blockers such as tocilizumab; hormone antagonists, such as tamoxifen, finasteride or LHRH antagonists; radioactive isotopes (e.g., At²¹¹, I¹³¹, I¹²⁵ Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18- OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma 1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as fenretinide, retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Additional chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide.

Additional chemotherapeutic agents include therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), lebrikizumab, tocilizumab (ACTEMRA®, Roche), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁ λ antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)prop oxy]-6-quinazolinyl]-, dihydrochloride, Pfizer Inc.); ZD1839, gefitinib (IRESSAJ) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N-8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6 [5 [[[2-methylsulfonyl)ethyl]amino]methyl]-2-furanyl]-4-quinazolinamine)

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVECJ, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVECJ); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

“Optionally substituted” unless otherwise specified means that a group may be unsubstituted or substituted by one or more (e.g. 0, 1, 2, 3 or 4) of the substituents listed for that group in which said substituents may be the same or different. In an embodiment an optionally substituted group has 1 substituent. In another embodiment an optionally substituted group has 2 substituents. In another embodiment an optionally substituted group has 3 substituents.

The term “prodrug” as used in this application refers to a precursor or derivative form of a pharmaceutically active substance that is less efficacious to the patient or cytotoxic to tumor cells compared to the parent drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, (β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

The term “package insert” is used to refer to instructions customarily included in commercial packages of therapeutic products, that contain information about the indications, usage, dosage, administration, contraindications and/or warnings concerning the use of such therapeutic products.

The term “stereoisomers” refers to compounds which have identical chemical constitution, but differ with regard to the arrangement of the atoms or groups in space. Stereoisomers include diastereomers, enantiomers, conformers and the like.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The phrase “pharmaceutically acceptable salt,” as used herein, refers to pharmaceutically acceptable organic or inorganic salts of a compound of Formulas Ia-Ib. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

A “solvate” refers to an association or complex of one or more solvent molecules and a compound of Formulas Ia-Ib. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

The term “leaving group” or “Lv” refers to a group or portion of a first reactant in a chemical reaction that is displaced from the first reactant in the chemical reaction. Examples of leaving groups include, but are not limited to, halogen atoms, alkoxy and sulfonyloxy groups. Example sulfonyloxy groups include, but are not limited to, alkylsulfonyloxy groups (for example methyl sulfonyloxy (mesylate group) and trifluoromethylsulfonyloxy (triflate group)) and arylsulfonyloxy groups (for example p-toluenesulfonyloxy (tosylate group) and p-nitrosulfonyloxy (nosylate group)).

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, phthalimido, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include acetyl, trialkylsilyl, dialkylphenylsilyl, benzoyl, benzyl, benzyloxymethyl, methyl, methoxymethyl, triarylmethyl, and tetrahydropyranyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene and P. Wuts, Protective Groups in Organic Synthesis, Third Ed., John Wiley & Sons, New York, 1999; and P. Kocienski, Protecting Groups, Third Ed., Verlag, 2003.

The term “patient” includes human patients and animal patients. The term “animal” includes companion animals (e.g., dogs, cats and horses), food-source animals, zoo animals, marine animals, birds and other similar animal species.

The phrase “pharmaceutically acceptable” indicates that the substance or composition must be compatible chemically and/or toxicologically, with the other ingredients comprising a formulation, and/or the mammal being treated therewith.

The terms “compound of this invention,” and “compounds of the present invention”, unless otherwise indicated, include compounds of Formulas Ia-Ib, stereoisomers, tautomers, solvates, prodrugs and salts (e.g., pharmaceutically acceptable salts) thereof. Unless otherwise stated, structures depicted herein are also meant to include compounds that differ only in the presence of one or more isotopically enriched atoms. For example, compounds of Formula Ia and Ib, wherein one or more hydrogen atoms are replaced deuterium or tritium, or one or more carbon atoms are replaced by a ¹³C- or ¹⁴C-enriched carbon are within the scope of this invention.

### TYK2 Inhibitor Compounds

In one embodiment, a compound of Formulas Ia-Ib, stereoisomers or pharmaceutically acceptable salts thereof, and pharmaceutical formulations thereof, are provided that are useful in the treatment of diseases, conditions and/or disorders responsive to the inhibition of TYK2.

Another embodiment includes compounds of Formulas Ia-Ib:

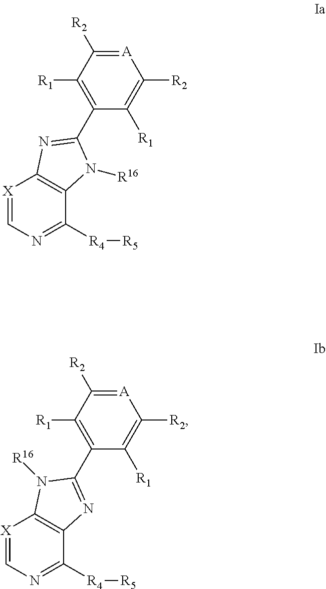

stereoisomers or pharmaceutically acceptable salts thereof, wherein:

### A is CR³ or N;

### X is CR¹⁵ or N;

R¹ is independently hydrogen, halogen, C₁-C₃ alkyl, C₃-C₄ cycloalkyl, —CF₃, —OR⁶, —SR⁶, —OCF₃, —CN, —NO₂, —NR⁶SO₂R⁷, —NR⁶C(O)R⁷ or —NR⁶R⁷, wherein both R¹ cannot be hydrogen at the same time, and wherein said alkyl and cycloalkyl are optionally substituted by halogen, OR⁶, —NR⁶R⁷ or phenyl;
R² and R³ are independently hydrogen, C₁-C₆ alkyl, C₂-C₆ alkenyl, C₂-C₆ alkynyl, halogen, —(C₀-C₃ alkyl)CN, —(C₀-C₃ alkyl)OR⁸, —(C₀-C₃ alkyl)SR⁸, —(C₀-C₃ alkyl)NR⁸R⁹, —(C₀-C₃ alkyl)CF₃, —O(C₀-C₃ alkyl)CF₃, —(C₀-C₃ alkyl)NO₂, —(C₀-C₃ alkyl)C(O)R⁸, —(C₀-C₃ alkyl)C(O)OR⁸, —(C₀-C₃ alkyl)C(O)NR⁸R⁹, —(C₀-C₃ alkyl)NR⁸C(O)R⁹, —(C₀-C₃ alkyl)S(O)₁₋₂R⁸, —(C₀-C₃ alkyl)NR⁸S(O)₁₋₂R⁹, —(C₀-C₃ alkyl)S(O)₁₋₂NR⁸R⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R² and R³ are independently optionally substituted by R¹⁰;
R⁴ is —NH₂, —NH—, —NR⁶R⁷, —NR⁶C(O)—, —NR⁶C(O)O—, —NR⁶C(O)NR⁷—, —NR⁶S(O)₁₋₂— or —NR⁶S(O)₁₋₂NR⁷—;
R⁵ is absent, hydrogen, C₁-C₆ alkyl, C₂-C₆ alkenyl, C₂-C₆ alkynyl, C₃-C₈ cycloalkyl, phenyl or 3-10-membered heterocyclyl, wherein R⁵ is optionally substituted by R¹⁰;
R⁶ and R⁷ are each independently hydrogen, C₁-C₃ alkyl or C₃-C₄ cycloalkyl, wherein said alkyl and cycloalkyl are independently optionally substituted by halogen, oxo, —OR¹¹ or —NR¹¹R¹²; or
R⁶ and R⁷ are independently taken together with the atom to which they are attached to form a 3-6 membered heterocyclyl optionally substituted by halogen, oxo, —NR¹¹R¹² or C₁-C₃ alkyl;
R⁸ and R⁹ are each independently hydrogen, C₁-C₃ alkyl, C₃-C₆ cycloalkyl, phenyl, 3-6-membered heterocyclyl or 5-6-membered heteroaryl, wherein said alkyl, cycloalkyl, phenyl, heterocyclyl or heteroaryl are independently optionally substituted by R¹⁰; or
R⁸ and R⁹ are independently taken together with the atom to which they are attached to form a 3-6 membered heterocyclyl optionally substituted by halogen, oxo, —NR¹¹R¹² or C₁-C₃ alkyl;
R¹⁰ is independently hydrogen, oxo, C₁-C₆ alkyl, C₂-C₆ alkenyl, C₂-C₆ alkynyl, halogen, —(C₀-C₃ alkyl)CN, —(C₀-C₃ alkyl)OR¹¹, —(C₀-C₃ alkyl)SR¹¹, —(C₀-C₃ alkyl)NR¹¹R¹², —(C₀-C₃ alkyl)CF₃, —(C₀-C₃ alkyl)NO₂, —C═NH(OR¹¹), —(C₀-C₃ alkyl)C(O)R¹¹, —(C₀-C₃ alkyl)C(O)OR¹¹, —(C₀-C₃ alkyl)OC(O)R¹¹, —(C₀-C₃ alkyl)OC(O)OR¹¹, —(C₀-C₃ alkyl)C(O)NR¹¹R¹², —(C₀-C₃ alkyl)NR¹¹C(O)R¹², —(C₀-C₃ alkyl)NR¹¹C(O)OR¹², —(C₀-C₃ alkyl)OC(O)NR¹¹R¹², —(C₀-C₃ alkyl)S(O)₁₋₂R¹¹, —(C₀-C₃ alkyl)NR¹¹S(O)₁₋₂R¹², —(C₀-C₃ alkyl)S(O)₁₋₂NR¹¹R¹², —(C₀-C₃ alkyl)(C₃-C₈ cycloalkyl), —(C₀-C₃ alkyl)(3-10-membered heterocyclyl), —(C₀-C₃ alkyl)C(O)(3-10-membered heterocyclyl) or —(C₀-C₃ alkyl)phenyl, wherein R¹⁰ is independently optionally substituted by halogen, oxo, —CF₃, —(C₀-C₃ alkyl)OR¹³, —(C₀-C₃ alkyl)NR¹³R¹⁴, —(C₀-C₃ alkyl)C(O)R¹³, —(C₀-C₃ alkyl)S(O)₁₋₂R¹³, 3-10-membered heterocyclyl or C₁-C₃ alkyl optionally substituted by oxo, halogen, —NR¹³R¹⁴ or —OR¹³.
R¹¹ and R¹² are independently hydrogen, C₁-C₆ alkyl or —(C₀-C₃ alkyl)phenyl, wherein said alkyl and phenyl are independently optionally substituted by halogen, oxo, —OR¹³, —NR¹³R¹⁴, C₁-C₃ alkyl, —(C₀-C₃ alkyl)(C₃-C₆ cycloalkyl), —(C₀-C₃ alkyl)phenyl, —(C₀-C₃ alkyl)(3-6-membered heterocyclyl) or —(C₀-C₃alkyl)(5-6-membered heteroaryl); or
R¹¹ and R¹² are taken together with the atom to which they attached to form a 3-6 membered heterocyclyl optionally substituted by halogen, oxo, —OR¹³, —NR¹³R¹⁴ or C₁-C₃ alkyl;
R¹³ and R¹⁴ are independently hydrogen, C₁-C₆ alkyl, OH or O(C₁-C₆ alkyl), wherein said alky is optionally substituted by halogen, —NH₂, —N(CH₃)₂ or oxo; or
R¹³ and R¹⁴ are taken together with the atom to which they attached to form a 3-6 membered heterocyclyl optionally substituted by halogen, oxo, —NH₂, —N(CH₃)₂ or C₁-C₃ alkyl;
R¹⁵ is hydrogen, halogen, C₁-C₆ alkyl, C₂-C₆ alkenyl, C₂-C₆ alkynyl, —(C₀-C₃ alkyl)CN, —(C₀-C₃ alkyl)OR¹⁸, —(C₀-C₃ alkyl)SR¹⁸, —(C₀-C₃ alkyl)NR¹⁸R¹⁹, —(C₀-C₃ alkyl)CF₃, —O(C₀-C₃ alkyl)CF₃, —(C₀-C₃ alkyl)NO₂, —(C₀-C₃ alkyl)C(O)R¹⁸, —(C₀-C₃ alkyl)C(O)OR¹⁸, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸, —(C₀-C₃ alkyl)NR¹⁸S(O)₁₋₂R¹⁹, —(C₀-C₃ alkyl)S(O)₁₋₂NR¹⁸R¹⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R¹⁵ is optionally substituted by R¹⁰;
R¹⁶ is hydrogen, C₁-C₆ alkyl, C₂-C₆ alkenyl, C₂-C₆ alkynyl, —(C₀-C₃ alkyl)CN, —(C₁-C₃ alkyl)OR¹⁸, —(C₁-C₃ alkyl)SR¹⁸, —(C₁-C₃ alkyl)NR¹⁸R¹⁹, —(C₁-C₃ alkyl)CF₃, —O(C₁-C₃ alkyl)CF₃, —(C₂-C₃ alkyl)NO₂, —(C₀-C₃ alkyl)C(O)R¹⁸, —(C₀-C₃ alkyl)C(O)OR¹⁸, —(C₀-C₃ alkyl)C(═NR¹⁸)NR¹⁸R¹⁹, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸—(C₀-C₃ alkyl)NR¹⁸S(O)₁₋₂R¹⁹, —(C₀-C₃ alkyl)S(O)₁₋₂NR¹⁸R¹⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R¹⁶ is optionally substituted by R¹⁰;
R¹⁸ and R¹⁹ are independently hydrogen or C₁-C₆ alkyl optionally substituted by halogen, oxo, CN, —OR²⁰, —SR²⁰ or —NR²⁰R²¹; or

R¹⁸ and R¹⁹ are taken together with the atom to which they attached to form a 3-6 membered heterocyclyl optionally substituted by halogen, oxo, C₁-C₃ alkyl, CN, —OR²⁰, —SR²⁰ or —NR²⁰R²¹; and

R²⁰ and R²¹ are independently hydrogen or C₁-C₆ alkyl optionally substituted by oxo, halogen, —OH or —NH₂.

Certain embodiments include compounds of Formulas Ia-Ib, stereoisomers or pharmaceutically acceptable salts thereof, wherein:

### A is CR³ or N;

### X is CR¹⁵ or N;

R¹ is independently hydrogen, halogen, C₁-C₃ alkyl, C₃-C₄ cycloalkyl, —CF₃, —OR⁶, —SR⁶, —OCF₃, —CN, —NO₂, —NR⁶SO₂R⁷, —NR⁶C(O)R⁷ or —NR⁶R⁷, wherein both R¹ cannot be hydrogen at the same time, and wherein said alkyl and cycloalkyl are optionally substituted by halogen, OR⁶, —NR⁶R⁷ or phenyl;
R² and R³ are independently hydrogen, C₁-C₆ alkyl, C₂-C₆ alkenyl, C₂-C₆ alkynyl, halogen, —(C₀-C₃ alkyl)CN, —(C₀-C₃ alkyl)OR⁸, —(C₀-C₃ alkyl)SR⁸, —(C₀-C₃ alkyl)NR⁸R⁹, —(C₀-C₃ alkyl)CF₃, —O(C₀-C₃ alkyl)CF₃, —(C₀-C₃ alkyl)NO₂, —(C₀-C₃ alkyl)C(O)R⁸, —(C₀-C₃ alkyl)C(O)OR⁸, —(C₀-C₃ alkyl)C(O)NR⁸R⁹, —(C₀-C₃ alkyl)NR⁸C(O)R⁹, —(C₀-C₃ alkyl)S(O)₁₋₂R⁸, —(C₀-C₃ alkyl)NR⁸S(O)₁₋₂R⁹, —(C₀-C₃ alkyl)S(O)₁₋₂NR⁸R⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R² and R³ are independently optionally substituted by R¹⁰;
R⁴ is —NH₂, —NH—, —NR⁶R⁷, —NR⁶C(O)—, —NR⁶C(O)O—, —NR⁶C(O)NR⁷—, —NR⁶S(O)₁₋₂— or —NR⁶S(O)₁₋₂NR⁷—;
R⁵ is absent, hydrogen, C₁-C₆ alkyl, C₂-C₆ alkenyl, C₂-C₆ alkynyl, C₃-C₆ cycloalkyl, phenyl, 3-7-membered heterocyclyl or 5-10-membered heteroaryl, wherein R⁵ is optionally substituted by R¹⁰;
R⁶ and R⁷ are each independently hydrogen, C₁-C₃ alkyl or C₃-C₄ cycloalkyl, wherein said alkyl and cycloalkyl are independently optionally substituted by halogen, oxo, —OR¹¹ or —NR¹¹R¹²; or
R⁶ and R⁷ are independently taken together with the atom to which they are attached to form a 3-6 membered heterocyclyl optionally substituted by halogen, oxo, —NR¹¹R¹² or C₁-C₃ alkyl;
R⁸ and R⁹ are each independently hydrogen, C₁-C₃ alkyl, C₃-C₆ cycloalkyl, phenyl, 3-6-membered heterocyclyl or 5-6-membered heteroaryl, wherein said alkyl, cycloalkyl, phenyl, heterocyclyl or heteroaryl are independently optionally substituted by R¹⁰; or
R⁸ and R⁹ are independently taken together with the atom to which they are attached to form a R¹⁰ is independently hydrogen, oxo, C₁-C₆ alkyl, C₂-C₆ alkenyl, C₂-C₆ alkynyl, halogen, —(C₀-C₃ alkyl)CN, —(C₀-C₃ alkyl)OR¹¹, —(C₀-C₃ alkyl)SR¹¹, —(C₀-C₃ alkyl)NR¹¹R¹², —(C₀-C₃ alkyl)CF₃, —(C₀-C₃ alkyl)NO₂, —C═NH(OR¹¹), —(C₀-C₃ alkyl)C(O)R¹¹, —(C₀-C₃ alkyl)C(O)OR¹¹, —(C₀-C₃ alkyl)C(O)NR¹¹R¹², —(C₀-C₃ alkyl)NR¹¹C(O)R¹², —(C₀-C₃ alkyl)S(O)₁₋₂R¹¹, —(C₀-C₃ alkyl)NR¹¹S(O)₁₋₂R¹², —(C₀-C₃ alkyl)S(O)₁₋₂NR¹¹R¹², —(C₀-C₃ alkyl)(C₃-C₆ cycloalkyl), —(C₀-C₃ alkyl)(3-6-membered heterocyclyl), —(C₀-C₃ alkyl)C(O)(3-6-membered heterocyclyl), —(C₀-C₃ alkyl)(5-6-membered heteroaryl) or —(C₀-C₃ alkyl)phenyl, wherein R¹⁰ is independently optionally substituted by halogen, C₁-C₃ alkyl, oxo, —CF₃, —(C₀-C₃ alkyl)OR¹³, —(C₀-C₃ alkyl)NR¹³R¹⁴, —(C₀-C₃ alkyl)C(O)R¹³ or —(C₀-C₃ alkyl)S(O)₁₋₂R¹³;
R¹¹ and R¹² are independently hydrogen, C₁-C₆ alkyl or —(C₀-C₃ alkyl)phenyl, wherein said alkyl and phenyl are independently optionally substituted by halogen, oxo, —OR¹³, —NR¹³R¹⁴, C₁-C₃ alkyl, —(C₀-C₃ alkyl)(C₃-C₆ cycloalkyl), —(C₀-C₃ alkyl)phenyl, —(C₀-C₃ alkyl)(3-6-membered heterocyclyl) or —(C₀-C₃ alkyl)(5-6-membered heteroaryl); or
R¹¹ and R¹² are taken together with the atom to which they attached to form a 3-6 membered heterocyclyl optionally substituted by halogen, oxo, —OR¹³, —NR¹³R¹⁴ or C₁-C₃ alkyl;
R¹³ and R¹⁴ are independently hydrogen, C₁-C₆ alkyl, OH or O(C₁-C₆ alkyl), wherein said alky is optionally substituted by halogen, —NH₂, —N(CH₃)₂ or oxo; or
R¹³ and R¹⁴ are taken together with the atom to which they attached to form a 3-6 membered heterocyclyl optionally substituted by halogen, oxo, —NH₂, —N(CH₃)₂ or C₁-C₃ alkyl;
R¹⁵ is hydrogen, halogen, C₁-C₆ alkyl, C₂-C₆ alkenyl, C₂-C₆ alkynyl, —(C₀-C₃ alkyl)CN, —(C₀-C₃ alkyl)OR¹⁸, —(C₀-C₃ alkyl)SR¹⁸, —(C₀-C₃ alkyl)NR¹⁸R¹⁹, —(C₀-C₃ alkyl)CF₃, —O(C₀-C₃ alkyl)CF₃, —(C₀-C₃ alkyl)NO₂, —(C₀-C₃ alkyl)C(O)R¹⁸, —(C₀-C₃ alkyl)C(O)OR¹⁸, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸, —(C₀-C₃ alkyl)NR¹⁸S(O)₁₋₂R¹⁹, —(C₀-C₃ alkyl)S(O)₁₋₂NR¹⁸R¹⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R¹⁵ is optionally substituted by R¹⁰;
R¹⁶ is hydrogen, C₁-C₆ alkyl, C₂-C₆ alkenyl, C₂-C₆ alkynyl, —(C₀-C₃ alkyl)CN, —(C₁-C₃ alkyl)OR¹⁸, —(C₁-C₃ alkyl)SR¹⁸, —(C₁-C₃ alkyl)NR¹⁸R¹⁹, —(C₁-C₃ alkyl)CF₃, —O(C₁-C₃ alkyl)CF₃, —(C₂-C₃ alkyl)NO₂, —(C₀-C₃ alkyl)C(O)R¹⁸, —(C₀-C₃ alkyl)C(O)OR¹⁸, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸, —(C₀-C₃ alkyl)NR¹⁸S(O)₁₋₂R¹⁹, —(C₀-C₃ alkyl)S(O)₁₋₂NR¹⁸R¹⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R¹⁶ is optionally substituted by R¹⁰;
R¹⁸ and R¹⁹ are independently hydrogen or C₁-C₆ alkyl optionally substituted by halogen, oxo, CN or —NR²⁰R²¹; or
R¹⁸ and R¹⁹ are taken together with the atom to which they attached to form a 3-6 membered heterocyclyl optionally substituted by halogen, oxo, C₁-C₃ alkyl, CN or —NR²⁰R²¹; and
R²⁰ and R²¹ are independently hydrogen or C₁-C₆ alkyl.

In certain embodiments, Formulas Ia-Ib include compounds other than:
2-(6-amino-7H-purin-8-yl)phenol;
2-(2-fluoro-3-methoxyphenyl)-N-(pyridin-4-ylmethyl)-3H-imidazo[4,5-c]pyridin-4-amine;
8-(2,4-dichlorophenyl)-7H-purin-6-amine;
2-(2,5-dimethoxyphenyl)-N-methyl-3H-imidazo[4,5-c]pyridin-4-amine;
8-(2,3,5,6-tetrafluoro-4-(1H-imidazol-1-yl)phenyl)-7H-purin-6-amine; and
8-o-tolyl-7H-purin-6-amine

In certain embodiments, A is CR³.

In certain embodiments, A is CR³ and X is CR¹⁵.

In certain embodiments, A is CR³ and X is N.

In certain embodiments, A is N.

In certain embodiments, A is N and X is CR¹⁵.

In certain embodiments, A is N and X is N.

In certain embodiments, R¹ is independently hydrogen, halogen, C₁-C₃ alkyl, —CF₃, —OR⁶, —SR⁶, —OCF₃, —NO₂ or —NR⁶R⁷, wherein both R¹ cannot be hydrogen at the same time, and wherein said alkyl is optionally substituted by halogen, OR⁶ or —NR⁶R⁷.

In certain embodiments, R¹ is independently hydrogen, F, Cl, Br, —OH, —CF₃, —OCF₃, —CH₃ or —OCH₃, wherein both R¹ cannot be hydrogen at the same time.

In certain embodiments, R¹ is independently halogen. In one embodiment, R¹ is independently F or Cl. In another embodiment, R¹ is Cl.

In certain embodiments, R¹ is independently halogen, R⁴ is —NHR⁵, —NR⁶C(O)R⁵, —NR⁶C(O)OR⁵ or —NR⁶C(O)NR⁷R⁵, wherein R⁵ is other than hydrogen.

In certain embodiments, one R¹ is halogen and R⁴ is —NHR⁵ or —NR⁶C(O)R⁵, wherein R⁵ is other than hydrogen.

In certain embodiments, one R¹ is halogen and the other R¹ is hydrogen, halogen, C₁-C₃ alkyl, C₃-C₄ cycloalkyl, —CF₃, —OH, —O(C₁-C₃ alkyl), —SH, —S(C₁-C₃ alkyl), —OCF₃, —CN, —NO₂, —NHSO₂CH₃, —NHC(O)R⁷ or —NR⁶R⁷, wherein said alkyl and cycloalkyl are optionally substituted by halogen, OR⁸, —NR⁸R⁹ or phenyl.

In certain embodiments, one R¹ is halogen and the other R¹ is halogen, C₁-C₃ alkyl, C₃-C₄ cycloalkyl, —CF₃, —OH, —O(C₁-C₃ alkyl), —SH, —S(C₁-C₃ alkyl), —OCF₃, —CN, —NO₂, —NHSO₂CH₃, —NHC(O)R⁷ or —NR⁶R⁷, wherein said alkyl and cycloalkyl are optionally substituted by halogen, OR¹, —NR⁸R⁹ or phenyl.

In certain embodiments, R¹ is independently halogen, C₁-C₃ alkyl, C₃-C₄ cycloalkyl, —CF₃, —OH, —O(C₁-C₃ alkyl), —SH, —S(C₁-C₃ alkyl), —OCF₃, —CN, —NO₂, —NHSO₂CH₃, —NHC(O)R⁷ or —NR⁶R⁷, wherein said alkyl and cycloalkyl are optionally substituted by halogen, OR^{B}, —NR⁸R⁹ or phenyl.

In certain embodiments, R¹ is independently hydrogen, F, Cl, —CF₃, —CH₃, or —OCF₃, wherein both R¹ cannot be hydrogen at the same time.

In certain embodiments, R² is independently hydrogen, halogen or C₁-C₆ alkyl optionally substituted by R¹⁰. In certain embodiments, R² is independently F, Cl, Br, —CH₂OH, —CH₂NH₂ or —CH₂morpholinyl.

In certain embodiments, R² is independently hydrogen or halogen.

In certain embodiments, R² is hydrogen.

In certain embodiments, R³ is hydrogen, C₁-C₆ alkyl, C₂-C₆ alkenyl, C₂-C₆ alkynyl, halogen, —(C₀-C₃ alkyl)CN, —(C₀-C₃ alkyl)OR⁸, —(C₀-C₃ alkyl)SR⁸, —(C₀-C₃ alkyl)NR⁸R⁹, —(C₀-C₃ alkyl)C(O)NR⁸R⁹, —(C₀-C₃ alkyl)S(O)₁₋₂R⁸ or —(C₀-C₃ alkyl)(3-6-membered heterocyclyl), wherein R³ is independently optionally substituted by R¹⁰.

In certain embodiments, R³ is hydrogen, Cl, F, Br, —CH₃, acetylenyl, —NH₂, —CN, —S(O)₂CH₃, —C(O)NH₂, —CH₂NH₂, —CH₂OH, —CH₂NH(CH₃), —CH₂N(CH₃)₂ or —CH₂morpholinyl,

In certain embodiments, R³ is hydrogen, halogen, —CN or —S(O)₁₋₂(C₁-C₃ alkyl). In one embodiment, R³ is hydrogen, —CN or —S(O)₂CH₃.

In certain embodiments, A is CR³, R² is hydrogen and R³ is hydrogen, halogen, —CN or —S(O)₁₋₂(C₁-C₃ alkyl).

In certain embodiments, the portion of Formula I having the structure:

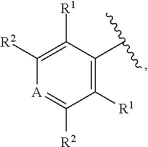

is selected from:

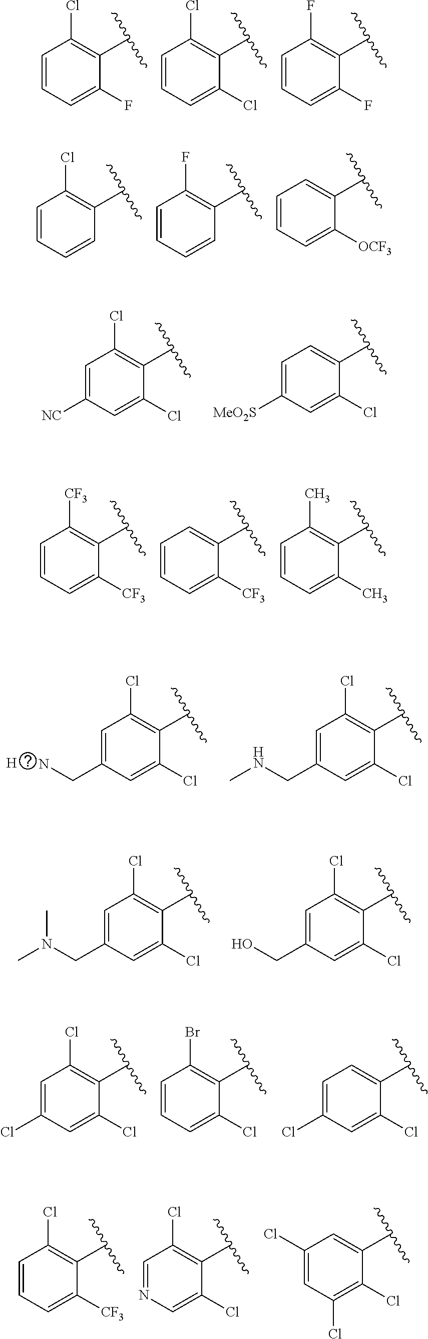

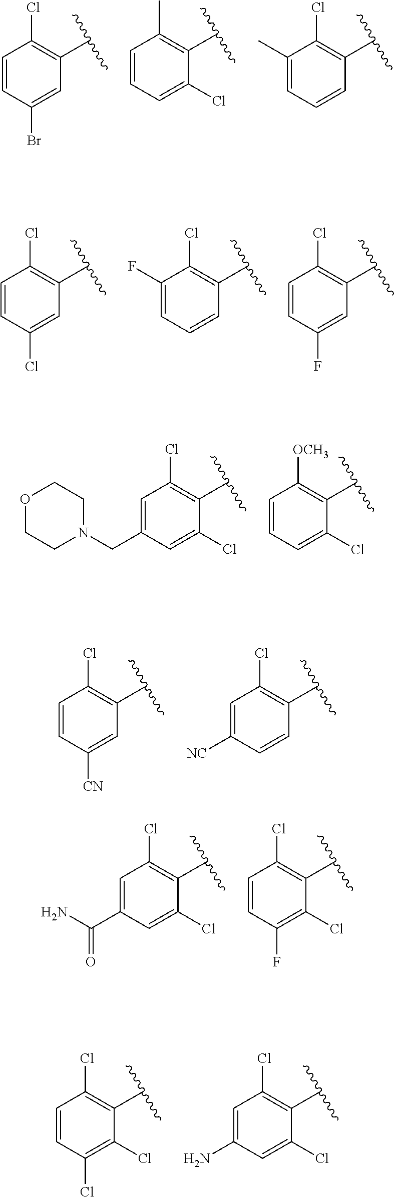

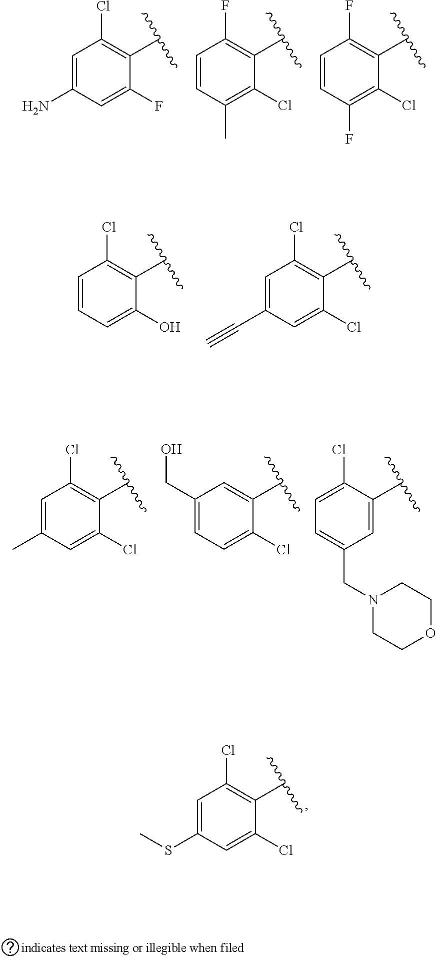

wherein the wavy lines represent the point of attachment in Formula I.

In certain embodiments, R⁴ is —NH—, —NR⁶C(O)—, —NR⁶C(O)O— or —NR⁶C(O)NR⁷—.

In certain embodiments, R⁴ is —NHR⁵, —NR⁶C(O)R⁵, —NR⁶C(O)OR⁵ or —NR⁶C(O)NR⁷R⁵.

In certain embodiments, R⁴ is —NHR⁵, —NR⁶C(O)R⁵, —NR⁶C(O)OR⁵ or —NR⁶C(O)NR⁷R⁵, wherein R⁵ is other than hydrogen.

In certain embodiments, X is CR¹⁵ and R⁴ is —NHR⁵, —NR⁶C(O)R⁵, —NR⁶C(O)OR⁵ or —NR⁶C(O)NR⁷R⁵.

In certain embodiments, R⁴ is —NR⁶C(O)R⁵, —NR⁶C(O)OR⁵ or —NR⁶C(O)NR⁷R⁵.

In certain embodiments, R⁴ is —NH₂ and R⁵ absent.

In certain embodiments, R⁴ is —NHR⁵ or —NR⁶C(O)R⁵, wherein R⁵ is other than hydrogen.

In certain embodiments, R⁵ is absent.

In certain embodiments, R⁵ is hydrogen.

In certain embodiments, R⁴ is —NR⁶R⁷, —NR⁶C(O)NR⁷— or —NR⁶S(O)₁₋₂NR⁷—; R⁵ is absent; and R⁶ and R⁷ are independently hydrogen, C₁-C₃ alkyl or C₃-C₄ cycloalkyl, wherein said alkyl and cycloalkyl are independently optionally substituted by halogen, oxo, —OR¹¹ or —NR¹¹R¹².

In certain embodiments, R⁵ is C₁-C₆ alkyl optionally substituted by R¹⁰. In certain embodiments, R⁵ is methyl, ethyl, isopropyl, tert-butyl, —CH₂OH, —CH₂N(CH₃)₂ or —CH₂NHC(O)OC(CH₃)₃.

In certain embodiments, R⁵ is C₁-C₆ alkyl optionally substituted by halogen. In certain embodiments, R⁵ is methyl, ethyl, isopropyl or tert-butyl.

In certain embodiments, R⁵ is C₃-C₆ cycloalkyl optionally substituted by R¹⁰.

In certain embodiments, R⁵ is cyclopropyl, cyclobutyl,

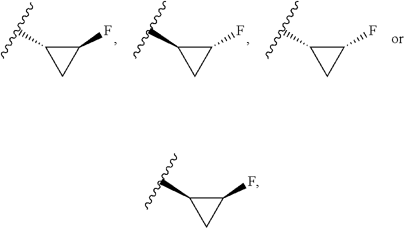

wherein the wavy line represents the point of attachment in Formulas Ia-Ib.

In certain embodiments, R⁵ is C₃-C₆ cycloalkyl optionally substituted by halogen. In certain embodiments, R⁵ is cyclopropyl optionally substituted by halogen. In certain embodiments, R⁵ is selected from:

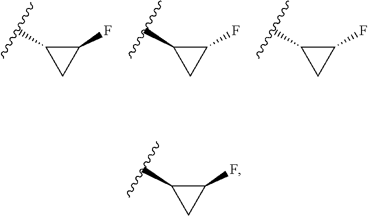

wherein the wavy line represents the point of attachment in Formulas Ia-Ib.

In certain embodiments, R⁵ is phenyl optionally substituted by R¹⁰. In certain embodiments, R⁵ is phenyl. In certain embodiments, R⁵ is phenyl optionally substituted by —O(CH₂)₂pyrrolidinyl.

In certain embodiments, R⁵ is 3-10-membered heterocyclyl optionally substituted by R¹⁰. In certain embodiments, R⁵ is pyridinyl, pyrimidinyl, pyrazinyl, pyridazinyl, pyrazolyl, triazinyl, pyrrolopyrimidinyl, pyrazolopyrimidinyl, pyrrolidinyl, pyrimidinonyl, oxazolyl, isoxazolyl, isothiazolyl or thiazolyl optionally substituted by R¹⁰.

In certain embodiments, R⁵ is 3-7-membered heterocyclyl optionally substituted by R¹⁰.

In certain embodiments, R⁵ is 5-10-membered heteroaryl optionally substituted by R¹⁰. In certain embodiments, R⁵ is pyridinyl, pyrimidinyl, pyrazinyl, pyridazinyl, pyrazolyl, triazinyl, pyrrolopyrimidinyl, pyrazolopyrimidinyl, oxazolyl, isoxazolyl, isothiazolyl or thiazolyl optionally substituted by R¹⁰. In certain embodiments, R⁵ is pyridinyl, pyrimidinyl, pyrazolyl, thiazolyl, pyrazinyl, pyridazinyl, oxazolyl or isoxazolyl, wherein said R⁵ is optionally substituted by R¹⁰.

In certain embodiments, R⁵ is pyridinyl, pyrimidinyl, pyrazinyl, pyridazinyl, pyrazolyl, triazinyl, pyrrolopyrimidinyl, pyrazolopyrimidinyl, oxazolyl, isoxazolyl, isothiazolyl or thiazolyl optionally substituted by C₁-C₆ alkyl, C₃-C₆ cycloalkyl, halogen, —CN, —OR¹¹, —SR¹¹, —CF₃, —NR¹¹R¹², —NR¹¹C(O)R¹², —C═NH(OR¹¹), —C(O)OR¹¹, —C(O)NR¹¹R¹², —C(O)R¹¹, —C(O)₃₋₆-membered heterocyclyl or 3-6-membered heterocyclyl, wherein said alkyl is optionally substituted by oxo, halogen, —NR¹³R¹⁴, —OR¹³ or 3-10 membered heterocyclyl, and said heterocyclyl is optionally substituted by oxo, halogen, —NR¹³R¹⁴, —OR¹³, 3-6-membered heterocyclyl or C₁-C₃ alkyl optionally substituted by halogen or OR¹¹.

In certain embodiments, R⁵ is pyridinyl, pyrimidinyl, pyrazolyl, thiazolyl, pyrazinyl, pyridazinyl, oxazolyl or isoxazolyl optionally substituted by C₁-C₆ alkyl, halogen, —CN, —O(C₀-C₃ alkyl), —CF₃, —NR¹¹R¹², —C═NH(OR¹¹), —C(O)OR¹¹, 3-6-membered heterocyclyl, wherein said alkyl is optionally substituted by halogen or OR¹¹ and said heterocyclyl is optionally substituted by oxo, halogen or C₁-C₃ alkyl optionally substituted by halogen or OR¹¹.

In certain embodiments, R⁵ is 5-6-membered heteroaryl, wherein R⁵ is optionally substituted by R¹⁰, wherein R¹⁰ is C₁-C₆ alkyl, halogen, —CN, —OR¹¹, —SR¹¹, —NR¹¹R¹², —CF₃, —C(O)R¹¹, —C(O)OR¹¹, —C(O)NR¹¹R¹², —NR¹¹C(O)R¹², —S(O)₁₋₂R¹¹, —NR¹¹S(O)₁₋₂R¹², —S(O)₁₋₂NR¹¹R¹², C₃-C₆ cycloalkyl, 3-6-membered heterocyclyl, —C(O)(3-6-membered heterocyclyl), 5-6-membered heteroaryl or phenyl, wherein R¹⁰ is independently optionally substituted by halogen, C₁-C₃ alkyl, oxo, —CF₃, —OR¹³, —NR¹³R¹⁴, —C(O)R¹³ or —S(O)₁₋₂R¹³. In an example, R⁵ is pyridinyl, pyrimidinyl, pyridazinyl, pyrazinyl, triazinyl, thienyl, pyrazolyl, pyranyl, triazolyl, isoxazolyl, oxazolyl, imidazolyl, thiazolyl or thiadiazolyl, wherein R⁵ is optionally substituted by 1, 2 or 3 R¹⁰.

In certain embodiments, R⁵ is pyridinyl optionally substituted by C₁-C₆ alkyl, C₂-C₆ alkenyl, C₂-C₆ alkynyl, halogen, —(C₀-C₃ alkyl)CN, —(C₀-C₃ alkyl)OR¹¹, —(C₀-C₃ alkyl)SR¹¹, —(C₀-C₃ alkyl)NR¹¹R¹², —(C₀-C₃ alkyl)CF₃, —(C₀-C₃ alkyl)NO₂, —C═NH(OR¹¹), —(C₀-C₃ alkyl)C(O)R¹¹, —(C₀-C₃ alkyl)C(O)OR¹¹, —(C₀-C₃ alkyl)C(O)NR¹¹R¹², —(C₀-C₃ alkyl)NR¹¹C(O)R¹², —(C₀-C₃ alkyl)S(O)₁₋₂R¹¹, —(C₀-C₃ alkyl)NR¹¹S(O)₁₋₂R¹², —(C₀-C₃ alkyl)S(O)₁₋₂NR¹¹R¹², —(C₀-C₃ alkyl)(C₃-C₆ cycloalkyl), —(C₀-C₃ alkyl)(3-6-membered heterocyclyl), —(C₀-C₃ alkyl)C(O)(3-6-membered heterocyclyl), —(C₀-C₃ alkyl)(5-6-membered heteroaryl) or —(C₀-C₃ alkyl)phenyl, wherein R¹⁰ is independently optionally substituted by halogen, C₁-C₃ alkyl, oxo, —CF₃, —(C₀-C₃ alkyl)OR¹³, —(C₀-C₃ alkyl)NR¹³R¹⁴, —(C₀-C₃ alkyl)C(O)R¹³ or —(C₀-C₃ alkyl)S(O)₁₋₂R¹³.

In certain embodiments, R⁵ is selected from:

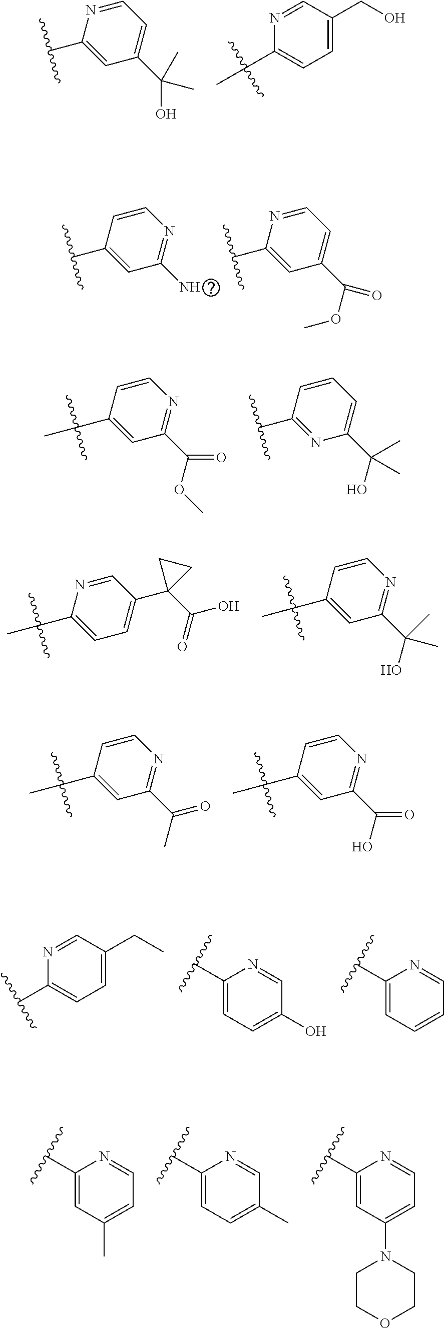

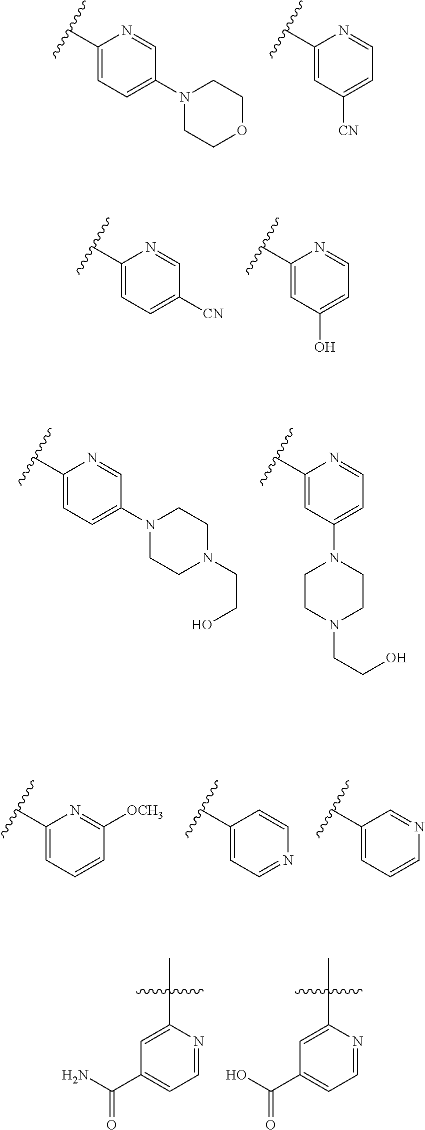

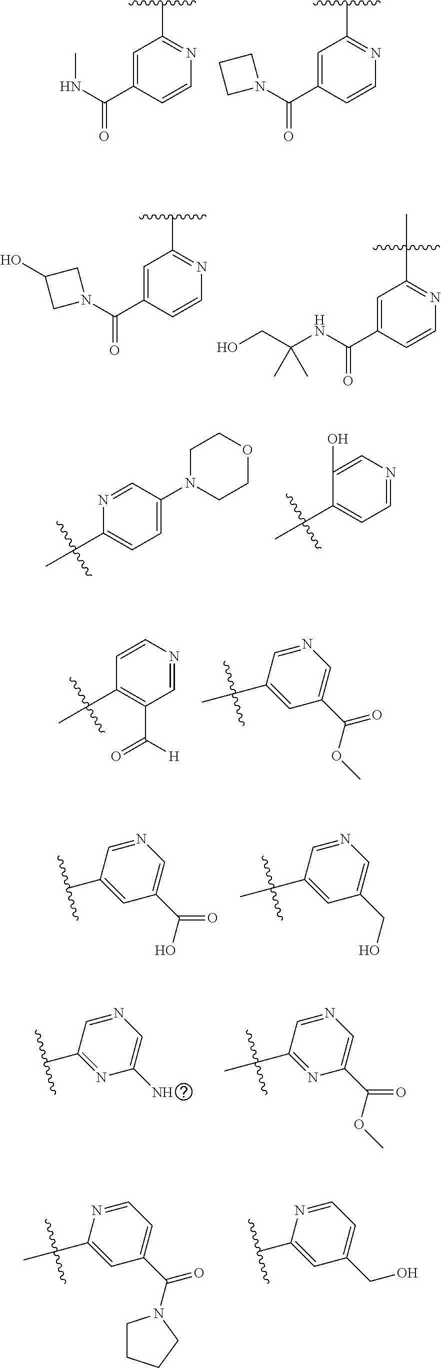

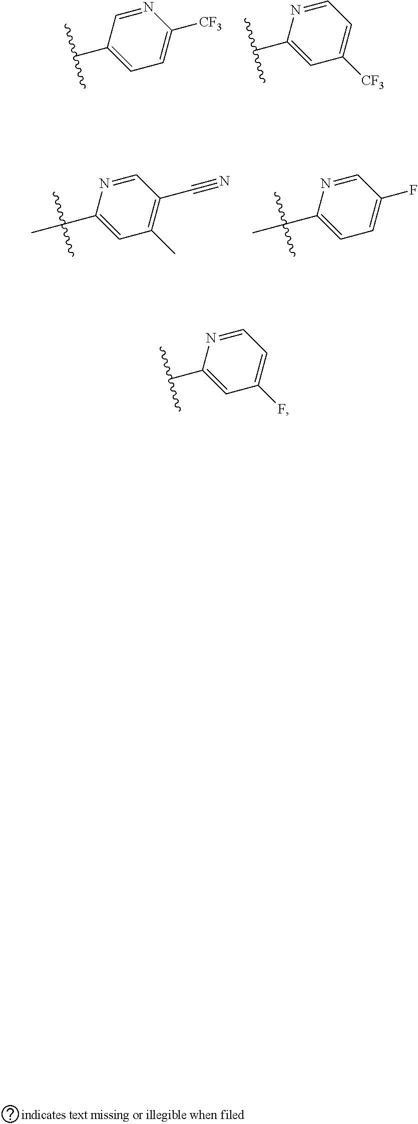

wherein the wavy lines represent the point of attachment in Formulas Ia-Ib.

In certain embodiments, R⁵ is pyrimidinyl, pyridazinyl, triazinyl or pyrazinyl, optionally substituted by R¹⁰.

In certain embodiments, R⁵ is pyrimidinyl, pyridazinyl, or pyrazinyl, optionally substituted by C₁-C₆ alkyl, C₂-C₆ alkenyl, C₂-C₆ alkynyl, halogen, —(C₀-C₃ alkyl)CN, —(C₀-C₃ alkyl)OR¹¹, —(C₀-C₃ alkyl)SR¹¹, —(C₀-C₃ alkyl)NR¹¹R¹², —(C₀-C₃ alkyl)CF₃, —(C₀-C₃ alkyl)NO₂, —C═NH(OR¹¹), —(C₀-C₃alkyl)C(O)R¹¹, —(C₀-C₃alkyl)C(O)OR¹¹, —(C₀-C₃ alkyl)C(O)NR¹¹R¹², —(C₀-C₃ alkyl)NR¹¹C(O)R¹², —(C₀-C₃ alkyl)S(O)₁₋₂R¹¹, —(C₀-C₃ alkyl)NR¹¹S(O)₁₋₂R¹², —(C₀-C₃ alkyl)S(O)₁₋₂NR¹¹R¹², —(C₀-C₃ alkyl)(C₃-C₆ cycloalkyl), —(C₀-C₃ alkyl)(3-6-membered heterocyclyl), —(C₀-C₃ alkyl)C(O)(3-6-membered heterocyclyl), —(C₀-C₃ alkyl)(5-6-membered heteroaryl) or —(C₀-C₃ alkyl)phenyl, wherein R¹⁰ is independently optionally substituted by halogen, C₁-C₃ alkyl, oxo, —CF₃, —(C₀-C₃ alkyl)OR¹³, —(C₀-C₃ alkyl)NR¹³R¹⁴, —(C₀-C₃alkyl)C(O)R¹³ or —(C₀-C₃alkyl)S(O)₁₋₂R¹³.

In certain embodiments, R⁵ is selected from:

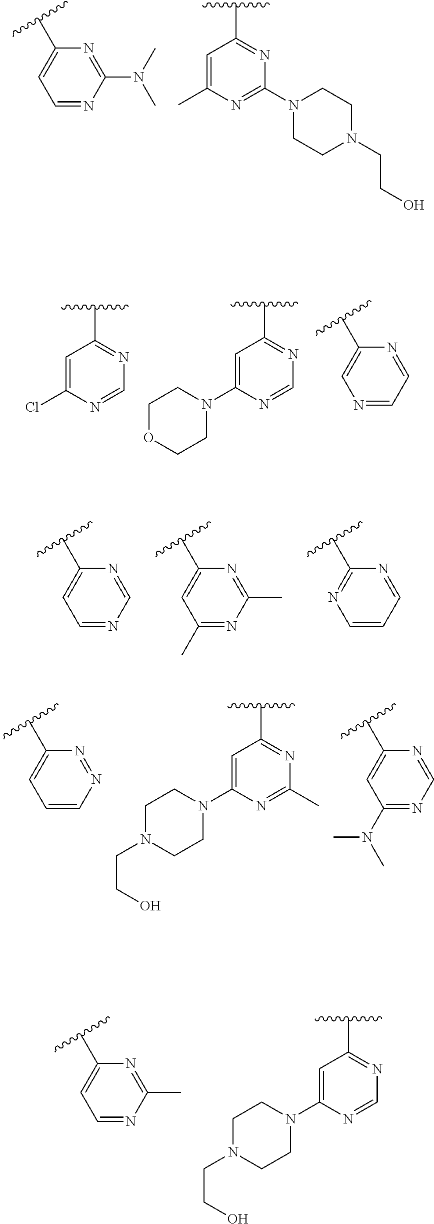

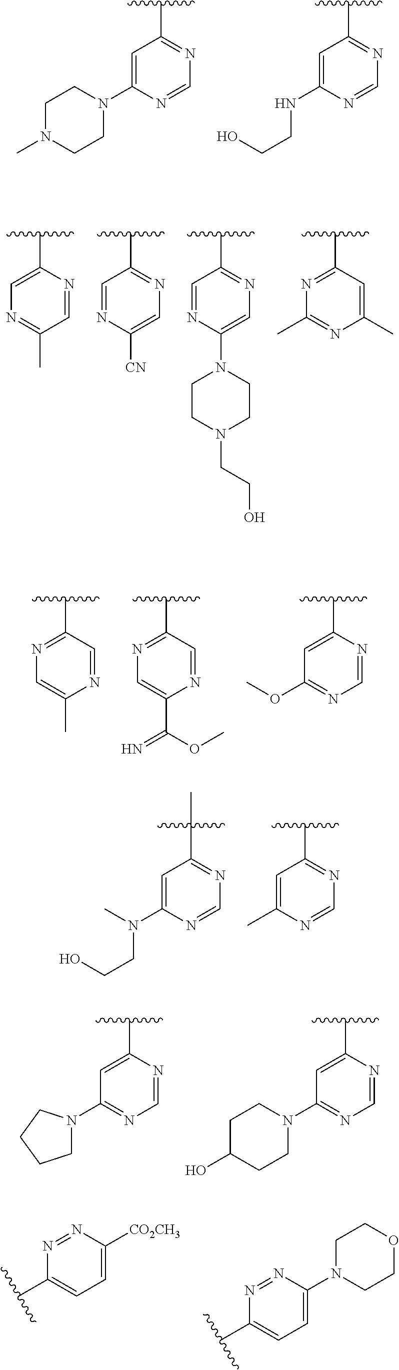

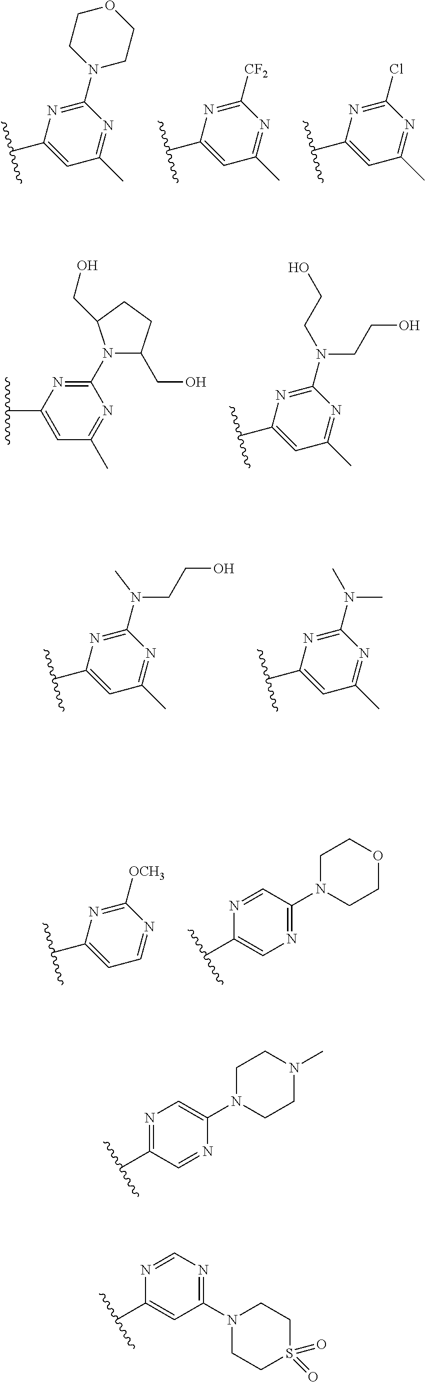

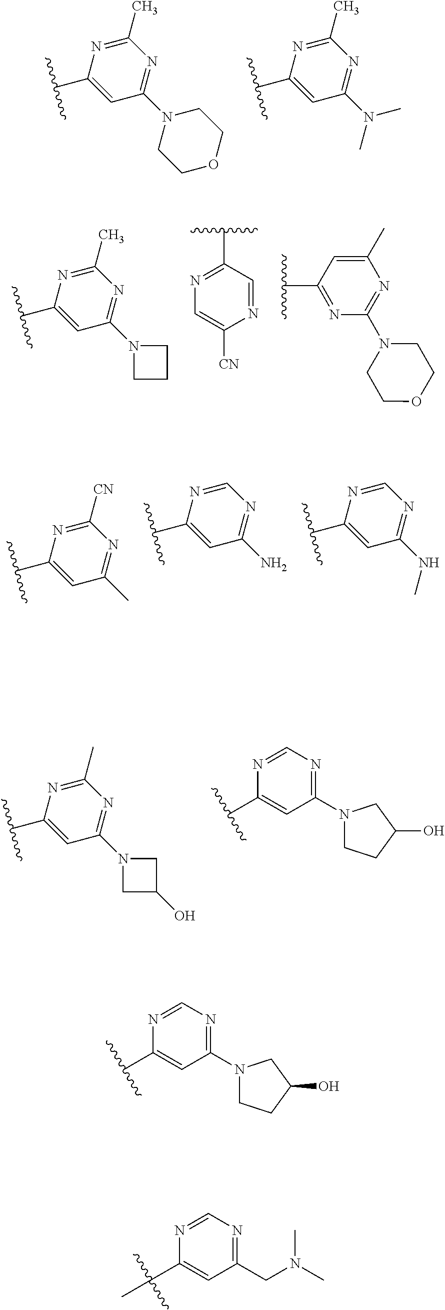

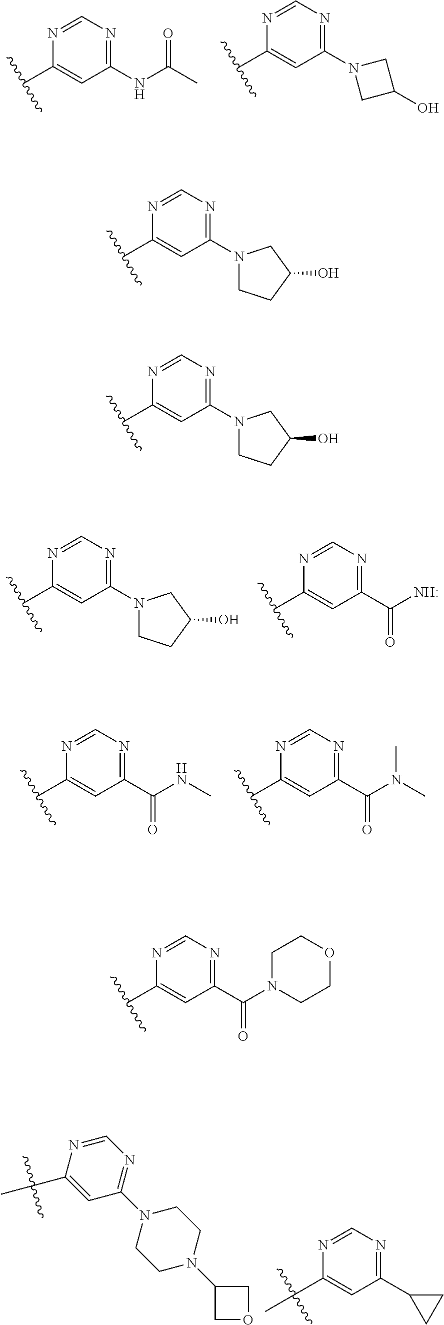

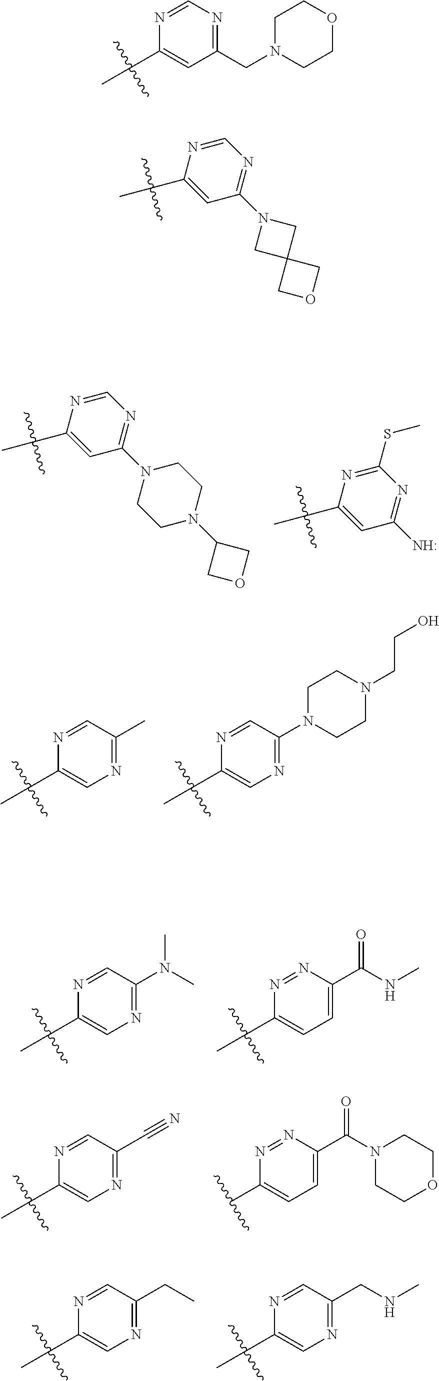

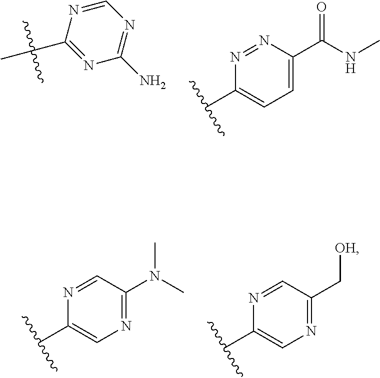

wherein the wavy lines represent the point of attachment in Formulas Ia-Ib.

In certain embodiments, R⁵ is pyrazolyl, isoxazolyl, oxazolyl, imidazolyl, thiazolyl, isothiazolyl or thiadiazolyl, wherein R⁵ is optionally substituted by R¹⁰.

In certain embodiments, R⁵ is pyrazolyl, isoxazolyl, oxazolyl, imidazolyl, thiazolyl or thiadiazolyl, wherein R⁵ is optionally substituted by R¹⁰, wherein R¹⁰ is C₁-C₆ alkyl, halogen, —CN, —OR¹¹, —SR¹¹, —NR¹¹R¹², —CF₃, —C(O)R¹¹, —C(O)OR¹¹, —C(O)NR¹¹R¹², —NR¹¹C(O)R¹², —S(O)₁₋₂R¹¹, —NR¹¹S(O)₁₋₂R¹², —S(O)₁₋₂NR¹¹R¹², C₃-C₆ cycloalkyl, 3-6-membered heterocyclyl, —C(O)(3-6-membered heterocyclyl), 5-6-membered heteroaryl or phenyl, wherein R¹⁰ is independently optionally substituted by halogen, C₁-C₃ alkyl, oxo, —CF₃, —OR¹³, —NR¹³R¹⁴, —C(O)R¹³ or —S(O)₁₋₂R¹³.

In certain embodiments, R⁵ is selected from:

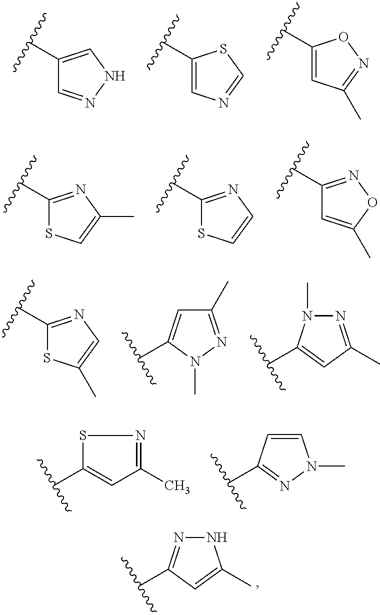

wherein the wavy lines represent the point of attachment in Formulas Ia-Ib.

In certain embodiments, R⁵ is pyrrolopyrimidinyl, pyrazolopyrimidinyl, wherein R⁵ is optionally substituted by R¹⁰. In certain embodiments, R⁵ is selected from:

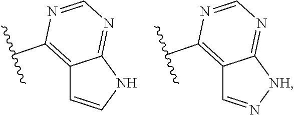 [SMILES: *c1ncnc2[nH]ccc12 ; *c1ncnc2[nH]ncc12]

wherein the wavy lines represent the point of attachment in Formulas Ia-Ib.

In certain embodiments, R⁵ is pyrimidinonyl optionally substituted by R¹⁰. In certain embodiments, R⁵ is

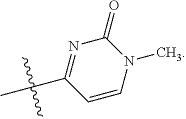 [SMILES: *c1ccn(C)c(=O)n1]

In certain embodiments, R⁵ is hydrogen, methyl, ethyl, isopropyl, tert-butyl, —CH₂OH, —CH₂N(CH₃)₂, —CH₂NHC(O)OC(CH₃)₃, cyclopropyl, cyclobutyl,

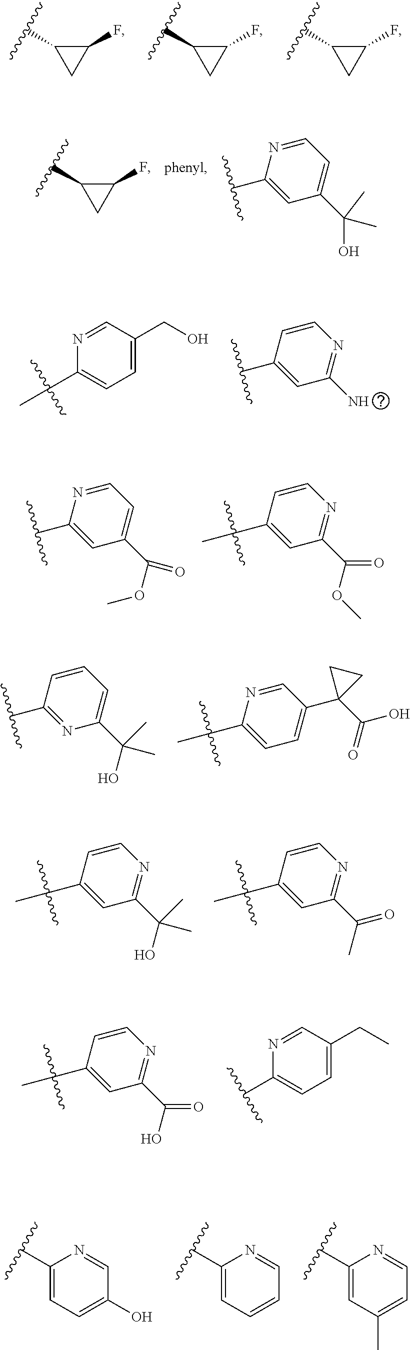

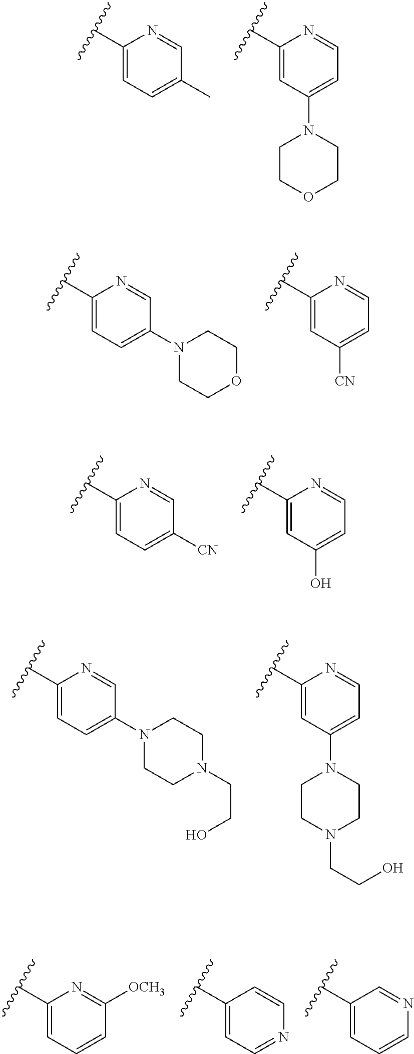

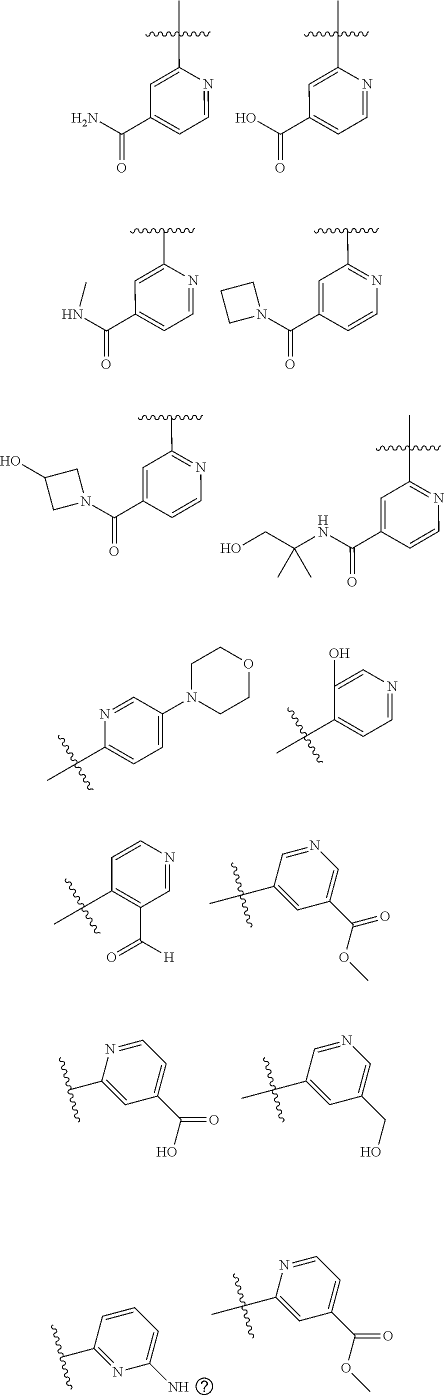

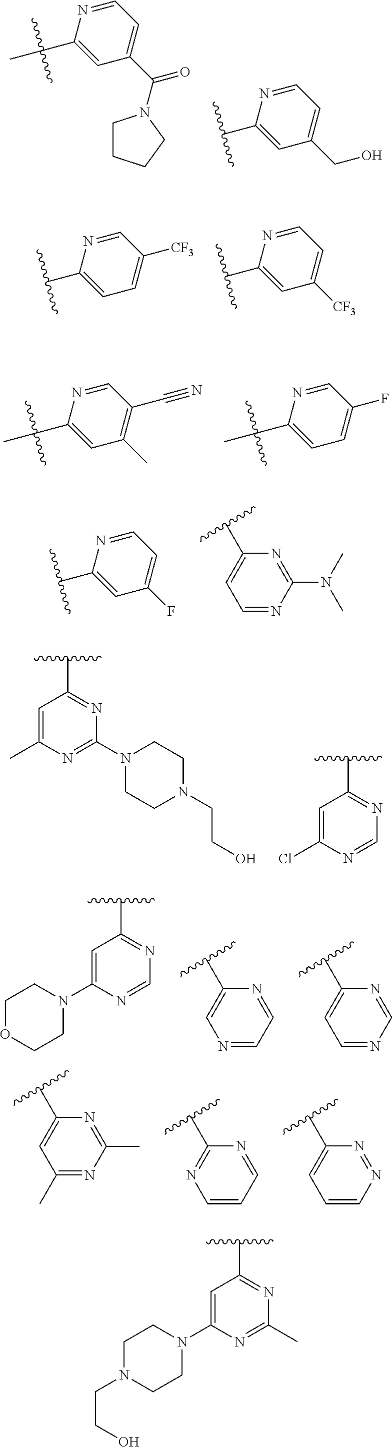

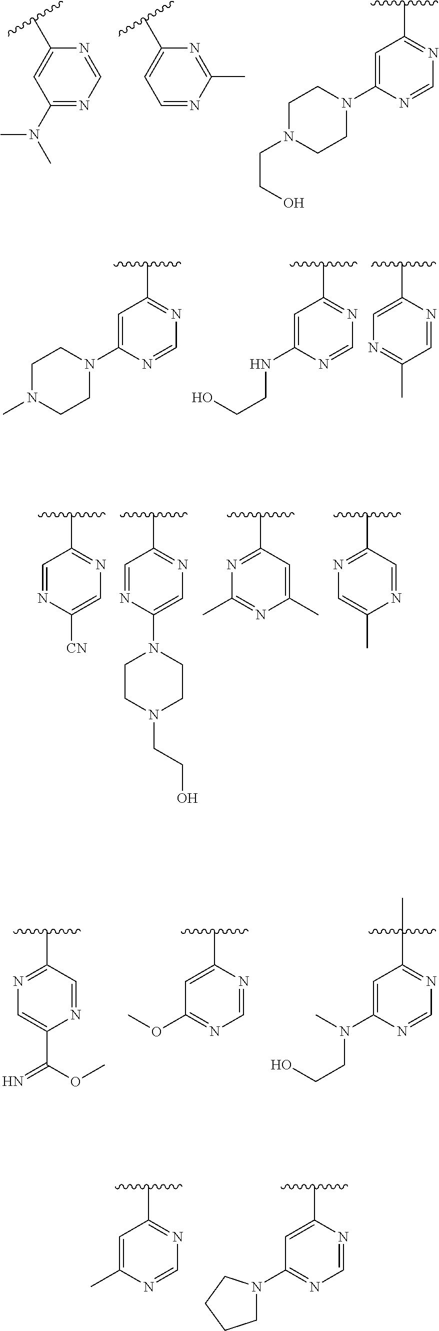

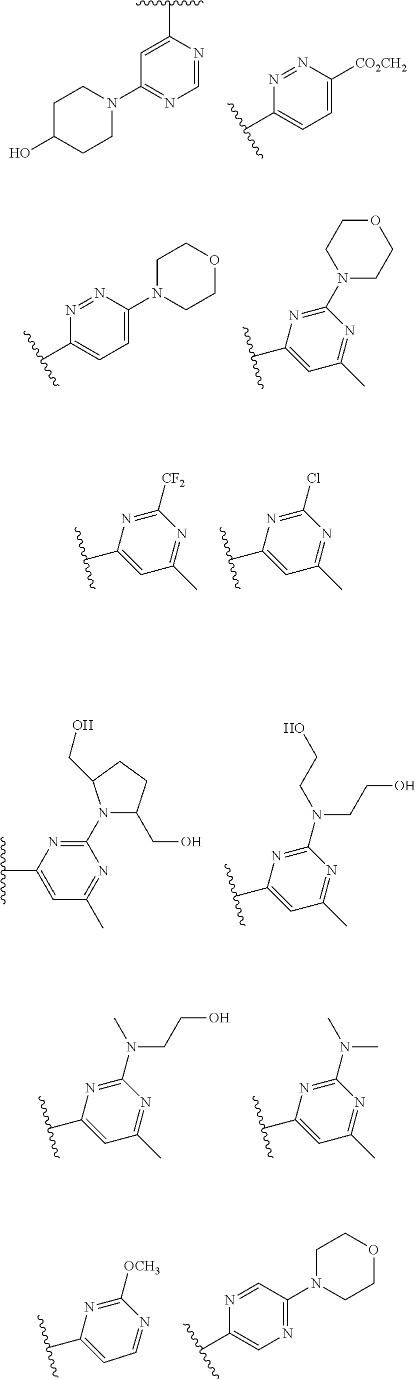

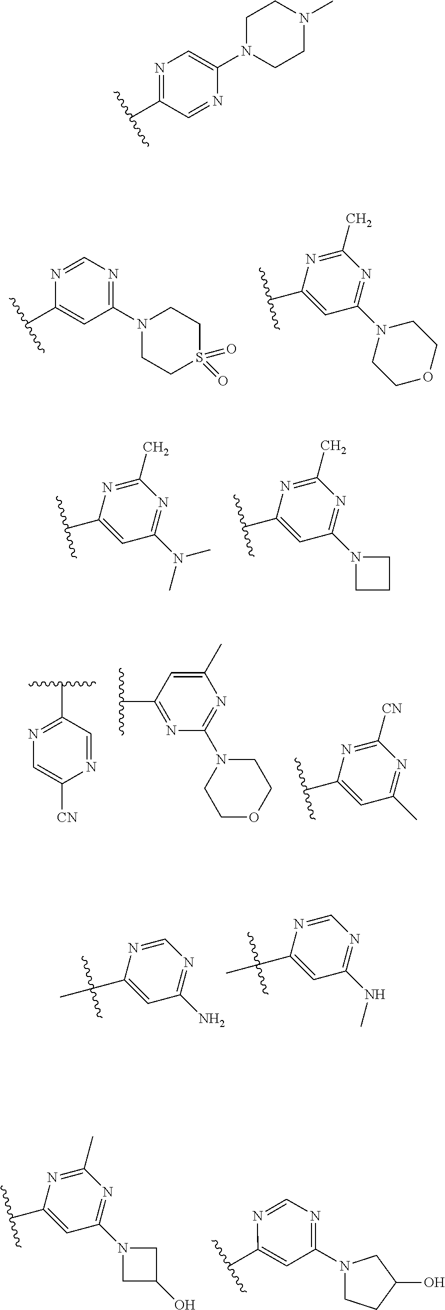

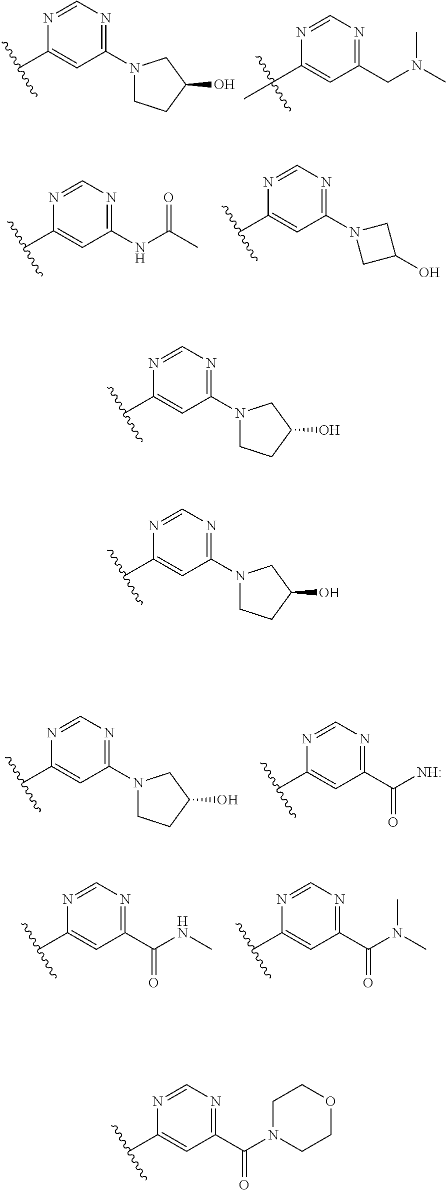

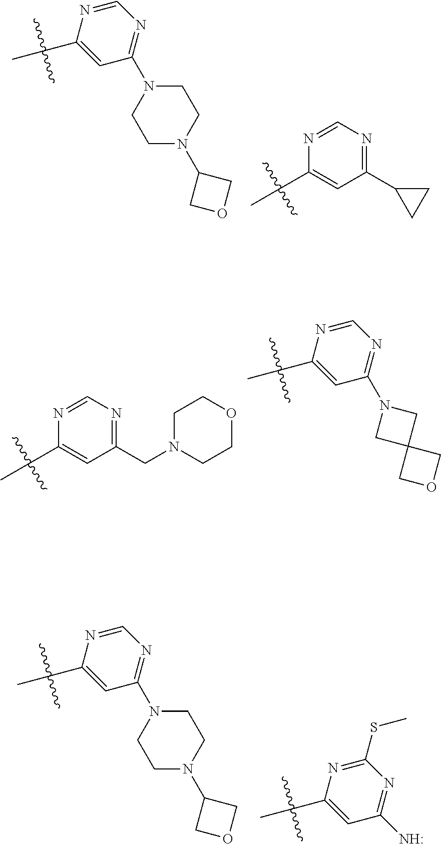

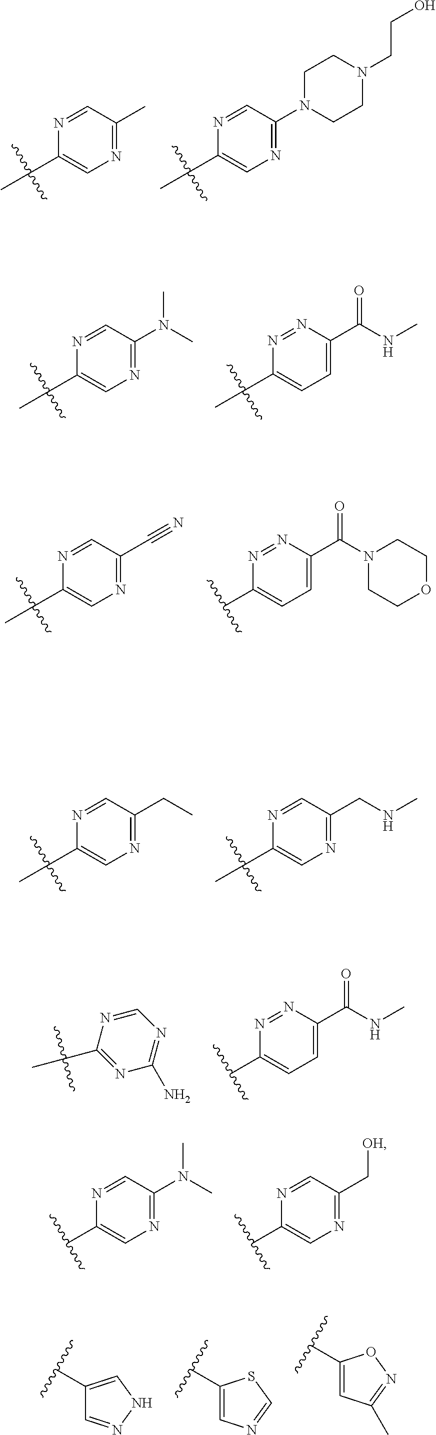

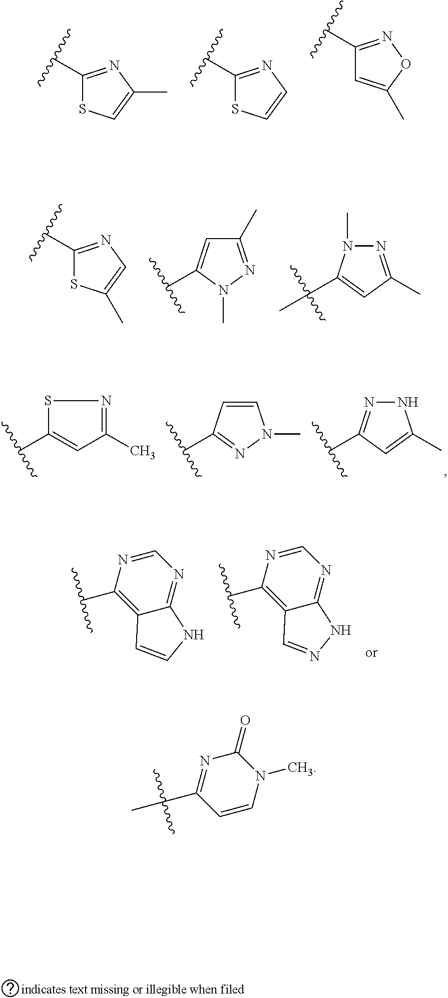

In certain embodiments, R¹⁰ is selected from C₁-C₆ alkyl, C₁-C₆ alkenyl, C₁-C₆ alkynyl, C₃-C₆ cycloalkyl, halogen, —CN, —OR¹¹, —CH₂OR¹¹, —SR¹¹, —CH₃—, —NR¹¹R¹², NR¹¹C(O)R¹², —C═NH(OR¹¹), —C(O)R¹¹, C(O)OR¹¹, NR¹¹C(O)OR¹², —C(O)NR¹¹R¹², —C(O)3-10-membered heterocyclyl or 3-10-membered heterocyclyl, wherein said alkyl, alkenyl, alkynyl are independently optionally substituted by oxo, halogen, —NR¹³R¹⁴, —OR¹³ or 3-10 membered heterocyclyl, and said cycloalkyl and heterocyclyl are independently optionally substituted by oxo, halogen, —NR¹³R¹⁴, —OR¹³, —C(O)R¹³, 3-10-membered heterocyclyl or C₁-C₃ alkyl optionally substituted by halogen, —NR¹³R¹⁴ or OR¹³.

In certain embodiments, R¹⁰ is C₁-C₆ alkyl, halogen, —CN, —OR¹¹, —SR¹¹, —NR¹¹R¹², —CF₃, —C═NH(OR¹¹), —C(O)OR¹¹, C₃-C₆ cycloalkyl, 3-6-membered heterocyclyl, 5-6-membered heteroaryl or phenyl, wherein R¹⁰ is independently optionally substituted by halogen, C₁-C₃ alkyl, oxo, —CF₃, —OR¹³, —NR¹³R¹⁴, —C(O)R¹³ or —S(O)₁₋₂R¹³.

In certain embodiments, R¹⁰ is oxo, methyl, ethyl, propyl, isopropyl, tert-butyl, —CH₂OH, F, Cl, —N(CH₃)₂, —CN, —C═NH(OCH₃), —OH, —OCH₃, —NH₂, —NHCH₃, —N(CH₃)₂, —CH₂NH₂, —CH₂NHCH₃, —CH₂N(CH₃)₂, —C(O)NH₂, —C(O)NHCH₃, —C(O)N(CH₃)₂, —C(O)morpholinyl, —CH₂morpholinyl, 1-hydroxyethyl, 4-oxetanylpiperazinyl, 2-oxa-6-azaspiro[3.3] heptanyl, —C(O)H, —CO₂H, —COCH₃, —SCH₃, 1-carboxycycloprop-1yl, —C(O)pyrrolidinyl, 4-fluoroazetidinyl, —NHC(O)CH₃, —NHC(O)CH₂CH₃, —C(O)azetidinyl, —C(O)(4-hydroxyazetidinyl), —C(O)NH(1,1-dimethyl-2-hydroxyethyl), —CO₂CH₃, —CF₃, morpholinyl, pyrrolidinyl, azetidinyl, 4-hydroxyazetidinyl, 1,1-dioxothiomorpholinyl, N-methylpiperazinyl, N-(2-hydroxyethyl)piperazinyl, 4-(2-hydroxyethyl)piperazinyl, 2-hydroxypropyl, 4-hydroxypiperidinyl, 1,2-dihydroxyethyl, 3-hydroxypyrrolidinyl, 2,5-dihydroxymethylpyrrolidinyl, 2,5-dihydroxyethylpyrrolidinyl, —NHC(O)Ot-butyl, —NH(CH₂)₂OH, —NCH₃(CH₂)₂OH, or —O(CH₂)₂pyrrolidinyl.

In certain embodiments, R¹⁰ is methyl, —CH₂OH, F, Cl, —N(CH₃)₂, —CN, —C═NH(OCH₃), —OCH₃, —CO₂CH₃, —CF₃, morpholinyl, pyrrolidinyl, azetidinyl, 1,1-dioxothiomorpholinyl, N-methylpiperazinyl, N-(2-hydroxyethyl)piperazinyl, 4-hydroxypiperidinyl, 2,5-dihydroxymethylpyrrolidinyl, 2,5-dihydroxyethylpyrrolidinyl, —NH(CH₂)₂OH, NCH₃(CH₂)₂OH, or —O(CH₂)₂pyrrolidinyl.

In certain embodiments, R¹⁰ is selected from:

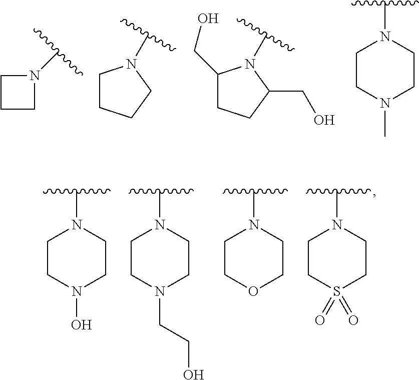

wherein the wavy line represents the point of attachment in Formulas Ia-Ib.

In certain embodiments, R¹¹ and R¹² are independently hydrogen or C₁-C₆ alkyl optionally substituted by halogen, oxo, —OR¹³, —NR¹³R¹⁴, C₃-C₆ cycloalkyl, phenyl, 3-6-membered heterocyclyl or 5-6-membered heteroaryl, or are taken together with the atom to which they attached to form a 3-6 membered heterocyclyl optionally substituted by halogen, oxo, —OR¹³, —NR¹³R¹⁴ or C₁-C₃ alkyl;

In certain embodiments, R¹¹ and R¹² are independently hydrogen, methyl or 2-hydroxyethyl, or are taken together with the atom to which they attached to form a azetidinyl, pyrrolidinyl, morpholinyl, piperazinyl or piperidinyl ring optionally substituted by halogen, oxo, —NR¹³R¹⁴ or C₁-C₃ alkyl.

In certain embodiments, R¹¹ and R¹² are independently hydrogen, methyl or 2-hydroxyethyl.

In certain embodiments, R¹³ and R¹⁴ are independently hydrogen or C₁-C₃ alkyl.

In certain embodiments, R¹⁵ is hydrogen, halogen, —CN, —OR¹⁸, —NR¹⁸R¹⁹, C₁-C₃ alkyl, C₁-C₃ alkenyl C₁-C₃ alkynyl, or C₃-C₆ cycloalkyl, wherein R¹⁵ is optionally substituted by halogen, oxo, CN or —NR¹⁸R¹⁹.

In certain embodiments, R¹⁵ is hydrogen, —CN, halogen or C₁-C₃ alkyl optionally substituted by halogen, oxo or —NR¹⁸R¹⁹. In certain embodiments, R¹⁵ is F, Cl, Br, —CN, —C(O)NH₂ or methyl.

In certain embodiments, R¹⁵ is hydrogen or halogen. In certain embodiments, R¹⁵ is halogen. In certain embodiments, R¹⁵ is F.

In certain embodiments, R¹⁶ is hydrogen, C₁-C₃ alkyl, C₁-C₃ alkenyl, C₁-C₃ alkynyl, C₃-C₆ cycloalkyl, phenyl, 5-6 membered heteroaryl or 3-6 membered heterocyclyl, wherein R¹⁶ is optionally substituted by halogen, oxo, —CN, —CF₃, —OR¹⁸, —NR¹⁸R¹⁹ or C₁-C₆ alkyl.

In certain embodiments, R¹⁶ is hydrogen or C₁-C₃ alkyl optionally substituted by oxo, (═NH), —NR¹⁸R¹⁹ or —OR¹⁸. In certain embodiments, R¹⁶ is methyl, ethyl, 2-hydroxyethyl, —CH₂C(O)NH₂, —CH₂(C═NH)NH₂ or —CH₂C(O)OH

In certain embodiments, R¹⁶ is hydrogen or C₁-C₃ alkyl. In certain embodiments, R¹⁶ is methyl.

In certain embodiments, R¹⁸ and R¹⁹ are independently hydrogen or C₁-C₃ alkyl.

In certain embodiments, R¹ is independently halogen and R⁴ is —NH— or NHC(O)—.

In certain embodiments, A is CR³; X is CH; R¹ is independently hydrogen, —OCH₃, —CF₃, —OCF₃, —CH₃, Cl or F, wherein both R¹ cannot be hydrogen at the same time; R² is hydrogen; R³ is hydrogen or —CN; R⁴ is —NH—, NHC(O)—, NHC(O)NH— or NHC(O)O—; and R⁵ is cyclopropyl optionally substituted by C₁-C₃ alkyl or halogen.

In certain embodiments, A is CR³; X is CH; R¹ is independently hydrogen, —OCH₃, —CF₃, —OCF₃, —CH₃, Cl or F, wherein both R¹ cannot be hydrogen at the same time; R² is hydrogen; R³ is hydrogen or —CN; R⁴ is —NH—, NHC(O)—, NHC(O)NH— or NHC(O)O—; and R⁵ is pyrimidinyl, pyridinyl, pyridazinyl or pyrazinyl optionally substituted by R¹⁹.

In certain embodiments, R¹ is independently hydrogen or halogen, R⁴ is —NHR⁵, —NR⁶C(O)R⁵, —NR⁶C(O)OR⁵ or —NR⁶C(O)NR⁷R⁵, R¹⁶ is hydrogen, C₁-C₆ alkyl, C₂-C₆ alkenyl, C₂-C₆ alkynyl, —(C₀-C₃ alkyl)CN, —(C₁-C₃ alkyl)OR¹⁸, —(C₁-C₃ alkyl)SR¹⁸, —(C₁-C₃ alkyl)NR¹⁸R¹⁹, —(C₁-C₃ alkyl)CF₃, —O(C₁-C₃ alkyl)CF₃, —(C₂-C₃ alkyl)NO₂, —(C₀-C₃ alkyl)C(O)R¹⁸, —(C₀-C₃ alkyl)C(O)OR¹⁸, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸, —(C₀-C₃ alkyl)NR¹⁸S(O)₁₋₂R¹⁹, or —(C₀-C₃ alkyl)S(O)₁₋₂NR¹⁸R¹⁹, and R¹⁸ and R¹⁹ are hydrogen or C₁-C₃ alkyl optionally substituted by halogen or oxo, and wherein both R¹ are not hydrogen at the same time and R⁵ is other than hydrogen.

In certain embodiments, A is CR³; X is CH; R¹ is independently Cl or F; R² is hydrogen; R³ is hydrogen or —CN; R⁴ is —NH—, NHC(O)—, NHC(O)NH— or NHC(O)O—; R⁵ is pyrimidinyl, pyridinyl, pyridazinyl or pyrazinyl optionally substituted by R¹⁹, and R¹⁶ is hydrogen or C₁-C₃ alkyl.

Another embodiment includes a compound of Formulas Ia-Ib, stereoisomers or pharmaceutically acceptable salts thereof, selected from:
2-(2,6-dichlorophenyl)-N-(pyridin-2-yl)-1H-imidazo[4,5-c]pyridin-4-amine;
2-(4-(4-(2-(2,6-dichlorophenyl)-3H-imidazo[4,5-c]pyridin-4-ylamino)-6-methylpyrimidin-2-yl)piperazin-1-yl)ethanol;
2-(2,6-dichlorophenyl)-N-(1H-pyrazol-4-yl)-3H-imidazo[4,5-c]-pyridin-4-amine;
N-(6-chloropyrimidin-4-yl)-2-(2,6-dichlorophenyl)-3H-imidazo-[4,5-c]pyridin-4-amine;
2-(2,6-dichlorophenyl)-N-(6-morpholinopyrimidin-4-yl)-3H-imidazo[4,5-c]pyridin-4-amine;
2-(2-chloro-6-fluorophenyl)-N-(pyrazin-2-yl)-3H-imidazo[4,5-c]-pyridin-4-amine;
N-(2-(2,6-dichlorophenyl)-3H-imidazo[4,5-c]pyridin-4-yl)thiazole-5-carboxamide;
(1R,2R)—N-(2-(2,6-dichlorophenyl)-3H-imidazo[4,5-c]pyridin-4-yl)-2-fluorocyclopropanecarboxamide;
(1S,2S)—N-(2-(2,6-dichlorophenyl)-3H-imidazo[4,5-c]pyridin-4-yl)-2-fluorocyclopropanecarboxamide;
N-(2-(2,6-dichloro-4-cyanophenyl)-3H-imidazo[4,5-c]pyridin-4-yl)cyclopropanecarboxamide;
1-(2-(2,6-dichlorophenyl)-3H-imidazo[4,5-c]pyridin-4-yl)-3-methylurea;
N-(2-(2,6-dichlorophenyl)-1-methyl-1H-imidazo[4,5-c]pyridine-4-yl)cyclopropanecarboxamide;
N-(2-(2,6-dichlorophenyl)-3-methyl-3H-imidazo[4,5-c]pyridine-4-yl)cyclopropanecarboxamide;
8-(2,6-dichlorophenyl)-N-(pyrimidin-4-yl)-9H-purin-6-amine;
N-(8-(2,6-dichlorophenyl)-7H-purin-6-yl)cyclopropanecarboxamide;
2-(2,6-dichlorophenyl)-N-(2,6-dimethylpyrimidin-4-yl)-7-fluoro-1H-imidazo[4,5-c]pyridin-4-amine;
N-(2-(2,6-dichlorophenyl)-7-fluoro-1H-imidazo[4,5-c]pyridin-4-yl)cyclopropanecarboxamide;
2-(2,6-dichlorophenyl)-N-(pyrimidin-4-yl)-1H-imidazo[4,5-c]pyridine-4-amine;
2-(2,6-dichlorophenyl)-N-(4-(2-(pyrrolidin-1-yl)ethoxy)phenyl)-3H-imidazo[4,5-c]pyridin-4-amine;
[2-(2,6-dichloro-phenyl)-1H-imidazo[4,5-c]pyridin-4-yl]-(2,6-dimethyl-pyrimidin-4-yl)-amine;
2-(2,6-dichlorophenyl)-N-(pyrimidin-2-yl)-1H-imidazo[4,5-c]-pyridin-4-amine;
2-(2,6-dichlorophenyl)-N-(pyrazin-2-yl)-3H-imidazo[4,5-c]pyridine-4-amine;
2-(2,6-dichlorophenyl)-N-(pyridazin-3-yl)-1H-imidazo[4,5-c]-pyridin-4-amine;
N-(2-(2,6-dichlorophenyl)-1H-imidazo[4,5-c]pyridin-4-yl)-3-methyl-isoxazol-5-amine;
N-(2-(2,6-dichlorophenyl)-3H-imidazo[4,5-c]pyridin-4-yl)-benzamide;
N-(2-(2,6-dichlorophenyl)-3H-imidazo[4,5-c]pyridin-4-ylacetamide;
N-(2-(2,6-dichlorophenyl)-3H-imidazo[4,5-c]pyridin-4-yl)propionamide;
N-(2-(2,6-dichlorophenyl)-3H-imidazo[4,5-c]pyridin-4-yl)-isobutyramide;
N-(2-(2,6-dichlorophenyl)-3H-imidazo[4,5-c]pyridin-4-yl)pivalamide;
2-(4-(6-(2-(2,6-dichlorophenyl)-3H-imidazo[4,5-c]pyridine-4-ylamino)-2-methylpyrimidin-4-yl)piperazin-1-yl)ethanol;
N⁴-(2-(2,6-dichlorophenyl)-3H-imidazo[4,5-c]pyridin-4-yl)-N⁶,N⁶-dimethylpyrimidine-4,6-diamine;
N⁴-(2-(2,6-dichlorophenyl)-3H-imidazo[4,5-c]pyridin-4-yl)-N²,N²-dimethylpyrimidine-2,4-diamine;
2-(2,6-dichlorophenyl)-N-(2-methylpyrimidin-4-yl)-3H-imidazo-[4,5-c]pyridin-4-amine;
2-(2,6-dichlorophenyl)-N-(4-methylpyridin-2-yl)-3H-imidazo[4,5-c]-pyridin-4-amine;
2-(2,6-dichlorophenyl)-N-(5-methylpyridin-2-yl)-3H-imidazo-[4,5-c]pyridin-4-amine;
2-(4-(6-(2-(2,6-dichlorophenyl)-3H-imidazo[4,5-c]pyridine-4-ylamino)pyrimidin-4-yl)piperazin-1-yl)ethanol;
2-(2,6-dichlorophenyl)-N-(4-morpholinopyridin-2-yl)-3H-imidazo[4,5-c]pyridin-4-amine;
2-(2,6-dichlorophenyl)-N-(5-morpholinopyridin-2-yl)-3H-imidazo[4,5-c]pyridin-4-amine;
2-(2,6-dichlorophenyl)-N-(6-(4-methylpiperazin-1-yl)pyrimidin-4-yl)-3H-imidazo[4,5-c]pyridin-4-amine;
2-(6-(2-(2,6-dichlorophenyl)-3H-imidazo[4,5-c]pyridin-4-ylamino)-pyrimidin-4-ylamino)ethanol;
2-(2-(2,6-dichlorophenyl)-3H-imidazo[4,5-c]pyridin-4-ylamino)isonicotinonitrile;
6-(2-(2,6-dichlorophenyl)-3H-imidazo[4,5-c]pyridin-4-ylamino)nicotinonitrile;
2-(2,6-dichlorophenyl)-N-(5-methylpyrazin-2-yl)-3H-imidazo[4,5-c]pyridin-4-amine;
5-(2-(2,6-dichlorophenyl)-3H-imidazo[4,5-c]pyridin-4-ylamino)pyrazine-2-carbonitrile;
2-(4-(5-(2-(2,6-dichlorophenyl)-3H-imidazo[4,5-c]pyridin-4-ylamino)pyrazin-2-yl)piperazin-1-yl)ethanol;
2-(2-chloro-6-fluorophenyl)-N-(2,6-dimethylpyrimidin-4-yl)-3H-imidazo[4,5-c]pyridin-4-amine;
N-(2-(2-chloro-6-fluorophenyl)-3H-imidazo[4,5-c]pyridin-4-yl)-cyclopropanecarboxamide;
2-(2-chloro-6-fluorophenyl)-N-(1H-pyrazol-4-yl)-3H-imidazo[4,5-c]pyridin-4-amine;
2-(2-chloro-6-fluorophenyl)-N-(pyridin-2-yl)-1H-imidazo[4,5-c]pyridin-4-amine;
2-(2-chloro-6-fluorophenyl)-N-(5-methylpyridin-2-yl)-3H-imidazo[4,5-c]pyridin-4-amine;
2-(2-(2-chloro-6-fluorophenyl)-3H-imidazo[4,5-c]pyridin-4-ylamino)isonicotinonitrile;
2-(2-chloro-6-fluorophenyl)-N-(4-methylpyridin-2-yl)-3H-imidazo[4,5-c]pyridin-4-amine;
6-(2-(2-chloro-6-fluorophenyl)-3H-imidazo[4,5-c]pyridin-4-ylamino)nicotinonitrile;
2-(2-chloro-6-fluorophenyl)-N-(pyridin-2-yl)-3H-imidazo[4,5-c]pyridin-4-amine;
2-(4-(2-(2-(2-chloro-6-fluorophenyl)-3H-imidazo[4,5-c]pyridin-4-ylamino)pyridin-4-yl)piperazin-1-yl)ethanol;
2-(2-chloro-6-fluorophenyl)-N-(5-morpholinopyridin-2-yl)-3H-imidazo[4,5-c]pyridin-4-amine;
2-(4-(6-(2-(2-chloro-6-fluorophenyl)-3H-imidazo[4,5-c]pyridin-4-ylamino)pyridin-3-yl)piperazin-1-yl)ethanol;
2-(2-chloro-6-fluorophenyl)-N-(5-methylpyrazin-2-yl)-3H-imidazo[4,5-c]pyridin-4-amine;
5-(2-(2-chloro-6-fluorophenyl)-3H-imidazo[4,5-c]pyridin-4-ylamino)pyrazine-2-carbonitrile;
methyl 5-(2-(2-chloro-6-fluorophenyl)-3H-imidazo[4,5-c]pyridin-4-ylamino)pyrazine-2-carbimidate;
1-(2-(2-chloro-6-fluorophenyl)-3H-imidazo[4,5-c]pyridin-4-yl)-3-methylurea;
2-(2-chloro-6-fluorophenyl)-N-(pyrimidin-4-yl)-3H-imidazo[4,5-c]pyridin-4-amine;
2-(2-chloro-6-fluorophenyl)-N-(6-morpholinopyrimidin-4-yl)-3H-imidazo[4,5-c]pyridin-4-amine;
2-(4-(6-(2-(2-chloro-6-fluorophenyl)-3H-imidazo[4,5-c]pyridine-4-ylamino)pyrimidin-4-yl)piperazin-1-yl)ethanol;
2-(2-chloro-6-fluorophenyl)-N-(6-(4-methylpiperazin-1-yl)-pyrimidin-4-yl)-3H-imidazo[4,5-c]pyridin-4-amine;
2-(6-(2-(2-chloro-6-fluorophenyl)-3H-imidazo[4,5-c]pyridin-4-ylamino)pyrimidin-4-ylamino)ethanol;
3,5-dichloro-4-(4-(4-methylpyridin-2-ylamino)-3H-imidazo[4,5-c]pyridin-2-yl)benzonitrile;
3,5-dichloro-4-(4-(2,6-dimethylpyrimidin-4-ylamino)-3H-imidazo[4,5-c]pyridin-2-yl)benzonitrile;
3,5-dichloro-4-(4-(5-(4-(2-hydroxyethyl)piperazin-1-yl)pyridin-2-ylamino)-3H-imidazo[4,5-c]pyridin-2-yl)benzonitrile;
3,5-dichloro-4-(4-(4-morpholinopyridin-2-ylamino)-3H-imidazo[4,5-c]pyridin-2-yl)benzonitrile;
3,5-dichloro-4-(4-(4-(4-(2-hydroxyethyl)piperazin-1-yl)pyridin-2-ylamino)-3H-imidazo[4,5-c]pyridin-2-yl)benzonitrile;
3,5-dichloro-4-(4-(5-morpholinopyridin-2-ylamino)-3H-imidazo[4,5-c]pyridin-2-yl)benzonitrile;
3,5-dichloro-4-(4-(5-methylpyridin-2-ylamino)-3H-imidazo[4,5-c]pyridin-2-yl)benzonitrile;
3,5-dichloro-4-(4-(6-morpholinopyrimidin-4-ylamino)-3H-imidazo[4,5-c]pyridin-2-yl)benzonitrile;
3,5-dichloro-4-(4-(6-(4-methylpiperazin-1-yl)pyrimidin-4-ylamino)-3H-imidazo[4,5-c]pyridin-2-yl)benzonitrile;
3,5-dichloro-4-(4-(6-methoxypyrimidin-4-ylamino)-3H-imidazo[4,5-c]pyridin-2-yl)benzonitrile;
2-(2-(2,6-dichloro-4-cyanophenyl)-3H-imidazo[4,5-c]pyridin-4-ylamino)isonicotinonitrile;
6-(2-(2,6-dichloro-4-cyanophenyl)-3H-imidazo[4,5-c]pyridin-4-ylamino)nicotinonitrile;
3,5-dichloro-4-(4-(5-methylthiazol-2-ylamino)-3H-imidazo[4,5-c]pyridin-2-yl)benzonitrile;
3,5-dichloro-4-(4-(4-methylthiazol-2-ylamino)-3H-imidazo[4,5-c]pyridin-2-yl)benzonitrile;
3,5-dichloro-4-(4-(thiazol-2-ylamino)-3H-imidazo[4,5-c]pyridin-2-yl)benzonitrile;
3,5-dichloro-4-(4-(6-methylpyrimidin-4-ylamino)-3H-imidazo[4,5-c]pyridin-2-yl)benzonitrile;
3,5-dichloro-4-(4-(6-((2-hydroxyethyl)(methyl)amino)pyrimidin-4-ylamino)-3H-imidazo[4,5-c]pyridin-2-yl)benzonitrile;
3,5-dichloro-4-(4-(6-(pyrrolidin-1-yl)pyrimidin-4-ylamino)-3H-imidazo[4,5-c]pyridin-2-yl)benzonitrile;
3,5-dichloro-4-(4-(6-(4-hydroxypiperidin-1-yl)pyrimidin-4-ylamino)-3H-imidazo[4,5-c]pyridin-2-yl)benzonitrile;
3,5-dichloro-4-(4-(6-(dimethylamino)pyrimidin-4-ylamino)-3H-imidazo[4,5-c]pyridin-2-yl)benzonitrile;
N-(2-(2-chloro-4-(methylsulfonyl)phenyl)-3H-imidazo[4,5-c]-pyridin-4-yl)cyclopropanecarboxamide;
2-(2-chloro-4-(methylsulfonyl)phenyl)-N-(2,6-dimethylpyrimidin-4-yl)-3H-imidazo[4,5-c]pyridin-4-amine;
N-(2-(2-(trifluoromethyl)phenyl)-1H-imidazo[4,5-c]pyridin-4-yl)cyclopropanecarboxamide;
N-(2,6-dimethylpyrimidin-4-yl)-2-(2-(trifluoromethyl)phenyl)-3H-imidazo[4,5-c]pyridin-4-amine;
N-(2-(2-(trifluoromethoxy)phenyl)-1H-imidazo[4,5-c]pyridin-4-yl)cyclopropanecarboxamide;
N-(2,6-dimethylpyrimidin-4-yl)-2-(2-(trifluoromethoxy)phenyl)-3H-imidazo[4,5-c]pyridin-4-amine;
N-(2-(2,6-dimethylphenyl)-3H-imidazo[4,5-c]pyridin-4-yl)cyclopropanecarboxamide;
2-(2,6-dimethylphenyl)-N-(2,6-dimethylpyrimidin-4-yl)-3H-imidazo[4,5-c]pyridin-4-amine;
N-(2-(2,6-bis(trifluoromethyl)phenyl)-3H-imidazo[4,5-c]pyridin-4-yl)cyclopropanecarboxamide;
2-(2,6-bis(trifluoromethyl)phenyl)-N-(2,6-dimethylpyrimidin-4-yl)-3H-imidazo[4,5-c]pyridin-4-amine;
N-(2-(2,6-difluorophenyl)-3H-imidazo[4,5-c]pyridin-4-yl)cyclopropanecarboxamide;
2-(2,6-difluorophenyl)-N-(2,6-dimethylpyrimidin-4-yl)-3H-imidazo[4,5-c]pyridin-4-amine;
2-(2-chlorophenyl)-N-(2,6-dimethylpyrimidin-4-yl)-3H-imidazo[4,5-c]pyridin-4-amine;
2-(2-chlorophenyl)-N-(pyrimidin-4-yl)-3H-imidazo[4,5-c]pyridin-4-amine;
2-(2-chlorophenyl)-N-(pyrazin-2-yl)-3H-imidazo[4,5-c]pyridin-4-amine;
2-(2-chlorophenyl)-N-(pyridin-2-yl)-3H-imidazo[4,5-c]pyridin-4-amine;
2-(2-chlorophenyl)-N-(1H-pyrazol-4-yl)-3H-imidazo[4,5-c]pyridin-4-amine;
N-(2-(2-chlorophenyl)-3H-imidazo[4,5-c]pyridin-4-yl)cyclopropanecarboxamide;
N-(2-(2-chlorophenyl)-3H-imidazo[4,5-c]pyridin-4-yl)acetamide;
1-(2-(2-chlorophenyl)-3H-imidazo[4,5-c]pyridin-4-yl)-3-methylurea; Cyclopropanecarboxylic acid [2-(2,6-dichloro-phenyl)-1H-imidazo[4,5-c]pyridin-4-yl]-amide;
(2-Chloro-6-methyl-pyrimidin-4-yl)-[2-(2,6-dichloro-phenyl)-3H-imidazo[4,5-c]pyridin-4-yl]-amine;
[2-(2,6-Dichloro-phenyl)-3H-imidazo[4,5-c]pyridin-4-yl]-(6-morpholin-4-yl-pyridazin-3-yl)-amine;
(1S,2S)-2-Fluoro-cyclopropanecarboxylic acid [2-(2,6-dichloro-phenyl)-1-methyl-1H-imidazo[4,5-c]pyridin-4-yl]-amide;
(1R,2R)-2-Fluoro-cyclopropanecarboxylic acid [2-(2,6-dichloro-phenyl)-1-methyl-1H-imidazo[4,5-c]pyridin-4-yl]-amide;
(1S,2S)-2-Fluoro-cyclopropanecarboxylic acid [2-(2-chloro-6-fluoro-phenyl)-3H-imidazo[4,5-c]pyridin-4-yl]-amide;
(1R,2R)-2-Fluoro-cyclopropanecarboxylic acid [2-(2-chloro-6-fluoro-phenyl)-3H-imidazo[4,5-c]pyridin-4-yl]-amide;
[2-(2,6-Dichloro-phenyl)-3H-imidazo[4,5-c]pyridin-4-yl]-(6-methyl-2-morpholin-4-yl-pyrimidin-4-yl)-amine;
[2-(2,6-Dichloro-phenyl)-1-methyl-1H-imidazo[4,5-c]pyridin-4-yl]-(6-methyl-2-morpholin-4-yl-pyrimidin-4-yl)-amine;
2-(2,6-dichlorophenyl)-N-(6-methyl-2-(trifluoromethyl)pyrimidin-4-yl)-3H-imidazo[4,5-c]pyridin-4-amine;
(2-Chloro-6-methyl-pyrimidin-4-yl)-[2-(2,6-dichloro-phenyl)-3H-imidazo[4,5-c]pyridin-4-yl]-amine;
(1-(4-(2-(2,6-dichlorophenyl)-3H-imidazo[4,5-c]pyridin-4-ylamino)-6-methylpyrimidin-2-yl)pyrrolidine-2,5-diyl)dimethanol;
2,2′-(4-(2-(2,6-dichlorophenyl)-3H-imidazo[4,5-c]pyridin-4-ylamino)-6-methylpyrimidin-2-ylazanediyl)diethanol;
2((4-(2-(2,6-dichlorophenyl)-3H-imidazo[4,5-c]pyridin-4-ylamino)-6-methylpyrimidin-2-yl)(methyl)amino)ethanol;

N⁴-(2-(2,6-dichlorophenyl)-3H-imidazo[4,5-c]pyridin-4-yl)-N²,N²′6-trimethylpyrimidine-2,4-diamine;
2-(2,6-dichlorophenyl)-N-(2-methoxypyrimidin-4-yl)-3H-imidazo[4,5-c]pyridin-4-amine;
2-(2,6-dichlorophenyl)-N-(6-methoxypyridin-2-yl)-3H-imidazo[4,5-c]pyridin-4-amine;
[2-(2-Chloro-6-fluoro-phenyl)-3H-imidazo[4,5-c]pyridin-4-yl]-(6-methyl-2-morpholin-4-yl-pyrimidin-4-yl)-amine;
[2-(2,6-Dichloro-phenyl)-1-methyl-1H-imidazo[4,5-c]pyridin-4-yl]-(2,6-dimethyl-pyrimidin-4-yl)-amine;
2-(2,6-dichlorophenyl)-N-phenyl-3H-imidazo[4,5-c]pyridin-4-amine;
methyl 2-(2,6-dichlorophenyl)-3H-imidazo[4,5-c]pyridin-4-ylcarbamate;
2-(2,6-dichlorophenyl)-N-(6-methoxypyrimidin-4-yl)-3H-imidazo[4,5-c]pyridin-4-amine;
2-(2,6-dichlorophenyl)-N-(5-morpholinopyrazin-2-yl)-3H-imidazo[4,5-c]pyridin-4-amine;
2-(2,6-dichlorophenyl)-N-(5-(4-methylpiperazin-1-yl)pyrazin-2-yl)-3H-imidazo[4,5-c]pyridin-4-amine;
2-(4-(6-(2-(2,6-dichlorophenyl)-3H-imidazo[4,5-c]pyridin-4-ylamino)pyridin-3-yl)piperazin-1-yl)ethanol;
2-(4-(2-(2-(2,6-dichlorophenyl)-3H-imidazo[4,5-c]pyridin-4-ylamino)pyridin-4-yl)piperazin-1-yl)ethanol;
2-(2,6-dichlorophenyl)-N-(pyridin-4-yl)-3H-imidazo[4,5-c]pyridin-4-amine;
2-(2,6-dichlorophenyl)-N-(pyridin-3-yl)-3H-imidazo[4,5-c]pyridin-4-amine;
N-(2-(2,6-dichlorophenyl)-3H-imidazo[4,5-c]pyridin-4-yl)-5-methylisoxazol-3-amine;
2-(2,6-dichlorophenyl)-N-(6-methylpyrimidin-4-yl)-3H-imidazo[4,5-c]pyridin-4-amine;
1-(6-(2-(2,6-dichlorophenyl)-3H-imidazo[4,5-c]pyridin-4-ylamino)pyrimidin-4-yl)piperidin-4-01;
2-(2,6-dichlorophenyl)-N-(6-(1,1-dioxothiomorpholin-4-yl)pyrimidin-4-yl)-3H-imidazo[4,5-c]pyridin-4-amine;
N-(2-(2,6-dichlorophenyl)-3H-imidazo[4,5-c]pyridin-4-yl)thiazol-2-amine;
2-(2,6-dichlorophenyl)-N-(2-methyl-6-morpholinopyrimidin-4-yl)-3H-imidazo[4,5-c]pyridin-4-amine;
N4-(2-(2,6-dichlorophenyl)-3H-imidazo[4,5-c]pyridin-4-yl)-N6,N6,2-trimethylpyrimidine-4,6-diamine;
N-(6-(azetidin-1-yl)-2-methylpyrimidin-4-yl)-2-(2,6-dichlorophenyl)-3H-imidazo[4,5-c]pyridin-4-amine;
2-(2-chloro-6-fluorophenyl)-N-(4-morpholinopyridin-2-yl)-3H-imidazo[4,5-c]pyridin-4-amine;
(2-(2-(2,6-dichlorophenyl)-3H-imidazo[4,5-c]pyridin-4-ylamino)pyridin-4-yl)methanol;
(6-(2-(2,6-dichlorophenyl)-3H-imidazo[4,5-c]pyridin-4-ylamino)pyridin-3-yl)methanol;
2-((6-(2-(2,6-dichlorophenyl)-3H-imidazo[4,5-c]pyridin-4-ylamino)pyrimidin-4-yl)(methyl)amino)ethanol;
2-(2,6-dichlorophenyl)-N-(6-(pyrrolidin-1-yl)pyrimidin-4-yl)-3H-imidazo[4,5-c]pyridin-4-amine;
N-(2-(2,6-dichlorophenyl)-3H-imidazo[4,5-c]pyridin-4-yl)-4-methylthiazol-2-amine;
N-(2-(2,6-dichlorophenyl)-3H-imidazo[4,5-c]pyridin-4-yl)-5-methylthiazol-2-amine;
2-(4-(6-(2-(2-chloro-6-fluorophenyl)-3H-imidazo[4,5-c]pyridin-4-ylamino)-2-methylpyrimidin-4-yl)piperazin-1-yl)ethanol;

N⁴-(2-(2-chloro-6-fluorophenyl)-3H-imidazo[4,5-c]pyridin-4-yl)-N⁶,N⁶-dimethylpyrimidine-4,6-diamine;
2-(2-chloro-6-fluorophenyl)-N-(6-methoxypyrimidin-4-yl)-3H-imidazo[4,5-c]pyridin-4-amine;
2-(2-chloro-6-fluorophenyl)-N-(6-methylpyrimidin-4-yl)-3H-imidazo[4,5-c]pyridin-4-amine;
2-(2-chloro-6-fluorophenyl)-N-(5-morpholinopyrazin-2-yl)-3H-imidazo[4,5-c]pyridin-4-amine;
(2-(2-(2-chloro-6-fluorophenyl)-3H-imidazo[4,5-c]pyridin-4-ylamino)pyridin-4-yl)methanol;
2-(2-chloro-6-fluorophenyl)-N-(2-methyl-6-morpholinopyrimidin-4-yl)-3H-imidazo[4,5-c]pyridin-4-amine;
N⁴-(2-(2-chloro-6-fluorophenyl)-3H-imidazo[4,5-c]pyridin-4-yl)-N⁶,N^{6,2}-trimethylpyrimidine-4,6-diamine;
2-(2-chloro-6-fluorophenyl)-N-(6-(pyrrolidin-1-yl)pyrimidin-4-yl)-3H-imidazo[4,5-c]pyridin-4-amine;
(6-(2-(2-chloro-6-fluorophenyl)-3H-imidazo[4,5-c]pyridin-4-ylamino)pyridin-3-yl)methanol;
1-(6-(2-(2-chloro-6-fluorophenyl)-3H-imidazo[4,5-c]pyridin-4-ylamino)pyrimidin-4-yl)piperidin-4-ol;
2-((6-(2-(2-chloro-6-fluorophenyl)-3H-imidazo[4,5-c]pyridin-4-ylamino)pyrimidin-4-yl)(methyl)amino)ethanol;
2-(2-chlorophenyl)-N-(6-methyl-2-morpholinopyrimidin-4-yl)-3H-imidazo[4,5-c]pyridin-4-amine;
4-[8-(2,6-Dichloro-phenyl)-7H-purin-6-ylamino]-pyrimidine-2-carbonitrile;
8-(2,6-dichlorophenyl)-N-(pyridin-2-yl)-9H-purin-6-amine;
8-(2,6-dichlorophenyl)-N-(pyridazin-3-yl)-7H-purin-6-amine;
[8-(2,6-Dichloro-phenyl)-7H-purin-6-yl]-(2,6-dimethyl-pyrimidin-4-yl)-amine;
2-(4-{6-[8-(2,6-Dichloro-phenyl)-7H-purin-6-ylamino]-2-methyl-pyrimidin-4-yl}-piperazin-1-yl)-ethanol;
8-(2-chlorophenyl)-N-(pyridin-2-yl)-7H-purin-6-amine;
8-(2-chlorophenyl)-N-(pyrazin-2-yl)-7H-purin-6-amine;
N-(8-(2,6-dichlorophenyl)-7H-purin-6-yl)isobutyramide;
1-(8-(2,6-dichlorophenyl)-7H-purin-6-yl)-3-methylurea;
8-(2,6-dichlorophenyl)-N-(pyrazin-2-yl)-7H-purin-6-amine;
8-(2,6-dichlorophenyl)-N-(5-morpholinopyridin-2-yl)-7H-purin-6-amine;
2-(4-(6-(8-(2,6-dichlorophenyl)-7H-purin-6-ylamino)pyridin-3-yl)piperazin-1-yl)ethanol;
8-(2,6-dichlorophenyl)-N-(4-morpholinopyridin-2-yl)-7H-purin-6-amine;
8-(2,6-dichlorophenyl)-N-(4-methylpyridin-2-yl)-7H-purin-6-amine;
8-(2,6-dichlorophenyl)-N-(6-methylpyrimidin-4-yl)-7H-purin-6-amine;
2-(8-(2,6-dichlorophenyl)-7H-purin-6-ylamino)isonicotinonitrile;
8-(2,6-dichlorophenyl)-N-(5-methylpyridin-2-yl)-7H-purin-6-amine;
6-(8-(2,6-dichlorophenyl)-7H-purin-6-ylamino)nicotinonitrile;
8-(2,6-dichlorophenyl)-N-(6-morpholinopyrimidin-4-yl)-9H-purin-6-amine;
8-(2,6-dichlorophenyl)-N-(6-(4-methylpiperazin-1-yl)pyrimidin-4-yl)-7H-purin-6-amine;
8-(2,6-dichlorophenyl)-N-(5-methylpyrazin-2-yl)-7H-purin-6-amine;
5-(8-(2,6-dichlorophenyl)-7H-purin-6-ylamino)pyrazine-2-carbonitrile;
2-(4-(2-(8-(2,6-dichlorophenyl)-7H-purin-6-ylamino)pyridin-4-yl)piperazin-1-yl)ethanol;
2-(4-(6-(8-(2,6-dichlorophenyl)-7H-purin-6-ylamino)pyrimidin-4-yl)piperazin-1-yl)ethanol;
2-(6-(8-(2,6-dichlorophenyl)-7H-purin-6-ylamino)pyrimidin-4-ylamino)ethanol;
3,5-dichloro-4-(6-(pyrimidin-4-ylamino)-7H-purin-8-yl)benzonitrile;
3,5-dichloro-4-(6-(pyrazin-2-ylamino)-7H-purin-8-yl)benzonitrile;
3,5-dichloro-4-(6-(pyridazin-3-ylamino)-7H-purin-8-yl)benzonitrile;
3,5-dichloro-4-(6-(6-methylpyrimidin-4-ylamino)-7H-purin-8-yl)benzonitrile;
3,5-dichloro-4-(6-(6-morpholinopyrimidin-4-ylamino)-7H-purin-8-yl)benzonitrile;
3,5-dichloro-4-(6-(6-(4-(2-hydroxyethyl)piperazin-1-yl)pyrimidin-4-ylamino)-7H-purin-8-yl)benzonitrile;
3,5-dichloro-4-(6-(6-(4-methylpiperazin-1-yl)pyrimidin-4-ylamino)-7H-purin-8-yl)benzonitrile;
3,5-dichloro-4-(6-(pyridin-2-ylamino)-7H-purin-8-yl)benzonitrile;
N-(8-(2,6-dichloro-4-cyanophenyl)-9H-purin-6-yl)cyclopropanecarboxamide;
3,5-dichloro-4-(6-(5-morpholinopyridin-2-ylamino)-7H-purin-8-yl)benzonitrile;
3,5-dichloro-4-(6-(4-methylpyridin-2-ylamino)-7H-purin-8-yl)benzonitrile;
2-(8-(2,6-dichloro-4-cyanophenyl)-7H-purin-6-ylamino)isonicotinonitrile;
3,5-dichloro-4-(6-(6-methoxypyrimidin-4-ylamino)-7H-purin-8-yl)benzonitrile;
8-(2-chloro-6-fluorophenyl)-N-(pyrimidin-4-yl)-7H-purin-6-amine;
8-(2-chloro-6-fluorophenyl)-N-(pyrazin-2-yl)-7H-purin-6-amine;
8-(2-chloro-6-fluorophenyl)-N-(pyridazin-3-yl)-7H-purin-6-amine;
8-(2-chloro-6-fluorophenyl)-N-(pyridin-2-yl)-7H-purin-6-amine;
8-(2-chloro-6-fluorophenyl)-N-(6-methylpyrimidin-4-yl)-7H-purin-6-amine;
8-(2-chloro-6-fluorophenyl)-N-(1H-pyrazol-4-yl)-7H-purin-6-amine;
N-(8-(2-chloro-6-fluorophenyl)-7H-purin-6-yl)isoxazol-3-amine;
methyl 8-(2-chloro-6-fluorophenyl)-7H-purin-6-ylcarbamate;
1-(8-(2-chloro-6-fluorophenyl)-7H-purin-6-yl)-3-methylurea;
N-(8-(2-chloro-6-fluorophenyl)-7H-purin-6-yl)cyclopropanecarboxamide;
8-(2-chloro-6-fluorophenyl)-N-(6-morpholinopyrimidin-4-yl)-7H-purin-6-amine;
2-(4-(6-(8-(2-chloro-6-fluorophenyl)-7H-purin-6-ylamino)pyrimidin-4-yl)piperazin-1-yl)ethanol;
8-(2-chloro-6-fluorophenyl)-N-(6-methoxypyrimidin-4-yl)-7H-purin-6-amine;
8-(2,6-dichlorophenyl)-9-methyl-N-phenyl-9H-purin-6-amine; and
8-(2,6-dichlorophenyl)-9-methyl-N-(pyridin-4-yl)-9H-purin-6-amine

Another embodiment includes a compound of Formulas Ia-Ib, stereoisomers or pharmaceutically acceptable salts thereof, selected from:
2-(2,6-dichlorophenyl)-N-(pyridin-2-yl)-1H-imidazo[4,5-c]pyridin-4-amine;
2-(4-(4-(2-(2,6-dichlorophenyl)-3H-imidazo[4,5-c]pyridin-4-ylamino)-6-methylpyrimidin-2-yl)piperazin-1-yl)ethanol;
2-(2,6-dichlorophenyl)-N-(1H-pyrazol-4-yl)-3H-imidazo[4,5-c]-pyridin-4-amine;
N-(6-chloropyrimidin-4-yl)-2-(2,6-dichlorophenyl)-3H-imidazo-[4,5-c]pyridin-4-amine;
2-(2,6-dichlorophenyl)-N-(6-morpholinopyrimidin-4-yl)-3H-imidazo[4,5-c]pyridin-4-amine;
2-(2-chloro-6-fluorophenyl)-N-(pyrazin-2-yl)-3H-imidazo[4,5-c]-pyridin-4-amine;
N-(2-(2,6-dichlorophenyl)-3H-imidazo[4,5-c]pyridin-4-yl)thiazole-5-carboxamide;
(1R,2R)—N-(2-(2,6-dichlorophenyl)-3H-imidazo[4,5-c]pyridin-4-yl)-2-fluorocyclopropanecarboxamide;
(1S,2S)—N-(2-(2,6-dichlorophenyl)-3H-imidazo[4,5-c]pyridin-4-yl)-2-fluorocyclopropanecarboxamide;
N-(2-(2,6-dichloro-4-cyanophenyl)-3H-imidazo[4,5-c]pyridin-4-yl)cyclopropanecarboxamide;
1-(2-(2,6-dichlorophenyl)-3H-imidazo[4,5-c]pyridin-4-yl)-3-methylurea;
N-(2-(2,6-dichlorophenyl)-1-methyl-1H-imidazo[4,5-c]pyridine-4-yl)cyclopropanecarboxamide;
N-(2-(2,6-dichlorophenyl)-3-methyl-3H-imidazo[4,5-c]pyridine-4-yl)cyclopropanecarboxamide;
8-(2,6-dichlorophenyl)-N-(pyrimidin-4-yl)-9H-purin-6-amine;
N-(8-(2,6-dichlorophenyl)-7H-purin-6-yl)cyclopropanecarboxamide;
2-(2,6-dichlorophenyl)-N-(2,6-dimethylpyrimidin-4-yl)-7-fluoro-1H-imidazo[4,5-c]pyridin-4-amine;
N-(2-(2,6-dichlorophenyl)-7-fluoro-1H-imidazo[4,5-c]pyridin-4-yl)cyclopropanecarboxamide;
2-(2,6-dichlorophenyl)-N-(pyrimidin-4-yl)-1H-imidazo[4,5-c]pyridine-4-amine;
2-(2,6-dichlorophenyl)-N-(4-(2-(pyrrolidin-1-yl)ethoxy)phenyl)-3H-imidazo[4,5-c]pyridin-4-amine;
[2-(2,6-dichloro-phenyl)-1H-imidazo[4,5-c]pyridin-4-yl]-(2,6-dimethyl-pyrimidin-4-yl)-amine;
2-(2,6-dichlorophenyl)-N-(pyrimidin-2-yl)-1H-imidazo[4,5-c]-pyridin-4-amine;
2-(2,6-dichlorophenyl)-N-(pyrazin-2-yl)-3H-imidazo[4,5-c]pyridine-4-amine;
2-(2,6-dichlorophenyl)-N-(pyridazin-3-yl)-1H-imidazo[4,5-c]-pyridin-4-amine;
N-(2-(2,6-dichlorophenyl)-1H-imidazo[4,5-c]pyridin-4-yl)-3-methyl-isoxazol-5-amine;
N-(2-(2,6-dichlorophenyl)-3H-imidazo[4,5-c]pyridin-4-yl)-benzamide;
N-(2-(2,6-dichlorophenyl)-3H-imidazo[4,5-c]pyridin-4-ylacetamide;
N-(2-(2,6-dichlorophenyl)-3H-imidazo[4,5-c]pyridin-4-yl)propionamide;
N-(2-(2,6-dichlorophenyl)-3H-imidazo[4,5-c]pyridin-4-yl)-isobutyramide;
N-(2-(2,6-dichlorophenyl)-3H-imidazo[4,5-c]pyridin-4-yl)pivalamide;
2-(4-(6-(2-(2,6-dichlorophenyl)-3H-imidazo[4,5-c]pyridine-4-ylamino)-2-methylpyrimidin-4-yl)piperazin-1-yl)ethanol;

N⁴-(2-(2,6-dichlorophenyl)-3H-imidazo[4,5-c]pyridin-4-yl)-N⁶,N⁶-dimethylpyrimidine-4,6-diamine;

N⁴-(2-(2,6-dichlorophenyl)-3H-imidazo[4,5-c]pyridin-4-yl)-N2,N2-dimethylpyrimidine-2,4-diamine;
2-(2,6-dichlorophenyl)-N-(2-methylpyrimidin-4-yl)-3H-imidazo-[4,5-c]pyridin-4-amine;
2-(2,6-dichlorophenyl)-N-(4-methylpyridin-2-yl)-3H-imidazo[4,5-c]-pyridin-4-amine;
2-(2,6-dichlorophenyl)-N-(5-methylpyridin-2-yl)-3H-imidazo-[4,5-c]pyridin-4-amine;
2-(4-(6-(2-(2,6-dichlorophenyl)-3H-imidazo[4,5-c]pyridine-4-ylamino)pyrimidin-4-yl)piperazin-1-yl)ethanol;
2-(2,6-dichlorophenyl)-N-(4-morpholinopyridin-2-yl)-3H-imidazo[4,5-c]pyridin-4-amine;
2-(2,6-dichlorophenyl)-N-(5-morpholinopyridin-2-yl)-3H-imidazo[4,5-c]pyridin-4-amine;
2-(2,6-dichlorophenyl)-N-(6-(4-methylpiperazin-1-yl)pyrimidin-4-yl)-3H-imidazo[4,5-c]pyridin-4-amine;
2-(6-(2-(2,6-dichlorophenyl)-3H-imidazo[4,5-c]pyridin-4-ylamino)-pyrimidin-4-ylamino)ethanol;
2-(2-(2,6-dichlorophenyl)-3H-imidazo[4,5-c]pyridin-4-ylamino)isonicotinonitrile;
6-(2-(2,6-dichlorophenyl)-3H-imidazo[4,5-c]pyridin-4-ylamino)nicotinonitrile;
2-(2,6-dichlorophenyl)-N-(5-methylpyrazin-2-yl)-3H-imidazo[4,5-c]pyridin-4-amine;
5-(2-(2,6-dichlorophenyl)-3H-imidazo[4,5-c]pyridin-4-ylamino)pyrazine-2-carbonitrile;
2-(4-(5-(2-(2,6-dichlorophenyl)-3H-imidazo[4,5-c]pyridin-4-ylamino)pyrazin-2-yl)piperazin-1-yl)ethanol;
2-(2-chloro-6-fluorophenyl)-N-(2,6-dimethylpyrimidin-4-yl)-3H-imidazo[4,5-c]pyridin-4-amine;
N-(2-(2-chloro-6-fluorophenyl)-3H-imidazo[4,5-c]pyridin-4-yl)-cyclopropanecarboxamide;
2-(2-chloro-6-fluorophenyl)-N-(1H-pyrazol-4-yl)-3H-imidazo[4,5-c]pyridin-4-amine;
2-(2-chloro-6-fluorophenyl)-N-(pyridin-2-yl)-1H-imidazo[4,5-c]pyridin-4-amine;
2-(2-chloro-6-fluorophenyl)-N-(5-methylpyridin-2-yl)-3H-imidazo[4,5-c]pyridin-4-amine;
2-(2-(2-chloro-6-fluorophenyl)-3H-imidazo[4,5-c]pyridin-4-ylamino)isonicotinonitrile;
2-(2-chloro-6-fluorophenyl)-N-(4-methylpyridin-2-yl)-3H-imidazo[4,5-c]pyridin-4-amine;
6-(2-(2-chloro-6-fluorophenyl)-3H-imidazo[4,5-c]pyridin-4-ylamino)nicotinonitrile;
2-(2-chloro-6-fluorophenyl)-N-(pyridin-2-yl)-3H-imidazo[4,5-c]pyridin-4-amine;
2-(4-(2-(2-(2-chloro-6-fluorophenyl)-3H-imidazo[4,5-c]pyridin-4-ylamino)pyridin-4-yl)piperazin-1-yl)ethanol;
2-(2-chloro-6-fluorophenyl)-N-(5-morpholinopyridin-2-yl)-3H-imidazo[4,5-c]pyridin-4-amine;
2-(4-(6-(2-(2-chloro-6-fluorophenyl)-3H-imidazo[4,5-c]pyridin-4-ylamino)pyridin-3-yl)piperazin-1-yl)ethanol;
2-(2-chloro-6-fluorophenyl)-N-(5-methylpyrazin-2-yl)-3H-imidazo[4,5-c]pyridin-4-amine;
5-(2-(2-chloro-6-fluorophenyl)-3H-imidazo[4,5-c]pyridin-4-ylamino)pyrazine-2-carbonitrile;
methyl 5-(2-(2-chloro-6-fluorophenyl)-3H-imidazo[4,5-c]pyridin-4-ylamino)pyrazine-2-carbimidate;
1-(2-(2-chloro-6-fluorophenyl)-3H-imidazo[4,5-c]pyridin-4-yl)-3-methylurea;
2-(2-chloro-6-fluorophenyl)-N-(pyrimidin-4-yl)-3H-imidazo[4,5-c]pyridin-4-amine;
2-(2-chloro-6-fluorophenyl)-N-(6-morpholinopyrimidin-4-yl)-3H-imidazo[4,5-c]pyridin-4-amine;
2-(4-(6-(2-(2-chloro-6-fluorophenyl)-3H-imidazo[4,5-c]pyridine-4-ylamino)pyrimidin-4-yl)piperazin-1-yl)ethanol;
2-(2-chloro-6-fluorophenyl)-N-(6-(4-methylpiperazin-1-yl)-pyrimidin-4-yl)-3H-imidazo[4,5-c]pyridin-4-amine;
2-(6-(2-(2-chloro-6-fluorophenyl)-3H-imidazo[4,5-c]pyridin-4-ylamino)pyrimidin-4-ylamino)ethanol;
3,5-dichloro-4-(4-(4-methylpyridin-2-ylamino)-3H-imidazo[4,5-c]pyridin-2-yl)benzonitrile;
3,5-dichloro-4-(4-(2,6-dimethylpyrimidin-4-ylamino)-3H-imidazo[4,5-c]pyridin-2-yl)benzonitrile;
3,5-dichloro-4-(4-(5-(4-(2-hydroxyethyl)piperazin-1-yl)pyridin-2-ylamino)-3H-imidazo[4,5-c]pyridin-2-yl)benzonitrile;
3,5-dichloro-4-(4-(4-morpholinopyridin-2-ylamino)-3H-imidazo[4,5-c]pyridin-2-yl)benzonitrile;
3,5-dichloro-4-(4-(4-(4-(2-hydroxyethyl)piperazin-1-yl)pyridin-2-ylamino)-3H-imidazo[4,5-c]pyridin-2-yl)benzonitrile;
3,5-dichloro-4-(4-(5-morpholinopyridin-2-ylamino)-3H-imidazo[4,5-c]pyridin-2-yl)benzonitrile;
3,5-dichloro-4-(4-(5-methylpyridin-2-ylamino)-3H-imidazo[4,5-c]pyridin-2-yl)benzonitrile;
3,5-dichloro-4-(4-(6-morpholinopyrimidin-4-ylamino)-3H-imidazo[4,5-c]pyridin-2-yl)benzonitrile;
3,5-dichloro-4-(4-(6-(4-methylpiperazin-1-yl)pyrimidin-4-ylamino)-3H-imidazo[4,5-c]pyridin-2-yl)benzonitrile;
3,5-dichloro-4-(4-(6-methoxypyrimidin-4-ylamino)-3H-imidazo[4,5-c]pyridin-2-yl)benzonitrile;
2-(2-(2,6-dichloro-4-cyanophenyl)-3H-imidazo[4,5-c]pyridin-4-ylamino)isonicotinonitrile;
6-(2-(2,6-dichloro-4-cyanophenyl)-3H-imidazo[4,5-c]pyridin-4-ylamino)nicotinonitrile;
3,5-dichloro-4-(4-(5-methylthiazol-2-ylamino)-3H-imidazo[4,5-c]pyridin-2-yl)benzonitrile;
3,5-dichloro-4-(4-(4-methylthiazol-2-ylamino)-3H-imidazo[4,5-c]pyridin-2-yl)benzonitrile;
3,5-dichloro-4-(4-(thiazol-2-ylamino)-3H-imidazo[4,5-c]pyridin-2-yl)benzonitrile;
3,5-dichloro-4-(4-(6-methylpyrimidin-4-ylamino)-3H-imidazo[4,5-c]pyridin-2-yl)benzonitrile;
3,5-dichloro-4-(4-(6-((2-hydroxyethyl)(methyl)amino)pyrimidin-4-ylamino)-3H-imidazo[4,5-c]pyridin-2-yl)benzonitrile;
3,5-dichloro-4-(4-(6-(pyrrolidin-1-yl)pyrimidin-4-ylamino)-3H-imidazo[4,5-c]pyridin-2-yl)benzonitrile;
3,5-dichloro-4-(4-(6-(4-hydroxypiperidin-1-yl)pyrimidin-4-ylamino)-3H-imidazo[4,5-c]pyridin-2-yl)benzonitrile;
3,5-dichloro-4-(4-(6-(dimethylamino)pyrimidin-4-ylamino)-3H-imidazo[4,5-c]pyridin-2-yl)benzonitrile;
N-(2-(2-chloro-4-(methylsulfonyl)phenyl)-3H-imidazo[4,5-c]-pyridin-4-yl)cyclopropanecarboxamide;
2-(2-chloro-4-(methylsulfonyl)phenyl)-N-(2,6-dimethylpyrimidin-4-yl)-3H-imidazo[4,5-c]pyridin-4-amine;
N-(2-(2-(trifluoromethyl)phenyl)-1H-imidazo[4,5-c]pyridin-4-yl)cyclopropanecarboxamide;
N-(2,6-dimethylpyrimidin-4-yl)-2-(2-(trifluoromethyl)phenyl)-3H-imidazo[4,5-c]pyridin-4-amine;
N-(2-(2-(trifluoromethoxy)phenyl)-1H-imidazo[4,5-c]pyridin-4-yl)cyclopropanecarboxamide;
N-(2,6-dimethylpyrimidin-4-yl)-2-(2-(trifluoromethoxy)phenyl)-3H-imidazo[4,5-c]pyridin-4-amine;
N-(2-(2,6-dimethylphenyl)-3H-imidazo[4,5-c]pyridin-4-yl)cyclopropanecarboxamide;
2-(2,6-dimethylphenyl)-N-(2,6-dimethylpyrimidin-4-yl)-3H-imidazo[4,5-c]pyridin-4-amine;
N-(2-(2,6-bis(trifluoromethyl)phenyl)-3H-imidazo[4,5-c]pyridin-4-yl)cyclopropanecarboxamide;
2-(2,6-bis(trifluoromethyl)phenyl)-N-(2,6-dimethylpyrimidin-4-yl)-3H-imidazo[4,5-c]pyridin-4-amine;
N-(2-(2,6-difluorophenyl)-3H-imidazo[4,5-c]pyridin-4-yl)cyclopropanecarboxamide;
2-(2,6-difluorophenyl)-N-(2,6-dimethylpyrimidin-4-yl)-3H-imidazo[4,5-c]pyridin-4-amine;
2-(2-chlorophenyl)-N-(2,6-dimethylpyrimidin-4-yl)-3H-imidazo[4,5-c]pyridin-4-amine;
2-(2-chlorophenyl)-N-(pyrimidin-4-yl)-3H-imidazo[4,5-c]pyridin-4-amine;
2-(2-chlorophenyl)-N-(pyrazin-2-yl)-3H-imidazo[4,5-c]pyridin-4-amine;
2-(2-chlorophenyl)-N-(pyridin-2-yl)-3H-imidazo[4,5-c]pyridin-4-amine;
2-(2-chlorophenyl)-N-(1H-pyrazol-4-yl)-3H-imidazo[4,5-c]pyridin-4-amine;
N-(2-(2-chlorophenyl)-3H-imidazo[4,5-c]pyridin-4-yl)cyclopropanecarboxamide;
N-(2-(2-chlorophenyl)-3H-imidazo[4,5-c]pyridin-4-yl)acetamide;
1-(2-(2-chlorophenyl)-3H-imidazo[4,5-c]pyridin-4-yl)-3-methylurea;
Cyclopropanecarboxylic acid [2-(2,6-dichloro-phenyl)-1H-imidazo[4,5-c]pyridin-4-yl]-amide;
(2-Chloro-6-methyl-pyrimidin-4-yl)-[2-(2,6-dichloro-phenyl)-3H-imidazo[4,5-c]pyridin-4-yl]-amine;
[2-(2,6-Dichloro-phenyl)-3H-imidazo[4,5-c]pyridin-4-yl]-(6-morpholin-4-yl-pyridazin-3-yl)-amine;
(1S,2S)-2-Fluoro-cyclopropanecarboxylic acid [2-(2,6-dichloro-phenyl)-1-methyl-1H-imidazo[4,5-c]pyridin-4-yl]-amide;
(1R,2R)-2-Fluoro-cyclopropanecarboxylic acid [2-(2,6-dichloro-phenyl)-1-methyl-1H-imidazo[4,5-c]pyridin-4-yl]-amide;
(1S,2S)-2-Fluoro-cyclopropanecarboxylic acid [2-(2-chloro-6-fluoro-phenyl)-3H-imidazo[4,5-c]pyridin-4-yl]-amide;
(1R,2R)-2-Fluoro-cyclopropanecarboxylic acid [2-(2-chloro-6-fluoro-phenyl)-3H-imidazo[4,5-c]pyridin-4-yl]-amide;
[2-(2,6-Dichloro-phenyl)-3H-imidazo[4,5-c]pyridin-4-yl]-(6-methyl-2-morpholin-4-yl-pyrimidin-4-yl)-amine;
[2-(2,6-Dichloro-phenyl)-1-methyl-1H-imidazo[4,5-c]pyridin-4-yl]-(6-methyl-2-morpholin-4-yl-pyrimidin-4-yl)-amine;
2-(2,6-dichlorophenyl)-N-(6-methyl-2-(trifluoromethyl)pyrimidin-4-yl)-3H-imidazo[4,5-c]pyridin-4-amine;
(2-Chloro-6-methyl-pyrimidin-4-yl)-[2-(2,6-dichloro-phenyl)-3H-imidazo[4,5-c]pyridin-4-yl]-amine;
(1-(4-(2-(2,6-dichlorophenyl)-3H-imidazo[4,5-c]pyridin-4-ylamino)-6-methylpyrimidin-2-yl)pyrrolidine-2,5-diyl)dimethanol;
2,2′-(4-(2-(2,6-dichlorophenyl)-3H-imidazo[4,5-c]pyridin-4-ylamino)-6-methylpyrimidin-2-ylazanediyl)diethanol;
2-((4-(2-(2,6-dichlorophenyl)-3H-imidazo[4,5-c]pyridin-4-ylamino)-6-methylpyrimidin-2-yl)(methyl)amino)ethanol;
N⁴-(2-(2,6-dichlorophenyl)-3H-imidazo[4,5-c]pyridin-4-yl)-N²,N²′6-trimethylpyrimidine-2,4-diamine;
2-(2,6-dichlorophenyl)-N-(2-methoxypyrimidin-4-yl)-3H-imidazo[4,5-c]pyridin-4-amine;
2-(2,6-dichlorophenyl)-N-(6-methoxypyridin-2-yl)-3H-imidazo[4,5-c]pyridin-4-amine;
[2-(2-Chloro-6-fluoro-phenyl)-3H-imidazo[4,5-c]pyridin-4-yl]-(6-methyl-2-morpholin-4-yl-pyrimidin-4-yl)-amine;
[2-(2,6-Dichloro-phenyl)-1-methyl-1H-imidazo[4,5-c]pyridin-4-yl]-(2,6-dimethyl-pyrimidin-4-yl)-amine;
2-(2,6-dichlorophenyl)-N-phenyl-3H-imidazo[4,5-c]pyridin-4-amine;
methyl 2-(2,6-dichlorophenyl)-3H-imidazo[4,5-c]pyridin-4-ylcarbamate;
2-(2,6-dichlorophenyl)-N-(6-methoxypyrimidin-4-yl)-3H-imidazo[4,5-c]pyridin-4-amine;
2-(2,6-dichlorophenyl)-N-(5-morpholinopyrazin-2-yl)-3H-imidazo[4,5-c]pyridin-4-amine;
2-(2,6-dichlorophenyl)-N-(5-(4-methylpiperazin-1-yl)pyrazin-2-yl)-3H-imidazo[4,5-c]pyridin-4-amine;
2-(4-(6-(2-(2,6-dichlorophenyl)-3H-imidazo[4,5-c]pyridin-4-ylamino)pyridin-3-yl)piperazin-1-yl)ethanol;
2-(4-(2-(2-(2,6-dichlorophenyl)-3H-imidazo[4,5-c]pyridin-4-ylamino)pyridin-4-yl)piperazin-1-yl)ethanol;
2-(2,6-dichlorophenyl)-N-(pyridin-4-yl)-3H-imidazo[4,5-c]pyridin-4-amine;
2-(2,6-dichlorophenyl)-N-(pyridin-3-yl)-3H-imidazo[4,5-c]pyridin-4-amine;
N-(2-(2,6-dichlorophenyl)-3H-imidazo[4,5-c]pyridin-4-yl)-5-methylisoxazol-3-amine;
2-(2,6-dichlorophenyl)-N-(6-methylpyrimidin-4-yl)-3H-imidazo[4,5-c]pyridin-4-amine;
1-(6-(2-(2,6-dichlorophenyl)-3H-imidazo[4,5-c]pyridin-4-ylamino)pyrimidin-4-yl)piperidin-4-01;
2-(2,6-dichlorophenyl)-N-(6-(1,1-dioxothiomorpholin-4-yl)pyrimidin-4-yl)-3H-imidazo[4,5-c]pyridin-4-amine;
N-(2-(2,6-dichlorophenyl)-3H-imidazo[4,5-c]pyridin-4-yl)thiazol-2-amine;
2-(2,6-dichlorophenyl)-N-(2-methyl-6-morpholinopyrimidin-4-yl)-3H-imidazo[4,5-c]pyridin-4-amine;
N4-(2-(2,6-dichlorophenyl)-3H-imidazo[4,5-c]pyridin-4-yl)-N6,N6,2-trimethylpyrimidine-4,6-diamine;
N-(6-(azetidin-1-yl)-2-methylpyrimidin-4-yl)-2-(2,6-dichlorophenyl)-3H-imidazo[4,5-c]pyridin-4-amine;
2-(2-chloro-6-fluorophenyl)-N-(4-morpholinopyridin-2-yl)-3H-imidazo[4,5-c]pyridin-4-amine;
(2-(2-(2,6-dichlorophenyl)-3H-imidazo[4,5-c]pyridin-4-ylamino)pyridin-4-yl)methanol;
(6-(2-(2,6-dichlorophenyl)-3H-imidazo[4,5-c]pyridin-4-ylamino)pyridin-3-yl)methanol;
2-((6-(2-(2,6-dichlorophenyl)-3H-imidazo[4,5-c]pyridin-4-ylamino)pyrimidin-4-yl)(methyl)amino);
2-(2,6-dichlorophenyl)-N-(6-(pyrrolidin-1-yl)pyrimidin-4-yl)-3H-imidazo[4,5-c]pyridin-4-amine;
N-(2-(2,6-dichlorophenyl)-3H-imidazo[4,5-c]pyridin-4-yl)-4-methylthiazol-2-amine;
N-(2-(2,6-dichlorophenyl)-3H-imidazo[4,5-c]pyridin-4-yl)-5-methylthiazol-2-amine;
2-(4-(6-(2-(2-chloro-6-fluorophenyl)-3H-imidazo[4,5-c]pyridin-4-ylamino)-2-methylpyrimidin-4-yl)piperazin-1-yl)ethanol;

N⁴-(2-(2-chloro-6-fluorophenyl)-3H-imidazo[4,5-c]pyridin-4-yl)-N⁶,N⁶-dimethylpyrimidine-4,6-diamine;
2-(2-chloro-6-fluorophenyl)-N-(6-methoxypyrimidin-4-yl)-3H-imidazo[4,5-c]pyridin-4-amine;
2-(2-chloro-6-fluorophenyl)-N-(6-methylpyrimidin-4-yl)-3H-imidazo[4,5-c]pyridin-4-amine;
2-(2-chloro-6-fluorophenyl)-N-(5-morpholinopyrazin-2-yl)-3H-imidazo[4,5-c]pyridin-4-amine;
(2-(2-(2-chloro-6-fluorophenyl)-3H-imidazo[4,5-c]pyridin-4-ylamino)pyridin-4-yl)methanol;
2-(2-chloro-6-fluorophenyl)-N-(2-methyl-6-morpholinopyrimidin-4-yl)-3H-imidazo[4,5-c]pyridin-4-amine;

N⁴-(2-(2-chloro-6-fluorophenyl)-3H-imidazo[4,5-c]pyridin-4-yl)-N⁶,N^{6,2}-trimethylpyrimidine-4,6-diamine;
2-(2-chloro-6-fluorophenyl)-N-(6-(pyrrolidin-1-yl)pyrimidin-4-yl)-3H-imidazo[4,5-c]pyridin-4-amine;
(6-(2-(2-chloro-6-fluorophenyl)-3H-imidazo[4,5-c]pyridin-4-ylamino)pyridin-3-yl)methanol;
1-(6-(2-(2-chloro-6-fluorophenyl)-3H-imidazo[4,5-c]pyridin-4-ylamino)pyrimidin-4-yl)piperidin-4-ol;
2-((6-(2-(2-chloro-6-fluorophenyl)-3H-imidazo[4,5-c]pyridin-4-ylamino)pyrimidin-4-yl)(methyl)amino)ethanol;
2-(2-chlorophenyl)-N-(6-methyl-2-morpholinopyrimidin-4-yl)-3H-imidazo[4,5-c]pyridin-4-amine;
4-[8-(2,6-Dichloro-phenyl)-7H-purin-6-ylamino]-pyrimidine-2-carbonitrile;
8-(2,6-dichlorophenyl)-N-(pyridin-2-yl)-9H-purin-6-amine;
8-(2,6-dichlorophenyl)-N-(pyridazin-3-yl)-7H-purin-6-amine;
[8-(2,6-Dichloro-phenyl)-7H-purin-6-yl]-(2,6-dimethyl-pyrimidin-4-yl)-amine;
2-(4-{6-[8-(2,6-Dichloro-phenyl)-7H-purin-6-ylamino]-2-methyl-pyrimidin-4-yl}-piperazin-1-yl)-ethanol;
8-(2-chlorophenyl)-N-(pyridin-2-yl)-7H-purin-6-amine;
8-(2-chlorophenyl)-N-(pyrazin-2-yl)-7H-purin-6-amine;
N-(8-(2,6-dichlorophenyl)-7H-purin-6-yl)isobutyramide;
1-(8-(2,6-dichlorophenyl)-7H-purin-6-yl)-3-methylurea;
8-(2,6-dichlorophenyl)-N-(pyrazin-2-yl)-7H-purin-6-amine;
8-(2,6-dichlorophenyl)-N-(5-morpholinopyridin-2-yl)-7H-purin-6-amine;
2-(4-(6-(8-(2,6-dichlorophenyl)-7H-purin-6-ylamino)pyridin-3-yl)piperazin-1-yl)ethanol;
8-(2,6-dichlorophenyl)-N-(4-morpholinopyridin-2-yl)-7H-purin-6-amine;
8-(2,6-dichlorophenyl)-N-(4-methylpyridin-2-yl)-7H-purin-6-amine;
8-(2,6-dichlorophenyl)-N-(6-methylpyrimidin-4-yl)-7H-purin-6-amine;
2-(8-(2,6-dichlorophenyl)-7H-purin-6-ylamino)isonicotinonitrile;
8-(2,6-dichlorophenyl)-N-(5-methylpyridin-2-yl)-7H-purin-6-amine;
6-(8-(2,6-dichlorophenyl)-7H-purin-6-ylamino)nicotinonitrile;
8-(2,6-dichlorophenyl)-N-(6-morpholinopyrimidin-4-yl)-9H-purin-6-amine;
8-(2,6-dichlorophenyl)-N-(6-(4-methylpiperazin-1-yl)pyrimidin-4-yl)-7H-purin-6-amine;
8-(2,6-dichlorophenyl)-N-(5-methylpyrazin-2-yl)-7H-purin-6-amine;
5-(8-(2,6-dichlorophenyl)-7H-purin-6-ylamino)pyrazine-2-carbonitrile;
2-(4-(2-(8-(2,6-dichlorophenyl)-7H-purin-6-ylamino)pyridin-4-yl)piperazin-1-yl)ethanol;
2-(4-(6-(8-(2,6-dichlorophenyl)-7H-purin-6-ylamino)pyrimidin-4-yl)piperazin-1-yl)ethanol;
24648-(2,6-dichlorophenyl)-7H-purin-6-ylamino)pyrimidin-4-ylamino)ethanol;
3,5-dichloro-4-(6-(pyrimidin-4-ylamino)-7H-purin-8-yl)benzonitrile;
3,5-dichloro-4-(6-(pyrazin-2-ylamino)-7H-purin-8-yl)benzonitrile;
3,5-dichloro-4-(6-(pyridazin-3-ylamino)-7H-purin-8-yl)benzonitrile;
3,5-dichloro-4-(6-(6-methylpyrimidin-4-ylamino)-7H-purin-8-yl)benzonitrile;
3,5-dichloro-4-(6-(6-morpholinopyrimidin-4-ylamino)-7H-purin-8-yl)benzonitrile;
3,5-dichloro-4-(6-(6-(4-(2-hydroxyethyl)piperazin-1-yl)pyrimidin-4-ylamino)-7H-purin-8-yl)benzonitrile;
3,5-dichloro-4-(6-(6-(4-methylpiperazin-1-yl)pyrimidin-4-ylamino)-7H-purin-8-yl)benzonitrile;
3,5-dichloro-4-(6-(pyridin-2-ylamino)-7H-purin-8-yl)benzonitrile;
N-(8-(2,6-dichloro-4-cyanophenyl)-9H-purin-6-yl)cyclopropanecarboxamide;
3,5-dichloro-4-(6-(5-morpholinopyridin-2-ylamino)-7H-purin-8-yl)benzonitrile;
3,5-dichloro-4-(6-(4-methylpyridin-2-ylamino)-7H-purin-8-yl)benzonitrile;
2-(8-(2,6-dichloro-4-cyanophenyl)-7H-purin-6-ylamino)isonicotinonitrile;
3,5-dichloro-4-(6-(6-methoxypyrimidin-4-ylamino)-7H-purin-8-yl)benzonitrile;
8-(2-chloro-6-fluorophenyl)-N-(pyrimidin-4-yl)-7H-purin-6-amine;
8-(2-chloro-6-fluorophenyl)-N-(pyrazin-2-yl)-7H-purin-6-amine;
8-(2-chloro-6-fluorophenyl)-N-(pyridazin-3-yl)-7H-purin-6-amine;
8-(2-chloro-6-fluorophenyl)-N-(pyridin-2-yl)-7H-purin-6-amine;
8-(2-chloro-6-fluorophenyl)-N-(6-methylpyrimidin-4-yl)-7H-purin-6-amine;
8-(2-chloro-6-fluorophenyl)-N-(1H-pyrazol-4-yl)-7H-purin-6-amine;
N-(8-(2-chloro-6-fluorophenyl)-7H-purin-6-yl)isoxazol-3-amine;
methyl 8-(2-chloro-6-fluorophenyl)-7H-purin-6-ylcarbamate;
1-(8-(2-chloro-6-fluorophenyl)-7H-purin-6-yl)-3-methylurea;
N-(8-(2-chloro-6-fluorophenyl)-7H-purin-6-yl)cyclopropanecarboxamide;
8-(2-chloro-6-fluorophenyl)-N-(6-morpholinopyrimidin-4-yl)-7H-purin-6-amine;
2-(4-(6-(8-(2-chloro-6-fluorophenyl)-7H-purin-6-ylamino)pyrimidin-4-yl)piperazin-1-yl)ethanol;
8-(2-chloro-6-fluorophenyl)-N-(6-methoxypyrimidin-4-yl)-7H-purin-6-amine;
8-(2,6-dichlorophenyl)-9-methyl-N-phenyl-9H-purin-6-amine;
8-(2,6-dichlorophenyl)-9-methyl-N-(pyridin-4-yl)-9H-purin-6-amine;
N-4-(8-(2,6-dichlorophenyl)-9H-purin-6-yl)pyrimidine-4,6-diamine;
N-(2-(2,4,6-trichlorophenyl)-3H-imidazo[4,5-c]pyridin-4-yl)cyclopropanecarboxamide;
N-(2-(4-(aminomethyl)-2,6-dichlorophenyl)-3H-imidazo[4,5-c]pyridin-4-yl)cyclopropanecarboxamide;
N-(2,6-dimethylpyrimidin-4-yl)-2-(2,4,6-trichlorophenyl)-3H-imidazo[4,5-c]pyridin-4-amine;
2-(4-(aminomethyl)-2,6-dichlorophenyl)-N-(2,6-dimethylpyrimidin-4-yl)-3H-imidazo[4,5-c]pyridin-4-amine;
N-(2-(2,6-dichlorophenyl)-3H-imidazo[4,5-c]pyridin-4-yl)-2-hydroxyacetamide;
N-(2-(2,6-dichlorophenyl)-3H-imidazo[4,5-c]pyridin-4-yl)-2-(dimethylamino)acetamide;
N-(2-(2,6-dichlorophenyl)-3H-imidazo[4,5-c]pyridin-4-yl)cyclobutanecarboxamide;
(3,5-dichloro-4-(4-(2,6-dimethylpyrimidin-4-ylamino)-3H-imidazo[4,5-c]pyridin-2-yl)phenyl)methanol;
methyl 2-(2-chloro-6-fluorophenyl)-3H-imidazo[4,5-c]pyridin-4-ylcarbamate;
2-(2-bromo-6-chlorophenyl)-N-(pyrimidin-4-yl)-3H-imidazo[4,5-c]pyridin-4-amine;
N-(2-(2-bromo-6-chlorophenyl)-3H-imidazo[4,5-c]pyridin-4-yl)cyclopropanecarboxamide;
2-(2-bromo-6-fluorophenyl)-N-(pyrimidin-4-yl)-3H-imidazo[4,5-c]pyridin-4-amine;
N-(2-(2-bromo-6-fluorophenyl)-3H-imidazo[4,5-c]pyridin-4-yl)cyclopropanecarboxamide;
6-(2-(2,6-dichloro-4-cyanophenyl)-3H-imidazo[4,5-c]pyridin-4-ylamino)pyrimidine-4-carbonitrile;
3,5-dichloro-4-(4-(5-methylpyrazin-2-ylamino)-3H-imidazo[4,5-c]pyridin-2-yl)benzonitrile;
3,5-dichloro-4-(4-(5-(hydroxymethyl)pyridin-2-ylamino)-3H-imidazo[4,5-c]pyridin-2-yl)benzonitrile;
N-(6-(azetidin-1-yl)-2-methylpyrimidin-4-yl)-2-(2-chloro-6-fluorophenyl)-3H-imidazo[4,5-c]pyridin-4-amine;
(6-(2-(2,6-dichlorophenyl)-3H-imidazo[4,5-c]pyridin-4-ylamino)pyrimidin-4-yl)methanol;
6-(2-(2,6-dichlorophenyl)-3H-imidazo[4,5-c]pyridin-4-ylamino)pyrimidine-4-carbonitrile;
2-(2,6-dichlorophenyl)-N-(6-(trifluoromethyl)pyrimidin-4-yl)-3H-imidazo[4,5-c]pyridin-4-amine;

N2-(2-(2,6-dichlorophenyl)-3H-imidazo[4,5-c]pyridin-4-yl)-N5,N5-dimethylpyrazine-2,5-diamine;
(5-(2-(2,6-dichlorophenyl)-3H-imidazo[4,5-c]pyridin-4-ylamino)pyrazin-2-yl)methanol;
(6-(2-(2-chloro-6-fluorophenyl)-3H-imidazo[4,5-c]pyridin-4-ylamino)pyrimidin-4-yl)methanol;
6-(2-(2-chloro-6-fluorophenyl)-3H-imidazo[4,5-c]pyridin-4-ylamino)pyrimidine-4-carbonitrile;
2-(2-chloro-6-fluorophenyl)-N-(6-(trifluoromethyl)pyrimidin-4-yl)-3H-imidazo[4,5-c]pyridin-4-amine;
2-(4-(5-(2-(2-chloro-6-fluorophenyl)-3H-imidazo[4,5-c]pyridin-4-ylamino)pyrazin-2-yl)piperazin-1-yl)ethanol;
N2-(2-(2-chloro-6-fluorophenyl)-3H-imidazo[4,5-c]pyridin-4-yl)-N5,N5-dimethylpyrazine-2,5-diamine;
2-(2,6-dichlorophenyl)-N-(pyrimidin-5-yl)-3H-imidazo[4,5-c]pyridin-4-amine;
N-4-(2-(2-chlorophenyl)-3H-imidazo[4,5-c]pyridin-4-yl)-N2,N2-dimethylpyrimidine-2,4-diamine;
2-(2-chlorophenyl)-N-(2-morpholinopyrimidin-4-yl)-3H-imidazo[4,5-c]pyridin-4-amine;
3,5-dichloro-4-(4-(4-(hydroxymethyl)pyridin-2-ylamino)-3H-imidazo[4,5-c]pyridin-2-yl)benzonitrile;
1-(6-(2-(2-chloro-6-fluorophenyl)-3H-imidazo[4,5-c]pyridin-4-ylamino)-2-methylpyrimidin-4-yl)azetidin-3-ol;
3,5-dichloro-4-(4-(6-(3-hydroxypyrrolidin-1-yl)pyrimidin-4-ylamino)-3H-imidazo[4,5-c]pyridin-2-yl)benzonitrile;
3,5-dichloro-4-(4-(6-thiomorpholinosulfonylpyrimidin-4-ylamino)-3H-imidazo[4,5-c]pyridin-2-yl)benzonitrile;
3,5-dichloro-4-(4-(2-methyl-6-morpholinopyrimidin-4-ylamino)-3H-imidazo[4,5-c]pyridin-2-yl)benzonitrile;
N-(6-(azetidin-1-yl)pyrimidin-4-yl)-2-(2,6-dichlorophenyl)-3H-imidazo[4,5-c]pyridin-4-amine;
1-(6-(2-(2,6-dichlorophenyl)-3H-imidazo[4,5-c]pyridin-4-ylamino)pyrimidin-4-yl)azetidin-3-01;
1-(6-(2-(2,6-dichlorophenyl)-3H-imidazo[4,5-c]pyridin-4-ylamino)pyrimidin-4-yl)ethane-1,2-diol;
N-(6-(azetidin-1-yl)pyrimidin-4-yl)-2-(2-chloro-6-fluorophenyl)-3H-imidazo[4,5-c]pyridin-4-amine;
1-(6-(2-(2-chloro-6-fluorophenyl)-3H-imidazo[4,5-c]pyridin-4-ylamino)pyrimidin-4-yl)azetidin-3-01;
2-(2-chloro-6-fluorophenyl)-N-(6-thiomorpholinosulfonylpyrimidin-4-yl)-3H-imidazo[4,5-c]pyridin-4-amine;
1-(6-(2-(2-chloro-6-fluorophenyl)-3H-imidazo[4,5-c]pyridin-4-ylamino)pyrimidin-4-yl)ethane-1,2-diol;
1-(6-(2-(2,6-dichlorophenyl)-3H-imidazo[4,5-c]pyridin-4-ylamino)-2-methylpyrimidin-4-yl)azetidin-3-01;
N-4-(2-(2-chlorophenyl)-3H-imidazo[4,5-c]pyridin-4-yl)-N2,N2,6-trimethylpyrimidine-2,4-diamine;
(R)-1-(6-(2-(2,6-dichlorophenyl)-3H-imidazo[4,5-c]pyridin-4-ylamino)pyrimidin-4-yl)pyrrolidin-3-01;
(S)-1-(6-(2-(2,6-dichlorophenyl)-3H-imidazo[4,5-c]pyridin-4-ylamino)pyrimidin-4-yl)pyrrolidin-3-01;
(R)-1-(6-(2-(2-chloro-6-fluorophenyl)-3H-imidazo[4,5-c]pyridin-4-ylamino)pyrimidin-4-yl)pyrrolidin-3-01;
(S)-1-(6-(2-(2-chloro-6-fluorophenyl)-3H-imidazo[4,5-c]pyridin-4-ylamino)pyrimidin-4-yl)pyrrolidin-3-01;
(3,5-dichloro-4-(4-(6-methylpyrimidin-4-ylamino)-3H-imidazo[4,5-c]pyridin-2-yl)phenyl)methanol;
2-(4-(aminomethyl)-2,6-dichlorophenyl)-N-(6-methylpyrimidin-4-yl)-3H-imidazo[4,5-c]pyridin-4-amine;
N4-(2-(2,6-dichlorophenyl)-3H-imidazo[4,5-c]pyridin-4-yl)pyrimidine-4,6-diamine;
N4-(2-(2-chloro-6-fluorophenyl)-3H-imidazo[4,5-c]pyridin-4-yl)pyrimidine-4,6-diamine;
N4-(2-(2,4,6-trichlorophenyl)-3H-imidazo[4,5-c]pyridin-4-yl)pyrimidine-4,6-diamine;
N4-(2-(2,6-dichlorophenyl)-3H-imidazo[4,5-c]pyridin-4-yl)-N6-methylpyrimidine-4,6-diamine;
2,2′-(6-(2-(2,6-dichlorophenyl)-3H-imidazo[4,5-c]pyridin-4-ylamino)pyrimidin-4-ylazanediyl)diethanol;
2,2′-(6-(2-(2-chloro-6-fluorophenyl)-3H-imidazo[4,5-c]pyridin-4-ylamino)pyrimidin-4-ylazanediyl)diethanol;
1-cyclopropyl-3-(2-(2,6-dichlorophenyl)-3H-imidazo[4,5-c]pyridin-4-yl)urea;
1-(2-(2-chloro-6-fluorophenyl)-3H-imidazo[4,5-c]pyridin-4-yl)-3-cyclopropylurea;
2-(2,6-dichlorophenyl)-N-(6-((dimethylamino)methyl)pyrimidin-4-yl)-3H-imidazo[4,5-c]pyridin-4-amine;
N-(2-(2,6-dichloro-4-((methylamino)methyl)phenyl)-3H-imidazo[4,5-c]pyridin-4-yl)cyclopropanecarboxamide;
2-(2,6-dichloro-4-((methylamino)methyl)phenyl)-N-(6-methylpyrimidin-4-yl)-3H-imidazo[4,5-c]pyridin-4-amine;
2-(2,6-dichloro-4-((methylamino)methyl)phenyl)-N-(2,6-dimethylpyrimidin-4-yl)-3H-imidazo[4,5-c]pyridin-4-amine;
N-(2-(2,6-dichloro-4-((dimethylamino)methyl)phenyl)-3H-imidazo[4,5-c]pyridin-4-yl)cyclopropanecarboxamide;
2-(2,6-dichloro-4-((dimethylamino)methyl)phenyl)-N-(6-methylpyrimidin-4-yl)-3H-imidazo[4,5-c]pyridin-4-amine;
2-(2,6-dichloro-4-((dimethylamino)methyl)phenyl)-N-(2,6-dimethylpyrimidin-4-yl)-3H-imidazo[4,5-c]pyridin-4-amine;
N-(2-(2,6-dichloro-4-(morpholinomethyl)phenyl)-3H-imidazo[4,5-c]pyridin-4-yl)cyclopropanecarboxamide;
2-(2,6-dichloro-4-(morpholinomethyl)phenyl)-N-(6-methylpyrimidin-4-yl)-3H-imidazo[4,5-c]pyridin-4-amine;
2-(2,6-dichloro-4-(morpholinomethyl)phenyl)-N-(2,6-dimethylpyrimidin-4-yl)-3H-imidazo[4,5-c]pyridin-4-amine;
2-(3,5-dichloropyridin-4-yl)-N-(2,6-dimethylpyrimidin-4-yl)-3H-imidazo[4,5-c]pyridin-4-amine;
N-(2-(3,5-dichloropyridin-4-yl)-3H-imidazo[4,5-c]pyridin-4-yl)cyclopropanecarboxamide;
2-(3,5-dichloropyridin-4-yl)-N-(6-methylpyrimidin-4-yl)-3H-imidazo[4,5-c]pyridin-4-amine;
(6-(2-(3,5-dichloropyridin-4-yl)-3H-imidazo[4,5-c]pyridin-4-ylamino)pyrimidin-4-yl)methanol;
6-(2-(3,5-dichloropyridin-4-yl)-3H-imidazo[4,5-c]pyridin-4-ylamino)pyrimidine-4-carbonitrile;

N4-(2-(3,5-dichloropyridin-4-yl)-3H-imidazo[4,5-c]pyridin-4-yl)pyrimidine-4,6-diamine;
4-(4-(6-aminopyrimidin-4-ylamino)-3H-imidazo[4,5-c]pyridin-2-yl)-3,5-dichlorobenzonitrile;
(1S,2R)—N-(2-(2,6-dichlorophenyl)-3H-imidazo[4,5-c]pyridin-4-yl)-2-fluorocyclopropanecarboxamide;
(1R,2S)—N-(2-(2,6-dichlorophenyl)-3H-imidazo[4,5-c]pyridin-4-yl)-2-fluorocyclopropanecarboxamide;
N-(6-(2-(2,6-dichlorophenyl)-3H-imidazo[4,5-c]pyridin-4-ylamino)pyrimidin-4-yl)acetamide;
2-(6-(2-(2,6-dichlorophenyl)-3H-imidazo[4,5-c]pyridin-4-ylamino)pyrimidin-4-yl)propan-2-ol;
N-(6-(2-(2-chloro-6-fluorophenyl)-3H-imidazo[4,5-c]pyridin-4-ylamino)pyrimidin-4-yl)acetamide;
2-(6-(2-(2-chloro-6-fluorophenyl)-3H-imidazo[4,5-c]pyridin-4-ylamino)pyrimidin-4-yl)propan-2-ol;
N-(2-(2-chloro-6-fluorophenyl)-3H-imidazo[4,5-c]pyridin-4-yl)-2-(dimethylamino)acetamide;
2-(2-chloro-5-fluorophenyl)-N-(6-methylpyrimidin-4-yl)-3H-imidazo[4,5-c]pyridin-4-amine;
2-(2-chloro-3-fluorophenyl)-N-(6-methylpyrimidin-4-yl)-3H-imidazo[4,5-c]pyridin-4-amine;
2-(2-chloro-6-methylphenyl)-N-(6-methylpyrimidin-4-yl)-3H-imidazo[4,5-c]pyridin-4-amine;
2-(2-chloro-6-methoxyphenyl)-N-(6-methylpyrimidin-4-yl)-3H-imidazo[4,5-c]pyridin-4-amine;
2-(2-chloro-6-(trifluoromethyl)phenyl)-N-(6-methylpyrimidin-4-yl)-3H-imidazo[4,5-c]pyridin-4-amine;
2-(2,5-dichlorophenyl)-N-(6-methylpyrimidin-4-yl)-3H-imidazo[4,5-c]pyridin-4-amine;
2-(2,4-dichlorophenyl)-N-(6-methylpyrimidin-4-yl)-3H-imidazo[4,5-c]pyridin-4-amine;
2-(2-chloro-3-methylphenyl)-N-(6-methylpyrimidin-4-yl)-3H-imidazo[4,5-c]pyridin-4-amine;
2-(2,3-dichlorophenyl)-N-(6-methylpyrimidin-4-yl)-3H-imidazo[4,5-c]pyridin-4-amine;
2-(5-bromo-2-chlorophenyl)-N-(6-methylpyrimidin-4-yl)-3H-imidazo[4,5-c]pyridin-4-amine;
1-(2-(2,6-dichlorophenyl)-3H-imidazo[4,5-c]pyridin-4-yl)-3-isopropylurea;
1-(2-(2-chloro-6-fluorophenyl)-3H-imidazo[4,5-c]pyridin-4-yl)-3-isopropylurea;
1-(2-(2-chloro-6-fluorophenyl)-3H-imidazo[4,5-c]pyridin-4-yl)-3-ethylurea;
3,5-dichloro-4-(4-(6-(hydroxymethyl)pyrimidin-4-ylamino)-3H-imidazo[4,5-c]pyridin-2-yl)benzonitrile;
3-chloro-4-(4-(6-methylpyrimidin-4-ylamino)-3H-imidazo[4,5-c]pyridin-2-yl)benzonitrile;
4-chloro-3-(4-(6-methylpyrimidin-4-ylamino)-3H-imidazo[4,5-c]pyridin-2-yl)benzonitrile;
(4-chloro-3-(4-(6-methylpyrimidin-4-ylamino)-3H-imidazo[4,5-c]pyridin-2-yl)phenyl)methanol;
2-(2-chloro-5-(morpholinomethyl)phenyl)-N-(6-methylpyrimidin-4-yl)-3H-imidazo[4,5-c]pyridin-4-amine;
tert-butyl 2-(2-(2-chloro-6-fluorophenyl)-3H-imidazo[4,5-c]pyridin-4-ylamino)-2-oxoethylcarbamate;
3,5-dichloro-4-(4-(6-(hydroxymethyl)pyrimidin-4-ylamino)-3H-imidazo[4,5-c]pyridin-2-yl)benzamide;
2-(2,6-dichloro-4-methylphenyl)-N-(6-methylpyrimidin-4-yl)-3H-imidazo[4,5-c]pyridin-4-amine;
2-(2,6-dichloro-4-(methylthio)phenyl)-N-(6-methylpyrimidin-4-yl)-3H-imidazo[4,5-c]pyridin-4-amine;
2-(2,6-dichloro-3-fluorophenyl)-N-(6-methylpyrimidin-4-yl)-3H-imidazo[4,5-c]pyridin-4-amine;
6-(2-(2,6-dichlorophenyl)-3H-imidazo[4,5-c]pyridin-4-ylamino)pyrimidine-4-carboxamide;
6-(2-(2,6-dichlorophenyl)-3H-imidazo[4,5-c]pyridin-4-ylamino)-N-methylpyrimidine-4-carboxamide;
(6-(2-(2,6-dichlorophenyl)-3H-imidazo[4,5-c]pyridin-4-ylamino)pyrimidin-4-yl)(morpholino)methanone;
6-(2-(2-chloro-6-fluorophenyl)-3H-imidazo[4,5-c]pyridin-4-ylamino)pyrimidine-4-carboxamide;
6-(2-(2-chloro-6-fluorophenyl)-3H-imidazo[4,5-c]pyridin-4-ylamino)-N-methylpyrimidine-4-carboxamide;
(6-(2-(2-chloro-6-fluorophenyl)-3H-imidazo[4,5-c]pyridin-4-ylamino)pyrimidin-4-yl)(morpholino)methanone;
6-(2-(2,6-dichlorophenyl)-3H-imidazo[4,5-c]pyridin-4-ylamino)-N-methylpyridazine-3-carboxamide;
(6-(2-(2,6-dichlorophenyl)-3H-imidazo[4,5-c]pyridin-4-ylamino)pyridazin-3-yl)(morpholino)methanone;
6-(2-(2-chloro-6-fluorophenyl)-3H-imidazo[4,5-c]pyridin-4-ylamino)-N-methylpyridazine-3-carboxamide;
3-chloro-2-(3-methyl-4-(6-methylpyrimidin-4-ylamino)-3H-imidazo[4,5-c]pyridin-2-yl)phenol;
2-(2-chlorophenyl)-N-(6-methylpyrimidin-4-yl)-3H-imidazo[4,5-c]pyridin-4-amine;
3,5-dichloro-4-(6-(6-(trifluoromethyl)pyrimidin-4-ylamino)-7H-purin-8-yl)benzonitrile;
8-(2,6-dichlorophenyl)-N-(6-(trifluoromethyl)pyrimidin-4-yl)-7H-purin-6-amine;
6-(8-(2-chloro-6-fluorophenyl)-7H-purin-6-ylamino)pyrimidine-4-carbonitrile;
8-(2-chloro-6-fluorophenyl)-N-(5-methylpyrazin-2-yl)-7H-purin-6-amine;
5-(8-(2-chloro-6-fluorophenyl)-7H-purin-6-ylamino)pyrazine-2-carbonitrile;
8-(2,6-dichlorophenyl)-9-methyl-N-(pyridin-3-yl)-9H-purin-6-amine;
8-(2-chloro-6-fluorophenyl)-9-methyl-N-phenyl-9H-purin-6-amine;
8-(2-chloro-6-fluorophenyl)-9-methyl-N-(pyridin-4-yl)-9H-purin-6-amine;
4-(6-(6-aminopyrimidin-4-ylamino)-7H-purin-8-yl)-3,5-dichlorobenzonitrile;
3,5-dichloro-4-(6-(6-ethylpyrimidin-4-ylamino)-7H-purin-8-yl)benzonitrile;
3,5-dichloro-4-(6-(6-cyclopropylpyrimidin-4-ylamino)-7H-purin-8-yl)benzonitrile;
3,5-dichloro-4-(6-(5-ethylpyrazin-2-ylamino)-7H-purin-8-yl)benzonitrile;
3,5-dichloro-4-(6-(5-ethylpyridin-2-ylamino)-7H-purin-8-yl)benzonitrile;
8-(2,6-dichlorophenyl)-N-(6-(morpholinomethyl)pyrimidin-4-yl)-7H-purin-6-amine;
(R)-1-(6-(8-(2,6-dichlorophenyl)-7H-purin-6-ylamino)pyrimidin-4-yl)ethanol;
(S)-1-(6-(8-(2,6-dichlorophenyl)-7H-purin-6-ylamino)pyrimidin-4-yl)ethanol;
5-(8-(2,6-dichloro-4-cyanophenyl)-7H-purin-6-ylamino)pyrazine-2-carbonitrile;
5-(8-(2,6-dichlorophenyl)-7H-purin-6-ylamino)pyrazine-2-carbonitrile;
8-(2,6-dichlorophenyl)-N-(5-((methylamino)methyl)pyrazin-2-yl)-7H-purin-6-amine;
3,5-dichloro-4-(6-(6-(1-hydroxyethyl)pyrimidin-4-ylamino)-7H-purin-8-yl)benzonitrile;
N-(6-methylpyrimidin-4-yl)-8-(2,4,6-trichlorophenyl)-7H-purin-6-amine;

N4-(8-(2,4,6-trichlorophenyl)-7H-purin-6-yl)pyrimidine-4,6-diamine;

N4-(8-(2,6-dichloro-4-methylphenyl)-7H-purin-6-yl)pyrimidine-4,6-diaminel;
8-(2,6-dichloro-4-methylphenyl)-N-(6-methylpyrimidin-4-yl)-7H-purin-6-amine;
8-(2,6-dichloro-4-ethynylphenyl)-N-(6-methylpyrimidin-4-yl)-7H-purin-6-amine;
8-(2,6-dichloro-3-fluorophenyl)-N-(6-methylpyrimidin-4-yl)-7H-purin-6-amine;
N4-(8-(2,6-dichloro-3-fluorophenyl)-7H-purin-6-yl)pyrimidine-4,6-diamine;
N4-(8-(2-chloro-6-fluoro-3-methylphenyl)-7H-purin-6-yl)pyrimidine-4,6-diamine;
8-(2-chloro-6-fluoro-3-methylphenyl)-N-(6-methylpyrimidin-4-yl)-7H-purin-6-amine;

N4-(8-(2-chloro-3,6-difluorophenyl)-7H-purin-6-yl)pyrimidine-4,6-diamine;
8-(2-chloro-3,6-difluorophenyl)-N-(6-methylpyrimidin-4-yl)-7H-purin-6-amine;
N4-(8-(2,3,6-trichlorophenyl)-7H-purin-6-yl)pyrimidine-4,6-diamine;
2-(2-chloro-6-fluorophenyl)-N-(6-(4-(oxetan-3-yl)piperazin-1-yl)pyrimidin-4-yl)-3H-imidazo[4,5-c]pyridin-4-amine;
2-(2-(2-(2,6-dichlorophenyl)-3H-imidazo[4,5-c]pyridin-4-ylamino)pyridin-4-yl)propan-2-ol;
N4-(2-(2,6-dichlorophenyl)-3H-imidazo[4,5-c]pyridin-4-yl)pyridine-2,4-diamine;
N4-(2-(2,6-dichlorophenyl)-3H-imidazo[4,5-c]pyridin-4-yl)-6-morpholinopyrimidine-2,4-diamine;
2-(2,6-dichlorophenyl)-N-(1,3-dimethyl-1H-pyrazol-5-yl)-3H-imidazo[4,5-c]pyridin-4-amine;
2-(2,6-dichlorophenyl)-N-(1-methyl-1H-pyrazol-3-yl)-3H-imidazo[4,5-c]pyridin-4-amine;
N-(6-(2-oxa-6-azaspiro[3.3]heptan-6-yl)pyrimidin-4-yl)-2-(2,6-dichlorophenyl)-3H-imidazo[4,5-c]pyridin-4-amine;
N4-(8-(2,6-dichlorophenyl)-7H-purin-6-yl)-N6,N6-dimethylpyrimidine-4,6-diamine;
8-(2,6-dichlorophenyl)-N-(6-(4-(oxetan-3-yl)piperazin-1-yl)pyrimidin-4-yl)-7H-purin-6-amine;
N-(6-(azetidin-1-yl)pyrimidin-4-yl)-8-(2,6-dichlorophenyl)-7H-purin-6-amine;
methyl 2-(8-(2,6-dichlorophenyl)-7H-purin-6-ylamino) isonicotinate;
2-(2-(8-(2,6-dichlorophenyl)-7H-purin-6-ylamino)pyridin-4-yl)propan-2-ol;
methyl 4-(8-(2,6-dichlorophenyl)-7H-purin-6-ylamino)picolinate;
2-(8-(2,6-dichlorophenyl)-7H-purin-6-ylamino)isonicotinic acid;
(5-(8-(2,6-dichlorophenyl)-7H-purin-6-ylamino)pyridin-3-yl)methanol;
6-(8-(2,6-dichlorophenyl)-7H-purin-6-ylamino)pyrimidin-4-ol;
N2-(8-(2,6-dichlorophenyl)-7H-purin-6-yl)-1,3,5-triazine-2,4-diamine;
N2-(8-(2,6-dichlorophenyl)-7H-purin-6-yl)pyridine-2,6-diamine;
4-(8-(2,6-dichlorophenyl)-7H-purin-6-ylamino)picolinic acid;
8-(2,6-dichlorophenyl)-N-(5-methyl-1H-pyrazol-3-yl)-7H-purin-6-amine;
N4-(8-(2,6-dichlorophenyl)-7H-purin-6-yl)pyridine-2,4-diamine;
(2-(8-(2,6-dichlorophenyl)-7H-purin-6-ylamino)pyridin-4-yl)methanol;
8-(2,6-dichlorophenyl)-N-(1,3-dimethyl-1H-pyrazol-5-yl)-7H-purin-6-amine;
8-(2,6-dichlorophenyl)-N-(1-methyl-1H-pyrazol-3-yl)-7H-purin-6-amine;
N4-(8-(2,6-dichlorophenyl)-7H-purin-6-yl)-2-morpholinopyrimidine-4,6-diamine;
1-(4-(8-(2,6-dichlorophenyl)-7H-purin-6-ylamino)pyridin-2-yl)ethanone;
2-(4-(8-(2,6-dichlorophenyl)-7H-purin-6-ylamino)pyridin-2-yl)propan-2-ol;
methyl 5-(8-(2,6-dichlorophenyl)-7H-purin-6-ylamino)nicotinate;
N-(6-chloropyrimidin-4-yl)-8-(2,6-dichlorophenyl)-7H-purin-6-amine;
2-(4-(8-(2,6-dichlorophenyl)-7H-purin-6-ylamino)pyridin-2-yl)propan-2-ol;
2-(6-(8-(2,6-dichlorophenyl)-7H-purin-6-ylamino)pyridin-2-yl)propan-2-ol;
2-(6-amino-8-(2,6-dichlorophenyl)-9H-purin-9-yl)ethanol;
2-(8-(2,6-dichlorophenyl)-6-(4-(hydroxymethyl)pyridin-2-ylamino)-9H-purin-9-yl)ethanol;

N5-(8-(2,6-dichlorophenyl)-7H-purin-6-yl)pyrimidine-4,5-diamine;
2-(6-amino-8-(2,6-dichlorophenyl)-9H-purin-9-yl)acetic acid;
2-(6-amino-8-(2,6-dichlorophenyl)-9H-purin-9-yl)acetamide;
2-(6-amino-8-(2,6-dichlorophenyl)-9H-purin-9-yl)acetimidamide;
N4-(8-(2,6-dichlorophenyl)-9H-purin-6-yl)pyrimidine-4,5-diamine;

N4-(8-(2,6-dichlorophenyl)-9H-purin-6-yl)pyrimidine-2,4-diamine;
N2-(8-(2,6-dichlorophenyl)-9H-purin-6-yl)pyrimidine-2,4-diamine;
4-amino-6-(8-(2,6-dichlorophenyl)-9H-purin-6-ylamino)pyrimidin-2-ol;

N4-(8-(2,6-dichlorophenyl)-9H-purin-6-yl)-2-(methylthio)pyrimidine-4,6-diamine;
N-(6-(2-oxa-6-azaspiro[3.3]heptan-6-yl)pyrimidin-4-yl)-8-(2,6-dichlorophenyl)-9H-purin-6-amine;
8-(2,6-dichlorophenyl)-N-(5-(trifluoromethyl)pyridin-2-yl)-9H-purin-6-amine;
8-(2,6-dichlorophenyl)-N-(4-(trifluoromethyl)pyridin-2-yl)-9H-purin-6-amine;
6-(8-(2,6-dichlorophenyl)-9H-purin-6-ylamino)-4-methylnicotinonitrile;
8-(2,6-dichlorophenyl)-N-(4-fluoropyridin-2-yl)-9H-purin-6-amine;
8-(2,6-dichlorophenyl)-N-(7H-pyrrolo[2,3-d]pyrimidin-4-yl)-9H-purin-6-amine;
8-(2,6-dichlorophenyl)-N-(5-fluoropyridin-2-yl)-9H-purin-6-amine;
N4-(8-(2,6-dichlorophenyl)-9H-purin-6-yl)-2-methylpyrimidine-4,6-diamine;
1-(6-(8-(2,6-dichlorophenyl)-9H-purin-6-ylamino)pyridin-3-yl)cyclopropanecarboxylic acid;
N4-(8-(2,6-dichlorophenyl)-9H-purin-6-yl)pyrimidine-2,4,6-triamine;
N-(8-(2,6-dichlorophenyl)-9H-purin-6-yl)-1H-pyrazolo[3,4-d]pyrimidin-4-amine;
2-(2-(2-chloro-6-fluorophenyl)-3H-imidazo[4,5-c]pyridin-4-ylamino)isonicotinamide;
(2-(2-(2-chloro-6-fluorophenyl)-3H-imidazo[4,5-c]pyridin-4-ylamino)pyridin-4-yl)(pyrrolidin-1-yl)methanone;
(2-(2-(2,6-dichlorophenyl)-3H-imidazo[4,5-c]pyridin-4-ylamino)pyridin-4-yl)(pyrrolidin-1-yl)methanone;
8-(2,6-dichlorophenyl)-7H-purin-6-amine;
8-(2,6-dichlorophenyl)-N-methyl-7H-purin-6-amine;
methyl 2-(8-(2,6-dichlorophenyl)-7H-purin-6-ylamino)isonicotinate; (2-(8-(2,6-dichlorophenyl)-7H-purin-6-ylamino)pyridin-4-yl)(pyrrolidin-1-yl)methanone;
N-(8-(2,6-dichlorophenyl)-7H-purin-6-yl)acetamide;
2-(8-(2,6-dichlorophenyl)-7H-purin-6-ylamino)isonicotinamide;
2-(8-(2,6-dichlorophenyl)-7H-purin-6-ylamino)isonicotinic acid;
2-(2,6-dichlorophenyl)-N-(6-(3-fluoroazetidin-1-yl)pyrimidin-4-yl)-3H-imidazo[4,5-c]pyridin-4-amine;
(6-(8-(2,6-dichlorophenyl)-7H-purin-6-ylamino)pyrimidin-4-yl)methanol;
8-(2,6-dichlorophenyl)-9-methyl-9H-purin-6-amine;
N-(8-(2,6-dichlorophenyl)-7H-purin-6-yl)propionamide;
2-(8-(2,6-dichlorophenyl)-7H-purin-6-ylamino)-N-methylisonicotinamide;
azetidin-1-yl(2-(8-(2,6-dichlorophenyl)-7H-purin-6-ylamino)pyridin-4-yl)methanone;
(2-(8-(2,6-dichlorophenyl)-7H-purin-6-ylamino)pyridin-4-yl)(3-hydroxyazetidin-1-yl)methanone;
2-(8-(2,6-dichlorophenyl)-7H-purin-6-ylamino)-N-(1-hydroxy-2-methylpropan-2-yl)isonicotinamide;
[2-(3-Amino-pyrazin-2-yl)-3H-imidazo[4,5-c]pyridin-4-yl]-pyrimidin-4-yl-amine;
[2-(3-Amino-pyridin-2-yl)-3H-imidazo[4,5-c]pyridin-4-yl]-pyrimidin-4-yl-amine;
[2-(3-Chloro-pyridin-2-yl)-1H-imidazo[4,5-c]pyridin-4-yl]-(2,6-dimethyl-pyrimidin-4-yl)-amine;
Cyclopropanecarboxylic acid [2-(3-chloro-pyridin-2-yl)-1H-imidazo[4,5-c]pyridin-4-yl]-amide;
[2-(2,6-Dichloro-phenyl)-3-methyl-3H-imidazo[4,5-c]pyridin-4-yl]-pyrimidin-4-yl-amine;
[2-(2,6-Dichloro-phenyl)-1-methyl-1H-imidazo[4,5-c]pyridin-4-yl]-pyrimidin-4-yl-amine;
[2-(2-Amino-pyridin-3-yl)-1H-imidazo[4,5-c]pyridin-4-yl]-(2,6-dimethyl-pyrimidin-4-yl)-amine;
2-[8-(2,6-Dichloro-phenyl)-6-(2,6-dimethyl-pyrimidin-4-ylamino)-purin-9-yl]-ethanol;
Cyclopropanecarboxylic acid [8-(2,6-dichloro-phenyl)-9-(2-hydroxy-ethyl)-9H-purin-6-yl]-amide;
(2,6-Dimethyl-pyrimidin-4-yl)-[2-(2,4,6-trichloro-phenyl)-1H-imidazo[4,5-c]pyridin-4-yl]-amine;
Cyclopropanecarboxylic acid [2-(2,4,6-trichloro-phenyl)-1H-imidazo[4,5-c]pyridin-4-yl]-amide;
2-[4-(2,6-Dimethyl-pyrimidin-4-ylamino)-1H-imidazo[4,5-c]pyridin-2-yl]-benzonitrile;
2-{8-(2,6-Dichloro-phenyl)-6-[6-(4-methyl-piperazin-1-yl)-pyrimidin-4-ylamino]-purin-9-yl}-ethanol;
[2-(2,6-Dichloro-phenyl)-7-fluoro-1H-imidazo[4,5-c]pyridin-4-yl]-(2,6-dimethyl-pyrimidin-4-yl)-amine;
[2-(2,6-Dichloro-phenyl)-7-fluoro-1H-imidazo[4,5-c]pyridin-4-yl]-(4-morpholin-4-yl-pyridin-2-yl)-amine;
[2-(2,6-Dichloro-phenyl)-7-fluoro-1H-imidazo[4,5-c]pyridin-4-yl]-(6-morpholin-4-yl-pyrimidin-4-yl)-amine;
[7-Chloro-2-(2,6-dichloro-phenyl)-1H-imidazo[4,5-c]pyridin-4-yl]-(2,6-dimethyl-pyrimidin-4-yl)-amine;
Cyclopropanecarboxylic acid [7-chloro-2-(2,6-dichloro-phenyl)-1H-imidazo[4,5-c]pyridin-4-yl]-amide;
Cyclopropanecarboxylic acid [2-(2,6-dichloro-phenyl)-7-methyl-1H-imidazo[4,5-c]pyridin-4-yl]-amide;
[2-(2,6-Dichloro-phenyl)-7-methyl-1H-imidazo[4,5-c]pyridin-4-yl]-(2,6-dimethyl-pyrimidin-4-yl)-amine;
[2-(2,6-Dichloro-phenyl)-7-fluoro-1H-imidazo[4,5-c]pyridin-4-yl]-(6-methyl-pyrimidin-4-yl)-amine;
(6-Cyclopropyl-pyrimidin-4-yl)-[2-(2,6-dichloro-phenyl)-7-fluoro-1H-imidazo[4,5-c]pyridin-4-yl]-amine;
[2-(2,6-Dichloro-phenyl)-7-fluoro-1H-imidazo[4,5-c]pyridin-4-yl]-(6-isopropyl-pyrimidin-4-yl)-amine;
[7-Bromo-2-(2,6-dichloro-phenyl)-1H-imidazo[4,5-c]pyridin-4-yl]-(2,6-dimethyl-pyrimidin-4-yl)-amine;
Cyclopropanecarboxylic acid [7-bromo-2-(2,6-dichloro-phenyl)-1H-imidazo[4,5-c]pyridin-4-yl]-amide;
[2-(2,6-Dichloro-phenyl)-7-fluoro-1H-imidazo[4,5-c]pyridin-4-yl]-(5-morpholin-4-yl-pyridin-2-yl)-amine;
[2-(2-Chloro-6-fluoro-phenyl)-7-fluoro-1H-imidazo[4,5-c]pyridin-4-yl]-(6-methyl-pyrimidin-4-yl)-amine;
Cyclopropanecarboxylic acid [2-(2-chloro-6-fluoro-phenyl)-7-fluoro-1H-imidazo[4,5-c]pyridin-4-yl]-amide;
[2-(2-Chloro-6-fluoro-phenyl)-7-fluoro-1H-imidazo[4,5-c]pyridin-4-yl]-(2,6-dimethyl-pyrimidin-4-yl)-amine;
[7-Bromo-2-(2,6-dichloro-phenyl)-1H-imidazo[4,5-c]pyridin-4-yl]-(6-methyl-pyrimidin-4-yl)-amine;
N-[7-Bromo-2-(2,6-dichloro-phenyl)-1H-imidazo[4,5-c]pyridin-4-yl]-pyrimidine-4,6-diamine;
2-(2,6-Dichloro-phenyl)-4-(6-methyl-pyrimidin-4-ylamino)-1H-imidazo[4,5-c]pyridine-7-carbonitrile;
4-[2-(2,6-Dichloro-phenyl)-3H-imidazo[4,5-c]pyridin-4-ylamino]-1-methyl-1H-pyrimidin-2-one;
[2-(4-Amino-2-chloro-6-fluoro-phenyl)-7-fluoro-1H-imidazo[4,5-c]pyridin-4-yl]-(6-methyl-pyrimidin-4-yl)-amine;
N-[7-Chloro-2-(2,6-dichloro-phenyl)-1H-imidazo[4,5-c]pyridin-4-yl]-pyrimidine-4,6-diamine;
2-(2,6-Dichloro-phenyl)-4-(6-methyl-pyrimidin-4-ylamino)-1H-imidazo[4,5-c]pyridine-7-carboxylic acid amide;
[2-(2,6-Dichloro-phenyl)-1-methyl-1H-imidazo[4,5-c]pyridin-4-yl]-thiazol-5-yl-amine;
[2-(2,6-Dichloro-phenyl)-1-methyl-1H-imidazo[4,5-c]pyridin-4-yl]-(6-methyl-pyrimidin-4-yl)-amine;
4-[2-(2,6-Dichloro-phenyl)-1-methyl-1H-imidazo[4,5-c]pyridin-4-ylamino]-pyridin-3-ol;
[2-(2,6-Dichloro-phenyl)-1-methyl-1H-imidazo[4,5-c]pyridin-4-yl]-(3-methyl-isothiazol-5-yl)-amine;
4-[2-(2,6-Dichloro-phenyl)-3H-imidazo[4,5-c]pyridin-4-ylamino]-pyridine-3-carbaldehyde;
N-[2-(2-Chloro-3,6-difluoro-phenyl)-3H-imidazo[4,5-c]pyridin-4-yl]-pyrimidine-4,6-diamine; and
N-[2-(4-Amino-2-chloro-6-fluoro-phenyl)-7-fluoro-1H-imidazo[4,5-c]pyridin-4-yl]-pyrimidine-4,6-diamine.

The compounds of Formulas Ia-Ib may contain asymmetric or chiral centers, and, therefore, exist in different stereoisomeric forms. It is intended that all stereoisomeric forms of the compounds of Formulas Ia-Ib, including but not limited to: diastereomers, enantiomers, and atropisomers as well as mixtures thereof such as racemic mixtures, form part of the present invention. In addition, the present invention embraces all geometric and positional isomers. For example, if a compound of Formulas Ia-Ib incorporates a double bond or a fused ring, both the cis- and trans-forms, as well as mixtures, are embraced within the scope of the invention. Both the single positional isomers and mixture of positional isomers, e.g., resulting from the N-oxidation of the pyrimidinyl and pyrrozolyl rings, or the E and Z forms of compounds of Formulas Ia-Ib (for example oxime moieties), are also within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention, as defined by the claims, embrace both solvated and unsolvated forms.

In an embodiment, compounds of Formulas Ia-Ib may exist in different tautomeric forms, and all such forms are embraced within the scope of the invention, as defined by the claims. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The present invention also embraces isotopically-labeled compounds of Formulas Ia-Ib, which are identical to those recited herein, but for the fact that one or more atoms are replaced by an atom having an atomic mass or mass number different from the atomic mass or mass number usually found in nature. All isotopes of any particular atom or element as specified are contemplated within the scope of the invention. Exemplary isotopes that can be incorporated into compounds of Formulas Ia-Ib include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of Formulas Ia-Ib (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of Formulas Ia-Ib can generally be prepared by following procedures analogous to those disclosed in the Schemes and/or in the Examples herein below, by substituting an isotopically labeled reagent for a non-isotopically labeled reagent.

### Synthesis of TYK2 Inhibitor Compounds

Compounds of Formulas Ia-Ib may be synthesized by synthetic routes described herein. In certain embodiments, processes well-known in the chemical arts can be used, in addition to, or in light of, the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), Beilsteins Handbuch der organischen Chemie, 4, Aufl. ed. Springer-Verlag, Berlin, including supplements (also available via the Beilstein online database)), or Comprehensive Heterocyclic Chemistry, Editors Katrizky and Rees, Pergamon Press, 1984.

Compounds of Formulas Ia-Ib may be prepared singly or as compound libraries comprising at least 2, for example 5 to 1,000 compounds, or 10 to 100 compounds of Formulas Ia-Ib.

Libraries of compounds of Formulas Ia-Ib may be prepared by a combinatorial ‘split and mix’ approach or by multiple parallel syntheses using either solution phase or solid phase chemistry, by procedures known to those skilled in the art. Thus according to a further aspect of the invention there is provided a compound library comprising at least 2 compounds of Formulas Ia-Ib, enantiomers, diasteriomers or pharmaceutically acceptable salts thereof.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Compounds of the invention may be prepared from commercially available starting materials using the general methods illustrated herein.

For illustrative purposes, reaction Schemes 1-8 depicted below provide routes for synthesizing the compounds of Formulas Ia-Ib, as well as key intermediates. For a more detailed description of the individual reaction steps, see the Examples section below. Those skilled in the art will appreciate that other synthetic routes may be available and used. Although specific starting materials and reagents are depicted in the Schemes and discussed below, other starting materials and reagents may be available for substitution to provide a variety of derivatives and/or reaction conditions. In addition, many of the compounds prepared by the methods described below can be further modified in light of this disclosure using conventional chemistry well known to those skilled in the art.

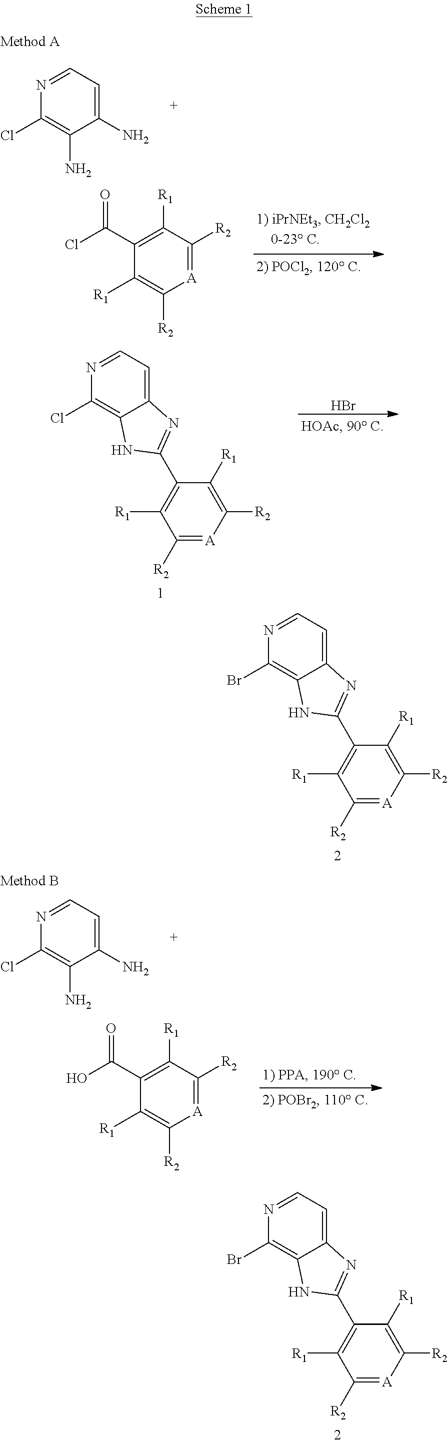

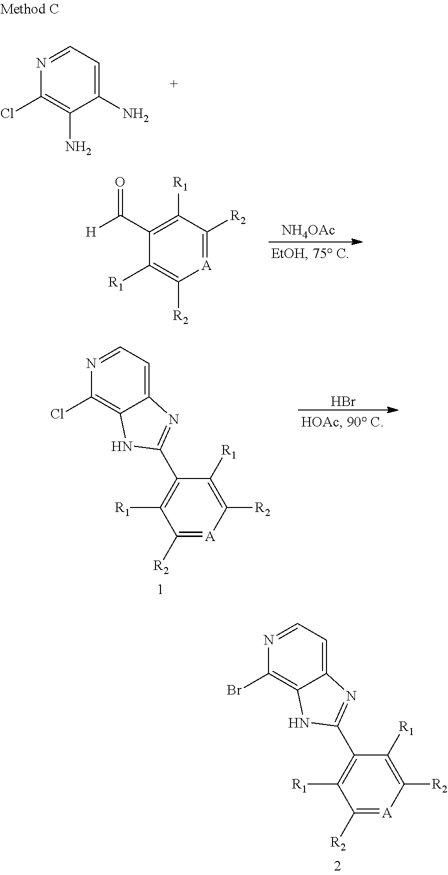

Scheme 1 depicts methods of preparing compounds 1 and 2 that can be used in further methods to prepare compounds of the present invention. Three methods are shown for the preparation of Compound 2. In the first method (Method A), 2-chloropyridine-3,4-diamine can be coupled with an acid chloride to form a mixture of regio-isomeric amides. Treatment of this amide mixture with POCl₃ gives compound 1. The chloride can be subsequently replaced with bromide when heated with HBr in acetic acid.

In the second method (Method B), 2-chloropyridine-3,4-diamine can be condensed with an acid in the presence of polyphosphoric acid (PPA). This transformation also hydrolyzes the chloride to provide a hydroxyl intermediate, which can be converted to bromide 2 when treated with POBr₃.

In the third method (Method C), 2-chloropyridine-3,4-diamine can be converted to compound 1 in the presence of an aldehyde and ammonium acetate. Replacement of chloride with bromide when heated with HBr in acetic acid gives bromide 2.

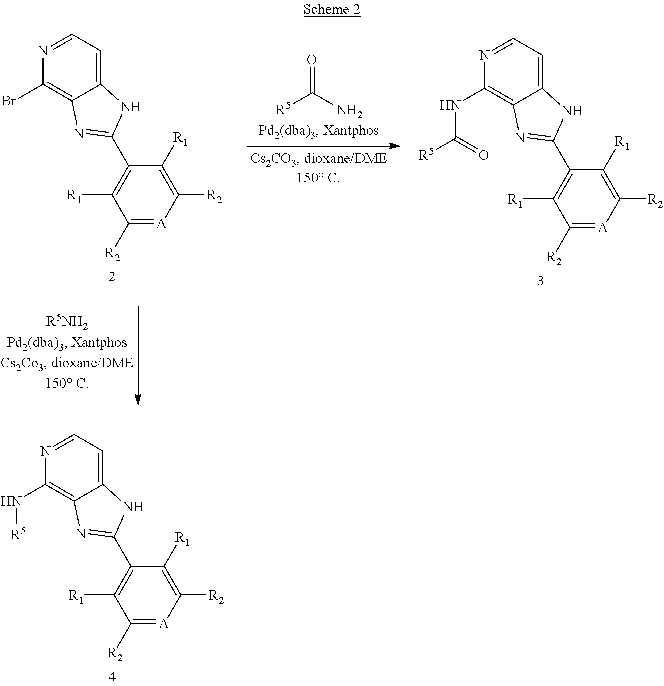

Scheme 2 depicts methods of transforming bromide 2 through a palladium-catalyzed coupling reaction to provide compounds 3 and 4. Heating of bromide 2 with an amide (R⁵CONH₂) or an amine (R⁵NH₂) at 150° C. for a couple of hours under nitrogen, in the presence of Pd₂(dba)₃, XantPhos, Cs₂CO₃ and 1,4-Dioxane/DME, gives the desired product. This Palldium-catalyzed coupling reaction can be carried out in a sealed tube in a microwave reactor.

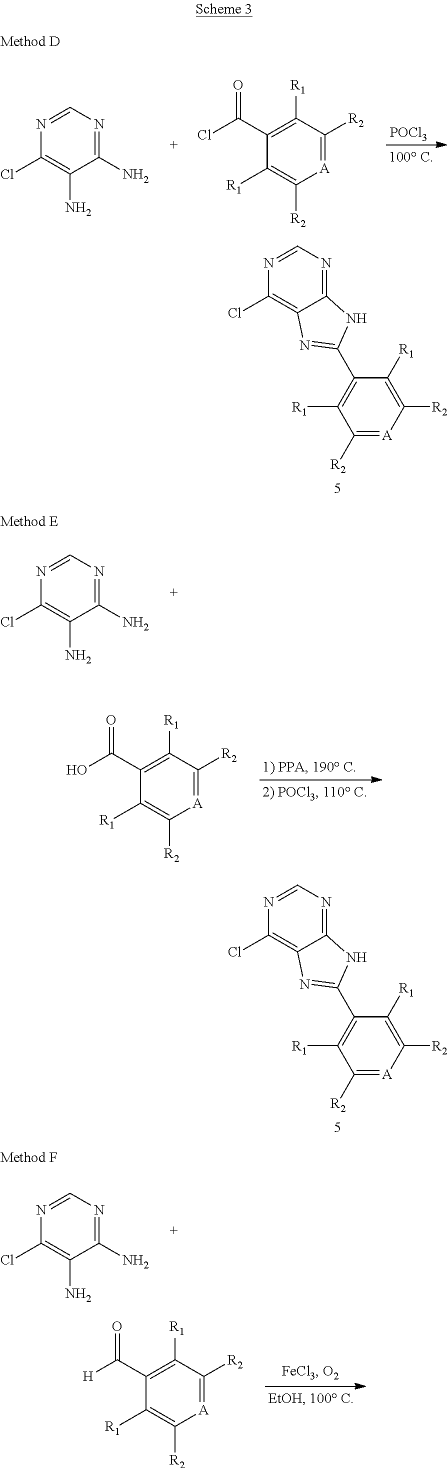

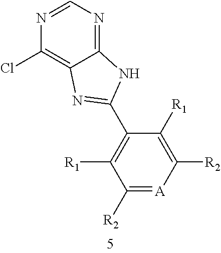

Scheme 3 describes a general method for preparing compound 5, which can be used in further methods in preparing compounds of the present invention. In Method D, 6-chloropyrimidine-4,5-diamine is treated with an acid chloride in the presence of POCl₃, to give intermediate 5. Alternatively, 6-chloropyrimidine-4,5-diamine is condensed with an acid when heated in PPA, as shown in Method E. This can be accompanied by the hydrolysis of chloride to give a hydroxyl intermediate, which can be subsequently converted to compound 5 when treated with POCl₃. In Method F, 6-chloropyrimidine-4,5-diamine can be transformed to compound 5 when heated with FeCl₃ and oxygen in ethanol.

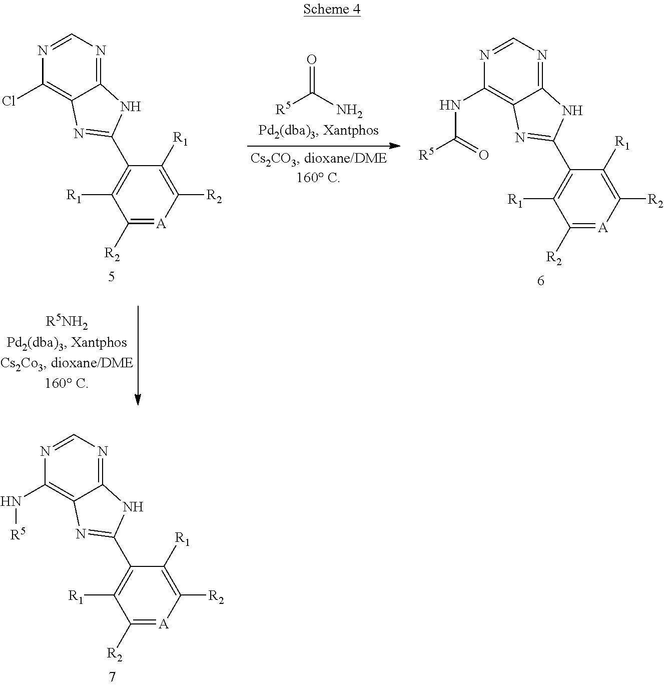

Scheme 4 describes general methods for preparing compounds 6 and 7, using compound 5, by palladium-catalyzed reactions. Heating of chloride 5 with an amide (R⁵CONH₂) or an amine (R⁵NH₂) at 160° C. for a couple of hours under nitrogen, in the presence of Pd₂(dba)₃, XantPhos, Cs₂CO₃ and 1,4-Dioxane/DME, gives the desired product. This Palldium-catalyzed coupling reaction can be carried out in a sealed tube in a microwave reactor.

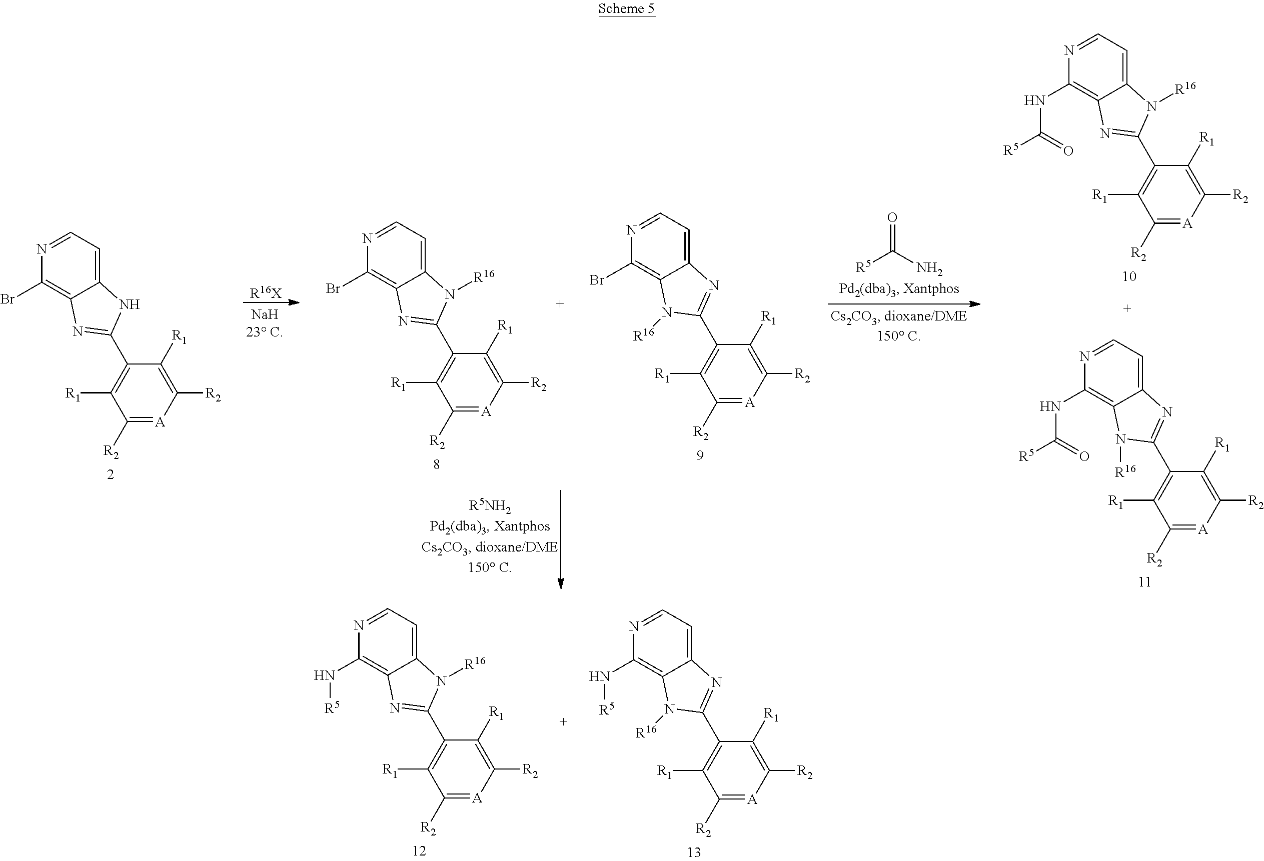

Scheme 5 shows general synthetic methods for preparing further compounds of the present invention. Bromide 2 can be alkylated by an electrophile to give a mixture of N-substituted imidazoles 8 and 9, which can be carried on to the next step without separation. The following palladium-catalyzed coupling reaction can be carried out in a sealed tube in a microwave reactor. Heating of a mixture of bromides 8 and 9 with an amide (R⁵CONH₂) or an amine (R⁵NH₂) at 150° C. for a couple of hours under nitrogen, in the presence of Pd₂(dba)₃, XantPhos, Cs₂CO₃ and 1,4-Dioxane/DME, gives the desired products, which could then be separated by rpHPLC or SFC.

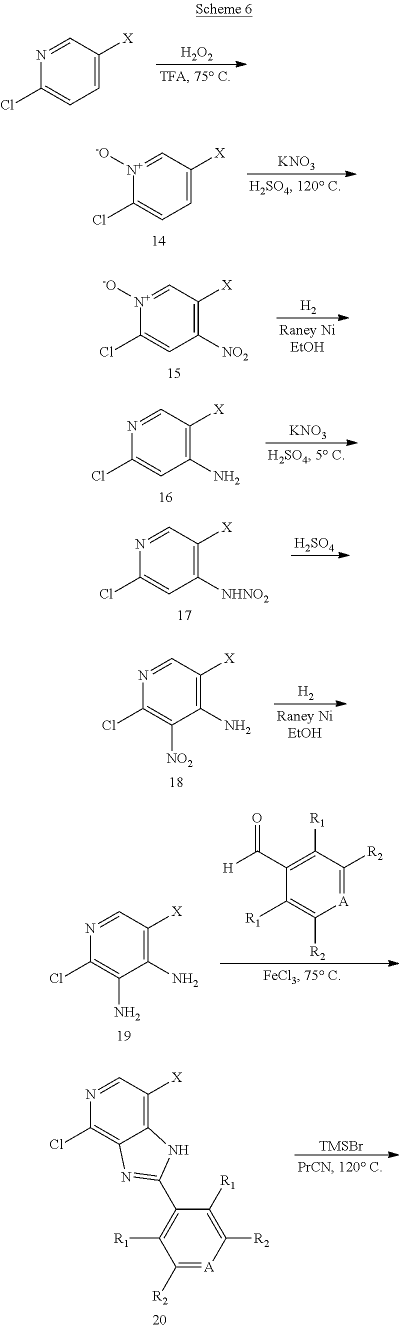

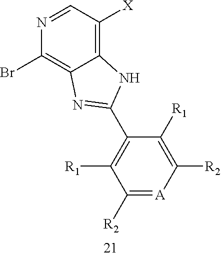

General preparation of intermediate 21 is shown in Scheme 6. Oxidation of a 2-Cl pyridine by hydrogen peroxide in TFA gives the N-oxide 14, which can be nitrated in concentrated sulfuric acid to provide compound 15. Hydrogenation of 15 gives 4-aminopyridine 16, which can be further nitrated to provide 17. Subsequent treatment of intermediate 16 with sulfuric acid gives compound 18, which can be reduced by hydrogen in the presence of Raney Ni to give diaminopyridine 19. Condensation of 19 with benaldehyde gives imidazopyridine 20, which can be transformed to bromide 21 when treated with TMSBr in propyl nitrile.

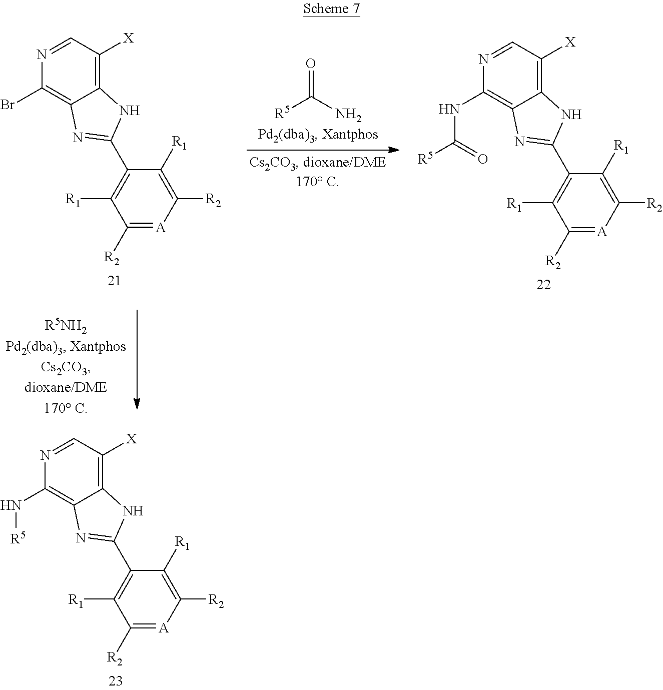

Scheme 7 describes general methods for preparing compounds 22 and 23, using bromide 21, by palladium-catalyzed reactions. Heating of bromide 21 with an amide (R⁵CONH₂) or an amine (R⁵NH₂) at 170° C. for a couple of hours, in the presence of Pd₂(dba)₃, XantPhos, Cs₂CO₃ and 1,4-Dioxane/DME, gives the desired product 22 or 23. This Palldium-catalyzed coupling reaction can be carried out in a sealed tube in a microwave reactor.

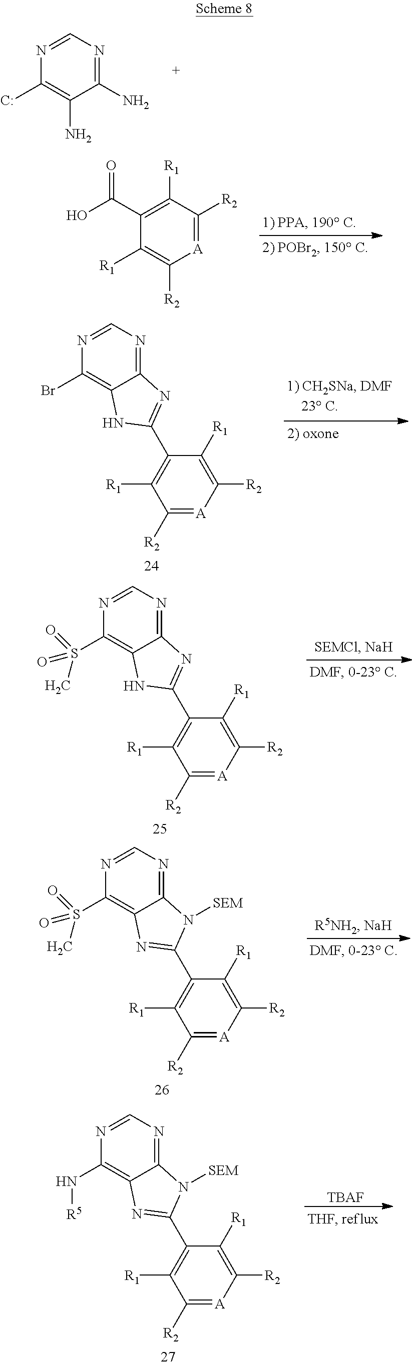

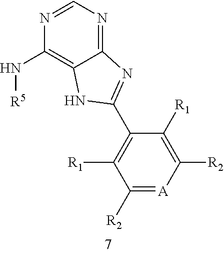

An alternative strategy to couple a purine chloride 5 and an amine (R⁵NH₂) via palladium-mediate reaction (Scheme 4) is described in Scheme 8. Condensation of 6-chloropyrimidine-4,5-diamine with an acid in the presence of PPA resulted in purin-6-ol, which was transformed to 24 with POBr₃. Displacement of bromide was carried out with sodium methanethiolate, followed by oxidation with oxone to give methylsulfone 25. Subsequent reaction with SEMCl provided 26, which reacted smoothly with R⁵NH₂ in the presence of a base (NaH) to give 27. Deprotection of 27 with TBAF in refluxing THF resulted in final product 7.

It will be appreciated that where appropriate functional groups exist, compounds of various formulae or any intermediates used in their preparation may be further derivatised by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

In each of the exemplary Schemes it may be advantageous to separate reaction products from one another and/or from starting materials. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

A single stereoisomer, e.g. an enantiomer, substantially free of its stereoisomer may be obtained by resolution of the racemic mixture using a method such as formation of diastereomers using optically active resolving agents (Eliel, E. and Wilen, S., Stereochemistry of Organic Compounds, John Wiley & Sons, Inc., New York, 1994; Lochmuller, C. H., J. Chromatogr., 113 (3):283-302 (1975)). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Drug Stereochemistry, Analytical Methods and Pharmacology, Irving W. Wainer, Ed., Marcel Dekker, Inc., New York (1993).

Diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, the substrate to be resolved is reacted with one enantiomer of a chiral compound to form a diastereomeric pair (Eliel, E. and Wilen, S., Stereochemistry of Organic Compounds, John Wiley & Sons, Inc., New York, 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g. (−) menthyl chloroformate in the presence of base, or Mosher ester, α-methoxy-α-(trifluoromethyl)phenyl acetate (Jacob, J. Org. Chem. 47:4165 (1982)), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (Chiral Liquid Chromatography W. J. Lough, Ed., Chapman and Hall, New York, (1989); Okamoto, J. of Chromatogr. 513:375-378 (1990)). Enriched or purified enantiomers can be distinguished by methods used to distinguish other chiral molecules with asymmetric carbon atoms, such as optical rotation and circular dichroism.

### Pharmaceutical Compositions and Administration

Another embodiment provides pharmaceutical compositions or medicaments containing the compounds of Formulas Ia-Ib and a therapeutically inert carrier, diluent or excipient, as well as methods of using the compounds of the invention to prepare such compositions and medicaments. In one example, compounds of Formulas Ia-Ib may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of Formulas Ia-Ib is formulated in an acetate buffer, at pH 5. In another embodiment, the compounds of Formulas Ia-Ib are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular patient being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit TYK2 kinase activity. For example, such amount may be below the amount that is toxic to normal cells, or the patient as a whole.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Sustained-release preparations may be prepared. Suitable examples of sustained-release preparations include semipermeable matrices of solid hydrophobic polymers containing a compound of Formulas Ia-Ib, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

In one example, the pharmaceutically effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, preferably contain from about 5-100 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 mg, 50 mg, 100 mg, 250 mg or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

In one embodiment, the pharmaceutical composition also includes an additional therapeutic agent selected from an anti-proliferative agent, an anti-inflammatory agent, an immunomodulatory agent, a neurotropic factor, an agent for treating cardiovascular disease, an agent for treating liver disease, an anti-viral agent, an agent for treating blood disorders, an agent for treating diabetes, or an agent for treating immunodeficiency disorders.

An embodiment, therefore, includes a pharmaceutical composition comprising a compound of Formulas Ia-Ib, or a stereoisomer or pharmaceutically acceptable salt thereof. In a further embodiment includes a pharmaceutical composition comprising a compound of Formulas Ia-Ib, or a stereoisomer or pharmaceutically acceptable salt thereof, together with a pharmaceutically acceptable carrier or excipient.

Another embodiment includes a pharmaceutical composition comprising a compound of Formulas Ia-Ib, or a stereoisomer or pharmaceutically acceptable salt thereof, for use in the treatment of an immunological or inflammatory disease. Another embodiment includes a pharmaceutical composition comprising a compound of Formulas Ia-Ib, or a stereoisomer or pharmaceutically acceptable salt thereof for use in the treatment of psoriasis or inflammatory bowel disease.

### Indications and Methods of Treatment

The compounds of Formulas Ia-Ib inhibit TYK2 kinase activity. Accordingly, the compounds of Formulas Ia-Ib are useful for reducing inflammation in particular patient tissue and cells. Compounds of the invention are useful for inhibiting TYK2 kinase activity in cells that overexpress TYK2 kinase. Alternatively, compounds of Formulas Ia-Ib are useful for inhibiting TYK2 kinase activity in cells in which the type I interferon, IL-6, IL-10, IL-12 and

IL-23 signaling pathway is disruptive or abnormal, for example by binding to TYK2 kinase and inhibiting its activity. More broadly, the compounds of Formulas Ia-Ib can be used for the treatment of immunological or inflammatory disorders.

Another embodiment includes a method of treating or lessening the severity of a disease or condition responsive to the inhibition of TYK2 kinase activity in a patient. The method includes the step of administering to a patient a therapeutically effective amount of a compound of Formulas Ia-Ib, stereoisomers, tautomers or salts thereof.

In one embodiment, a compound of Formulas Ia-Ib is administered to a patient in a therapeutically effective amount to treat or lessen the severity of a disease or condition responsive to the inhibition of TYK2 kinase activity, and said compound is at least 15 fold, alternatively 10 fold, alternatively 5 fold or more selective in inhibiting TYK2 kinase activity over inhibiting each of the other Janus kinase activities.

Another embodiment includes a compound of Formulas Ia-Ib, stereoisomers, tautomers or salts thereof for use in therapy.

Another embodiment includes a compound of Formulas Ia-Ib, stereoisomers, tautomers or salts thereof for use in treating an immunological or inflammatory disease.

Another embodiment includes a compound of Formulas Ia-Ib, stereoisomers, tautomers or salts thereof for use in treating psoriasis or inflammatory bowel disease.

Another embodiment includes the use of a compound of Formulas Ia-Ib, stereoisomers, tautomers or salts thereof for treating an immunological or inflammatory disease.

Another embodiment includes the use of a compound of Formulas Ia-Ib, stereoisomers, tautomers or salts thereof for treating psoriasis or inflammatory bowel disease.

Another embodiment includes the use of a compound of Formulas Ia-Ib, stereoisomers, tautomers or salts thereof in the preparation of a medicament for the treatment of an immunological or inflammatory disease.

Another embodiment includes the use of a compound of Formulas Ia-Ib, stereoisomers, tautomers or salts thereof in the preparation of a medicament for the treatment of psoriasis or inflammatory bowel disease.

In one embodiment, the disease or condition is cancer, stroke, diabetes, hepatomegaly, cardiovascular disease, multiple sclerosis, Alzheimer's disease, cystic fibrosis, viral disease, autoimmune diseases, immunological disease, atherosclerosis, restenosis, psoriasis, allergic disorders, inflammatory disease, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, liver disease, pathologic immune conditions involving T cell activation, CNS disorders or a myeloproliferative disorder.

In one embodiment, the disease or condition is cancer.

In one embodiment, the disease or condition is an immunological disorder.

In one embodiment, the disease is a myeloproliferative disorder.

In one embodiment, the myeloproliferative disorder is polycythemia vera, essential thrombocytosis, myelofibrosis or chronic myelogenous leukemia (CML).

In one embodiment, the disease is asthma.

In one embodiment, the cancer is breast, ovary, cervix, prostate, testis, penile, genitourinary tract, seminoma, esophagus, larynx, gastric, stomach, gastrointestinal, skin, keratoacanthoma, follicular carcinoma, melanoma, lung, small cell lung carcinoma, non-small cell lung carcinoma (NSCLC), lung adenocarcinoma, squamous carcinoma of the lung, colon, pancreas, thyroid, papillary, bladder, liver, biliary passage, kidney, bone, myeloid disorders, lymphoid disorders, hairy cells, buccal cavity and pharynx (oral), lip, tongue, mouth, salivary gland, pharynx, small intestine, colon, rectum, anal, renal, prostate, vulval, thyroid, large intestine, endometrial, uterine, brain, central nervous system, cancer of the peritoneum, hepatocellular cancer, head cancer, neck cancer, Hodgkin's or leukemia.

In one embodiment, the cardiovascular disease is restenosis, cardiomegaly, atherosclerosis, myocardial infarction or congestive heart failure.

In one embodiment, the neurodegenerative disease is Alzheimer's disease, Parkinson's disease, amyotrophic lateral sclerosis, Huntington's disease, and cerebral ischemia, and neurodegenerative disease caused by traumatic injury, glutamate neurotoxicity or hypoxia.

In one embodiment, the inflammatory disease is inflammatory bowel disease, Crohn's disease, ulcerative colitis, rheumatoid arthritis, psoriasis, contact dermatitis or delayed hypersensitivity reactions.

In one embodiment, the autoimmune disease is lupus or multiple sclerosis.

Evaluation of drug-induced immunosuppression by the compounds of the invention may be performed using in vivo functional tests, such as rodent models of induced arthritis and therapeutic or prophylactic treatment to assess disease score, T cell-dependent antibody response (TDAR), and delayed-type hypersensitivity (DTH). Other in vivo systems including murine models of host defense against infections or tumor resistance (Burleson G R, Dean J H, and Munson A E. Methods in Immunotoxicology, Vol. 1. Wiley-Liss, New York, 1995) may be considered to elucidate the nature or mechanisms of observed immunosuppression. The in vivo test systems can be complemented by well-established in vitro or ex vivo functional assays for the assessment of immune competence. These assays may comprise B or T cell proliferation in response to mitogens or specific antigens, measurement of signaling through one or more of the Janus kinase pathways in B or T cells or immortalized B or T cell lines, measurement of cell surface markers in response to B or T cell signaling, natural killer (NK) cell activity, mast cell activity, mast cell degranulation, macrophage phagocytosis or kill activity, and neutrophil oxidative burst and/or chemotaxis. In each of these tests determination of cytokine production by particular effector cells (e.g., lymphocytes, NK, monocytes/macrophages, neutrophils) may be included. The in vitro and ex vivo assays can be applied in both preclinical and clinical testing using lymphoid tissues and/or peripheral blood (House R V. “Theory and practice of cytokine assessment in immunotoxicology” (1999) Methods 19:17-27; Hubbard A K. “Effects of xenobiotics on macrophage function: evaluation in vitro” (1999) Methods; 19:8-16; Lebrec H, et al (2001) Toxicology 158:25-29).

Collagen-Induced Arthritis (CIA) 6-week detailed study using an autoimmune mechanism to mimic human arthritis; rat and mouse models (Example 68). Collagen-induced arthritis (CIA) is one of the most commonly used animal models of human rheumatoid arthritis (RA). Joint inflammation, which develops in animals with CIA, strongly resembles inflammation observed in patients with RA. Blocking tumor necrosis factor (TNF) is an efficacious treatment of CIA, just as it is a highly efficacious therapy in treatment of RA patients. CIA is mediated by both T-cells and antibodies (B-cells). Macrophages are believed to play an important role in mediating tissue damage during disease development. CIA is induced by immunizing animals with collagen emulsified in Complete Freund's Adjuvant (CFA). It is most commonly induced in the DBA/1 mouse strain, but the disease can also be induced in Lewis rats.

There is good evidence that B-cells play a key role in the pathogenesis of autoimmune and/or inflammatory disease. Protein-based therapeutics that deplete B cells such as Rituxan are effective against autoantibody-driven inflammatory diseases such as rheumatoid arthritis (Rastetter et al. (2004) Annu Rev Med 55:477). CD69 is the early activation marker in leukocytes including T cells, thymocytes, B cells, NK cells, neutrophils, and eosinophils. The CD69 human whole blood assay (Example 69) determines the ability of compounds to inhibit the production of CD69 by B lymphocytes in human whole blood activated by crosslinking surface IgM with goat F(ab′)2 anti-human IgM.

The T-cell Dependent Antibody Response (TDAR) is a predictive assay for immune function testing when potential immunotoxic effects of compounds need to be studied. The IgM-Plaque Forming Cell (PFC) assay, using Sheep Red Blood Cells (SRBC) as the antigen, is currently a widely accepted and validated standard test. TDAR has proven to be a highly predictable assay for adult exposure immunotoxicity detection in mice based on the US National Toxicology Program (NTP) database (M. I. Luster et al (1992) Fundam. Appl. Toxicol. 18:200-210). The utility of this assay stems from the fact that it is a holistic measurement involving several important components of an immune response. A TDAR is dependent on functions of the following cellular compartments: (1) antigen-presenting cells, such as macrophages or dendritic cells; (2) T-helper cells, which are critical players in the genesis of the response, as well as in isotype switching; and (3) B-cells, which are the ultimate effector cells and are responsible for antibody production. Chemically-induced changes in any one compartment can cause significant changes in the overall TDAR (M. P. Holsapple In: G. R. Burleson, J. H. Dean and A. E. Munson, Editors, Modern Methods in Immunotoxicology, Volume 1, Wiley-Liss Publishers, New York, N.Y. (1995), pp. 71-108). Usually, this assay is performed either as an ELISA for measurement of soluble antibody (R. J. Smialowizc et al (2001) Toxicol. Sci. 61:164-175) or as a plaque (or antibody) forming cell assay (L. Guo et al (2002) Toxicol. Appl. Pharmacol. 181:219-227) to detect plasma cells secreting antigen specific antibodies. The antigen of choice is either whole cells (e.g. sheep erythrocytes) or soluble protein antigens (T. Miller et al (1998) Toxicol. Sci. 42:129-135).

A compound of Formulas Ia-Ib may be administered by any route appropriate to the disease or condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary, and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound of Formulas Ia-Ib is administered orally, it may be formulated as a pill, capsule, tablet, etc. with a pharmaceutically acceptable carrier or excipient. Where the compound of Formulas Ia-Ib is administered parenterally, it may be formulated with a pharmaceutically acceptable parenteral vehicle and in a unit dosage injectable form, as detailed below.

A dose to treat human patients may range from about 5 mg to about 1000 mg of a compound of Formulas Ia-Ib. A typical dose may be about 5 mg to about 300 mg of a compound of Formulas Ia-Ib. A dose may be administered once a day (QD), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### Combination Therapy

The compounds of Formulas Ia-Ib may be employed alone or in combination with other therapeutic agents for the treatment of a disease or disorder described herein, such as an immunologic disorder (e.g. psoriasis or inflammation) or a hyperproliferative disorder (e.g., cancer). In certain embodiments, a compound of Formulas Ia-Ib is combined in a pharmaceutical combination formulation, or dosing regimen as combination therapy, with a second therapeutic compound that has anti-inflammatory or anti-hyperproliferative properties or that is useful for treating an inflammation, immune-response disorder, or hyperproliferative disorder (e.g., cancer). The second therapeutic agent may be a NSAID or other anti-inflammatory agent. The second therapeutic agent may be a chemotherapeutic agent. The second therapeutic agent of the pharmaceutical combination formulation or dosing regimen preferably has complementary activities to the compound of Formulas Ia-Ib such that they do not adversely affect each other. Such compounds are suitably present in combination in amounts that are effective for the purpose intended. In one embodiment, a composition of this invention comprises a compound of Formulas Ia-Ib, or a stereoisomer, geometric isomer, tautomer, solvate, metabolite, or pharmaceutically acceptable salt or prodrug thereof, in combination with a therapeutic agent such as an NSAID.

Another embodiment, therefore, includes a method of treating or lessening the severity of a disease or condition responsive to the inhibition of TYK2 kinase in a patient, comprising administering to said patient a therapeutically effective amount of a compound of Formulas Ia-Ib, and further comprising, administering a second therapeutic agent.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

In a particular embodiment of therapy, a compound of Formulas Ia-Ib, or a stereoisomer, geometric isomer, tautomer, solvate, metabolite, or pharmaceutically acceptable salt or prodrug thereof, may be combined with other therapeutic, hormonal or antibody agents such as those described herein, as well as combined with surgical therapy and radiotherapy. Combination therapies according to the present invention thus comprise the administration of at least one compound of Formulas Ia-Ib, or a stereoisomer, geometric isomer, tautomer, solvate, metabolite, or pharmaceutically acceptable salt or prodrug thereof, and the use of at least one other cancer treatment method, or immunological disorder method. The amounts of the compound(s) of Formulas Ia-Ib and the other pharmaceutically active immunologic or chemotherapeutic agent(s) and the relative timings of administration will be selected in order to achieve the desired combined therapeutic effect.

### Methods and Articles of Manufacture

Another embodiment includes a method of manufacturing a compound of Formulas Ia-Ib. The method includes: (a) reacting a compound of the formula:

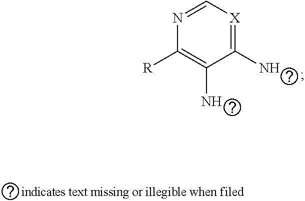

wherein R is halogen or other leaving group, and X is as defined for Formulas Ia-Ib, with a compound of the formula:

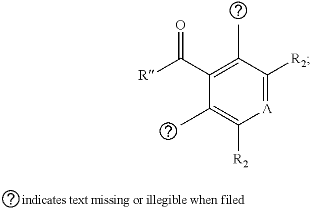

wherein R″ is halogen or other leaving group, R¹, R² and A are as defined for Formulas Ia-Ib, to prepare a compound of the formula:

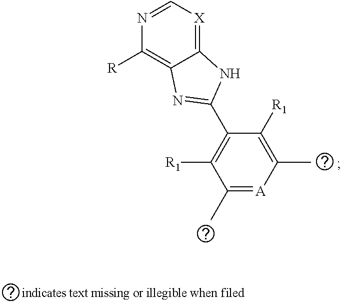

(b) optionally reacting a compound of the above formula with a compound of formula Lv-R¹⁶, wherein Lv is a leaving group, for example halogen, to form a compound of the formulas:

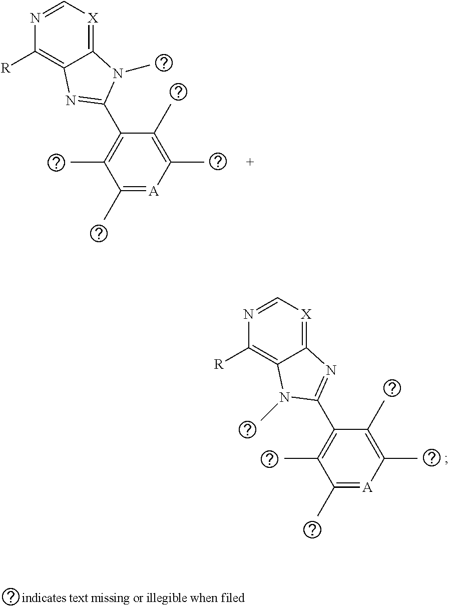

wherein R¹⁶ is as defined for Formulas Ia-Ib;
(c) reacting the above said compounds with a compound of the formula R⁴-R⁵ to form a compound of Formulas Ia-Ib, having the formula:

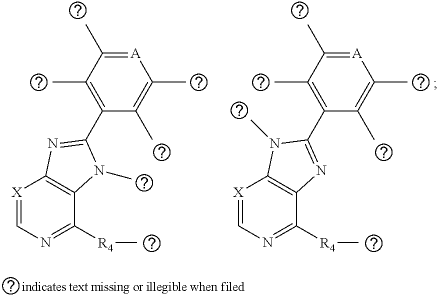

and
(d) optionally further functionalizing said above compound.

Another embodiment includes a kit for treating a disease or disorder responsive to the inhibition of aTYK2 kinase. The kit includes:
(a) a first pharmaceutical composition comprising a compound of Formulas Ia-Ib; and
(b) instructions for use.

In another embodiment, the kit further includes:
(c) a second pharmaceutical composition, which includes a chemotherapeutic agent.

In one embodiment, the instructions include instructions for the simultaneous, sequential or separate administration of said first and second pharmaceutical compositions to a patient in need thereof.

In one embodiment, the first and second compositions are contained in separate containers.

In one embodiment, the first and second compositions are contained in the same container.

Containers for use include, for example, bottles, vials, syringes, blister pack, etc. The containers may be formed from a variety of materials such as glass or plastic. The container includes a compound of Formulas Ia-Ib or formulation thereof which is effective for treating the condition and may have a sterile access port (for example the container may be an intravenous solution bag or a vial having a stopper pierceable by a hypodermic injection needle). The container includes a composition comprising at least one compound of Formulas Ia-Ib. The label or package insert indicates that the composition is used for treating the condition of choice, such as cancer. In one embodiment, the label or package inserts indicates that the composition comprising the compound of Formulas Ia-Ib can be used to treat a disorder. In addition, the label or package insert may indicate that the patient to be treated is one having a disorder characterized by overactive or irregular kinase activity. The label or package insert may also indicate that the composition can be used to treat other disorders.

The article of manufacture may comprise (a) a first container with a compound of Formulas Ia-Ib contained therein; and (b) a second container with a second pharmaceutical formulation contained therein, wherein the second pharmaceutical formulation comprises a chemotherapeutic agent. The article of manufacture in this embodiment of the invention may further comprise a package insert indicating that the first and second compounds can be used to treat patients at risk of stroke, thrombus or thrombosis disorder. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare other compounds of Formulas Ia-Ib, and alternative methods for preparing the compounds of Formulas Ia-Ib are within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

### Biological Examples

Compounds of Formulas Ia-Ib may be assayed for the ability to modulate the activity of protein kinases, tyrosine kinases, additional serine/threonine kinases, and/or dual specificity kinases in vitro and in vivo. In vitro assays include biochemical and cell-based assays that determine inhibition of the kinase activity. Alternate in vitro assays quantify the ability of the compound of Formulas Ia-Ib to bind to kinases and may be measured either by radiolabelling the compound of Formulas Ia-Ib prior to binding, isolating the compound of Formulas Ia-Ib/kinase complex and determining the amount of radiolabel bound, or by running a competition experiment where a compound of Formulas Ia-Ib is incubated with known radiolabeled ligands. These and other useful in vitro assays are well known to those of skill in the art.

In an embodiment, the compounds of Formulas Ia-Ib can be used to control, modulate or inhibit tyrosine kinase activity, for example TYK2 kinase activity, additional serine/threonine kinases, and/or dual specificity kinases. Thus, they are useful as pharmacological standards for use in the development of new biological tests, assays and in the search for new pharmacological agents.

### Example A

### JAK1, JAK2 and TYK2 Inhibition Assay Protocol

The activity of the isolated JAK1, JAK2 or TYK2 kinase domain was measured by monitoring phosphorylation of a peptide derived from JAK3 (Val-Ala-Leu-Val-Asp-Gly-Tyr-Phe-Arg-Leu-Thr-Thr) fluorescently labeled on the N-terminus with 5-carboxyfluorescein using the Caliper LabChip technology (Caliper Life Sciences, Hopkinton, Mass.). To determine the inhibition constants (Ki) of Examples 1-472, compounds were diluted serially in DMSO and added to 50 uL kinase reactions containing 1.5 nM JAK1, 0.2 nM purified JAK2 or 1 nM purified TYK2 enzyme, 100 mM Hepes pH7.2, 0.015% Brij-35, 1.5 uM peptide substrate, 25 uM ATP, 10 mM MgCl₂, 4 mM DTT at a final DMSO concentration of 2%. Reactions were incubated at 22° C. in 384-well polypropylene microtiter plates for 30 minutes and then stopped by addition of 25 uL of an EDTA containing solution (100 mM Hepes pH 7.2, 0.015% Brij-35, 150 mM EDTA), resulting in a final EDTA concentration of 50 mM. After termination of the kinase reaction, the proportion of phosphorylated product was determined as a fraction of total peptide substrate using the Caliper LabChip 3000 according to the manufacturer's specifications. Ki values were then determined using the Morrison tight binding model. Morrison, J. F., Biochim. Biophys. Acta. 185:269-296 (1969); William, J. W. and Morrison, J. F., Meth. Enzymol., 63:437-467 (1979).

### Example B

### JAK3 Inhibition Assay Protocol

The activity of the isolated JAK3 kinase domain was measured by monitoring phosphorylation of a peptide derived from JAK3 (Leu-Pro-Leu-Asp-Lys-Asp-Tyr-Tyr-Val-Val-Arg) fluorescently labeled on the N-terminus with 5-carboxyfluorescein using the Caliper LabChip technology (Caliper Life Sciences, Hopkinton, Mass.). To determine the inhibition constants (Ki) of Examples 1-472, compounds were diluted serially in DMSO and added to 50 uL kinase reactions containing 5 nM purified JAK3 enzyme, 100 mM Hepes pH7.2, 0.015% Brij-35, 1.5 uM peptide substrate, 5 uM ATP, 10 mM MgCl₂, 4 mM DTT at a final DMSO concentration of 2%. Reactions were incubated at 22° C. in 384-well polypropylene microtiter plates for 30 minutes and then stopped by addition of 25 uL of an EDTA containing solution (100 mM Hepes pH 7.2, 0.015% Brij-35, 150 mM EDTA), resulting in a final EDTA concentration of 50 mM. After termination of the kinase reaction, the proportion of phosphorylated product was determined as a fraction of total peptide substrate using the Caliper LabChip 3000 according to the manufacturer's specifications. Ki values were then determined using the Morrison tight binding model. Morrison, J. F., Biochim. Biophys. Acta. 185:269-296 (1969); William, J. W. and Morrison, J. F., Meth. Enzymol., 63:437-467 (1979).

### Example C

### Cell-Based Pharmacology Assays

The activities of compounds 1-472 were determined in cell-based assays that are designed to measure Janus kinase dependent signaling. Compounds were serially diluted in DMSO and incubated with Set-2 cells (German Collection of Microorganisms and Cell Cultures (DSMZ); Braunschweig, Germany), which express the JAK2V617F mutant protein, in 96-well microtiter plates for 1 hr at 37° C. in RPMI medium at a final cell density of 10⁵ cells per well and a final DMSO concentration of 0.57%. Compound-mediated effects on STATS phosphorylation were then measured in the lysates of incubated cells using the Meso Scale Discovery (MSD) technology (Gaithersburg, Md.) according to the manufacturer's protocol and EC₅₀ values were determined Alternatively, serially diluted compounds were added to NK92 cells (American Type Culture Collection (ATCC); Manassas, Va.) in 96-well microtiter plates in RPMI medium at a final cell density of 10⁵ cells per well and a final DMSO concentration of 0.57%. Human recombinant IL-12 (R&D systems; Minneapolis, Minn.) was then added at a final concentration of 10 ng/ml to the microtiter plates containing the NK92 cells and compound and the plates were incubated for 1 hr at 37° C. Compound-mediated effects on STAT4 phosphorylation were then measured in the lysates of incubated cells using the Meso Scale Discovery (MSD) technology (Gaithersburg, Md.) according to the manufacturer's protocol and EC₅₀ values were determined

The compounds of Examples 1-472 were tested in the above assays and found to have IC₅₀ values for TYK2 inhibition of less than about 5 uM. The compounds of Examples 1-28, 30-102, 104-134 and 136-215 were tested in the above assays and found to have IC₅₀ values for TYK2 inhibition of less than about 1 uM. The compounds of Examples 1-27, 30-86, 93-95, 97-102, 104, 106-114, 116-134 and 136-215 were tested in the above assays and found to have IC₅₀ values for TYK2 inhibition of less than about 100 nM.

Activities for certain compounds of Formula Ia-Ib in the above assay (Example A) are shown in Table A below.

TABLE A

| Example no. | Tyk2 Ki (nM) |
|---|---|
| 1 | 1.7 |
| 3 | 14.5 |
| 5 | 0.6 |
| 6 | 3.1 |
| 7 | 7.8 |
| 10 | 2.5 |
| 11 | 4.9 |
| 12 | 13.3 |
| 13 | 19.5 |
| 14 | 2.8 |
| 15 | 8.1 |
| 16 | 2.3 |
| 167 | 7.2 |
| 171 | 27.2 |
| 179 | 1.5 |
| 197 | 1.4 |
| 208 | 56 |
| 210 | 27.8 |
| 215 | 56.5 |
| 439 | 835 |
| 447 | 0.8 |

### Preparative Examples

### Abbreviations

### CD₃OD Deuterated Methanol

### DCM Dichloromethane

### DIPEA Diisopropylethylamine

### DMSO Dimethylsulfoxide

### DMF Dimethylformamide

### EtOAc Ethyl Acetate

### EtOH Ethanol

HCl Hydrochloric acid
HM-N Isolutet HM-N is a modified form of diatomaceous earth
IMS industrial methylated spirits

### MeOH Methanol

POCl₃ Phosphorus oxychloride

### NaH Sodium Hydride

### Na₂SO₄Sodium Sulfate

NaHCO₃ Sodium bicarbonate
NaOH Sodium hydroxide

### Pd(PPh₃)₄ Tetrakis(triphenylphosphine)palladium(0)

### NEt₃ Triethylamine

Pd₂ dba₃ Tris-(dibenzylideneacetone)dipalladium(0)
Si-SPE Pre-packed Isolute® silica flash chromatography cartridge
Si-ISCO Pre-packed ISCO® silica flash chromatography cartridge

### THF Tetrahydrofuran

### General Experimental Conditions

Compounds of this invention may be prepared from commercially available starting materials using the general methods illustrated herein. Specifically, 2,6-dichlorobenzoic acid, 2,6-dichlorobenzoyl chloride, 2-choro-6-fluorobenzoic acid, 2,6-bis(trifluoromethyl)benzoic acid, 2,6-dimethylbenzoic acid, 2-chloro-4-(methylsulfonyl)benzoic acid, 2-chlorobenzoic acid, 2-(trifluoromethyl)benzoic acid, 2-(trifluoromethoxy)benzoic acid, 2,6-difluorobenzoic acid, were purchased from Aldrich (St. Louis, Mo.). 2-chloropyridine-3,4-diamine was purchased from Synthonix (West Forest, N.C.). 6-chloropyrimidine-4,5-diamine was purchased from Princeton Biomolecular Research (Monmouth Junction, N.J.). All commercial chemicals, including reagents and solvents, were used as received.

High Pressure Liquid Chromatography—Mass Spectrometry (LCMS) experiments to determine retention times (RT) and associated mass ions were performed using one of the following methods, with UV detector monitoring at 220 nm and 254 nm, and mass spectrometry scanning 110-800 amu in ESI+ ionization mode.

LC/MS Method A: column: XBridge C18, 4.6×50 mm, 3.5 mm; mobile phase: A water (0.01% ammonia), B CH₃CN; gradient: 5%-95% B in 8.0 min; flow rate: 1.2 mL/min; oven temperature 40° C. LC/MS.

Method B: column: AgilentSD-C18, 2.1×30 mm, 1.8 um; mobile phase: A water with 0.5% TFA, B CH₃CN with 0.5% TFA in 8.5 min; flowrate 0.4 mL/min; oven temperature 40° C.

LCMS Method C: column. XBridge C18, 4.6×50 mm, 3.5 mm; mobile phase: A water (0.01% NH₄HCO₃), B CH₃CN; gradient: 5%-95% B in 8.0 min; flow rate: 1.2 mL/min; oven temperature 40° C.

¹H NMR spectra were recorded at ambient temperature using a Varian Unity Inova (400 MHz) spectrometer with a triple resonance 5 mm probe. Chemical shifts are expressed in ppm relative to tetramethylsilane. The following abbreviations have been used: br=broad signal, s=singlet, d=doublet, dd=double doublet, t=triplet, q=quartet, m=multiplet. Microwave experiments were carried out using a Biotage Initiator 60™ which uses a single-mode resonator and dynamic field tuning. Temperature from 40-250° C. can be achieved, and pressures of up to 30 bar can be reached.

### Example i

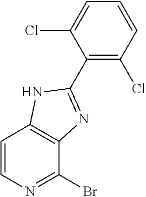 [SMILES: Clc1cccc(Cl)c1-c1nc2c(Br)nccc2[nH]1]

### 4-bromo-2-(2,6-dichlorophenyl)-1H-imidazo[4,5-c]pyridine

### Method A:

Step 1: To a mixture of 2-chloropyridine-3,4-diamine (2.0 g, 13.93 mmol), diisopropylethylamine (7.29 mL, 41.79 mmol) in CH₂Cl₂ (100 mL) and CH₃CN (10 mL) cooled to 0° C., was added 2,6-dichlorobenzoyl chloride (2.92 g, 13.93 mmol) portionwise. Then 50 mL of CH₃CN was added to dissolve the reaction mixture and the reaction was warmed to 23° C. overnight. Monitoring the reaction by LC-MS showed complete conversion.

The mixture was concentrated to dryness via rotavap to give a brown residue, which was then treated with POCl₃ (10.68 g, 69.65 mmol). The reaction mixture was heated at 120° C. overnight. Then the reaction mixture was cooled to 23° C., concentrated via rotavap, and carefully poured onto ice slowly. After neutralization with a saturated aqueous NaHCO₃ solution to pH 7.0, the mixture was extracted with EtOAc (2×100 mL), washed with brine, and dried over anhydrous Na₂SO₄. Crude mixture was purified by column chromatography on silica gel with 0-10% CH₂Cl₂/MeOH to give desired product (2.9 g, 70% yield). ¹H NMR (DMSO-d₆, 500 MHz): δ 13.75 (s, 1H), 8.20 (m, 1H), 7.73-7.65 (m, 5H). LCMS (ESI) m/z: 298.0 [M+H⁺]

Step 2: 2-(2,6-dichlorophenyl)-1H-imidazo[4,5-c]pyridin-4-ol (0.2 g, 0.6 mmol) and a HBr solution in AcOH (33%, 2 mL) was heated at 90° C. overnight. The reaction was cooled to room temperature and poured into ice followed by addition of an aqueous K₂CO₃ to neutralize to pH 7.0. The mixture was extracted with EtOAc (3×50 mL), washed with brine, dried over Na₂SO₄. Concentration via rotavap gave desired product as a pale yellow solid (0.14 g, 70% yield). ¹H NMR (DMSO-d₆, 500 MHz): δ 13.78 (s, 1H), 8.18 (m, 1H), 7.69 (m, 4H). LCMS (ESI) m/z: 343 [M+H⁺]

### Method B:

Step 1: A mixture of 2-chloropyridine-3,4-diamine (2.80 g, 19.4 mmol), 2,6-dichlorobenzoic acid (3.71 g, 19.4 mmol) and polyphosphoric acid (50 g) was heated at 190° C. for 3 hours with stirring. Then the mixture was cooled to ambient temperature and poured into ice/water. The resulting mixture was neutralized by addition of saturated Na₂CO₃. The crude product was collected by filtration, washed with water, and dried to afford a brown solid (5.4 g, 97% yield). ¹H NMR (DMSO-d₆, 500 MHz): δ 13.08 (s, 1H), 11.21 (s, 1H), 7.67-7.58 (m, 3H), 7.15 (m, 1H), 6.49 (d, J=7.0 Hz, 1H). LCMS (ESI) m/z: 280.0 [M+H⁺]

Step 2: 2-(2,6-dichlorophenyl)-1H-imidazo[4,5-c]pyridin-4-ol (5.4 g, 19.3 mmol) was added to the POBr₃ solution (20 mL) at 70° C. and the resulting mixture was then heated at 110° C. for 3 hours. The reaction was cooled to room temperature and poured into ice followed by addition of saturated Na₂CO₃ to neutralize. The precipitate was collected by filtration to afford the crude product which was then purified by column chromatography (EtOAc/hexanes=1:1) to give the desired product as a pale yellow solid (3.2 g, 48% yield). ¹H NMR (DMSO-d₆, 500 MHz): δ 13.78 (s, 1H), 8.18 (m, 1H), 7.69 (m, 4H). LCMS (ESI) m/z: 343 [M+H⁺]

### Method C:

Step 1: To a 250 mL round-bottomed flask with a magnetic stirrer were added 2-chloropyridine-3,4-diamine (1.00 g, 6.97 mmol), 2,6-dichlorobenzaldehyde (1.28 g, 7.32 mmol), and ammonium acetate (537 mg, 6.97 mmol) in absolute ethanol. The flask, loosely capped, was heated at 75° C. for 3 days. After the reaction was completed, the solvent was removed by vacuum and the residue was purified by column chromatography with petroleum ether/ethyl acetate (5:1) to give the desired product as a white solid (900 mg, 43% yield). ¹H NMR (DMSO-d₆, 500 MHz): δ 13.75 (s, 1H), 8.20 (m, 1H), 7.73-7.65 (m, 5H). LCMS (ESI) m/z: 298.0 [M+H⁺]

Step 2: A mixture of 4-chloro-2-(2,6-dichlorophenyl)-1H-imidazo[4,5-c]pyridine (500 mg, 1.70 mmol) and 33% solution of HBr in HOAc (5.0 mL) was heated at 90° C. for 15 hours. The reaction was then cooled to 25° C. and poured into ice-water. The resulting mixture was neutralized with an aqueous K₂CO₃ and the solid was collected by filtration, washed with water and dried by vacuum to give the desired product as a pale yellow solid (400 mg, 68% yield). ¹H NMR (DMSO-d₆, 500 MHz): δ 13.78 (s, 1H), 8.18 (m, 1H), 7.69 (m, 4H). LCMS (ESI) m/z: 343.0 [M+H⁺].

### Example 11

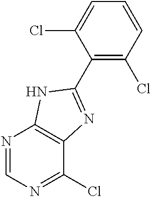 [SMILES: Clc1cccc(Cl)c1-c1nc2c(Cl)ncnc2[nH]1]

### 6-chloro-8-(2,6-dichlorophenyl)-9H-purine

### Method D:

A mixture of 6-chloropyrimidine-4,5-diamine (200 mg, 1.4 mmol), 2,6-dichlorobenzoyl chloride (288 mg, 1.4 mmol), ammonium chloride (0.44 g, 8.4 mmol) in POCl₃ (10 mL) was heated at 100° C. for 15 hours. The mixture was cooled to room temperature and carefully poured into ice/water slowly, neutralized with NH₄OH (25%) to basic (pH 7-8). The mixture was extracted with EtOAc (3×50 mL) and the organic layer was washed with brine (3×20 mL), dried with Na₂SO₄ and concentrated by vacuum. The residue was purified by column chromatography on silica gel with EtOAc and petroleum ether (1:5) to afford the desired product (96 mg, 22% yield). ¹H NMR (DMSO-d₆, 500 MHz): δ 14.46 (s, 1H), 8.83 (s, 1H), 7.68-7.76 (m, 3H). LCMS (ESI) m/z: 299.4 [M+H⁺].

### Method E:

Step 1: A mixture of 6-chloropyrimidine-4,5-diamine (3 g, 20.8 mmol), 2,6-dichlorobenzoic acid (4.33 g, 20.8 mmol) in polyphosphoric acid (PPA, 20 mL) was heated at 190° C. for 4 hours. The mixture was cooled to room temperature and carefully poured into ice/water slowly, neutralized with Na₂CO₃ to basic (pH 7-8). The resulting mixture was extracted with EtOAc (3×100 mL) and the organic layer was washed with brine (3×50 mL), dried over Na₂SO₄, and concentrated by vacuum. The residue was purified by column chromatography (EA=100%) to afford the desired product (2 g, 34% yield). ¹H NMR (DMSO-d₆, 500 MHz): δ 13.82 (s, 0.5H), 13.56 (s, 0.5H), 12.41 (s, 0.5H), 12.33 (s, 0.5H), 12.37 (d, J=38 Hz, 1H), 8.04 (s, 1H), 7.60-7.68 (m, 3H). LCMS (ESI) m/z: 281.0 [M+H⁺]

Step 2: 8-(2,6-dichlorophenyl)-9H-purin-6-ol (2.0 g, 7.14 mmol) in POCl₃ (20 mL) was heated at 100° C. for 15 hours. The solvent was removed by vacuum and the residue was carefully poured into ice/water, neutralized with NH₄OH (25%) to basic (pH 7-8). The resulting mixture was extracted with EtOAc (3×100 mL) and the organic layer was washed with brine (3×50 mL), dried over Na₂SO₄, and concentrated by vacuum. The residue was purified by column chromatography (EA=100%) to afford the desired product (1.8 g, 85% yield). ¹H NMR (DMSO-d₆, 500 MHz): δ 14.46 (s, 1H), 8.83 (s, 1H), 7.68-7.76 (m, 3H). LCMS (ESI) m/z: 299.4 [M+H⁺].

### Method F:

To a 500 mL flask with a condenser were added 6-chloropyrimidine-4,5-diamine (1.44 g, 10.0 mmol, 2-chloro-6-fluorobenzaldehyde (1.58 g, 10.0 mmol) and FeCl₃ (81 mg, 0.5 mmol) followed by addition of EtOH (100 mL). The mixture was evacuated by vacuum and refilled with O₂ three times. The reaction was heated under O₂ at 85° C. for 12 hours. Then the solvent was removed by vacuum and the residue was purified by column chromatography (MeOH: CH₂Cl₂=1:20) to give a yellow solid (4.3 g, 73%). ¹H NMR (DMSO-d₆, 500 MHz): δ 14.54 (br, 1H), 8.86 (s, 1H), 7.77 (dd, J=18.0, 10.0 Hz, 1H), 7.64 (d, J=10.5 Hz, 1H), 7.56 (t, J=11.0 Hz, 1H). LCMS (ESI) m/z: 283.0 [M+H⁺].

### Example iii

### Error! Objects Cannot be Created from Editing Field Codes

### 4-bromo-2-(2,6-dichlorophenyl)-7-fluoro-1H-imidazo[4,5-c]pyridine

Step 1: A mixture of 2-chloro-5-fluoropyridine (5.0 g, 38 mmol) and 12.5 mL of 50% hydrogen peroxide in 102 mL of trifluoroacetic acid was heated at 70-75° C. with stirring until TLC showed no strating material left. The reaction mixture was evaporated to dryness in vacuo and co-evaporated with toluene (20 mL) twice. The residue was diluted with 20 mL of water and 100 mL of methylene chloride and neutralized by dropwise addition of 28% ammonium hydroxide solution. The aqueous layer was further extracted with methylene chloride (100 mL) and the combined organics were dried, filtered, and evaporated. The crude product was purified by silica gel column chromatography (MeOH/CH₂Cl₂, 1:10) to give 2-Chloro-5-fluoropyridine-1-oxide (4.2 g, yield: 74%) ¹H NMR (CDCl₃, 400 MHz) δ 8.31 (dd, J=2.0, 8.5 Hz, 1H), 7.49 (m, 1H), 7.07 (m, 1H). LCMS (ESI) m/z: 149.5 [M+H⁺]

Step 2: 2-Chloro-5-fluoropyridine-N-oxide (1.65 g, 11.0 mmol) was gradually added to 22 mL of concentrated sulfuric acid. Then 4.7 g of potassium nitrate was added while stirring. The reaction mixture was heated at 120° C. for 2 hrs, cooled to room temperature, and carefully poured onto 80 g of crushed ice. The solution was neutralized by dropwise addition of 28% ammonium hydroxide with stirring, while the temperature was maintained below 15° C. with an ice bath. The light yellow crystals precipitated were collected by filtration, washed with ice water, and dried to give the desired product (1.2 g, yield: 59%). ¹H NMR (DMSO-d₆, 400 MHz) δ 9.12 (m, 1H), 8.70 (m, 1H). LCMS (ESI) m/z: 194.5 [M+H⁺]

Step 3: A mixture of 2-chloro-5-fluoro-4-nitropyridine-1-oxide (0.75 g, 3.9 mmol) and 0.8 g of Raney nickel in 40 mL of anhydrous ethyl alcohol was hydrogenated at 40 psi in a Parr hydrogenation apparatus for 3 hrs when TLC showed that the starting material had disappeared and a new product spot was detected. The catalyst was removed by filtration and washed carefully with ethyl alcohol (2×20 mL). Combined filtrate was evaporated in vacuo to give the desired product 4-Amino-2-chloro-5-fluoropyridine (0.25 g, yield: 44%). ¹H NMR (CDCl₃, 400 MHz) δ 7.95 (d, J=2.0 Hz, 1H), 6.65 (d, J=6.0 Hz, 1H), 4.52 (br s, 2H). LCMS (ESI) m/z: 113.4 [M+H⁺].

Step 4: 4-Amino-2-chloro-5-fluoropyridine (0.77 g, 4.7 mmol) was carefully added to 7.5 mL of concentrated sulfuric acid at 0-5° C. (ice-bath) with stirring to give a solution. Potassium nitrate (1.1 g, 10 mmol) was gradually added to the solution during a period of 10 min while the internal temperature was maintained below 5° C. The reaction mixture was further stirred at 0-5° C. for 1 hr and at room temperature for 10 min when TLC showed that all starting material had disappeared and a new product was formed. The mixture was carefully poured onto 30 g of crushed ice. The resulting solution was extracted with methylene chloride (2×20 mL). Combined organics were dried, filtered, and concentrated to give the crude product (0.56 g, yield: 61%), which was directly carried to the next step without purification. LCMS (ESI) m/z: 192.6 [M+H⁺].

Step 5: 2-Chloro-5-fluoro-4-nitroaminopyridine (560 mg, 2.9 mmol) was carefully added to 4.5 mL of concentrated sulfuric acid. The mixture was stirred at room temperature for 12 hrs when TLC showed that the starting material had disappeared and a new product had formed. The mixture was then poured onto 11 g of crushed ice with stirring. The resulting solution was mixed with 20 mL of methylene chloride and neutralized by dropwise addition of 28% ammonium hydroxide with stirring while the internal temperature was maintained below 5° C. in a salted ice bath. The organic layer was separated and the aqueous layer was extracted with methylene chloride (2×20 mL). The combined organic extracts were dried over MgSO₄, filtered, evaporated in vacuo to give the crude product (450 mg, yield: 80%), which was carried to the next step without purification. LCMS (ESI) m/z: 192.6 [M+H⁺].

Step 6: A mixture of 4-amino-2-chloro-5-fluoro-3-nitropyridine (0.45 g, 2.3 mmol) and 0.6 g of Raney nickel in 10 mL of anhydrous ethyl alcohol was hydrogenated at 36 psi in a Parr hydrogenation apparatus for 2 hrs when TLC showed that the starting material had disappeared. The catalyst was removed by filtration and washed carefully with ethyl alcohol (2×10 mL). The combined filtrate was evaporated in vacuo to give a residue, which was purified by silica gel chromatography (CH₂Cl₂:EtOH, 20:1) to give the desired product (0.26 g, yield: 68%) ¹HNMR (DMSO-d₆, 400 MHz) δ 7.45 (d, J=2.0 Hz, 1H), 5.80 (br s, 2H), 4.98 (br s, 2H).

Step 7: To a 50 mL round-bottomed flask with a magnetic stirrer were added 2-chloro-3,4-diamino-5-fluoropyridine (200 mg, 1.2 mmol), 2,6-dichlorobenzaldehyde (220 mg, 1.2 mmol), and FeCl₃ (10 mg, 0.06 mmol) in absolute ethanol (2 mL) and dioxane (10 mL). The mixture was heated at 75° C. with oxygen balloon for 10 hrs. After the reaction was completed, the solvent was partially removed by vacuum and the residue was poured into ice and sat. K₂CO₃ (20 mL). The mixture was extracted with methylene chloride (20 mL). The organic layer was separated and the aqueous layer was extracted with methylene chloride (20 mL) again. The combined organic extracts were dried over MgSO₄, filtered, evaporated in vacuo to give the crude product (250 mg, yield: 64%), which was used in the next step without purification. LCMS (ESI) m/z: 317.6 [M+H⁺].

Step 8: A mixture of 4-chloro-7-fluoro-2-(2,6-dichlorophenyl)-1H-imidazo[4,5-c]pyridine (250 mg, 0.8 mmol) and TMSBr (0.2 mL, 1.5 mmol) in propyl nitrile (10 mL) was heated at 120° C. for 12 hrs. The reaction was then cooled to 23° C. and poured into sat. K₂CO₃-ice water (20 mL). The mixture was extracted with dichloromethane (20 mL). The organic layer was separated, washed with brine (10 mL), dried over MgSO4, filtered, evaporated in vacuo. Crude product was purified by silica gel chromatography (CH₂Cl₂:MeOH, 10:1) to give the desired product (240 mg, yield: 84%) ¹H NMR (DMSO-d₆, 500 MHz): δ 14.42 (s, 1H), 8.20 (d, 1H), 7.71 (m, 3H). LCMS (ESI) m/z: 362.1 [M+H⁺].

### Example 1

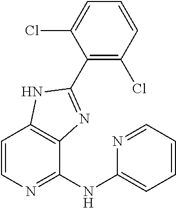 [SMILES: Clc1cccc(Cl)c1-c1nc2c(Nc3ccccn3)nccc2[nH]1]

### 2-(2,6-dichlorophenyl)-N-(pyridin-2-yl)-1H-imidazo[4,5-e]pyridin-4-amine

The mixture of 4-bromo-2-(2,6-dichlorophenyl)-1H-imidazo[4,5-c]pyridine (20 mg, 0.058 mmol), pyridin-2-amine (7.1 mg, 0.076 mmol), Pd₂(dba)₃ (1.6 mg, 0.0040 mmol), XantPhos (0.70 mg, 0.0010 mmol), Cs₂CO₃ (38 mg, 0.12 mmol) in 1,4-Dioxane (1.5 mL) and DME (0.50 mL) was degassed with N₂ for 1 min. The resulting mixture was irradiated in a microwave reactor at 150° C. for 2 hours and cooled to room temperature. The mixture was filtered with Celite and the filtrate was concentrated and purified by prep-HPLC (Gilson GX 281, Shim-pack PRC-ODS 250 mm×20 mm×2, gradient: CH₃CN/10 mm/L NH₄HCO₃, 17 min) to give the desired product as a white solid (5 mg, 24% yield). ¹H NMR (MeOH-d₄, 500 MHz): δ 8.34 (d, J=6.0 Hz, 1H), 7.90 (d, J=8.0 Hz, 1H), 7.82 (m, 2H), 7.55 (m, 2H), 7.51 (m, 1H), 7.29 (d, J=8.0 Hz, 1H), 7.06 (m, 1H). LC-MS (ESI) m/z: 356.1 [M+H⁺].

### Example 2

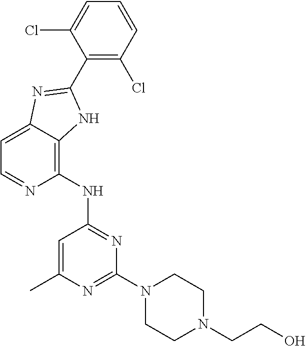 [SMILES: Cc1cc(Nc2nccc3nc(-c4c(Cl)cccc4Cl)[nH]c23)nc(N2CCN(CCO)CC2)n1]

### 2-(4-(4-(2-(2,6-dichlorophenyl)-3H-imidazo[4,5-e]pyridin-4-ylamino)-6-methylpyrimidin-2-yl)piperazin-1-yl)ethanol

Step 1: To the suspension of 2-chloro-6-methylpyrimidin-4-amine (0.300 g, 2.08 mmol) in ethanol (2.0 mL) were added 2-(piperazin-1-yl)ethanol (0.810 g, 6.25 mmol) and diisopropylethylamine (1.5 mL). The mixture was then heated at 150° C. under microwave for 3 hours. After concentration via rotavap, the crude product was purified by column chromatography on silica gel (EtOAc/hexanes=1:1) to give the desired product (0.45 g, 90% yield). ¹H NMR (DMSO-d₆, 500 MHz): δ 6.24 (s, 2H), 5.58 (s, 1H), 4.43 (m, 1H), 3.60-3.35 (m, 6H), 2.40-2.37 (m, 6H), 2.03 (s, 3H). LCMS (ESI) m/z: 238.2 [M+H⁺]

Step 2: Coupling reaction of 4-bromo-2-(2,6-dichlorophenyl)-1H-imidazo[4,5-c]pyridine with 2-(4-(4-amino-6-methylpyrimidine-2-yl)piperazin-1-yl)ethanol followed the same procedure as in Example 1

68 mg, 47% yield. ¹H NMR (DMSO-d₆, 500 MHz): δ 13.34 (s, 1H), 8.35 (s, 1H), 8.07 (d, J=7.0 Hz, 1H), 7.66 (m, 4H), 7.26 (m, 1H), 4.42 (s, 1H), 3.68 (m, 4H), 3.52 (t, J=7.0 Hz, 2H), 2.40 (m, 6H), 2.27 (m, 3H). LCMS (ESI) m/z: 499.4 [M+H⁺]

### Example 3

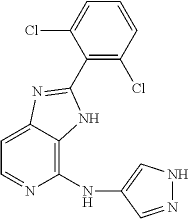 [SMILES: Clc1cccc(Cl)c1-c1nc2ccnc(Nc3cn[nH]c3)c2[nH]1]

### 2-(2,6-dichlorophenyl)-N-(1H-pyrazol-4-yl)-3H-imidazo[4,5-e]-pyridin-4-amine

Step 1: A mixture of 4-nitro-1H-pyrazole (1.13 g, 10 mmol), DMAP (0.24 g, 2.0 mmol), (Boc)₂O (2.0 g, 9.0 mmol)) in CH₂Cl₂ (150 mL) was stirred at 25° C. for 3 hours. The solvent was evaporated and the residue was purified by column chromatography on silica gel with EtOAc/petroleum ether (1/6) to afford the crude product tert-butyl 4-nitro-1H-pyrazole-1-carboxylate (1.3 g, 70% yield). ¹H NMR (DMSO-d₆, 500 MHz): δ 8.78 (s, 1H), 8.22 (s, 1H), 1.69 (s, 9H).

Step 2: To a 500 mL flask were added tert-butyl-4-nitro-1H-pyrazole-1-carboxylate (1.15 g, 5.00 mmol), 10% Pd/C (300 mg) followed by addition of MeOH (150 mL). The resulting mixture was stirred at 60° C. under H₂ balloon for 15 hours. The mixture was filtered with Celite and the filtrate was concentrated by vacuum. The residue was purified by column chromatography on silica gel with EtOAc/petroleum ether (1/6) to afford the desired product as a yellow solid (0.6 g, 62% yield). ¹H NMR (DMSO-d₆, 500 MHz): δ 7.34 (s, 1H), 7.32 (s, 1H), 4.40 (s, 2H), 1.53 (s, 9H).

Step 3: To a microwave tube was added 4-bromo-2-(2,6-dichlorophenyl)-1H-imidazo-[4,5-c]-pyridine (0.050 g, 0.15 mmol), tert-butyl-4-amino-1H-pyrazole-1-carboxylate (0.032 g, 0.17 mmol), Pd₂(dba)₃ (0.013 g, 0.015 mmol), XantPhos (0.017 g, 0.03 mmol), Cs₂CO₃ (0.098 g, 0.03 mmol) and dioxane (1.2 mL). The mixture was degassed with N₂ for 1 min. The resulting mixture was irradiated in a microwave reactor at 160° C. for 2 hours and then cooled to room temperature. The mixture was filtered with Celite and the filtrate was concentrated by vacuum. The residue was purified by prep-HPLC (Gilson GX 281, Shim-pack PRC-ODS 250 mm×20 mm×2, gradient: CH₃CN/10 mm/L NH₄HCO₃, 17 min) to give the desired product (15 mg, 15% yield). ¹H NMR (MeOD-d₄, 500 MHz): δ 8.09 (s, 1H), 7.90 (d, J=5.5 Hz, 1H), 7.76 (s, 1H), 7.59 (m, 3H), 6.97 (d, J=5.5 Hz, 1H). LCMS (ESI) m/z: 345.2 [M+H⁺].

### Example 4

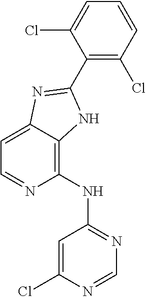 [SMILES: Clc1cc(Nc2nccc3nc(-c4c(Cl)cccc4Cl)[nH]c23)ncn1]

### N-(6-chloropyrimidin-4-yl)-2-(2,6-dichlorophenyl)-3H-imidazo-[4,5-c]pyridin-4-amine

To a microwave tube was added 4-bromo-2-(2,6-dichlorophenyl)-1H-imidazo[4,5-c]-pyridine (0.100 g, 0.29 mmol), 2-chloro-6-isopropylpyrimidin-4-amine (0.034 g, 0.26 mmol), Pd₂(dba)₃ (0.026 g, 0.029 mmol), XantPhos (0.034 g, 0.058 mmol), Cs₂CO₃ (0.189 g, 0.58 mmol) and dioxane (3 mL). The mixture was degassed with N₂ for 1 min. The resulting mixture was irradiated in a microwave reactor at 140° C. for 1.5 hours and cooled to room temperature. The mixture was filtered and the filtrate was concentrated and purified by column chromatography on silica gel with EtOAc/petroleum ether (1:1.5) to give the desired product (50 mg, 44% yield). ¹H NMR (MeOH-d₄, 500 MHz): δ 8.56 (s, 1H), 8.22 (m, 1H), 7.66-7.59 (m, 4H), 7.37 (d, J=5.0 Hz, 1H). LCMS (ESI) m/z: 391.6 [M+H⁺].

### Example 5

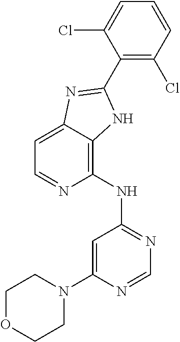 [SMILES: Clc1cccc(Cl)c1-c1nc2ccnc(Nc3cc(N4CCOCC4)ncn3)c2[nH]1]

### 2-(2,6-dichlorophenyl)-N-(6-morpholinopyrimidin-4-yl)-3H-imidazo[4,5-e]pyridin-4-amine

To a suspension of N-(6-chloropyrimidin-4-yl)-2-(2,6-dichlorophenyl)-3H-imidazo[4,5-c]pyridin-4-amine (0.080 g, 0.20 mmol) in ethanol (1.5 mL) were added diisopropylethylamine (0.2 mL) and morpholine (0.088 g, 1.0 mmol). The resulting mixture was heated to 130° C. under microwave for 30 min. The solvent was removed by vacuum and the residue was purified by prep-HPLC (Gilson GX 281, Shim-pack PRC-ODS 250 mm×20 mm×2, gradient: CH₃CN/10 mm/L NH₄HCO₃, 17 min) to give the desired product (25 mg, 28% yield). ¹H NMR (DMSO-d₆, 500 MHz): δ 13.36 (s, 1H), 8.38 (s, 1H), 8.26 (s, 1H), 8.09 (m, 1H), 7.88 (m, 1H), 7.67 m, 3H), 7.26 (s, 1H), 3.70 (m, 4H), 3.56 (m, 4H). LCMS (ESI) m/z: 442.3 [M+H⁺].

### Example 6

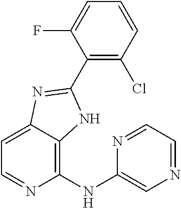 [SMILES: Fc1cccc(Cl)c1-c1nc2ccnc(Nc3cnccn3)c2[nH]1]

### 2-(2-chloro-6-fluorophenyl)-N-(pyrazin-2-yl)-3H-imidazo[4,5-e]-pyridin-4-amine

Step 1: Preparation of 4-bromo-2-(2-chloro-6-fluorophenyl)-1H-imidazo[4,5-c]pyridine, starting with 2-chloropyridine-3,4-diamine and 2-chloro-6-fluoro benzoic acid, followed the same procedure as in Method B. 2.6 g, 80% yield for 2 steps. ¹H NMR (DMSO-d₆, 500 MHz): δ 13.82 (s, 1H), 8.19 (d, J=5.5 Hz, 1H), 7.61 (m, 4H). LCMS (ESI) m/z: 326.6

[M+H⁺]

Step 2: Coupling reaction of 4-bromo-2-(2-chloro-6-fluorophenyl)-1H-imidazo[4,5-c]-pyridine with pyrazi-2-amine followed the same procedure as in Example 1. 58 mg, 56% yield. ¹HNMR (DMSO-d₆, 500 MHz): δ 9.74 (s, 1H), 8.91 (s, 1H), 8.28 (m, 1H), 8.17 (d, J=2.5 Hz, 1H), 7.94 (d, J=5.5 Hz, 1H), 7.53 (m, 3H), 7.20 (d, J=5.5 Hz, 1H). LCMS (ESI) m/z: 340.7 [M+H⁺].

### Example 7

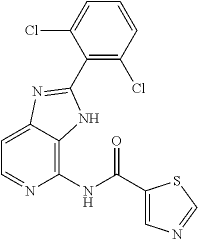 [SMILES: O=C(Nc1nccc2nc(-c3c(Cl)cccc3Cl)[nH]c12)c1cncs1]

### N-(2-(2,6-dichlorophenyl)-3H-imidazo[4,5-c]pyridin-4-yl) thiazole-5-carboxamide

Step 1: To the suspension of thiazole-5-carboxylic acid (0.30 g, 2.32 mmol) in toluene (10 mL) was added thionyl chloride (2 mL). The resulting mixture was refluxed for 1.5 hours and the solvent was then removed by vacuum. The residue was dissolved in anhydrous THF (10 mL) and NH₃ was then bubbled in solution for 10 min. The mixture was concentrated by vacuum and washed with toluene to give the crude product (0.23 g, yield: 77%). ¹H NMR (DMSO-d₆, 500 MHz): δ 9.21 (d, J=2.0 Hz, 1H), 8.45 (d, J=2.0 Hz, 1H), 8.18 (s, 1H), 7.68 (s, 1H). LCMS (ESI) m/z: 128.15 [M+H⁺].

Step 2: To a microwave tube was added 4-bromo-2-(2,6-dichlorophenyl)-1H-imidazo-[4,5-c]pyridine (0.050 g, 0.15 mmol), thiazole-5-carboxamide (0.036 g, 0.28 mmol), Pd₂(dba)₃ (0.013 g, 0.015 mmol), XantPhos (0.034 g, 0.03 mmol), Cs₂CO₃ (0.098 g, 0.3 mmol) and dioxane (1.2 mL). The mixture was degassed with N₂ for 1 min. The resulting mixture was irradiated in a microwave reactor at 150° C. for 1 hour and then cooled to room temperature. The mixture was filtered and the filtrate was concentrated by vacuum. The residue was purified by prep-HPLC (Gilson GX 281, Shim-pack PRC-ODS 250 mm×20 mm×2, gradient: CH₃CN/10 mm/L NH₄HCO₃, 17 min) to give the desired product (12 mg, 14% yield). ¹H NMR (DMSO-d₆, 500 MHz): δ 9.17 (s, 1H), 8.71 (s, 1H), 8.10 (s, 1H), 7.64-7.55 (m, 3H), 7.52 (m, 1H). LCMS (ESI) m/z: 390.0 [M+H⁺].

### Examples 8 and 9

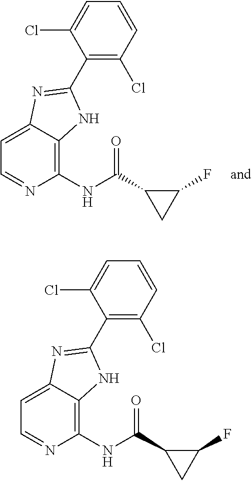 [SMILES: O=C(Nc1nccc2nc(-c3c(Cl)cccc3Cl)[nH]c12)[C@H]1C[C@H]1F ; O=C(Nc1nccc2nc(-c3c(Cl)cccc3Cl)[nH]c12)[C@@H]1C[C@@H]1F]

### (1R,2R) and (1S,2S)—N-(2-(2,6-dichlorophenyl)-3H-imidazo[4,5-e]pyridin-4-yl)-2-fluorocyclopropanecarboxamide

Step 1: To a solution of cis-2-fluorocyclopropanecarboxylic acid (1.32 g, 12.7 mmol) and Et₃N (1.92 g, 19.0 mmol) in dry acetone (25 mL) at 0° C. was added ethyl chloroformate (2.06 g, 19.0 mmol) dropwise. The reaction mixture was then stirred at room temperature for 1 hour. The precipitated white solid was then filtered off. To the filtration was added ammonium hydroxide (7.94 g, 63.4 mmol). The mixture was stirred at room temperature overnight. The mixture was then concentrated to a semi-solid which was titrated with EtOAc to give the mixture of (1R,2R) and (1S,2S)-2-fluorocyclopropanecarboxamide as an off-white solid (1.01 g, 78% yield). ¹H NMR (400 MHz, MeOH-d₄) δ 4.87-4.79 (m, 0.5H), 4.70-4.62 (m, 0.5H), 1.85-1.73 (m, 1H), 1.61 (dtd, J=22.9, 7.1, 3.6 Hz, 1H), 1.07 (m, 1H).

Step 2: To a microwave tube was added 4-bromo-2-(2,6-dichlorophenyl)-1H-imidazo-[4,5-c]pyridine (0.070 g, 0.20 mmol), cis-2-fluorocyclopropanecarboxamide (0.032 g, 0.31 mmol), Pd₂(dba)₃ (0.018 g, 0.020 mmol), XantPhos (0.023 g, 0.04 mmol), CS₂CO₃ (0.163 g, 0.50 mmol) and dioxane (1.2 mL). The mixture was degassed with N₂ for 1 min. The resulting mixture was irradiated in a microwave reactor at 150° C. for 1 hour and then cooled to room temperature. The mixture was filtered and the filtrate was concentrated by vacuum. The residue was purified by prep-TLC (EtOAc/PE=1:1) to give the crude mixture which was then purified by chiral prep-HPLC (AS-H, SFC with CH₃OH/0.1% DEA as co-solvent, 10 min) to give two desired product as following.

First eluting peak: 12 mg, 16% yield. >98% ee (3.14 min, AS-H, SFC with CH₃OH/0.1% DEA as co-solvent, 5 min) ¹H NMR (MeOH-d₄, 500 MHz): δ 8.04 (d, J=5.5 Hz, 1H), 7.49 (m, 3H), 7.42 (d J=5.5 Hz, 1H), 4.88-4.74 (m, 1H), 2.11 (m, 1H), 1.76 (m, 1H), 1.19 (m, 2H). LCMS (ESI) m/z: 365.2 [M+H⁺].

Second eluting peak: 8 mg, 10% yield. >97% ee (3.75 min, AS-H, SFC with CH₃OH/0.1% DEA as co-solvent, 5 min) ¹H NMR (MeOH-d₄, 500 MHz): δ 8.04 (d, J=5.5 Hz, 1H), 7.51-7.48 (m, 3H), 7.42 (d, J=5.5 Hz, 1H), 4.88 (m, 1H), 2.11 (m, 1H), 1.76 (m, 1H), 1.25-1.12 (m, 2H). LCMS (ESI) m/z: 365.0 [M+H⁺].

### Example 10

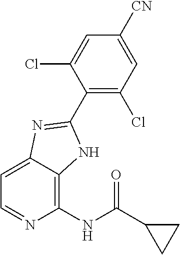 [SMILES: N#Cc1cc(Cl)c(-c2nc3ccnc(NC(=O)C4CC4)c3[nH]2)c(Cl)c1]

### N-(2-(2,6-dichloro-4-cyanophenyl)-3H-imidazo[4,5-c]pyridin-4-yl)cyclopropanecarboxamide

Step 1: 4-amino-2,6-dichlorophenol (100 g, 0.56 mol) and di-tert-butyl dicarbonate (146 g, 0.73 mol) were dissolved in dioxane (1.5 L), and the mixture was stirred at 110° C. for overnight. The solvent was evaporated to afford the crude tert-butyl 3,5-dichloro-4-hydroxyphenylcarbamate (220 g) which was directly used in the next step.

Tert-butyl 3,5-dichloro-4-hydroxyphenylcarbamate (220 g, crude) and 2,6-dimethylpyridine (78 g, 0.73 mol) were dissolved in dichloromethane (2.0 L). Trifluoromethanesulfonic anhydride (174 g, 0.62 mol) was added dropwise at −78° C., and the mixture was stirred at room temperature for 1 hour. The solvent was evaporated, and the residue was purified by column chromatography on silica gel (petroleum ether/EtOAc 40:1) to give the desired compound (196 g, yield 84%). ¹H NMR (CDCl₃) δ7.51 (s, 2H), 6.60 (brs, 1H), 1.52 (s, 9H).

Step 2: A mixture of 4-(tert-butoxycarbonylamino)-2,6-dichlorophenyl trifluoromethanesulfonate (14 g, 34 mmol), dppp (1.4 g, 3.4 mmol), Pd(OAc)₂ (0.84 g, 3.4 mol) and Et₃N (19.6 ml) in MeOH (112 mL) and DMF (224 mL) was refluxed under an atmosphere of carbon monoxide (10 atm) overnight. The solvent was evaporated, and the residue was purified by column chromatography on silica gel (petroleum ether/EtOAc 80:1) to give the desired compound (6.5 g, yield 60%). ¹H NMR (CDCl₃) δ 7.40 (s, 2H), 6.56 (brs, 1H), 3.94 (s, 3H), 1.51 (s, 9H).

Step 3: A solution of methyl 4-(tert-butoxy carbonylamino)-2,6-dichlorobenzoate (65 g, 0.49 mol) in H₂SO₄/EtOAc (3 N, 20 mL) was stirred at room temperature for 2 hrs. The reaction mixture was filtered, and the solid was collected to afford the product (60 g, 92% yield). ¹H NMR (DMSO) δ 8.40 (brs, 3H), 6.61 (s, 2H), 3.80 (s, 3H).

Step 4: Methyl 4-amino-2,6-dichlorobenzoate (104 g, 0.14 mol) was added to conc. HCl (884 mL) in a 5-L round-bottomed flask, and the mixture was cooled in an ice/alcohol bath. A solution of sodium nitrite (55.2 g, 0.8 mol) in water (312 mL) was dropped in slowly at the temperature range of −5° C. and 0° C. with vigorous stirring. 30 min later, the mixture was filtered and the filtrate was added into a pre-cooled solution of potassium iodide (352.3 g, 2.12 mol) with mechanical stirring. A black precipitate appeared with the addition. The mixture was warmed to room temperature and stirred overnight. The mixture was diluted with ethyl acetate (1 L) and the organic phase was separated. The aqueous phase was extracted with ethyl acetate (1 L) again. Combined dark organic phase was washed to be clear with saturated solution of NaHSO₃. After being dried over Na₂SO₄ and being concentrated via rotavap, the crude product further purified by column chromatography on silica gel (petroleum ether/EtOAc 50/1) to give the desired product (140 g, yield 90%) as a yellow oil. ¹H NMR (CDCl₃) δ 7.70 (s, 2H), 3.97 (s, 3H).

Step 5: Methyl 2,6-dichloro-4-iodobenzoate (46 g, 0.14 mol) was dissolved in pyridine (1380 mL) and water (230 mL). Lithium iodide (37.2 g, 0.28 mol) was added in one portion. The resulting mixture was heated at 130° C. for 30 hours. The solvent was removed. The residue was dissolved in 2N HCl (500 mL) and extracted with ethyl acetate (3×1 L). Combined organic phase was dried over Na₂SO₄ and concentrated via rotavap. The residue was dissolved in N-methyl morpholine (5 mL) and concentrated again. The residue was diluted with 2N HCl (100 mL) and extracted with dichloromethane (3×100 mL). The organic phases were combined, dried and concentrated to give the target product (39 g, 88%). ¹H NMR (DMSO) δ 14.26 (brs, 1H), 7.99 (s, 2H).

Step 6: Preparation of 4-bromo-2-(2,6-dichloro-4-iodophenyl)-3H-imidazo[4,5-c]pyridine, starting with 2-chloropyridine-3,4-diamine and 2,6-dichloro-4-iodobenzoic acid, followed procedure as in Method B. 13 g, 40% yield for 2 steps. ¹H NMR (DMSO-d₆, 500 MHz): δ 13.77 (br, 1H), 8.19-8.17 (m, 3H), 7.70 (d, J=5.0 Hz, 1H). LCMS (ESI) m/z: 469.8 [M+H+].

Step 7: A mixture of 4-bromo-2-(2,6-dichloro-4-iodophenyl)-3H-imidazo[4,5-c]pyridine (5.0 g, 10.7 mmol), Zn(CN)₂ (2.5 g, 21 mmol), Pd(PPh₃)₄ (1.1 g, 0.94 mmol) in dry DMF (100 ml) was heated to 80° C. under nitrogen atmosphere for 2.5 hours. TLC showed complete conversion. Ethyl acetate (100 mL) was added to quench the reaction. After filtration to remove the insoluble solid, water (100 mL) was added. The resulting mixture was extracted with ethyl acetate (50 mL) for three times. The combined organic phase was washed with brine, dried over Na₂SO₄, and then concentrated. The residue was purified on silica gel to give product as a pale yellow solid (2.7 g, 67% yield). ¹HNMR: (DMSO-d₆, 500 MHz): δ 14.00 (br, 1H), 8.39 (s, 2H), 8.19 (s, 1H), 7.74 (s, 1H). LCMS (ESI) m/z: 368.9 [M+H⁺].

Step 8: To a microwave tube was added 4-(4-bromo-1H-imidazo[4,5-c]pyridin-2-yl)-3,5-dichlorobenzonitrile (0.10 g, 0.27 mmol), cyclopropanecarboxamide (0.069 g, 0.81 mmol), Pd₂(dba)₃ (0.036 g, 0.040 mmol), XantPhos (0.017 g, 0.030 mmol), Cs₂CO₃ (0.26 g, 0.81 mmol) and dioxane (3.0 mL). The mixture was degassed with N₂ for 10 min. The resulting mixture was irradiated in a microwave reactor at 160° C. for 2 hours and then cooled to room temperature. The mixture was filtered with Celite and the filtrate was concentrated and purified by prep-TLC to give the desired product as a white solid (15 mg, 15% yield). ¹H NMR (MeOH-d₄, 500 MHz): δ 7.95 (s, 2H), 7.54 (m, 1H), 7.45 (m, 1H), 1.90 (m, 1H), 1.23-1.18 (m, 2H), 1.00-0.88 (m, 2H). LCMS (ESI) m/z: 372.0 [M+H⁺].

### Example 11

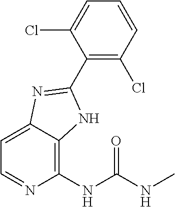 [SMILES: CNC(=O)Nc1nccc2nc(-c3c(Cl)cccc3Cl)[nH]c12]

### 1-(2-(2,6-dichlorophenyl)-3H-imidazo[4,5-e]pyridin-4-yl)-3-methylurea

To a microwave tube was added 4-bromo-2-(2,6-dichlorophenyl)-1H-imidazo-[4,5-c]pyridine (0.050 g, 0.15 mmol), 1-methylurea (0.031 g, 0.3 mmol), Pd₂(dba)₃ (0.013 g, 0.015 mmol), XantPhos (0.034 g, 0.030 mmol), Cs₂CO₃ (0.147 g, 0.45 mmol) and dioxane (1.0 mL). The mixture was degassed with N₂ for 1 min. The resulting mixture was irradiated in a microwave reactor at 150° C. for 1 hour and then cooled to room temperature. The mixture was filtered and the filtrate was concentrated by vacuum. The residue was purified by prep-HPLC (Gilson GX 281, Shim-pack PRC-ODS 250 mm×20 mm×2, gradient: CH₃CN/10 mm/L NH₄HCO₃, 17 min) to give the desired product (21 mg, 40% yield). ¹H NMR (DMSO-d₆, 500 MHz): δ 12.70 (s, 1H), 9.34 (s, 1H), 8.48 (s, 1H), 7.97 (d, J=7.0 Hz, 1H), 7.67 (m, 3H), 7.26 (d, J=5.5 Hz, 1H), 2.83 (d, J=5.0 Hz, 3H). LCMS (ESI) m/z: 336.2 [M+H⁺].

### Examples 12 and 13

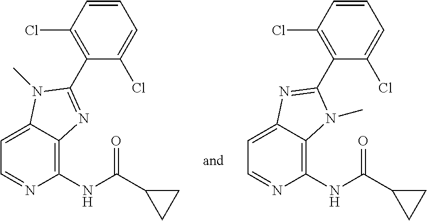 [SMILES: Cn1c(-c2c(Cl)cccc2Cl)nc2c(NC(=O)C3CC3)nccc21 ; Cn1c(-c2c(Cl)cccc2Cl)nc2ccnc(NC(=O)C3CC3)c21]

### N-(2-(2,6-dichlorophenyl)-1-methyl-1H-imidazo[4,5-c]pyridine-4-yl)cyclopropanecarboxamide and N-(2-(2,6-dichlorophenyl)-3-methyl-3H-imidazo[4,5-c]pyridine-4-yl)cyclopropanecarboxamide

Step 1: To a solution of 4-bromo-2-(2,6-dichlorophenyl)-1H-imidazo[4,5-c]pyridine (1.0 g, 2.9 mmol) in DMF (5 mL) at 23° C. was added sodium hydride (0.11 g, 4.4 mmol). The mixture was allowed to stir at 23° C. for 30 min before iodomethane (0.46 g, 3.2 mmol) was added in one portion and the mixture was stirred at 23° C. overnight. The reaction mixture was quenched with water (50 mL), extracted with ethyl acetate (3×50 ml). The combined organic phase was washed with brine, dried over Na₂SO₄, and then concentrated. The residue was purified by column chromatography on silica gel with dichloromethane/methanol (10:1) to give the desired products as a mixture (0.65 g, 62% yield). LCMS (ESI) m/z: 358.1 [M+H⁺].

Step 2: Buchwald coupling reaction followed the same procedure as in Example 1. The mixture was separated by Chiral SFC (Berger MG2 Chiral Technology Prep Column (250×21.2 mm, 5 um; Conditions: isocratic at % Methanol at 50 ml/min, 100 bars, 40° C., 254 nm) to give the two desired products as following.

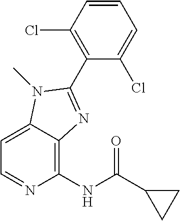 [SMILES: Cn1c(-c2c(Cl)cccc2Cl)nc2c(NC(=O)C3CC3)nccc21]

35 mg, 38% yield. ¹H NMR: (DMSO-d₆, 500 MHz): δ 10.41 (s, 1H), 8.18 (d, J=5.5 Hz, 1H), 7.72 (m, 3H), 7.55 (d, J=5.5 Hz, 1H), 3.63 (s, 3H), 2.15 (m, 1H), 0.80 (m, 4H). LCMS (ESI) m/z: 361.1 [M+H⁺].

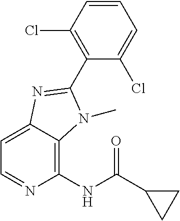 [SMILES: Cn1c(-c2c(Cl)cccc2Cl)nc2ccnc(NC(=O)C3CC3)c21]

3 mg, 3% yield. ¹H NMR: (DMSO-d₆, 400 MHz): δ 10.69 (s, 1H), 8.21 (d, J=5.5 Hz, 1H), 7.70 (m, 4H), 3.59 (s, 3H), 1.94 (m, 1H), 0.84 (m, 4H). LCMS (ESI) m/z: 361.0 [M+H⁺].

### Example 14

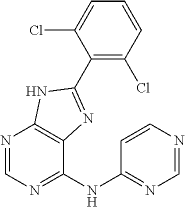 [SMILES: Clc1cccc(Cl)c1-c1nc2c(Nc3ccncn3)ncnc2[nH]1]

### 8-(2,6-dichlorophenyl)-N-(pyrimidin-4-yl)-9H-purin-6-amine

To a microwave tube was added 6-chloro-8-(2,6-dichlorophenyl)-9H-purine (0.12 g, 0.40 mmol), pyrimidin-4-amine (0.048 g, 0.48 mmol), Pd₂(dba)₃ (0.036 g, 0.040 mmol), XantPhos (0.048 g, 0.080 mmol), Cs₂CO₃ (0.26 g, 0.80 mmol) and dioxane (3.0 mL). The mixture was degassed with N₂ for 10 min. The resulting mixture was irradiated in a microwave reactor at 160° C. for 2 hours and then cooled to room temperature. The mixture was filtered with Celite and the filtrate was concentrated and re-dissolved in DMF for preparative HPLC (Gilson GX 281, Shim-pack PRC-ODS 250 mm×20 mm×2, gradient: CH₃CN/10 mm/L NH₄HCO₃, 17 min) to give the desired product as a white solid (14 mg, 9.8% yield). ¹H NMR (DMSO-d₆, 500 MHz,): δ 13.91 (s, 1H), 10.37 (s, 1H), 8.85 (s, 1H), 8.67 (s, 1H), 8.64 (d, J=6.0 Hz, 1H), 8.42 (s, 1H), 7.65-7.73 (m, 3H). LCMS (ESI) m/z: 358.0 [M+H⁺].

### Example 15

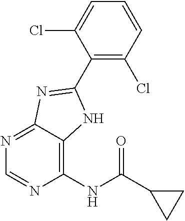 [SMILES: O=C(Nc1ncnc2nc(-c3c(Cl)cccc3Cl)[nH]c12)C1CC1]

### N-(8-(2,6-dichlorophenyl)-7H-purin-6-yl)cyclopropanecarboxamide

To a microwave tube was added 6-chloro-8-(2,6-dichlorophenyl)-9H-purine (0.12 g, 0.40 mmol), cyclopropanecarboxamide (0.041 g, 0.48 mmol), Pd2(dba)₃ (0.036 g, 0.040 mmol), XantPhos (0.048 g, 0.080 mmol), Cs2CO3 (0.260 g, 0.80 mmol) and dioxane (3.0 mL). The mixture was degassed with N2 for 10 min. The resulting mixture was irradiated in a microwave reactor at 160° C. for 2 hours and then cooled to room temperature. The mixture was filtered with Celite and the filtrate was concentrated and re-dissolved in DMF for preparative HPLC (Gilson GX 281, Shim-pack PRC-ODS 250 mm×20 mm×2, gradient: CH3CN/10 mm/L NH4HCO3, 17 min) to give the desired product as a white solid (12 mg, 8.5% yield). ¹H NMR (DMSO-d6, 500 MHz): δ 12.42 (s, 1H), 11.55 (s, 1H), 8.69 (s, 1H), 7.60 7.66 (m, 3H), 2.17 (s, 1H), 0.92-0.96 (m, 4H). LCMS (ESI) m/z: 348.1 [M+H+].

### Example 16

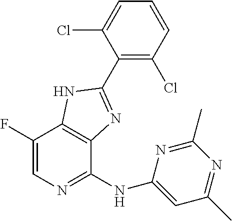 [SMILES: Cc1cc(Nc2ncc(F)c3[nH]c(-c4c(Cl)cccc4Cl)nc23)nc(C)n1]

### 2-(2,6-dichlorophenyl)-N-(2,6-dimethylpyrimidin-4-yl)-7-fluoro-1H-imidazo[4,5-c]pyridin-4-amine

The mixture of 4-bromo-7-fluoro-2-(2,6-dichlorophenyl)-1H-imidazo[4,5-c]pyridine (50 mg, 0.14 mmol), 2,6-dimethylpyrimidine-2-amine (22 mg, 0.18 mmol), Pd₂(dba)₃ (4 mg, 0.0040 mmol), XantPhos (2 mg, 0.002 mmol), Cs₂CO₃ (90 mg, 0.28 mmol) in 1,4-Dioxane (10 mL) and DME (2 mL) was degassed with N₂ for 1 min. The resulting mixture was irradiated in a microwave reactor at 170° C. for 2 hrs and cooled to room temperature. The mixture was filtered with Celite and the filtrate was concentrated and purified by prep-HPLC (Gilson GX 281, Shim-pack PRC-ODS 250 mm×20 mm×2, gradient: CH₃CN/10 mm/L NH₄HCO₃, 17 min) to give the desired product (27 mg, yield: 48%). ¹H NMR (DMSO-d₆, 500 MHz): δ 8.30 (d, J=6.0 Hz, 1H), 7.95 (s, 1H), 7.61 (m, 4H), 2.39 (s, 3H), 2.34 (s, 3H). LC-MS (ESI) m/z: 404.4 [M+H⁺].

### Example 17

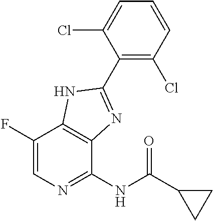 [SMILES: O=C(Nc1ncc(F)c2[nH]c(-c3c(Cl)cccc3Cl)nc12)C1CC1]

### N-(2-(2,6-dichlorophenyl)-7-fluoro-1H-imidazo[4,5-c]pyridin-4-yl)cyclopropanecarboxamide

The mixture of 4-bromo-7-fluoro-2-(2,6-dichlorophenyl)-1H-imidazo[4,5-c]pyridine (50 mg, 0.14 mmol), cyclopropyl amide (15 mg, 0.18 mmol), Pd₂(dba)₃ (4 mg, 0.0040 mmol), XantPhos (2 mg, 0.002 mmol), Cs₂CO₃ (90 mg, 0.28 mmol) in 1,4-Dioxane (10 mL) and DME (2 mL) was degassed with N₂ for 1 min. The resulting mixture was irradiated in a microwave reactor at 170° C. for 2 hrs and cooled to room temperature. The mixture was filtered with Celite and the filtrate was concentrated and purified by prep-HPLC (Gilson GX 281, Shim-pack PRC-ODS 250 mm×20 mm×2, gradient: CH₃CN/10 mm/L NH₄HCO₃, 17 min) to give the desired product (17 mg, yield: 33%). ¹H NMR (DMSO-d₆, 500 MHz): δ 10.76 (s, 2H), 8.37 (s, 1H), 7.60 (m, 3H), 2.06 (m, 1H), 1.24 (m, 2H), 0.81 (m, 2H). LCMS (ESI) m/z: 366.3 [M+H⁺].

Additional examples 18-215 shown in Table 1 can be made according to the above methods. The column titled Synthetic Methods gives the method from Scheme 1 or 3 used to prepare the intermediate.

TABLE 1

| | | | | LCMS | | |
|---|---|---|---|---|---|---|
| | | | Synth. | (ESI) | LCMS | RT |
| Ex | Structure | Name | Method | m/z | Method | (min) |
| 18 | 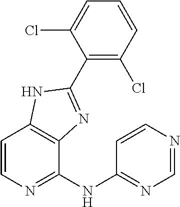 [SMILES: Clc1cccc(Cl)c1-c1nc2c(Nc3ccncn3)nccc2[nH]1] | 2-(2,6- dichlorophenyl)- N-(pyrimidin-4- yl)-1H- imidazo[4,5- c]pyridine-4- amine | B | 357.0 | A | 3.40 |
| 19 | 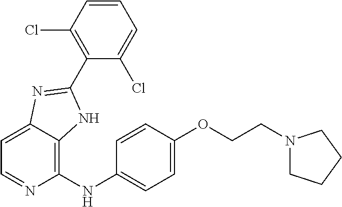 [SMILES: Clc1cccc(Cl)c1-c1nc2ccnc(Nc3ccc(OCCN4CCCC4)cc3)c2[nH]1] | 2-(2,6- dichlorophenyl)- N-(4-(2- (pyrrolidin-1- yl)ethoxy) phenyl)-3H- imidazo[4,5- c]pyridin-4- amine | A | 468.4 | B | 3.11 |
| 20 | 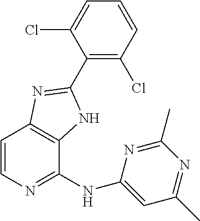 [SMILES: Cc1cc(Nc2nccc3nc(-c4c(Cl)cccc4Cl)[nH]c23)nc(C)n1] | [2-(2,6-Dichloro- phenyl)-1H- imidazo[4,5- c]pyridin-4-yl]- (2,6-dimethyl- pyrimidin-4-yl)- amine | C | 385.2 | B | 3.24 |
| 21 | 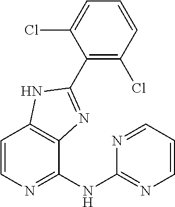 [SMILES: Clc1cccc(Cl)c1-c1nc2c(Nc3ncccn3)nccc2[nH]1] | 2-(2,6- dichlorophenyl)- N-(pyrimidin-2- yl)-1H- imidazo[4,5-c]- pyridin-4-amine | C | 357.0 | A | 3.72 |
| 22 | 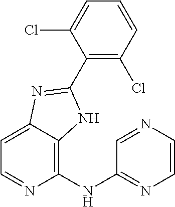 [SMILES: Clc1cccc(Cl)c1-c1nc2ccnc(Nc3cnccn3)c2[nH]1] | 2-(2,6- dichlorophenyl)- N-(pyrazin-2-yl)- 3H-imidazo[4,5- c]pyridine-4- amine | B | 357.0 | A | 3.69 |
| 23 | 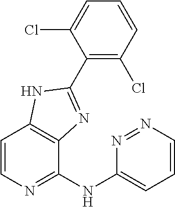 [SMILES: Clc1cccc(Cl)c1-c1nc2c(Nc3cccnn3)nccc2[nH]1] | 2-(2,6- dichlorophenyl)- N-(pyridazin-3- yl)-1H- imidazo[4,5-c]- pyridin-4-amine | C | 357.1 | A | 3.78 |
| 24 | 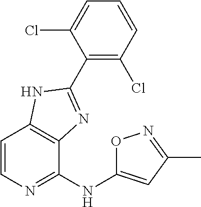 [SMILES: Cc1cc(Nc2nccc3[nH]c(-c4c(Cl)cccc4Cl)nc23)on1] | N-(2-(2,6- dichlorophenyl)- 1H-imidazo[4,5- c]pyridin-4-yl)-3- methyl-isoxazol- 5-amine | C | 360.0 | A | 4.42 |
| 25 | 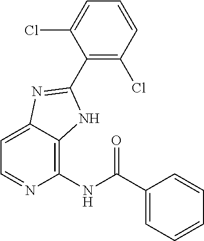 [SMILES: O=C(Nc1nccc2nc(-c3c(Cl)cccc3Cl)[nH]c12)c1ccccc1] | N-(2-(2,6- dichlorophenyl)- 3H-imidazo[4,5- c]pyridin-4-yl)- benzamide | B | 383.0 | A | 4.44 |
| 26 | 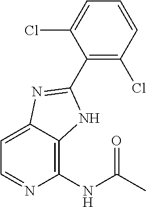 [SMILES: CC(=O)Nc1nccc2nc(-c3c(Cl)cccc3Cl)[nH]c12] | N-(2-(2,6- dichlorophenyl)- 3H-imidazo[4,5- c]pyridin-4- ylacetamide | B | 321.0 | A | 2.69 |
| 27 | 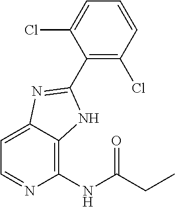 [SMILES: CCC(=O)Nc1nccc2nc(-c3c(Cl)cccc3Cl)[nH]c12] | N-(2-(2,6- dichlorophenyl)- 3H-imidazo[4,5- c]pyridin-4- yl)propionamide | B | 335.1 | A | 3.37 |
| 28 | 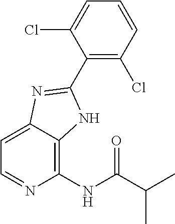 [SMILES: CC(C)C(=O)Nc1nccc2nc(-c3c(Cl)cccc3Cl)[nH]c12] | N-(2-(2,6- dichlorophenyl)- 3H-imidazo[4,5- c]pyridin-4-yl)- isobutyramide | B | 349.0 | A | 3.95 |
| 29 | 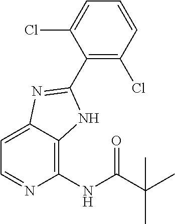 [SMILES: CC(C)(C)C(=O)Nc1nccc2nc(-c3c(Cl)cccc3Cl)[nH]c12] | N-(2-(2,6- dichlorophenyl)- 3H-imidazo[4,5- c]pyridin-4- yl)pivalamide | B | 363.0 | A | 3.77 |
| 30 | 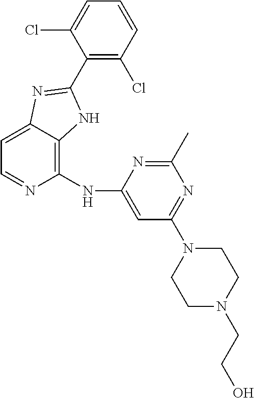 [SMILES: Cc1nc(Nc2nccc3nc(-c4c(Cl)cccc4Cl)[nH]c23)cc(N2CCN(CCO)CC2)n1] | 2-(4-(6-(2-(2,6- dichlorophenyl)- 3H-imidazo[4,5- c]pyridine-4- ylamino)-2- methylpyrimidin- 4-yl)piperazin-1- yl)ethanol | B | 499.1 | A | 3.82 |
| 31 | 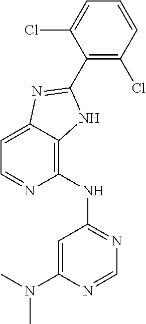 [SMILES: CN(C)c1cc(Nc2nccc3nc(-c4c(Cl)cccc4Cl)[nH]c23)ncn1] | N⁴-(2-(2,6- dichlorophenyl)- 3H-imidazo[4,5- c]pyridin-4-yl)- N⁶,N⁶- dimethyl- pyrimidine- 4,6-diamine | B | 400.0 | A | 3.72 |
| 32 | 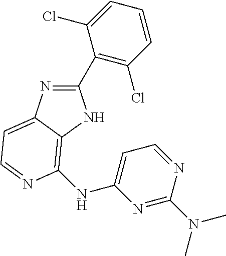 [SMILES: CN(C)c1nccc(Nc2nccc3nc(-c4c(Cl)cccc4Cl)[nH]c23)n1] | N⁴-(2-(2,6- dichlorophenyl)- 3H-imidazo[4,5- c]pyridin-4-yl)- N²,N²- dimethyl- pyrimidine- 2,4-diamine | B | 400.0 | A | 4.78 |
| 33 | 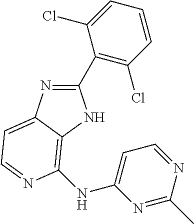 [SMILES: Cc1nccc(Nc2nccc3nc(-c4c(Cl)cccc4Cl)[nH]c23)n1] | 2-(2,6- dichlorophenyl)- N-(2- methylpyrimidin- 4-yl)-3H- imidazo-[4,5- c]pyridin-4- amine | B | 371.0 | A | 3.59 |
| 34 | 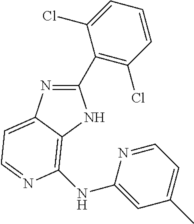 [SMILES: Cc1ccnc(Nc2nccc3nc(-c4c(Cl)cccc4Cl)[nH]c23)c1] | 2-(2,6- dichlorophenyl)- N-(4- methylpyridin-2- yl)-3H- imidazo[4,5-c]- pyridin-4-amine | B | 370.0 | A | 5.04 |
| 35 | 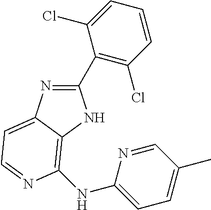 [SMILES: Cc1ccc(Nc2nccc3nc(-c4c(Cl)cccc4Cl)[nH]c23)nc1] | 2-(2,6- dichlorophenyl)- N-(5- methylpyridin-2- yl)-3H-imidazo- [4,5-c]pyridin-4- amine | B | 370.0 | A | 5.15 |
| 36 | 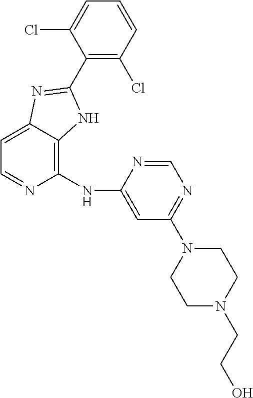 [SMILES: OCCN1CCN(c2cc(Nc3nccc4nc(-c5c(Cl)cccc5Cl)[nH]c34)ncn2)CC1] | 2-(4-(6-(2-(2,6- dichlorophenyl)- 3H-imidazo[4,5- c]pyridine-4- ylamino) pyrimidin- 4-yl)piperazin- 1-yl)ethanol | B | 485.1 | A | 3.54 |
| 37 | 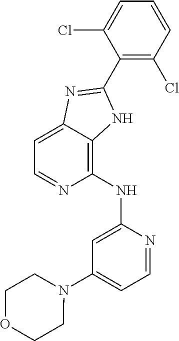 [SMILES: Clc1cccc(Cl)c1-c1nc2ccnc(Nc3cc(N4CCOCC4)ccn3)c2[nH]1] | 2-(2,6- dichlorophenyl)- N-(4- morpholino- pyridin- 2-yl)-3H- imidazo[4,5- c]pyridin-4- amine | B | 441.1 | A | 4.72 |
| 38 | 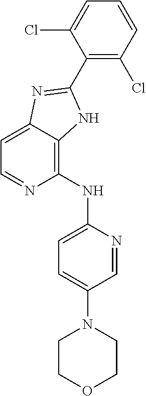 [SMILES: Clc1cccc(Cl)c1-c1nc2ccnc(Nc3ccc(N4CCOCC4)cn3)c2[nH]1] | 2-(2,6- dichlorophenyl)- N-(5- morpholino- pyridin- 2-yl)-3H- imidazo[4,5- c]pyridin-4- amine | B | 441.1 | A | 4.58 |
| 39 | 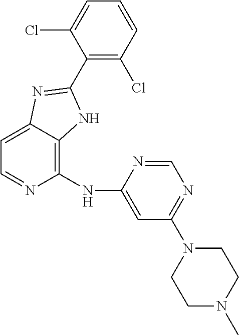 [SMILES: CN1CCN(c2cc(Nc3nccc4nc(-c5c(Cl)cccc5Cl)[nH]c34)ncn2)CC1] | 2-(2,6- dichlorophenyl)- N-(6-(4- methylpiperazin- 1-yl)pyrimidin-4- yl)-3H- imidazo[4,5- c]pyridin-4- amine | B | 455.1 | A | 3.40 |
| 40 | 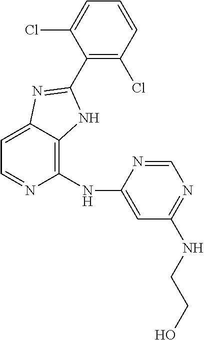 [SMILES: OCCNc1cc(Nc2nccc3nc(-c4c(Cl)cccc4Cl)[nH]c23)ncn1] | 2-(6-(2-(2,6- dichlorophenyl)- 3H-imidazo[4,5- c]pyridin-4- ylamino)- pyrimidin-4- ylamino)ethanol | B | 416.1 | A | 3.21 |
| 41 | 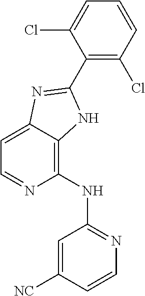 [SMILES: N#Cc1ccnc(Nc2nccc3nc(-c4c(Cl)cccc4Cl)[nH]c23)c1] | 2-(2-(2,6- dichlorophenyl)- 3H-imidazo[4,5- c]pyridin-4- ylamino) isonicotinonitrile | B | 381.0 | A | 4.89 |
| 42 | 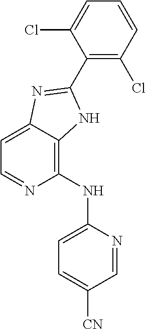 [SMILES: N#Cc1ccc(Nc2nccc3nc(-c4c(Cl)cccc4Cl)[nH]c23)nc1] | 6-(2-(2,6- dichlorophenyl)- 3H-imidazo[4,5- c]pyridin-4- ylamino) nicotinonitrile | B | 381.1 | A | 4.57 |
| 43 | 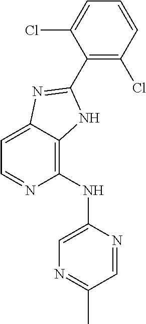 [SMILES: Cc1cnc(Nc2nccc3nc(-c4c(Cl)cccc4Cl)[nH]c23)cn1] | 2-(2,6- dichlorophenyl)- N-(5- methylpyrazin-2- yl)-3H- imidazo[4,5- c]pyridin-4- amine | B | 371.0 | A | 4.15 |
| 44 | 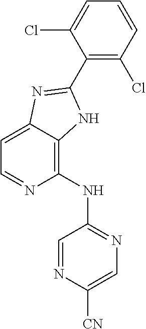 [SMILES: N#Cc1cnc(Nc2nccc3nc(-c4c(Cl)cccc4Cl)[nH]c23)cn1] | 5-(2-(2,6- dichlorophenyl)- 3H-imidazo[4,5- c]pyridin-4- ylamino)pyrazine- 2-carbonitrile | B | 382.0 | A | 3.85 |
| 45 | 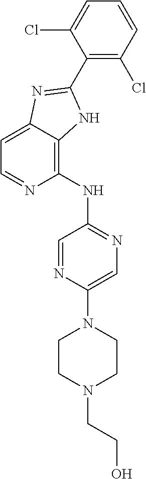 [SMILES: OCCN1CCN(c2cnc(Nc3nccc4nc(-c5c(Cl)cccc5Cl)[nH]c34)cn2)CC1] | 2-(4-(5-(2-(2,6- dichlorophenyl)- 3H-imidazo[4,5- c]pyridin-4- ylamino)pyrazin- 2-yl)piperazin-1- yl)ethanol | B | 485.2 | A | 3.98 |
| 46 | 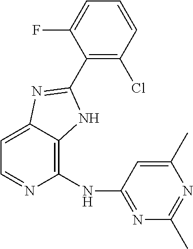 [SMILES: Cc1cc(Nc2nccc3nc(-c4c(F)cccc4Cl)[nH]c23)nc(C)n1] | 2-(2-chloro-6- fluorophenyl)-N- (2,6- dimethylpyrimidin- 4-yl)-3H- imidazo[4,5- c]pyridin-4- amine | B | 369.1 | A | 3.87 |
| 47 | 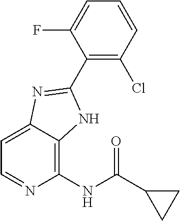 [SMILES: O=C(Nc1nccc2nc(-c3c(F)cccc3Cl)[nH]c12)C1CC1] | N-(2-(2-chloro-6- fluorophenyl)- 3H-imidazo[4,5- c]pyridin-4-yl)- cyclopropane- carboxamide | B | 331.1 | A | 3.80 |
| 48 | 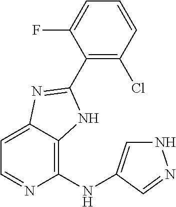 [SMILES: Fc1cccc(Cl)c1-c1nc2ccnc(Nc3cn[nH]c3)c2[nH]1] | 2-(2-chloro-6- fluorophenyl)-N- (1H-pyrazol-4- yl)-3H- imidazo[4,5- c]pyridin-4- amine | B | 329.1 | A | 3.52 |
| 49 | 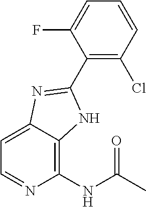 [SMILES: CC(=O)Nc1nccc2nc(-c3c(F)cccc3Cl)[nH]c12] | 2-(2-chloro-6- fluorophenyl)-N- (pyridin-2-yl)- 1H-imidazo[4,5 c]pyridin-4- amine | B | 305.1 | A | 2.58 |
| 50 | 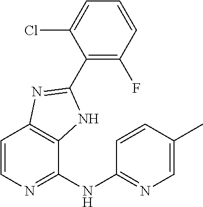 [SMILES: Cc1ccc(Nc2nccc3nc(-c4c(F)cccc4Cl)[nH]c23)nc1] | 2-(2-chloro-6- fluorophenyl)-N- (5-methylpyridin- 2-yl)-3H- imidazo[4,5- c]pyridin-4- amine | B | 354.1 | A | 4.99 |
| 51 | 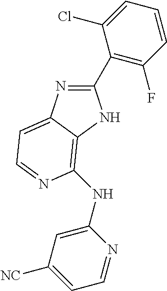 [SMILES: N#Cc1ccnc(Nc2nccc3nc(-c4c(F)cccc4Cl)[nH]c23)c1] | 2-(2-(2-chloro-6- fluorophenyl)- 3H-imidazo[4,5- c]pyridin-4- ylamino) isonicotinonitrile | B | 365.1 | A | 4.64 |
| 52 | 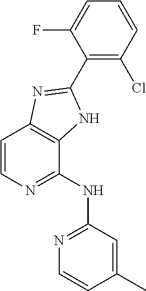 [SMILES: Cc1ccnc(Nc2nccc3nc(-c4c(F)cccc4Cl)[nH]c23)c1] | 2-(2-chloro-6- fluorophenyl)-N- (4-methylpyridin- 2-yl)-3H- imidazo[4,5- c]pyridin-4- amine | B | 354.1 | A | 4.93 |
| 53 | 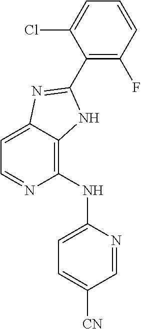 [SMILES: N#Cc1ccc(Nc2nccc3nc(-c4c(F)cccc4Cl)[nH]c23)nc1] | 6-(2-(2-chloro-6- fluorophenyl)- 3H-imidazo[4,5- c]pyridin-4- ylamino) nicotinonitrile | B | 365.0 | A | 4.33 |
| 54 | 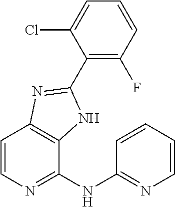 [SMILES: Fc1cccc(Cl)c1-c1nc2ccnc(Nc3ccccn3)c2[nH]1] | 2-(2-chloro-6- fluorophenyl)-N- (pyridin-2-yl)- 3H-imidazo[4,5- c]pyridin-4- amine | B | 340.1 | A | 4.52 |
| 55 | 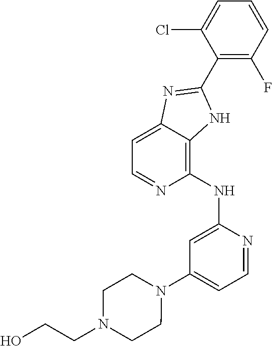 [SMILES: OCCN1CCN(c2ccnc(Nc3nccc4nc(-c5c(F)cccc5Cl)[nH]c34)c2)CC1] | 2-(4-(2-(2-(2- chloro-6- fluorophenyl)- 3H-imidazo[4,5- c]pyridin-4- ylamino)pyridin- 4-yl)piperazin-1- yl)ethanol | B | 468.2 | A | 4.05 |
| 56 | 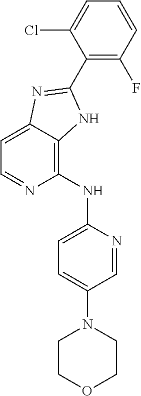 [SMILES: Fc1cccc(Cl)c1-c1nc2ccnc(Nc3ccc(N4CCOCC4)cn3)c2[nH]1] | 2-(2-chloro-6- fluorophenyl)-N- (5- morpholinopyridin- 2-yl)-3H- imidazo[4,5- c]pyridin-4- amine | B | 425.1 | A | 4.44 |
| 57 | 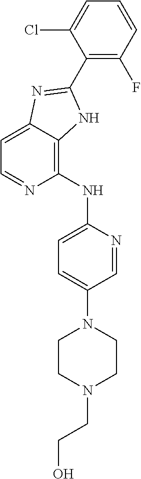 [SMILES: OCCN1CCN(c2ccc(Nc3nccc4nc(-c5c(F)cccc5Cl)[nH]c34)nc2)CC1] | 2-(4-(6-(2-(2- chloro-6- fluorophenyl)- 3H-imidazo[4,5- c]pyridin-4- ylamino)pyridin- 3-yl)piperazin-1- yl)ethanol | B | 468.1 | A | 3.84 |
| 58 | 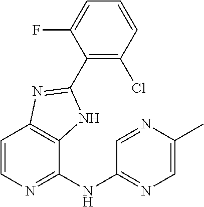 [SMILES: Cc1cnc(Nc2nccc3nc(-c4c(F)cccc4Cl)[nH]c23)cn1] | 2-(2-chloro-6- fluorophenyl)-N- (5- methylpyrazin-2- yl)-3H- imidazo[4,5- c]pyridin-4- amine | B | 355.1 | A | 3.52 |
| 59 | 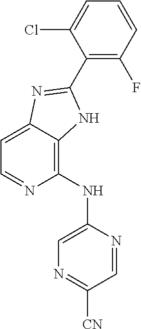 [SMILES: N#Cc1cnc(Nc2nccc3nc(-c4c(F)cccc4Cl)[nH]c23)cn1] | 5-(2-(2-chloro-6- fluorophenyl)- 3H-imidazo[4,5- c]pyridin-4- ylamino)pyrazine- 2-carbonitrile | B | 366.0 | A | 3.69 |
| 60 | 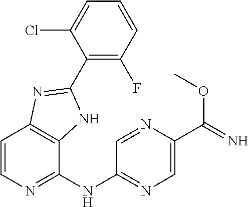 [SMILES: COC(=N)c1cnc(Nc2nccc3nc(-c4c(F)cccc4Cl)[nH]c23)cn1] | methyl 5-(2-(2- chloro-6- fluorophenyl)- 3H-imidazo[4,5- c]pyridin-4- ylamino)pyrazine- 2-carbimidate | B | 398.1 | A | 3.89 |
| 61 | 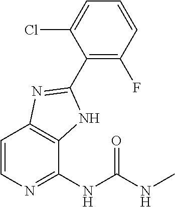 [SMILES: CNC(=O)Nc1nccc2nc(-c3c(F)cccc3Cl)[nH]c12] | 1-(2-(2-chloro-6- fluorophenyl)- 3H-imidazo[4,5- c]pyridin-4-yl)-3- methylurea | B | 320.1 | A | 3.29 |
| 62 | 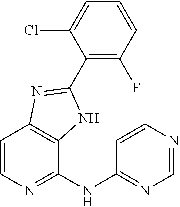 [SMILES: Fc1cccc(Cl)c1-c1nc2ccnc(Nc3ccncn3)c2[nH]1] | 2-(2-chloro-6- fluorophenyl)-N- (pyrimidin-4-yl)- 3H-imidazo[4,5- c]pyridin-4- amine | B | 341.0 | A | 3.18 |
| 63 | 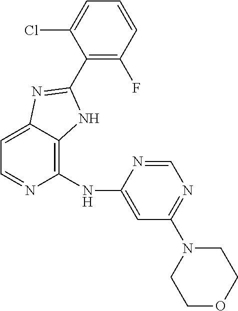 [SMILES: Fc1cccc(Cl)c1-c1nc2ccnc(Nc3cc(N4CCOCC4)ncn3)c2[nH]1] | 2-(2-chloro-6- fluorophenyl)-N- (6- morpholino- pyrimidin- 4-yl)-3H- imidazo[4,5- c]pyridin-4- amine | B | 426.1 | A | 3.89 |
| 64 | 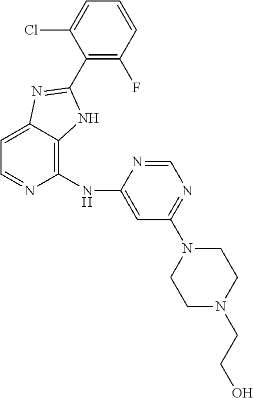 [SMILES: OCCN1CCN(c2cc(Nc3nccc4nc(-c5c(F)cccc5Cl)[nH]c34)ncn2)CC1] | 2-(4-(6-(2-(2- chloro-6- fluorophenyl)- 3H-imidazo[4,5- c]pyridine-4- ylamino)pyrimidin- 4-yl)piperazin- 1-yl)ethanol | B | 469.2 | A | 4.42 |
| 65 | 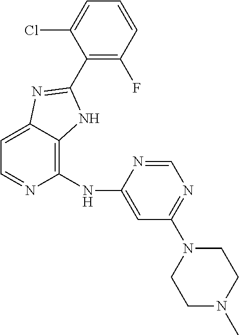 [SMILES: CN1CCN(c2cc(Nc3nccc4nc(-c5c(F)cccc5Cl)[nH]c34)ncn2)CC1] | 2-(2-chloro-6- fluorophenyl)-N- (6-(4- methylpiperazin- 1-yl)-pyrimidin- 4-yl)-3H- imidazo[4,5- c]pyridin-4- amine | B | 439.1 | A | 3.84 |
| 66 | 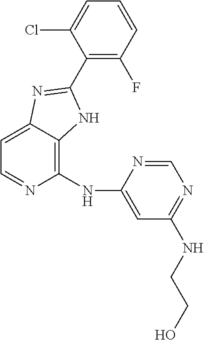 [SMILES: OCCNc1cc(Nc2nccc3nc(-c4c(F)cccc4Cl)[nH]c23)ncn1] | 2-(6-(2-(2- chloro-6- fluorophenyl)- 3H-imidazo[4,5- c]pyridin-4- ylamino)pyrimidin- 4- ylamino)ethanol | B | 400.1 | A | 3.08 |
| 67 | 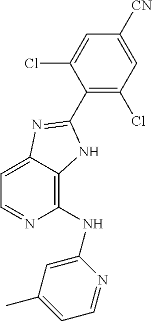 [SMILES: Cc1ccnc(Nc2nccc3nc(-c4c(Cl)cc(C#N)cc4Cl)[nH]c23)c1] | 3,5-dichloro-4- (4-(4- methylpyridin-2- ylamino)-3H- imidazo[4,5- c]pyridin-2- yl)benzonitrile | B | 395.0 | A | 4.80 |
| 68 | 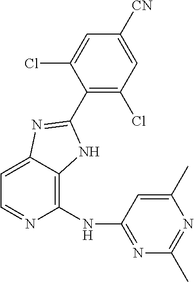 [SMILES: Cc1cc(Nc2nccc3nc(-c4c(Cl)cc(C#N)cc4Cl)[nH]c23)nc(C)n1] | 3,5-dichloro-4- (4-(2,6- dimethylpyrimidin- 4-ylamino)- 3H-imidazo[4,5- c]pyridin-2- yl)benzonitrile | B | 410.1 | A | 3.22 |
| 69 | 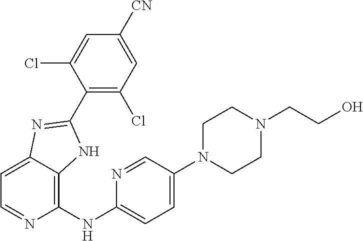 [SMILES: N#Cc1cc(Cl)c(-c2nc3ccnc(Nc4ccc(N5CCN(CCO)CC5)cn4)c3[nH]2)c(Cl)c1] | 3,5-dichloro-4- (4-(5-(4-(2- hydroxyethyl) piperazin-1- yl)pyridin-2- ylamino)-3H- imidazo[4,5- c]pyridin-2- yl)benzonitrile | B | 509.2 | A | 3.92 |
| 70 | 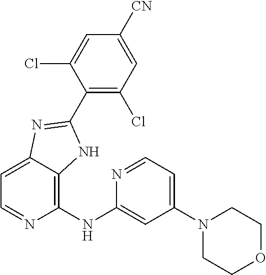 [SMILES: N#Cc1cc(Cl)c(-c2nc3ccnc(Nc4cc(N5CCOCC5)ccn4)c3[nH]2)c(Cl)c1] | 3,5-dichloro-4- (4-(4- morpholinopyridin- 2-ylamino)- 3H-imidazo[4,5- c]pyridin-2- yl)benzonitrile | B | 466.1 | A | 4.59 |
| 71 | 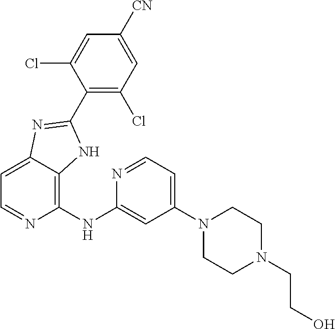 [SMILES: N#Cc1cc(Cl)c(-c2nc3ccnc(Nc4cc(N5CCN(CCO)CC5)ccn4)c3[nH]2)c(Cl)c1] | 3,5-dichloro-4- (4-(4-(4-(2- hydroxyethyl) piperazin-1- yl)pyridin-2- ylamino)-3H- imidazo[4,5- c]pyridin-2- yl)benzonitrile | B | 509.1 | A | 4.05 |
| 72 | 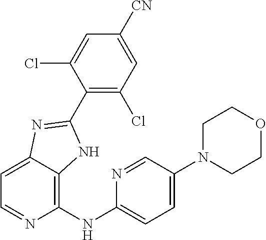 [SMILES: N#Cc1cc(Cl)c(-c2nc3ccnc(Nc4ccc(N5CCOCC5)cn4)c3[nH]2)c(Cl)c1] | 3,5-dichloro-4- (4-(5- morpholinopyridin- 2-ylamino)- 3H-imidazo[4,5- c]pyridin-2- yl)benzonitrile | B | 466.0 | A | 4.45 |
| 73 | 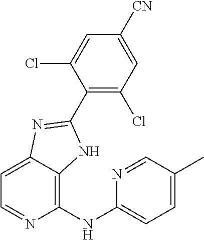 [SMILES: Cc1ccc(Nc2nccc3nc(-c4c(Cl)cc(C#N)cc4Cl)[nH]c23)nc1] | 3,5-dichloro-4- (4-(5- methylpyridin-2- ylamino)-3H- imidazo[4,5- c]pyridin-2- yl)benzonitrile | B | 395.0 | A | 4.95 |
| 74 | 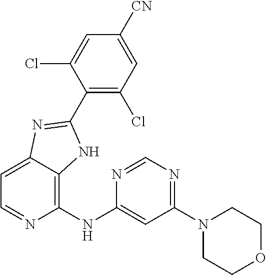 [SMILES: N#Cc1cc(Cl)c(-c2nc3ccnc(Nc4cc(N5CCOCC5)ncn4)c3[nH]2)c(Cl)c1] | 3,5-dichloro-4- (4-(6- morpholino- pyrimidin- 4-ylamino)- 3H-imidazo[4,5- c]pyridin-2- yl)benzonitrile | B | 467.1 | A | 3.53 |
| 75 | 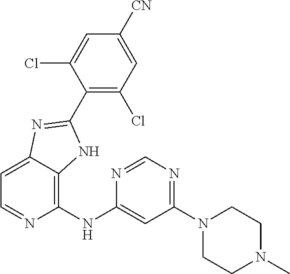 [SMILES: CN1CCN(c2cc(Nc3nccc4nc(-c5c(Cl)cc(C#N)cc5Cl)[nH]c34)ncn2)CC1] | 3,5-dichloro-4- (4-(6-(4- methylpiperazin- 1-yl)pyrimidin-4- ylamino)-3H- imidazo[4,5- c]pyridin-2- yl)benzonitrile | B | 480.1 | A | 3.46 |
| 76 | 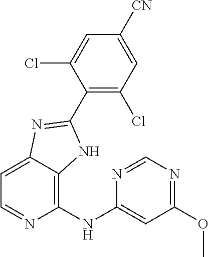 [SMILES: COc1cc(Nc2nccc3nc(-c4c(Cl)cc(C#N)cc4Cl)[nH]c23)ncn1] | 3,5-dichloro-4- (4-(6- methoxypyrimidin- 4-ylamino)- 3H-imidazo[4,5- c]pyridin-2- yl)benzonitrile | B | 412.0 | A | 3.47 |
| 77 | 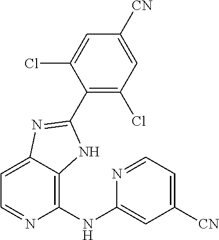 [SMILES: N#Cc1ccnc(Nc2nccc3nc(-c4c(Cl)cc(C#N)cc4Cl)[nH]c23)c1] | 2-(2-(2,6- dichloro-4- cyanophenyl)- 3H-imidazo[4,5- c]pyridin-4- ylamino) isonicotinonitrile | B | 406.0 | A | 3.98 |
| 78 | 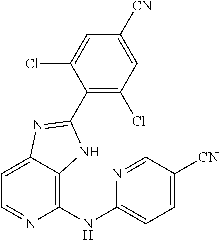 [SMILES: N#Cc1ccc(Nc2nccc3nc(-c4c(Cl)cc(C#N)cc4Cl)[nH]c23)nc1] | 6-(2-(2,6- dichloro-4- cyanophenyl)- 3H-imidazo[4,5- c]pyridin-4- ylamino) nicotinonitrile | B | 406.0 | A | 3.72 |
| 79 | 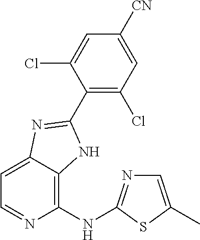 [SMILES: Cc1cnc(Nc2nccc3nc(-c4c(Cl)cc(C#N)cc4Cl)[nH]c23)s1] | 3,5-dichloro-4- (4-(5- methylthiazol-2- ylamino)-3H- imidazo[4,5- c]pyridin-2- yl)benzonitrile | B | 401.0 | A | 4.04 |
| 80 | 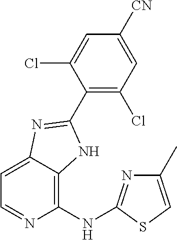 [SMILES: Cc1csc(Nc2nccc3nc(-c4c(Cl)cc(C#N)cc4Cl)[nH]c23)n1] | 3,5-dichloro-4- (4-(4- methylthiazol-2- ylamino)-3H- imidazo[4,5- c]pyridin-2- yl)benzonitrile | B | 401.0 | A | 4.23 |
| 81 | 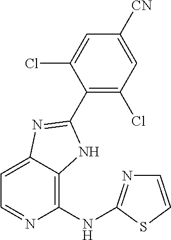 [SMILES: N#Cc1cc(Cl)c(-c2nc3ccnc(Nc4nccs4)c3[nH]2)c(Cl)c1] | 3,5-dichloro-4- (4-(thiazol-2- ylamino)-3H- imidazo[4,5- c]pyridin-2- yl)benzonitrile | B | 387.0 | A | 3.79 |
| 82 | 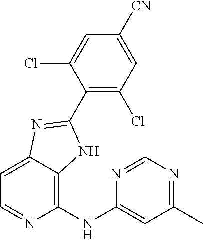 [SMILES: Cc1cc(Nc2nccc3nc(-c4c(Cl)cc(C#N)cc4Cl)[nH]c23)ncn1] | 3,5-dichloro-4- (4-(6- methylpyrimidin- 4-ylamino)-3H- imidazo[4,5- c]pyridin-2- yl)benzonitrile | B | 396.0 | A | 3.04 |
| 83 | 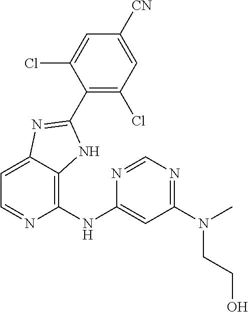 [SMILES: CN(CCO)c1cc(Nc2nccc3nc(-c4c(Cl)cc(C#N)cc4Cl)[nH]c23)ncn1] | 3,5-dichloro-4- (4-(6-((2- hydroxyethyl) (methyl)amino) pyrimidin-4- ylamino)-3H- imidazo[4,5- c]pyridin-2- yl)benzonitrile | B | 455.1 | A | 3.13 |
| 84 | 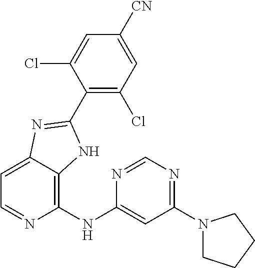 [SMILES: N#Cc1cc(Cl)c(-c2nc3ccnc(Nc4cc(N5CCCC5)ncn4)c3[nH]2)c(Cl)c1] | 3,5-dichloro-4- (4-(6-(pyrrolidin- 1-yl)pyrimidin-4- ylamino)-3H- imidazo[4,5- c]pyridin-2- yl)benzonitrile | B | 451.1 | A | 4.09 |
| 85 | 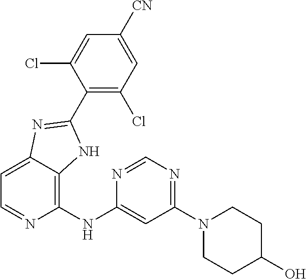 [SMILES: N#Cc1cc(Cl)c(-c2nc3ccnc(Nc4cc(N5CCC(O)CC5)ncn4)c3[nH]2)c(Cl)c1] | 3,5-dichloro-4- (4-(6-(4- hydroxypiperidin- 1-yl)pyrimidin- 4-ylamino)-3H- imidazo[4,5- c]pyridin-2- yl)benzonitrile | B | 481.1 | A | 3.25 |
| 86 | 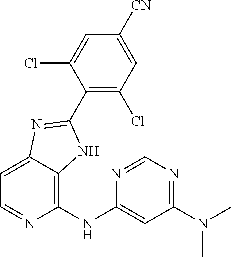 [SMILES: CN(C)c1cc(Nc2nccc3nc(-c4c(Cl)cc(C#N)cc4Cl)[nH]c23)ncn1] | 3,5-dichloro-4- (4-(6- (dimethylamino) pyrimidin-4- ylamino)-3H- imidazo[4,5- c]pyridin-2- yl)benzonitrile | B | 425.1 | A | 3.70 |
| 87 | 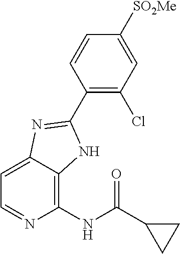 [SMILES: CS(=O)(=O)c1ccc(-c2nc3ccnc(NC(=O)C4CC4)c3[nH]2)c(Cl)c1] | N-(2-(2-chloro-4- (methylsulfonyl) phenyl)-3H- imidazo[4,5-c]- pyridin-4- yl)cyclopropane carboxamide | B | 391.0 | A | 3.29 |
| 88 | 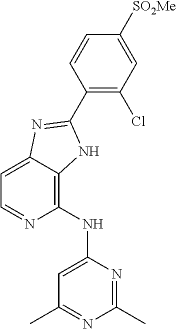 [SMILES: Cc1cc(Nc2nccc3nc(-c4ccc(S(C)(=O)=O)cc4Cl)[nH]c23)nc(C)n1] | 2-(2-chloro-4- (methylsulfonyl) phenyl)-N-(2,6- dimethylpyrimidin- 4-yl)-3H- imidazo[4,5- c]pyridin-4- amine | B | 429.1 | A | 3.27 |
| 89 | 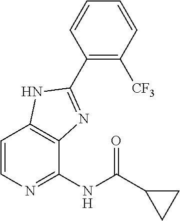 [SMILES: O=C(Nc1nccc2[nH]c(-c3ccccc3C(F)(F)F)nc12)C1CC1] | N-(2-(2- (trifluoromethyl) phenyl)-1H- imidazo[4,5- c]pyridin-4- yl)cyclopropane carboxamide | B | 347.0 | A | 3.66 |
| 90 | 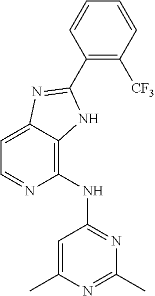 [SMILES: Cc1cc(Nc2nccc3nc(-c4ccccc4C(F)(F)F)[nH]c23)nc(C)n1] | N-(2,6- dimethylpyrimidin- 4-yl)-2-(2- (trifluoromethyl) phenyl)-3H- imidazo[4,5- c]pyridin-4- amine | B | 385.1 | A | 4.27 |
| 91 | 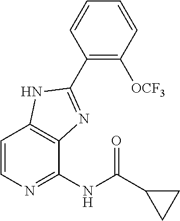 [SMILES: O=C(Nc1nccc2[nH]c(-c3ccccc3OC(F)(F)F)nc12)C1CC1] | N-(2-(2- (trifluoromethoxy) phenyl)-1H- imidazo[4,5- c]pyridin-4- yl)cyclopropane carboxamide | B | 363.1 | A | 3.76 |
| 92 | 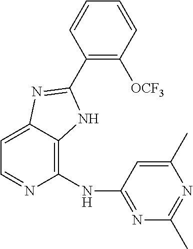 [SMILES: Cc1cc(Nc2nccc3nc(-c4ccccc4OC(F)(F)F)[nH]c23)nc(C)n1] | N-(2,6- dimethylpyrimidin- 4-yl)-2-(2- (trifluoromethoxy) phenyl)-3H- imidazo[4,5- c]pyridin-4- amine | B | 401.1 | A | 4.99 |
| 93 | 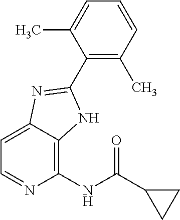 [SMILES: Cc1cccc(C)c1-c1nc2ccnc(NC(=O)C3CC3)c2[nH]1] | N-(2-(2,6- dimethylphenyl)- 3H-imidazo[4,5- c]pyridin-4- yl)cyclopropane carboxamide | B | 307.1 | A | 4.62 |
| 94 | 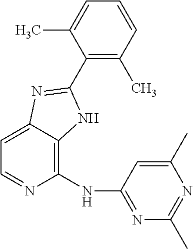 [SMILES: Cc1cc(Nc2nccc3nc(-c4c(C)cccc4C)[nH]c23)nc(C)n1] | 2-(2,6- dimethylphenyl)- N-(2,6- dimethylpyrimidin- 4-yl)-3H- imidazo[4,5- c]pyridin-4- amine | B | 345.2 | A | 4.57 |
| 95 | 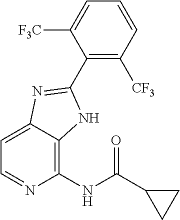 [SMILES: O=C(Nc1nccc2nc(-c3c(C(F)(F)F)cccc3C(F)(F)F)[nH]c12)C1CC1] | N-(2-(2,6- bis(trifluoromethyl) phenyl)-3H- imidazo[4,5- c]pyridin-4- yl)cyclopropane carboxamide | B | 415.1 | A | 4.39 |
| 96 | 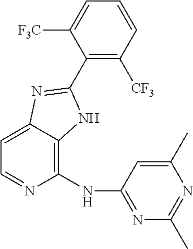 [SMILES: Cc1cc(Nc2nccc3nc(-c4c(C(F)(F)F)cccc4C(F)(F)F)[nH]c23)nc(C)n1] | 2-(2,6- bis(trifluoromethyl) phenyl)-N- (2,6- dimethylpyrimidin- 4-yl)-3H- imidazo[4,5- c]pyridin-4- amine | B | 453.1 | A | 4.21 |
| 97 | 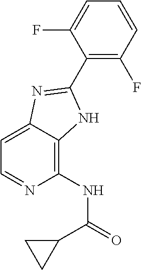 [SMILES: O=C(Nc1nccc2nc(-c3c(F)cccc3F)[nH]c12)C1CC1] | N-(2-(2,6- difluorophenyl)- 3H-imidazo[4,5- c]pyridin-4- yl)cyclopropane carboxamide | B | 315.1 | A | 3.84 |
| 98 | 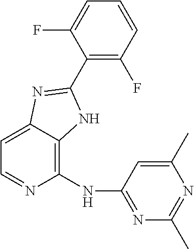 [SMILES: Cc1cc(Nc2nccc3nc(-c4c(F)cccc4F)[nH]c23)nc(C)n1] | 2-(2,6- difluorophenyl)- N-(2,6- dimethylpyrimidin- 4-yl)-3H- imidazo[4,5- c]pyridin-4- amine | B | 353.1 | A | 3.61 |
| 99 | 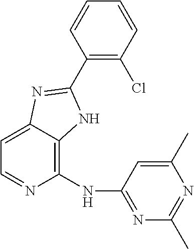 [SMILES: Cc1cc(Nc2nccc3nc(-c4ccccc4Cl)[nH]c23)nc(C)n1] | 2-(2- chlorophenyl)-N- (2,6- dimethylpyrimidin- 4-yl)-3H- imidazo[4,5- c]pyridin-4- amine | B | 351.1 | A | 4.44 |
| 100 | 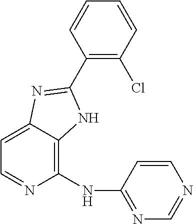 [SMILES: Clc1ccccc1-c1nc2ccnc(Nc3ccncn3)c2[nH]1] | 2-(2- chlorophenyl)-N- (pyrimidin-4-yl)- 3H-imidazo[4,5- c]pyridin-4- amine | B | 323.1 | A | 3.96 |
| 101 | 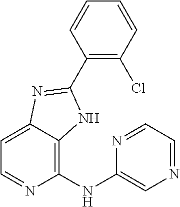 [SMILES: Clc1ccccc1-c1nc2ccnc(Nc3cnccn3)c2[nH]1] | 2-(2- chlorophenyl)-N- (pyrazin-2-yl)- 3H-imidazo[4,5- c]pyridin-4- amine | B | 323.1 | A | 4.33 |
| 102 | 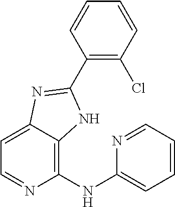 [SMILES: Clc1ccccc1-c1nc2ccnc(Nc3ccccn3)c2[nH]1] | 2-(2- chlorophenyl)-N- (pyridin-2-yl)- 3H-imidazo[4,5- c]pyridine- amine | B | 322.1 | A | 5.09 |
| 103 | 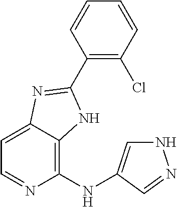 [SMILES: Clc1ccccc1-c1nc2ccnc(Nc3cn[nH]c3)c2[nH]1] | 2-(2- chlorophenyl)-N- (1H-pyrazol-4- yl)-3H- imidazo[4,5- c]pyridin-4- amine | B | 311.1 | A | 3.90 |
| 104 | 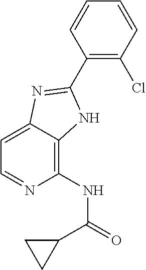 [SMILES: O=C(Nc1nccc2nc(-c3ccccc3Cl)[nH]c12)C1CC1] | N-(2-(2- chlorophenyl)- 3H-imidazo[4,5- c]pyridin-4- yl)cyclopropane carboxamide | B | 313.1 | A | 4.88 |
| 105 | 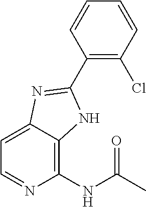 [SMILES: CC(=O)Nc1nccc2nc(-c3ccccc3Cl)[nH]c12] | N-(2-(2- chlorophenyl)- 3H-imidazo[4,5- c]pyridin-4- yl)acetamide | B | 287.1 | A | 3.70 |
| 106 | 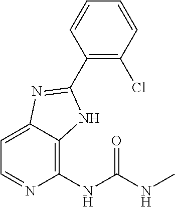 [SMILES: CNC(=O)Nc1nccc2nc(-c3ccccc3Cl)[nH]c12] | 1-(2-(2- chlorophenyl)- 3H-imidazo[4,5- c]pyridin-4-yl)-3- methylurea | B | 302.0 | A | 2.94 |
| 107 | 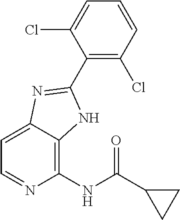 [SMILES: O=C(Nc1nccc2nc(-c3c(Cl)cccc3Cl)[nH]c12)C1CC1] | Cyclopropane- carboxylic acid [2- (2,6-dichloro- phenyl)-1H- imidazo[4,5- c]pyridin-4-yl]- amide | A | 347.2 | B | 4.00 |
| 108 | 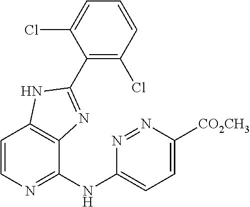 [SMILES: COC(=O)c1ccc(Nc2nccc3[nH]c(-c4c(Cl)cccc4Cl)nc23)nn1] | (2-Chloro-6- methyl- pyrimidin-4-yl)- [2-(2,6-dichloro- phenyl)-3H- imidazo[4,5- c]pyridin-4-yl]- amine | A | 415.2 | B | 3.43 |
| 109 | 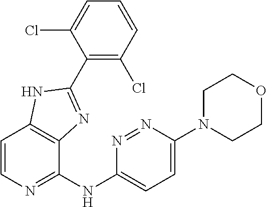 [SMILES: Clc1cccc(Cl)c1-c1nc2c(Nc3ccc(N4CCOCC4)nn3)nccc2[nH]1] | [2-(2,6-Dichloro- phenyl)-3H- imidazo[4,5- c]pyridin-4-yl]- (6-morpholin-4- yl-pyridazin-3- yl)-amine | A | 442.3 | B | 4.52 |
| 110 | 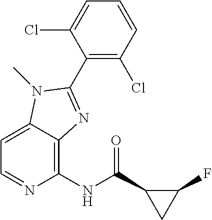 [SMILES: Cn1c(-c2c(Cl)cccc2Cl)nc2c(NC(=O)[C@@H]3C[C@@H]3F)nccc21] | (1S,2S)-2- Fluoro- cyclopropane- carboxylic acid [2- (2,6-dichloro- phenyl)-1- methyl-1H- imidazo[4,5- c]pyridin-4-yl]- amide | A | 379.2 | B | 3.52 |
| 111 | 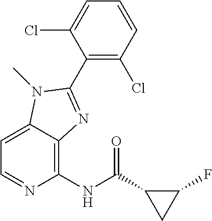 [SMILES: Cn1c(-c2c(Cl)cccc2Cl)nc2c(NC(=O)[C@H]3C[C@H]3F)nccc21] | (1R,2R)-2- Fluoro- cyclopropane- carboxylic acid [2- (2,6-dichloro- phenyl)-1- methyl-1H- imidazo[4,5- c]pyridin-4-yl]- amide | A | 379.2 | B | 3.47 |
| 112 | 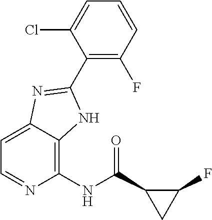 [SMILES: O=C(Nc1nccc2nc(-c3c(F)cccc3Cl)[nH]c12)[C@@H]1C[C@@H]1F] | (1S,2S)-2- Fluoro- cyclopropane- carboxylic acid [2- (2-chloro-6- fluoro-phenyl)- 3H-imidazo[4,5- c]pyridin-4-yl]- amide | B | 348.7 | A | 3.25 |
| 113 | 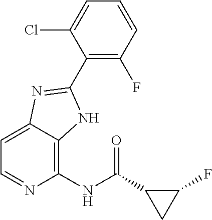 | (1R,2R)-2- Fluoro- cyclopropane- carboxylic acid [2- (2-chloro-6- fluoro-phenyl)- 3H-imidazo[4,5- c]pyridin-4-yl]- amide | B | 348.7 | A | 3.26 |
| 114 | 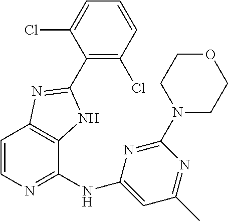 [SMILES: Cc1cc(Nc2nccc3nc(-c4c(Cl)cccc4Cl)[nH]c23)nc(N2CCOCC2)n1] | [2-(2,6-Dichloro- phenyl)-3H- imidazo[4,5- c]pyridin-4-yl]- (6-methyl-2- morpholin-4-yl- pyrimidin-4-yl)- amine | A | 456.3 | B | 3.73 |
| 115 | 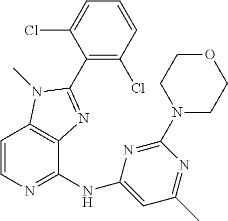 [SMILES: Cc1cc(Nc2nccc3c2nc(-c2c(Cl)cccc2Cl)n3C)nc(N2CCOCC2)n1] | [2-(2,6-Dichloro- phenyl)-1- methyl-1H- imidazo[4,5- c]pyridin-4-yl]- (6-methyl-2- morpholin-4-yl- pyrimidin-4-yl)- amine | A | 470.3 | B | 3.87 |
| 116 | 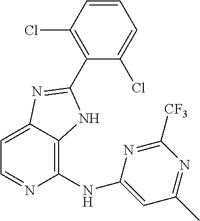 [SMILES: Cc1cc(Nc2nccc3nc(-c4c(Cl)cccc4Cl)[nH]c23)nc(C(F)(F)F)n1] | 2-(2,6- dichlorophenyl)- N-(6-methyl-2- (trifluoromethyl) pyrimidin-4-yl)- 3H-imidazo[4,5- c]pyridin-4- amine | A | 439.2 | B | 11.04 |
| 117 | 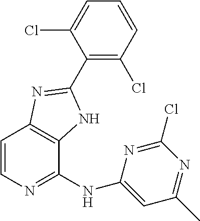 [SMILES: Cc1cc(Nc2nccc3nc(-c4c(Cl)cccc4Cl)[nH]c23)nc(Cl)n1] | (2-Chloro-6- methyl- pyrimidin-4-yl)- [2-(2,6-dichloro- phenyl)-3H- imidazo[4,5- c]pyridin-4-yl]- amine | A | 405.7 | B | 1.49 |
| 118 | 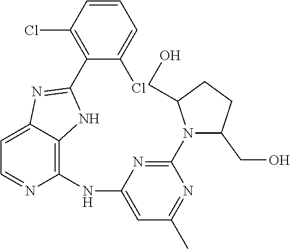 [SMILES: Cc1cc(Nc2nccc3nc(-c4c(Cl)cccc4Cl)[nH]c23)nc(N2C(CO)CCC2CO)n1] | (1-(4-(2-(2,6- dichlorophenyl)- 3H-imidazo[4,5- c]pyridin-4- ylamino)-6- methylpyrimidin- 2-yl)pyrrolidine- 2,5- diyl)dimethanol | A | 500.4 | B | 3.36 |
| 119 | 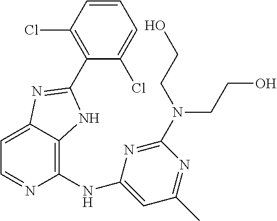 [SMILES: Cc1cc(Nc2nccc3nc(-c4c(Cl)cccc4Cl)[nH]c23)nc(N(CCO)CCO)n1] | 2,2′-(4-(2-(2,6- dichlorophenyl)- 3H-imidazo[4,5- c]pyridin-4- ylamino)-6- methylpyrimidin- 2- ylazanediyl) diethanol | A | 474.3 | B | 3.16 |
| 120 | 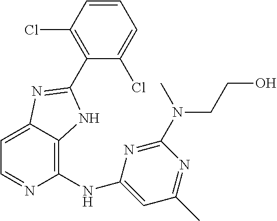 [SMILES: Cc1cc(Nc2nccc3nc(-c4c(Cl)cccc4Cl)[nH]c23)nc(N(C)CCO)n1] | 2-((4-(2-(2,6- dichlorophenyl)- 3H-imidazo[4,5- c]pyridin-4- ylamino)-6- methylpyrimidin- 2- yl)(methyl)amino) ethanol | A | 444.3 | B | 3.22 |
| 121 | 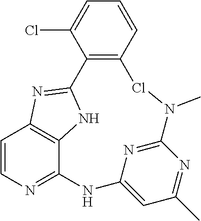 | N⁴-(2-(2,6- dichlorophenyl)- 3H-imidazo[4,5- c]pyridin-4-yl)- N²,N²,6- trimethyl- pyrimidine- 2,4-diamine | A | 414.3 | B | 8.26 |
| 122 | 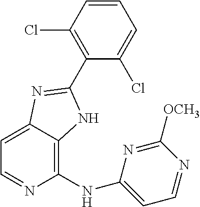 [SMILES: COc1nccc(Nc2nccc3nc(-c4c(Cl)cccc4Cl)[nH]c23)n1] | 2-(2,6- dichlorophenyl)- N-(2- methoxypyrimidin- 4-yl)-3H- imidazo[4,5- c]pyridin-4- amine | A | 387.2 | B | 3.49 |
| 123 | 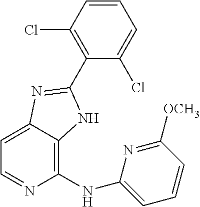 [SMILES: COc1cccc(Nc2nccc3nc(-c4c(Cl)cccc4Cl)[nH]c23)n1] | 2-(2,6- dichlorophenyl)- N-(6- methoxypyridin- 2-yl)-3H- imidazo[4,5- c]pyridin-4- amine | A | 386.2 | B | 4.00 |
| 124 | 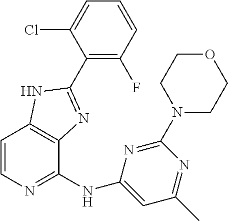 [SMILES: Cc1cc(Nc2nccc3[nH]c(-c4c(F)cccc4Cl)nc23)nc(N2CCOCC2)n1] | [2-(2-Chloro-6- fluoro-phenyl)- 3H-imidazo[4,5- c]pyridin-4-yl]- (6-methyl-2- morpholin-4-yl- pyrimidin-4-yl)- amine | A | 439.9 | B | 3.73 |
| 125 | 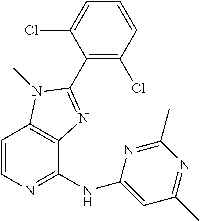 [SMILES: Cc1cc(Nc2nccc3c2nc(-c2c(Cl)cccc2Cl)n3C)nc(C)n1] | [2-(2,6-Dichloro- phenyl)-1- methyl-1H- imidazo[4,5- c]pyridin-4-yl]- (2,6-dimethyl- pyrimidin-4-yl)- amine | A | 399.3 | B | 4.96 |
| 126 | 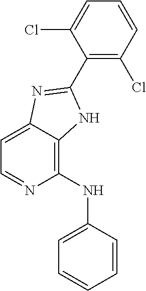 [SMILES: Clc1cccc(Cl)c1-c1nc2ccnc(Nc3ccccc3)c2[nH]1] | 2-(2,6- dichlorophenyl)- N-phenyl-3H- imidazo[4,5- c]pyridin-4- amine | B | 355.0 | A | 5.75 |
| 127 | 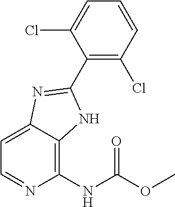 [SMILES: COC(=O)Nc1nccc2nc(-c3c(Cl)cccc3Cl)[nH]c12] | methyl 2-(2,6- dichlorophenyl)- 3H-imidazo[4,5- c]pyridin-4- ylcarbamate | B | 337.0 | A | 3.04 |
| 128 | 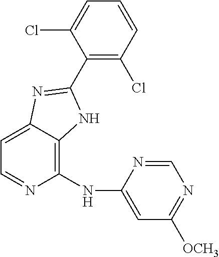 [SMILES: COc1cc(Nc2nccc3nc(-c4c(Cl)cccc4Cl)[nH]c23)ncn1] | 2-(2,6- dichlorophenyl)- N-(6- methoxypyrimidin- 4-yl)-3H- imidazo[4,5- c]pyridin-4- amine | B | 387.0 | A | 4.19 |
| 129 | 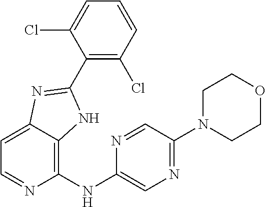 [SMILES: Clc1cccc(Cl)c1-c1nc2ccnc(Nc3cnc(N4CCOCC4)cn3)c2[nH]1] | 2-(2,6- dichlorophenyl)- N-(5- morpholino- pyrazin-2-yl)-3H- imidazo[4,5- c]pyridin-4- amine | B | 442.1 | A | 4.69 |
| 130 | 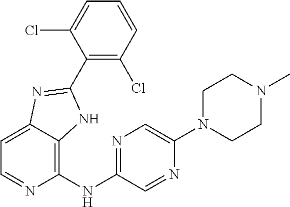 [SMILES: CN1CCN(c2cnc(Nc3nccc4nc(-c5c(Cl)cccc5Cl)[nH]c34)cn2)CC1] | 2-(2,6- dichlorophenyl)- N-(5-(4- methylpiperazin- 1-yl)pyrazin-2- yl)-3H- imidazo[4,5- c]pyridin-4- amine | B | 455.1 | A | 4.48 |
| 131 | 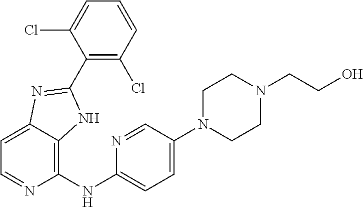 [SMILES: OCCN1CCN(c2ccc(Nc3nccc4nc(-c5c(Cl)cccc5Cl)[nH]c34)nc2)CC1] | 2-(4-(6-(2-(2,6- dichlorophenyl)- 3H-imidazo[4,5- c]pyridin-4- ylamino)pyridin- 3-yl)piperazin-1- yl)ethanol | B | 484.1 | A | 4.00 |
| 132 | 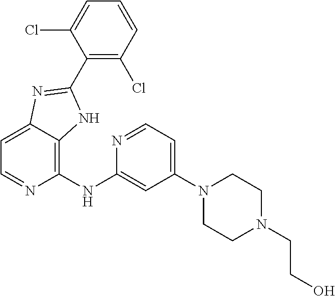 [SMILES: OCCN1CCN(c2ccnc(Nc3nccc4nc(-c5c(Cl)cccc5Cl)[nH]c34)c2)CC1] | 2-(4-(2-(2-(2,6- dichlorophenyl)- 3H-imidazo[4,5- c]pyridin-4- ylamino)pyridin- 4-yl)piperazin-1- yl)ethanol | B | 484.1 | A | 4.17 |
| 133 | 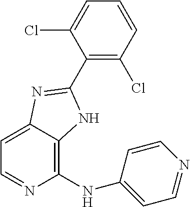 [SMILES: Clc1cccc(Cl)c1-c1nc2ccnc(Nc3ccncc3)c2[nH]1] | 2-(2,6- dichlorophenyl)- N-(pyridin-4-yl)- 3H-imidazo[4,5- c]pyridin-4- amine | B | 356.0 | A | 3.59 |
| 134 | 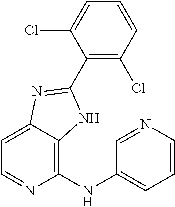 [SMILES: Clc1cccc(Cl)c1-c1nc2ccnc(Nc3cccnc3)c2[nH]1] | 2-(2,6- dichlorophenyl)- N-(pyridin-3-yl)- 3H-imidazo[4,5- c]pyridin-4- amine | B | 356.0 | A | 4.27 |
| 135 | 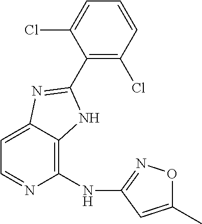 [SMILES: Cc1cc(Nc2nccc3nc(-c4c(Cl)cccc4Cl)[nH]c23)no1] | N-(2-(2,6- dichlorophenyl)- 3H-imidazo[4,5- c]pyridin-4-yl)-5- methylisoxazol- 3-amine | B | 360.0 | A | 2.44 |
| 136 | 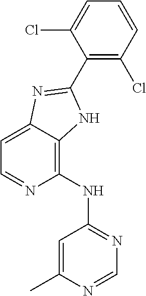 [SMILES: Cc1cc(Nc2nccc3nc(-c4c(Cl)cccc4Cl)[nH]c23)ncn1] | 2-(2,6- dichlorophenyl)- N-(6- methylpyrimidin- 4-yl)-3H- imidazo[4,5- c]pyridin-4- amine | B | 371.0 | A | 3.63 |
| 137 | 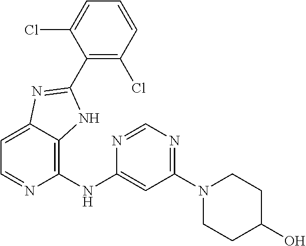 [SMILES: OC1CCN(c2cc(Nc3nccc4nc(-c5c(Cl)cccc5Cl)[nH]c34)ncn2)CC1] | 1-(6-(2-(2,6- dichlorophenyl)- 3H-imidazo[4,5- c]pyridin-4- ylamino)pyrimidin- 4-yl)piperidin- 4-ol | B | 456.1 | A | 3.75 |
| 138 | 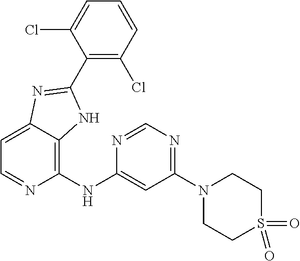 [SMILES: O=S1(=O)CCN(c2cc(Nc3nccc4nc(-c5c(Cl)cccc5Cl)[nH]c34)ncn2)CC1] | 2-(2,6- dichlorophenyl)- N-(6-(1,1- dioxothio- morpholin-4- yl)pyrimidin-4- yl)-3H- imidazo[4,5- c]pyridin-4- amine | B | 490.0 | A | 3.70 |
| 139 | 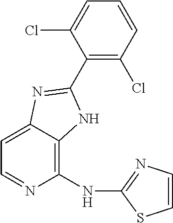 [SMILES: Clc1cccc(Cl)c1-c1nc2ccnc(Nc3nccs3)c2[nH]1] | N-(2-(2,6- dichlorophenyl)- 3H-imidazo[4,5- c]pyridin-4- yl)thiazol-2- amine | B | 362.0 | A | 4.61 |
| 140 | 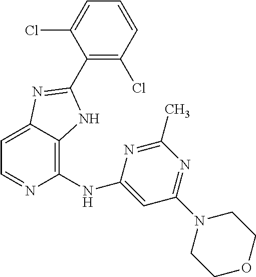 [SMILES: Cc1nc(Nc2nccc3nc(-c4c(Cl)cccc4Cl)[nH]c23)cc(N2CCOCC2)n1] | 2-(2,6- dichlorophenyl)- N-(2-methyl-6- morpholino- pyrimidin-4-yl)-3H- imidazo[4,5- c]pyridin-4- amine | B | 456.1 | A | 4.52 |
| 141 | 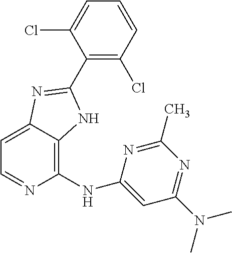 [SMILES: Cc1nc(Nc2nccc3nc(-c4c(Cl)cccc4Cl)[nH]c23)cc(N(C)C)n1] | N⁴-(2-(2,6- dichlorophenyl)- 3H-imidazo[4,5- c]pyridin-4-yl)- N⁶,N⁶,2- trimethyl- pyrimidine- 4,6-diamine | B | 414.1 | A | 4.08 |
| 142 | 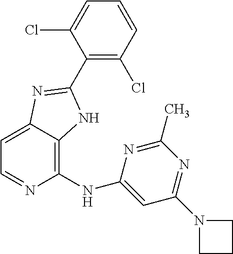 [SMILES: Cc1nc(Nc2nccc3nc(-c4c(Cl)cccc4Cl)[nH]c23)cc(N2CCC2)n1] | N-(6-(azetidin-1- yl)-2- methylpyrimidin- 4-yl)-2-(2,6- dichlorophenyl)- 3H-imidazo[4,5- c]pyridin-4- amine | B | 426.1 | A | 4.35 |
| 143 | 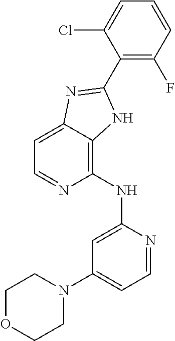 [SMILES: Fc1cccc(Cl)c1-c1nc2ccnc(Nc3cc(N4CCOCC4)ccn3)c2[nH]1] | 2-(2-chloro-6- fluorophenyl)-N- (4- morpholinopyridin- 2-yl)-3H- imidazo[4,5- c]pyridin-4- amine | B | 425.1 | A | 4.63 |
| 144 | 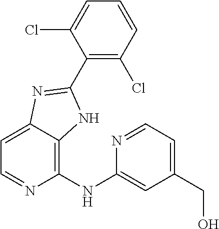 [SMILES: OCc1ccnc(Nc2nccc3nc(-c4c(Cl)cccc4Cl)[nH]c23)c1] | (2-(2-(2,6- dichlorophenyl)- 3H-imidazo[4,5- c]pyridin-4- ylamino)pyridin- 4-yl)methanol | B | 386.0 | A | 3.78 |
| 145 | 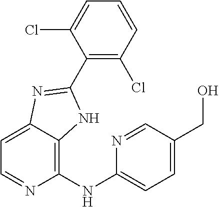 [SMILES: OCc1ccc(Nc2nccc3nc(-c4c(Cl)cccc4Cl)[nH]c23)nc1] | (6-(2-(2,6- dichlorophenyl)- 3H-imidazo[4,5- c]pyridin-4- ylamino)pyridin- 3-yl)methanol | B | 386.0 | A | 3.81 |
| 146 | 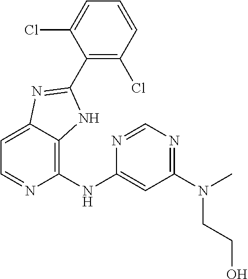 [SMILES: CN(CCO)c1cc(Nc2nccc3nc(-c4c(Cl)cccc4Cl)[nH]c23)ncn1] | 2-((6-(2-(2,6- dichlorophenyl)- 3H-imidazo[4,5- c]pyridin-4- ylamino) pyrimidin-4- yl)(methyl)amino) ethanol | B | 430.1 | A | 3.66 |
| 147 | 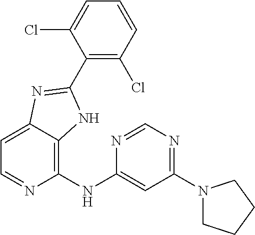 [SMILES: Clc1cccc(Cl)c1-c1nc2ccnc(Nc3cc(N4CCCC4)ncn3)c2[nH]1] | 2-(2,6- dichlorophenyl)- N-(6-(pyrrolidin- 1-yl)pyrimidin-4- yl)-3H- imidazo[4,5- c]pyridine- amine | B | 426.1 | A | 4.58 |
| 148 | 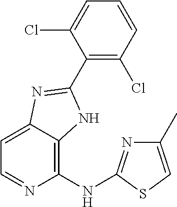 [SMILES: Cc1csc(Nc2nccc3nc(-c4c(Cl)cccc4Cl)[nH]c23)n1] | N-(2-(2,6- dichlorophenyl)- 3H-imidazo[4,5- c]pyridin-4-yl)-4- methylthiazol-2- amine | B | 376.0 | A | 5.08 |
| 149 | 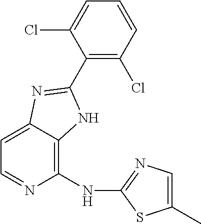 [SMILES: Cc1cnc(Nc2nccc3nc(-c4c(Cl)cccc4Cl)[nH]c23)s1] | N-(2-(2,6- dichlorophenyl)- 3H-imidazo[4,5- c]pyridin-4-yl)-5- methylthiazol-2- amine | B | 376.0 | A | 5.05 |
| 150 | 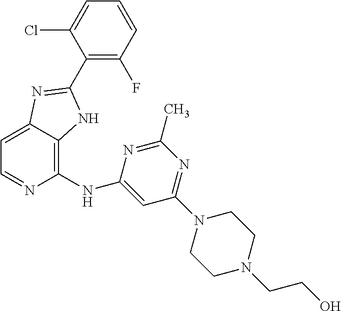 [SMILES: Cc1nc(Nc2nccc3nc(-c4c(F)cccc4Cl)[nH]c23)cc(N2CCN(CCO)CC2)n1] | 2-(4-(6-(2-(2- chloro-6- fluorophenyl)- 3H-imidazo[4,5- c]pyridin-4- ylamino)-2- methylpyrimidin- 4-yl)piperazin-1- yl)ethanol | B | 483.1 | A | 3.82 |
| 151 | 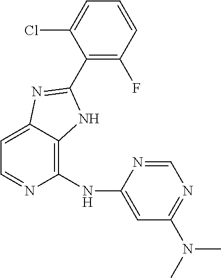 [SMILES: CN(C)c1cc(Nc2nccc3nc(-c4c(F)cccc4Cl)[nH]c23)ncn1] | N⁴-(2-(2-chloro- 6-fluorophenyl)- 3H-imidazo[4,5- c]pyridin-4-yl)- N⁶,N⁶- dimethyl- pyrimidine- 4,6-diamine | B | 384.1 | A | 4.19 |
| 152 | 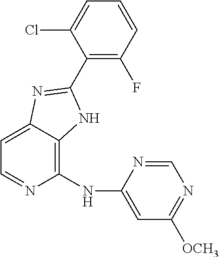 [SMILES: COc1cc(Nc2nccc3nc(-c4c(F)cccc4Cl)[nH]c23)ncn1] | 2-(2-chloro-6- fluorophenyl)-N- (6- methoxypyrimidin- 4-yl)-3H- imidazo[4,5- c]pyridin-4- amine | B | 371.1 | A | 4.01 |
| 153 | 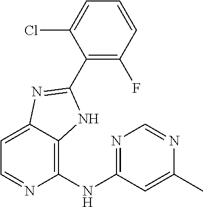 [SMILES: Cc1cc(Nc2nccc3nc(-c4c(F)cccc4Cl)[nH]c23)ncn1] | 2-(2-chloro-6- fluorophenyl)-N- (6- methylpyrimidin- 4-yl)-3H- imidazo[4,5- c]pyridin-4- amine | B | 355.1 | A | 3.49 |
| 154 | 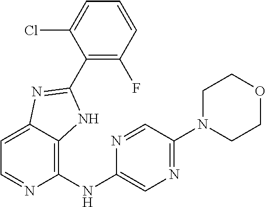 [SMILES: Fc1cccc(Cl)c1-c1nc2ccnc(Nc3cnc(N4CCOCC4)cn3)c2[nH]1] | 2-(2-chloro-6- fluorophenyl)-N- (5- morpholinopyrazin- 2-yl)-3H- imidazo[4,5- c]pyridin-4- amine | B | 426.1 | A | 4.03 |
| 155 | 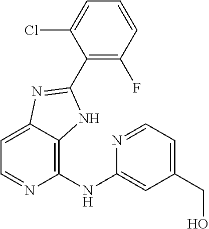 [SMILES: OCc1ccnc(Nc2nccc3nc(-c4c(F)cccc4Cl)[nH]c23)c1] | (2-(2-(2-chloro- 6-fluorophenyl)- 3H-imidazo[4,5- c]pyridin-4- ylamino)pyridin- 4-yl)methanol | B | 370.1 | A | 3.73 |
| 156 | 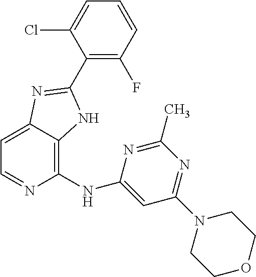 [SMILES: Cc1nc(Nc2nccc3nc(-c4c(F)cccc4Cl)[nH]c23)cc(N2CCOCC2)n1] | 2-(2-chloro-6- fluorophenyl)-N- (2-methyl-6- morpholino- pyrimidin-4-yl)-3H- imidazo[4,5- c]pyridin-4- amine | B | 440.1 | A | 4.59 |
| 157 | 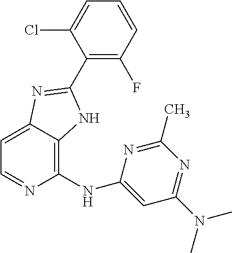 [SMILES: Cc1nc(Nc2nccc3nc(-c4c(F)cccc4Cl)[nH]c23)cc(N(C)C)n1] | N⁴-(2-(2-chloro- 6-fluorophenyl)- 3H-imidazo[4,5- c]pyridin-4-yl)- N⁶,N⁶,2- trimethyl- pyrimidine- 4,6-diamine | B | 398.1 | A | 4.72 |
| 158 | 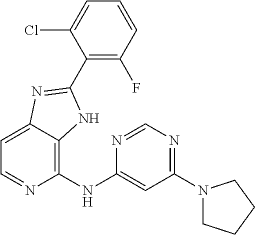 [SMILES: Fc1cccc(Cl)c1-c1nc2ccnc(Nc3cc(N4CCCC4)ncn3)c2[nH]1] | 2-(2-chloro-6- fluorophenyl)-N- (6-(pyrrolidin-1- yl)pyrimidin-4- yl)-3H- imidazo[4,5- c]pyridin-4- amine | B | 410.0 | A | 4.73 |
| 159 | 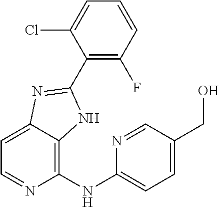 [SMILES: OCc1ccc(Nc2nccc3nc(-c4c(F)cccc4Cl)[nH]c23)nc1] | (6-(2-(2-chloro- 6-fluorophenyl)- 3H-imidazo[4,5- c]pyridin-4- ylamino)pyridin- 3-yl)methanol | B | 370.1 | A | 3.60 |
| 160 | 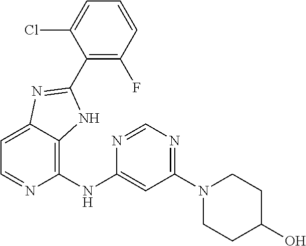 [SMILES: OC1CCN(c2cc(Nc3nccc4nc(-c5c(F)cccc5Cl)[nH]c34)ncn2)CC1] | 1-(6-(2-(2- chloro-6- fluorophenyl)- 3H-imidazo[4,5- c]pyridin-4- ylamino)pyrimidin- 4-yl)piperidin- 4-ol | B | 440.2 | A | 3.55 |
| 161 | 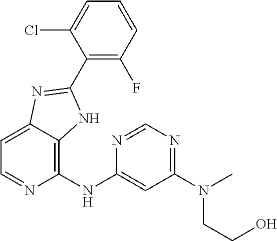 [SMILES: CN(CCO)c1cc(Nc2nccc3nc(-c4c(F)cccc4Cl)[nH]c23)ncn1] | 2-((6-(2-(2- chloro-6- fluorophenyl)- 3H-imidazo[4,5- c]pyridin-4- ylamino)pyrimidin- 4- yl)(methyl)amino) ethanol | B | 414.1 | A | 3.51 |
| 162 | 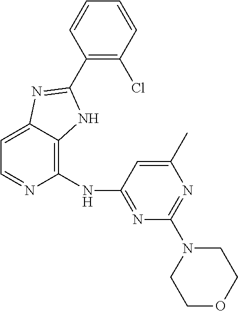 [SMILES: Cc1cc(Nc2nccc3nc(-c4ccccc4Cl)[nH]c23)nc(N2CCOCC2)n1] | 2-(2- chlorophenyl)-N- (6-methyl-2- morpholino- pyrimidin- 4-yl)-3H- imidazo[4,5- c]pyridin-4- amine | B | 422.1 | A | 5.54 |
| 163 | 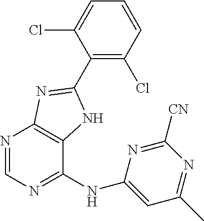 [SMILES: Cc1cc(Nc2ncnc3nc(-c4c(Cl)cccc4Cl)[nH]c23)nc(C#N)n1] | 4-[8-(2,6- Dichloro- phenyl)-7H- purin-6- ylamino]- pyrimidine-2- carbonitrile | D | 397.2 | B | 4.17 |
| 164 | 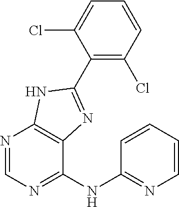 [SMILES: Clc1cccc(Cl)c1-c1nc2c(Nc3ccccn3)ncnc2[nH]1] | 8-(2, 6- dichlorophenyl)- N-(pyridin-2-yl)- 9H-purin-6- amine | E | 357.0 | A | 2.86 |
| 165 | 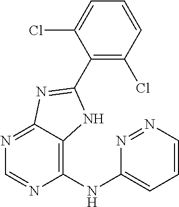 [SMILES: Clc1cccc(Cl)c1-c1nc2ncnc(Nc3cccnn3)c2[nH]1] | 8-(2,6- dichlorophenyl)- N-(pyridazin-3- yl)-7H-purin-6- amine | E | 358.0 | A | 2.19 |
| 166 | 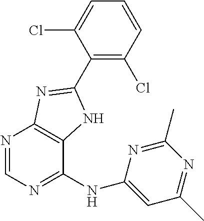 [SMILES: Cc1cc(Nc2ncnc3nc(-c4c(Cl)cccc4Cl)[nH]c23)nc(C)n1] | [8-(2,6-Dichloro- phenyl)-7H- purin-6-yl]-(2,6- dimethyl- pyrimidin-4-yl)- amine | D | 386.2 | B | 3.14 |
| 167 | 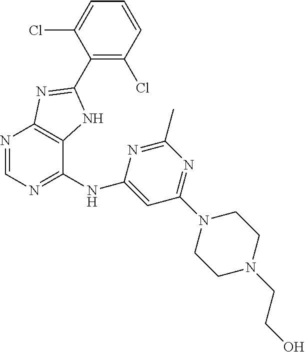 [SMILES: Cc1nc(Nc2ncnc3nc(-c4c(Cl)cccc4Cl)[nH]c23)cc(N2CCN(CCO)CC2)n1] | 2-(4-{6-[8-(2,6- Dichloro- phenyl)-7H- purin-6- ylamino]-2- methyl- pyrimidin-4-yl}- piperazin-1-yl)- ethanol | D | 500.4 | B | 2.88 |
| 168 | 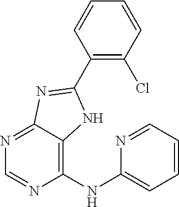 [SMILES: Clc1ccccc1-c1nc2ncnc(Nc3ccccn3)c2[nH]1] | 8-(2- chlorophenyl)-N- (pyridin-2-yl)- 7H-purin-6- amine | E | 323.1 | A | 3.67 |
| 169 | 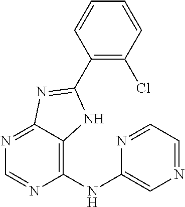 [SMILES: Clc1ccccc1-c1nc2ncnc(Nc3cnccn3)c2[nH]1] | 8-(2- chlorophenyl)-N- (pyrazin-2-yl)- 7H-purin-6- amine | E | 324.1 | A | 2.72 |
| 170 | 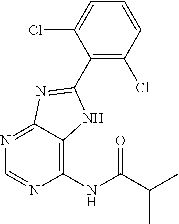 [SMILES: CC(C)C(=O)Nc1ncnc2nc(-c3c(Cl)cccc3Cl)[nH]c12] | N-(8-(2,6- dichlorophenyl)- 7H-purin-6- yl)isobutyramide | E | 350.0 | A | 2.59 |
| 171 | 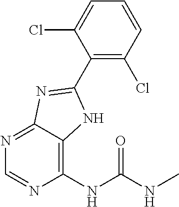 [SMILES: CNC(=O)Nc1ncnc2nc(-c3c(Cl)cccc3Cl)[nH]c12] | 1-(8-(2,6- dichlorophenyl)- 7H-purin-6-yl)-3- methylurea | E | 337.0 | A | 3.65 |
| 172 | 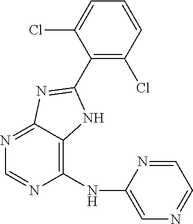 [SMILES: Clc1cccc(Cl)c1-c1nc2ncnc(Nc3cnccn3)c2[nH]1] | 8-(2,6- dichlorophenyl)- N-(pyrazin-2-yl)- 7H-purin-6- amine | E | 358.1 | A | 2.34 |
| 173 | 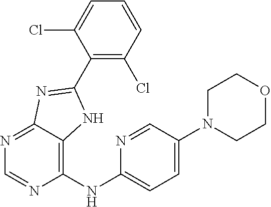 [SMILES: Clc1cccc(Cl)c1-c1nc2ncnc(Nc3ccc(N4CCOCC4)cn3)c2[nH]1] | 8-(2,6- dichlorophenyl)- N-(5- morpholinopyridin- 2-yl)-7H-purin- 6-amine | E | 442.1 | A | 2.92 |
| 174 | 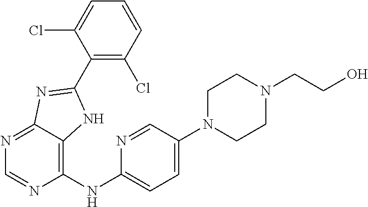 [SMILES: OCCN1CCN(c2ccc(Nc3ncnc4nc(-c5c(Cl)cccc5Cl)[nH]c34)nc2)CC1] | 2-(4-(6-(8-(2,6- dichlorophenyl)- 7H-purin-6- ylamino)pyridin- 3-yl)piperazin-1- yl)ethanol | E | 485.1 | A | 2.82 |
| 175 | 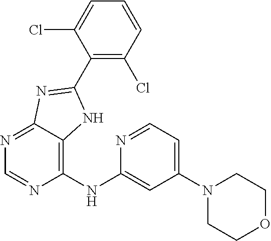 [SMILES: Clc1cccc(Cl)c1-c1nc2ncnc(Nc3cc(N4CCOCC4)ccn3)c2[nH]1] | 8-(2,6- dichlorophenyl)- N-(4- morpholinopyridin- 2-yl)-7H-purin- 6-amine | E | 442.0 | A | 4.04 |
| 176 | 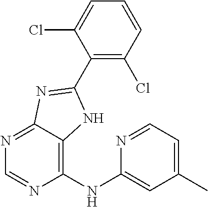 [SMILES: Cc1ccnc(Nc2ncnc3nc(-c4c(Cl)cccc4Cl)[nH]c23)c1] | 8-(2,6- dichlorophenyl)- N-(4- methylpyridin-2- yl)-7H-purin-6- amine | E | 371.0 | A | 3.13 |
| 177 | 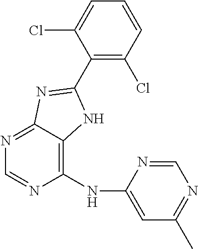 [SMILES: Cc1cc(Nc2ncnc3nc(-c4c(Cl)cccc4Cl)[nH]c23)ncn1] | 8-(2,6- dichlorophenyl)- N-(6- methylpyrimidin- 4-yl)-7H-purin-6- amine | E | 372.0 | A | 3.72 |
| 178 | 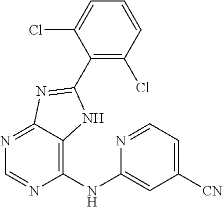 [SMILES: N#Cc1ccnc(Nc2ncnc3nc(-c4c(Cl)cccc4Cl)[nH]c23)c1] | 2-(8-(2,6- dichlorophenyl)- 7H-purin-6- ylamino) isonicotinonitrile | E | 382.0 | A | 2.99 |
| 179 | 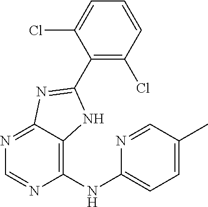 [SMILES: Cc1ccc(Nc2ncnc3nc(-c4c(Cl)cccc4Cl)[nH]c23)nc1] | 8-(2,6- dichlorophenyl)- N-(5- methylpyridin-2- yl)-7H-purin-6- amine | E | 371.0 | A | 3.22 |
| 180 | 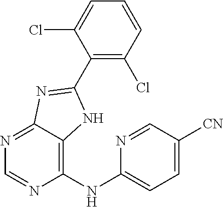 [SMILES: N#Cc1ccc(Nc2ncnc3nc(-c4c(Cl)cccc4Cl)[nH]c23)nc1] | 6-(8-(2,6- dichlorophenyl)- 7H-purin-6- ylamino) nicotinonitrile | E | 382.0 | A | 2.81 |
| 181 | 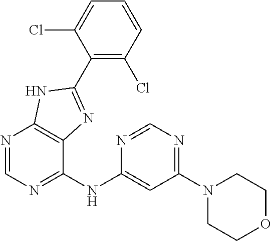 [SMILES: Clc1cccc(Cl)c1-c1nc2c(Nc3cc(N4CCOCC4)ncn3)ncnc2[nH]1] | 8-(2,6- dichlorophenyl)- N-(6- morpholino- pyrimidin-4-yl)-9H- purin-6-amine | E | 443.0 | A | 2.73 |
| 182 | 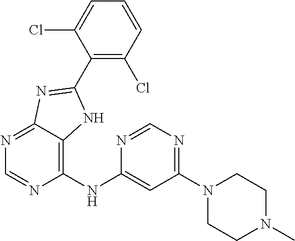 [SMILES: CN1CCN(c2cc(Nc3ncnc4nc(-c5c(Cl)cccc5Cl)[nH]c34)ncn2)CC1] | 8-(2,6- dichlorophenyl)- N-(6-(4- methylpiperazin- 1-yl)pyrimidin-4- yl)-7H-purin-6- amine | E | 456.1 | A | 2.77 |
| 183 | 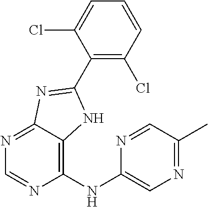 [SMILES: Cc1cnc(Nc2ncnc3nc(-c4c(Cl)cccc4Cl)[nH]c23)cn1] | 8-(2,6- dichlorophenyl)- N-(5- methylpyrazin-2- yl)-7H-purin-6- amine | E | 372.0 | A | 2.62 |
| 184 | 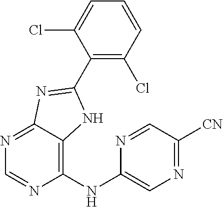 [SMILES: N#Cc1cnc(Nc2ncnc3nc(-c4c(Cl)cccc4Cl)[nH]c23)cn1] | 5-(8-(2,6- dichlorophenyl)- 7H-purin-6- ylamino)pyrazine- 2-carbonitrile | E | 383.0 | A | 2.66 |
| 185 | 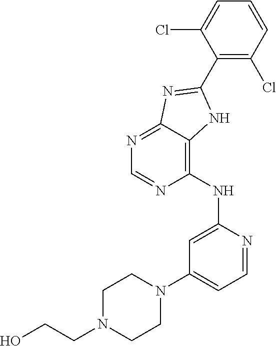 [SMILES: OCCN1CCN(c2ccnc(Nc3ncnc4nc(-c5c(Cl)cccc5Cl)[nH]c34)c2)CC1] | 2-(4-(2-(8-(2,6- dichlorophenyl)- 7H-purin-6- ylamino)pyridin- 4-yl)piperazin-1- yl)ethanol | E | 485.1 | A | 3.13 |
| 186 | 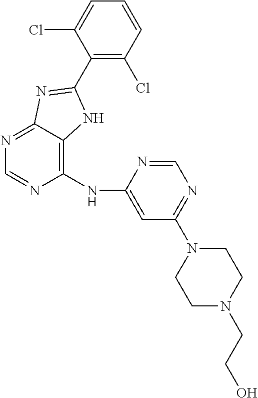 [SMILES: OCCN1CCN(c2cc(Nc3ncnc4nc(-c5c(Cl)cccc5Cl)[nH]c34)ncn2)CC1] | 2-(4-(6-(8-(2,6- dichlorophenyl)- 7H-purin-6- ylamino)pyrimidin- 4-yl)piperazin- 1-yl)ethanol | E | 486.1 | A | 2.60 |
| 187 | 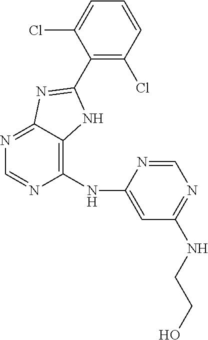 [SMILES: OCCNc1cc(Nc2ncnc3nc(-c4c(Cl)cccc4Cl)[nH]c23)ncn1] | 2-(6-(8-(2,6- dichlorophenyl)- 7H-purin-6- ylamino)pyrimidin- 4- ylamino)ethanol | E | 417.0 | A | 2.29 |
| 188 | 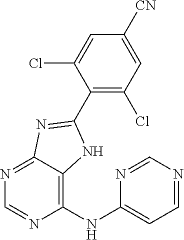 [SMILES: N#Cc1cc(Cl)c(-c2nc3ncnc(Nc4ccncn4)c3[nH]2)c(Cl)c1] | 3,5-dichloro-4- (6-(pyrimidin-4- ylamino)-7H- purin-8- yl)benzonitrile | E | 383.0 | A | 2.35 |
| 189 | 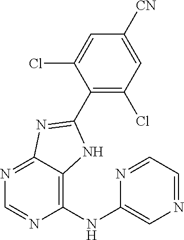 [SMILES: N#Cc1cc(Cl)c(-c2nc3ncnc(Nc4cnccn4)c3[nH]2)c(Cl)c1] | 3,5-dichloro-4- (6-(pyrazin-2- ylamino)-7H- purin-8- yl)benzonitrile | E | 383.0 | A | 4.28 |
| 190 | 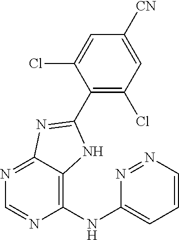 [SMILES: N#Cc1cc(Cl)c(-c2nc3ncnc(Nc4cccnn4)c3[nH]2)c(Cl)c1] | 3,5-dichloro-4- (6-(pyridazin-3- ylamino)-7H- purin-8- yl)benzonitrile | E | 383.0 | A | 3.70 |
| 191 | 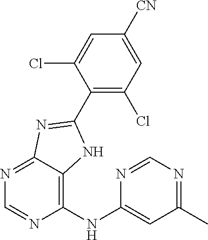 [SMILES: Cc1cc(Nc2ncnc3nc(-c4c(Cl)cc(C#N)cc4Cl)[nH]c23)ncn1] | 3,5-dichloro-4- (6-(6- methylpyrimidin- 4-ylamino)-7H- purin-8- yl)benzonitrile | E | 397.0 | A | 3.71 |
| 192 | 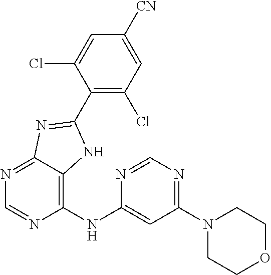 [SMILES: N#Cc1cc(Cl)c(-c2nc3ncnc(Nc4cc(N5CCOCC5)ncn4)c3[nH]2)c(Cl)c1] | 3,5-dichloro-4- (6-(6- morpholino- pyrimidin- 4-ylamino)- 7H-purin-8- yl)benzonitrile | E | 468.0 | A | 2.70 |
| 193 | 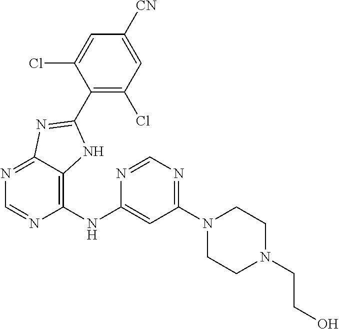 [SMILES: N#Cc1cc(Cl)c(-c2nc3ncnc(Nc4cc(N5CCN(CCO)CC5)ncn4)c3[nH]2)c(Cl)c1] | 3,5-dichloro-4- (6-(6-(4-(2- hydroxyethyl) piperazin-1- yl)pyrimidin-4- ylamino)-7H- purin-8- yl)benzonitrile | E | 511.1 | A | 2.60 |
| 194 | 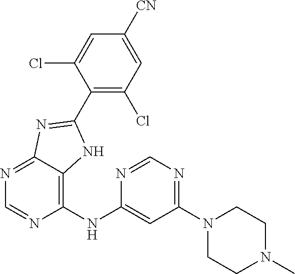 [SMILES: CN1CCN(c2cc(Nc3ncnc4nc(-c5c(Cl)cc(C#N)cc5Cl)[nH]c34)ncn2)CC1] | 3,5-dichloro-4- (6-(6-(4- methylpiperazin- 1-yl)pyrimidin-4- ylamino)-7H- purin-8- yl)benzonitrile | E | 481.1 | A | 2.81 |
| 195 | 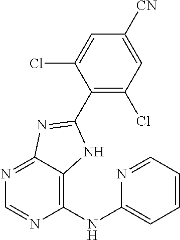 [SMILES: N#Cc1cc(Cl)c(-c2nc3ncnc(Nc4ccccn4)c3[nH]2)c(Cl)c1] | 3,5-dichloro-4- (6-(pyridin-2- ylamino)-7H- purin-8- yl)benzonitrile | E | 382.0 | A | 2.65 |
| 196 | 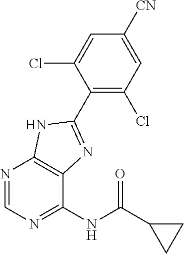 [SMILES: N#Cc1cc(Cl)c(-c2nc3c(NC(=O)C4CC4)ncnc3[nH]2)c(Cl)c1] | N-(8-(2,6- dichloro-4- cyanophenyl)- 9H-purin-6- yl)cyclopropane carboxamide | E | 373.0 | A | 2.38 |
| 197 | 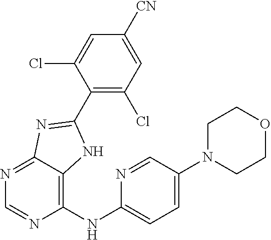 [SMILES: N#Cc1cc(Cl)c(-c2nc3ncnc(Nc4ccc(N5CCOCC5)cn4)c3[nH]2)c(Cl)c1] | 3,5-dichloro-4- (6-(5- morpholinopyridin- 2-ylamino)- 7H-purin-8- yl)benzonitrile | E | 467.1 | A | 3.61 |
| 198 | 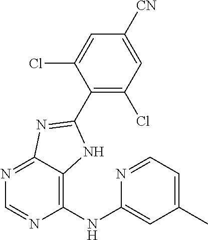 [SMILES: Cc1ccnc(Nc2ncnc3nc(-c4c(Cl)cc(C#N)cc4Cl)[nH]c23)c1] | 3,5-dichloro-4- (6-(4- methylpyridin-2- ylamino)-7H- purin-8- yl)benzonitrile | E | 396.0 | A | 3.44 |
| 199 | 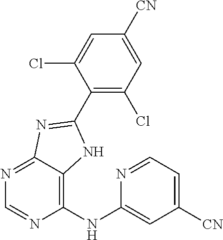 [SMILES: N#Cc1ccnc(Nc2ncnc3nc(-c4c(Cl)cc(C#N)cc4Cl)[nH]c23)c1] | 2-(8-(2,6- dichloro-4- cyanophenyl)- 7H-purin-6- ylamino) isonicotinonitrile | E | 407.0 | A | 2.86 |
| 200 | 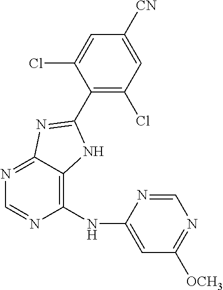 [SMILES: COc1cc(Nc2ncnc3nc(-c4c(Cl)cc(C#N)cc4Cl)[nH]c23)ncn1] | 3,5-dichloro-4- (6-(6- methoxypyrimidin- 4-ylamino)- 7H-purin-8- yl)benzonitrile | E | 413.0 | A | 2.68 |
| 201 | 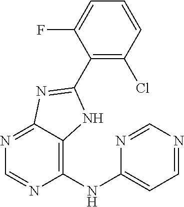 [SMILES: Fc1cccc(Cl)c1-c1nc2ncnc(Nc3ccncn3)c2[nH]1] | 8-(2-chloro-6- fluorophenyl)-N- (pyrimidin-4-yl)- 7H-purin-6- amine | F | 342.1 | A | 2.19 |
| 202 | 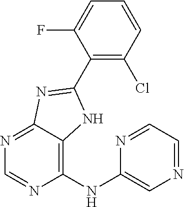 [SMILES: Fc1cccc(Cl)c1-c1nc2ncnc(Nc3cnccn3)c2[nH]1] | 8-(2-chloro-6- fluorophenyl)-N- (pyrazin-2-yl)- 7H-purin-6- amine | F | 342.0 | A | 2.32 |
| 203 | 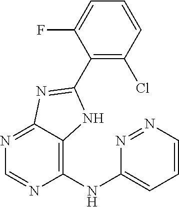 [SMILES: Fc1cccc(Cl)c1-c1nc2ncnc(Nc3cccnn3)c2[nH]1] | 8-(2-chloro-6- fluorophenyl)-N- (pyridazin-3-yl)- 7H-purin-6- amine | F | 342.1 | A | 2.19 |
| 204 | 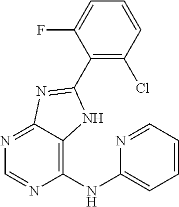 [SMILES: Fc1cccc(Cl)c1-c1nc2ncnc(Nc3ccccn3)c2[nH]1] | 8-(2-chloro-6- fluorophenyl)-N- (pyridin-2-yl)- 7H-purin-6- amine | F | 341.1 | A | 2.77 |
| 205 | 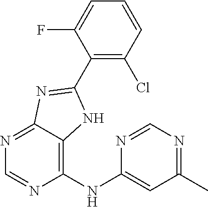 [SMILES: Cc1cc(Nc2ncnc3nc(-c4c(F)cccc4Cl)[nH]c23)ncn1] | 8-(2-chloro-6- fluorophenyl)-N- (6- methylpyrimidin- 4-yl)-7H-purin-6- amine | F | 356.0 | A | 2.55 |
| 206 | 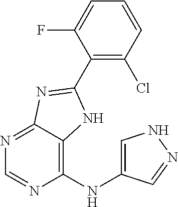 [SMILES: Fc1cccc(Cl)c1-c1nc2ncnc(Nc3cn[nH]c3)c2[nH]1] | 8-(2-chloro-6- fluorophenyl)-N- (1H-pyrazol-4- yl)-7H-purin-6- amine | F | 330.1 | A | 2.47 |
| 207 | 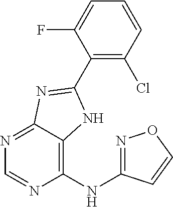 [SMILES: Fc1cccc(Cl)c1-c1nc2ncnc(Nc3ccon3)c2[nH]1] | N-(8-(2-chloro-6- fluorophenyl)- 7H-purin-6- yl)isoxazol-3- amine | F | 331.1 | A | 2.20 |
| 208 | 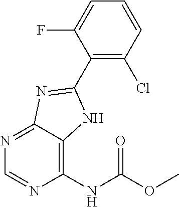 [SMILES: COC(=O)Nc1ncnc2nc(-c3c(F)cccc3Cl)[nH]c12] | methyl 8-(2- chloro-6- fluorophenyl)- 7H-purin-6- ylcarbamate | F | 322.0 | A | 2.02 |
| 209 | 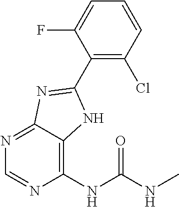 [SMILES: CNC(=O)Nc1ncnc2nc(-c3c(F)cccc3Cl)[nH]c12] | 1-(8-(2-chloro-6- fluorophenyl)- 7H-purin-6-yl)-3- methylurea | F | 321.1 | A | 2.02 |
| 210 | 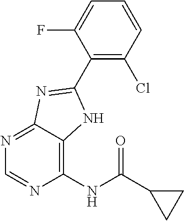 [SMILES: O=C(Nc1ncnc2nc(-c3c(F)cccc3Cl)[nH]c12)C1CC1] | N-(8-(2-chloro-6- fluorophenyl)- 7H-purin-6- yl)cyclopropane carboxamide | F | 332.1 | A | 2.42 |
| 211 | 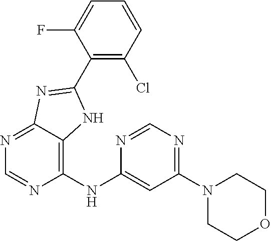 [SMILES: Fc1cccc(Cl)c1-c1nc2ncnc(Nc3cc(N4CCOCC4)ncn3)c2[nH]1] | 8-(2-chloro-6- fluorophenyl)-N- (6- morpholino- pyrimidin- 4-yl)-7H- purin-6-amine | F | 427.1 | A | 2.69 |
| 212 | 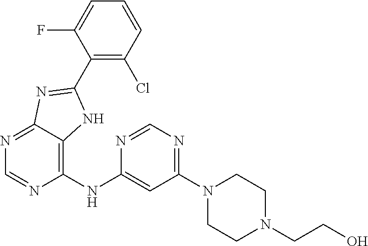 [SMILES: OCCN1CCN(c2cc(Nc3ncnc4nc(-c5c(F)cccc5Cl)[nH]c34)ncn2)CC1] | 2-(4-(6-(8-(2- chloro-6- fluorophenyl)- 7H-purin-6- ylamino)pyrimidin- 4-yl)piperazin- 1-yl)ethanol | F | 470.1 | A | 2.58 |
| 213 | 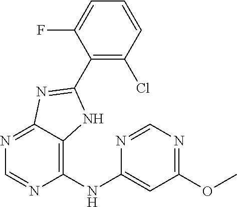 [SMILES: COc1cc(Nc2ncnc3nc(-c4c(F)cccc4Cl)[nH]c23)ncn1] | 8-(2-chloro-6- fluorophenyl)-N- (6- methoxypyrimidin- 4-yl)-7H-purin- 6-amine | F | 372.1 | A | 2.62 |
| 214 | 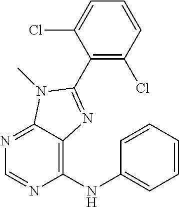 [SMILES: Cn1c(-c2c(Cl)cccc2Cl)nc2c(Nc3ccccc3)ncnc21] | 8-(2,6- dichlorophenyl)- 9-methyl-N- phenyl-9H-purin- 6-amine | E | 370.1 | A | 6.21 |
| 215 | 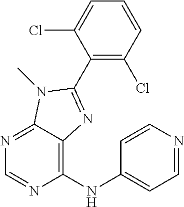 [SMILES: Cn1c(-c2c(Cl)cccc2Cl)nc2c(Nc3ccncc3)ncnc21] | 8-(2,6- dichlorophenyl)- 9-methyl-N- (pyridin-4-yl)- 9H-purin-6- amine | E | 371.0 | A | 5.09 |

### Example 216

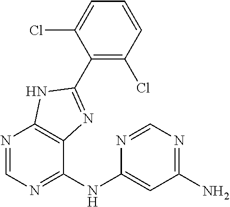 [SMILES: Nc1cc(Nc2ncnc3[nH]c(-c4c(Cl)cccc4Cl)nc23)ncn1]

### N4-(8-(2,6-dichlorophenyl)-9H-purin-6-yl)pyrimidine-4,6-diamine

Step 1: To a 100 mL round bottom flask was charged 8-(2,6-dichlorophenyl)-9H-purin-6-ol (1.0 g, 3.56 mmol), followed by anhydrous acetonitrile (6.34 mL), POBr₃ (3.34 g, 10.67 mmol) was added dropwise, and the mixture was heated at 150° C. for 40 min before being cooled to 23° C. The mixture was carefully poured into ice water (20 mL), and then extracted with EtOAc (3×20 mL). Combined organics were dried over Na₂SO₄, concentrated and purified by silica gel column chromatography (30% EtOAc/petroleum ether) to give pure 6-bromo-8-(2,6-dichlorophenyl)-9H-purine (700 mg, 57% yield). ¹H NMR (DMSO-d₆, 400 MHz) δ 14.51 (s, 1H), 8.80 (s, 1H), 7.78-7.69 (m, 3H). LCMS (ESI) m/z: 344.9 [M+H⁺].

Step 2: To a solution of 6-bromo-8-(2,6-dichlorophenyl)-7H-purine (480 mg, 1.4 mmol) in DMF (10 mL) was added sodium methanethiolate (391 mg, 5.58 mmol). The reaction mixture was stirred at room temperature under N₂ for 16 hrs. The mixture was then concentrated. The residue was partitioned between chloroform/isopropanol (3/1) and brine. The aqueous layer was extracted with chloroform/isopropanol (3/1) four more times. The combined organics were dried (Na₂SO₄), filtered and concentrated. The crude product was purified by silica gel chromatography (30-50% EtOAc/hexane) to give 8-(2,6-dichlorophenyl)-6-(methylthio)-7H-purine as a white solid (368 mg, 85% yield). ¹H NMR (DMSO-d₆, 400 MHz) δ 13.97 (s, 1H), 8.77 (s, 1H), 7.78-7.60 (m, 3H), 2.69 (s, 3H). LCMS (ESI) m/z: 311.1 [M+H⁺].

Step 3: To a solution of 8-(2,6-dichlorophenyl)-6-(methylthio)-7H-purine (232 mg, 0.75 mmol) in DMF (5 mL) was added Oxone (1.15 g, 1.86 mmol). The reaction mixture was stirred at room temperature for 16 hrs. The reaction was quenched with saturated NH₄Cl (20 mL), extracted with EtOAc (3×20 mL). The combined organics were dried (Na₂SO₄), filtered and concentrated to give 8-(2,6-dichlorophenyl)-6-(methylsulfonyl)-7H-purine as a yellow gel which was used in the next step without purification. LCMS (ESI) m/z: 343.0 [M+H⁺].

Step 4: To a solution of 8-(2,6-dichlorophenyl)-6-(methylsulfonyl)-7H-purine (343 mg, 1.0 mmol) in DMF (5 mL) at 0° C. was added NaH (60% in mineral oil, 80 mg, 2.0 mmol) in one portion. The reaction mixture was warmed up to room temperature and stirred for 1.5 hr. The mixture was then cooled to 0° C. SEM-C1 (250 mg, 1.5 mmol) was added. The reaction mixture was warmed up to room temperature and stirred for 0.5 h. The reaction was quenched with ice water (20 mL), extracted with EtOAc (3×20 mL). The combined organics were dried (Na₂SO₄), filtered and concentrated. The crude product was purified by silica gel chromatography (10-40% EtOAc/hexane) to give 8-(2,6-dichlorophenyl)-6-(methylsulfonyl)-9-((2-(trimethylsilyl)ethoxy)methyl)-9H-purine as a white solid (263 mg, 56% yield). ¹H NMR (CDCl₃, 400 MHz) δ 9.23 (s, 1H), 7.57 (s, 3H), 5.65 (s, 2H), 3.62 (s, 3H), 3.59-3.53 (m, 2H), 0.95 (dd, J=9.9, 7.0 Hz, 2H), 0.07 (s, 9H). LCMS (ESI) m/z: 475.1 [M+H⁺].

Step 5: To a solution of 4,6-diaminopyrimidine (33 mg, 0.3 mmol) in DMF (2 mL) at 0° C. was added NaH (60% in mineral oil, 16 mg, 0.4 mmol) in one portion. The reaction mixture was warmed up to room temperature and stirred for 1 hr. The mixture was then cooled to 0° C. A solution of 8-(2,6-dichlorophenyl)-6-(methylsulfonyl)-9-((2-(trimethylsilyl)ethoxy)methyl)-9H-purine (47 mg, 0.1 mmol) in DMF (0.5 mL) was added. The reaction mixture was warmed up to room temperature and stirred for 0.5 h. The reaction was quenched with ice water (30 mL), extracted with EtOAc (3×20 mL). The combined organics were dried (Na₂SO₄), filtered and concentrated to give crude N-4-(8-(2,6-dichlorophenyl)-9-((2-(trimethylsilyl)ethoxy)methyl)-9H-purin-6-yl)pyrimidine-4,6-diamine (44 mg, 88% yield) which was used in the next step without purification. LCMS (ESI) m/z: 503.2 [M+H⁺].

Step 6: To a solution of N-4-(8-(2,6-dichlorophenyl)-9-((2-(trimethylsilyl)ethoxy)methyl)-9H-purin-6-yl)pyrimidine-4,6-diamine (44 mg, 0.09 mmol) in THF (2 mL) was added TBAF (700 mg, 2.68 mmol). The reaction mixture was heated at reflux for 18 hrs. The mixture was cooled to room, partitioned between EtOAc (10 mL) and water (10 mL). The aqueous layer was extracted with EtOAc (2×10 mL). The combined organics were dried (Na₂SO₄), filtered and concentrated. The crude product was purified by prep-HPLC (Gilson GX 281, Shim-pack PRC-ODS 250 mm×20 mm×2, gradient: CH₃CN/10 mm/L NH₄HCO₃, 17 min) to give N4-(8-(2,6-dichlorophenyl)-7H-purin-6-yl)pyrimidine-4,6-diamine as a white solid (8 mg, 24% yield). ¹H NMR (CDCl₃, 400 MHz) δ 13.76 (s, 1H), 8.98-8.76 (m, 1H), 8.56 (s, 1H), 8.15 (s, 1H), 7.76-7.58 (m, 3H), 7.51 (s, 1H), 6.81 (s, 2H). LCMS (ESI) m/z: 373.0 [M+H⁺].

Additional examples 217-478 shown in Table 2 can be made according to the above methods. The column titled Synthetic Methods (Synth Met) gives the method from Scheme 1 or 3 used to prepare the intermediate.

TABLE 2

| | | | Synth | LCMS | LCMS | RT |
|---|---|---|---|---|---|---|
| Ex # | Structure | Name | Met | (ESI) m/z | Method | min |
| 217 | 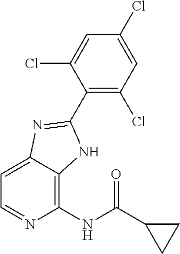 [SMILES: O=C(Nc1nccc2nc(-c3c(Cl)cc(Cl)cc3Cl)[nH]c12)C1CC1] | N-(2-(2,4,6- trichlorophenyl)- 3H-imidazo[4,5- c]pyridin-4- yl)cyclopropane- carboxamide | B | 383.0 | A | 4.28 |
| 218 | 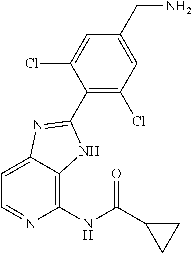 [SMILES: NCc1cc(Cl)c(-c2nc3ccnc(NC(=O)C4CC4)c3[nH]2)c(Cl)c1] | N-(2-(4- (aminomethyl)- 2,6-dichlorophenyl)- 3H-imidazo[4,5- c]pyridin-4- yl)cyclopropane- carboxamide | B | 376.1 | A | 2.88 |
| 219 | 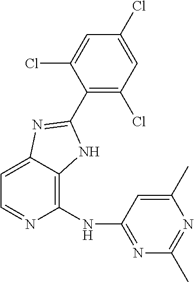 [SMILES: Cc1cc(Nc2nccc3nc(-c4c(Cl)cc(Cl)cc4Cl)[nH]c23)nc(C)n1] | N-(2,6- dimethylpyrimidin- 4-yl)-2-(2,4,6- trichlorophenyl)- 3H-imidazo[4,5- c]pyridin-4- amine | B | 421.0 | A | 3.99 |
| 220 | 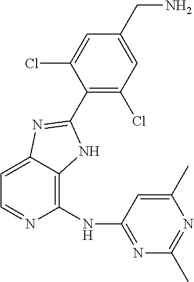 [SMILES: Cc1cc(Nc2nccc3nc(-c4c(Cl)cc(CN)cc4Cl)[nH]c23)nc(C)n1] | 2-(4-(aminomethyl)- 2,6-dichlorophenyl)- N-(2,6- dimethylpyrimidin- 4-yl)-3H-imidazo[4,5- c]pyridin-4-amine | B | 414.1 | C | 3.80 |
| 221 | 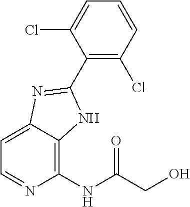 [SMILES: O=C(CO)Nc1nccc2nc(-c3c(Cl)cccc3Cl)[nH]c12] | N-(2-(2,6- dichlorophenyl)- 3H-imidazo[4,5- c]pyridin-4-yl)- 2-hydroxyacetamide | B | 337.1 | C | 3.39 |
| 222 | 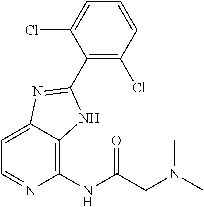 [SMILES: CN(C)CC(=O)Nc1nccc2nc(-c3c(Cl)cccc3Cl)[nH]c12] | N-(2-(2,6- dichlorophenyl)-3H- imidazo[4,5- c]pyridin-4-yl)- 2-(dimethylamino)- acetamide | B | 364.1 | A | 3.60 |
| 223 | 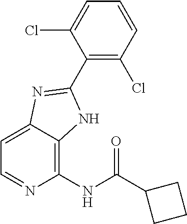 [SMILES: O=C(Nc1nccc2nc(-c3c(Cl)cccc3Cl)[nH]c12)C1CCC1] | N-(2-(2,6- dichlorophenyl)-3H- imidazo[4,5- c]pyridin-4- yl)cyclobutane- carboxamide | B | 361.1 | A | 4.41 |
| 224 | 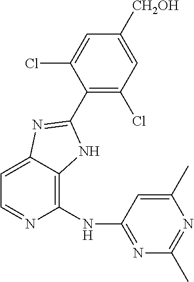 [SMILES: Cc1cc(Nc2nccc3nc(-c4c(Cl)cc(CO)cc4Cl)[nH]c23)nc(C)n1] | (3,5-dichloro-4- (4-(2,6- dimethylpyrimidin- 4-ylamino)-3H- imidazo[4,5- c]pyridin-2- yl)phenyl)methanol | B | 415.1 | C | 4.11 |
| 225 | 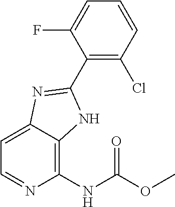 [SMILES: COC(=O)Nc1nccc2nc(-c3c(F)cccc3Cl)[nH]c12] | methyl 2-(2- chloro-6- fluorophenyl)-3H- imidazo[4,5- c]pyridin-4- ylcarbamate | B | 321.1 | A | 2.96 |
| 226 | 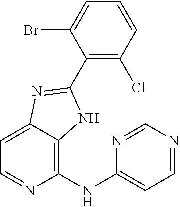 [SMILES: Clc1cccc(Br)c1-c1nc2ccnc(Nc3ccncn3)c2[nH]1] | 2-(2-bromo-6- chlorophenyl)- N-(pyrimidin-4- yl)-3H-imidazo[4,5- c]pyridin-4-amine | B | 401.0 | A | 3.29 |
| 227 | 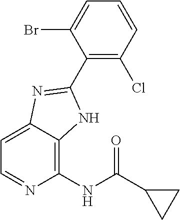 [SMILES: O=C(Nc1nccc2nc(-c3c(Cl)cccc3Br)[nH]c12)C1CC1] | N-(2-(2-bromo-6- chlorophenyl)- 3H-imidazo[4,5- c]pyridin-4- yl)cyclopropane- carboxamide | B | 393.0 | A | 3.97 |
| 228 | 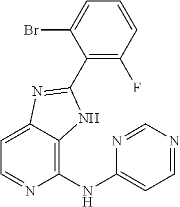 [SMILES: Fc1cccc(Br)c1-c1nc2ccnc(Nc3ccncn3)c2[nH]1] | 2-(2-bromo-6- fluorophenyl)- N-(pyrimidin-4- yl)-3H-imidazo[4,5- c]pyridin-4- amine | B | 385.0 | A | 3.15 |
| 229 | 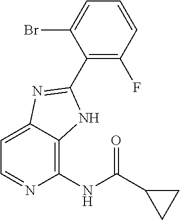 [SMILES: O=C(Nc1nccc2nc(-c3c(F)cccc3Br)[nH]c12)C1CC1] | N-(2-(2-bromo-6- fluorophenyl)-3H- imidazo[4,5- c]pyridin-4- yl)cyclopropane- carboxamide | B | 375.0 | A | 3.20 |
| 230 | 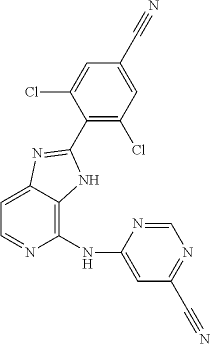 [SMILES: N#Cc1cc(Cl)c(-c2nc3ccnc(Nc4cc(C#N)ncn4)c3[nH]2)c(Cl)c1] | 6-(2-(2,6- dichloro-4- cyanophenyl)-3H- imidazo[4,5- c]pyridin-4- ylamino)pyrimidine- 4-carbonitrile | B | 407.0 | A | 2.92 |
| 231 | 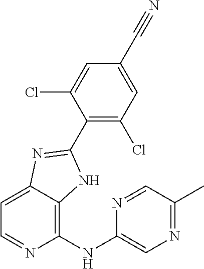 [SMILES: Cc1cnc(Nc2nccc3nc(-c4c(Cl)cc(C#N)cc4Cl)[nH]c23)cn1] | 3,5-dichloro-4- (4-(5-methylpyrazin- 2-ylamino)-3H- imidazo[4,5- c]pyridin-2- yl)benzonitrile | B | 396.0 | A | 3.53 |
| 232 | 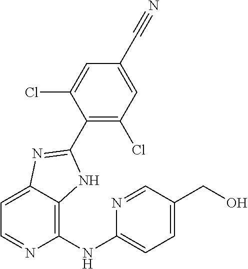 [SMILES: N#Cc1cc(Cl)c(-c2nc3ccnc(Nc4ccc(CO)cn4)c3[nH]2)c(Cl)c1] | 3,5-dichloro-4-(4-(5- (hydroxymethyl) pyridin-2- ylamino)-3H- imidazo[4,5- c]pyridin-2- yl)benzonitrile | B | 411.0 | A | 3.51 |
| 233 | 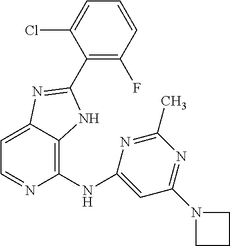 [SMILES: Cc1nc(Nc2nccc3nc(-c4c(F)cccc4Cl)[nH]c23)cc(N2CCC2)n1] | N-(6-(azetidin-1-yl)-2- methylpyrimidin- 4-yl)-2-(2-chloro-6- fluorophenyl)- 3H-imidazo[4,5- c]pyridin-4- amine | B | 410.1 | A | 4.07 |
| 234 | 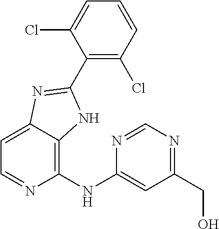 [SMILES: OCc1cc(Nc2nccc3nc(-c4c(Cl)cccc4Cl)[nH]c23)ncn1] | (6-(2-(2,6- dichlorophenyl)-3H- imidazo[4,5- c]pyridin-4- ylamino)pyrimidin- 4-yl)methanol | B | 387.1 | C | 3.78 |
| 235 | 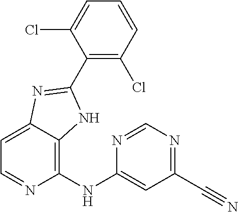 [SMILES: N#Cc1cc(Nc2nccc3nc(-c4c(Cl)cccc4Cl)[nH]c23)ncn1] | 6-(2-(2,6- dichlorophenyl)- 3H-imidazo[4,5- c]pyridin-4- ylamino)pyrimidine- 4-carbonitrile | B | 382.1 | C | 5.12 |
| 236 | 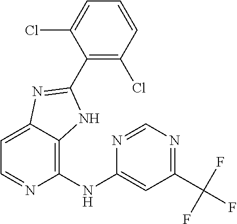 [SMILES: FC(F)(F)c1cc(Nc2nccc3nc(-c4c(Cl)cccc4Cl)[nH]c23)ncn1] | 2-(2,6-dichlorophenyl)- N-(6-(trifluoromethyl)- pyrimidin-4-yl)- 3H-imidazo[4,5- c]pyridin-4- amine | B | 425.0 | A | 4.61 |
| 237 | 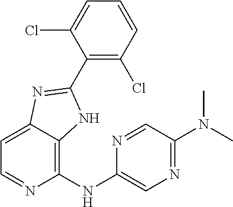 [SMILES: CN(C)c1cnc(Nc2nccc3nc(-c4c(Cl)cccc4Cl)[nH]c23)cn1] | N2-(2-(2,6- dichlorophenyl)- 3H-imidazo[4,5- c]pyridin-4-yl)- N5,N5- dimethylpyrazine- 2,5-diamine | B | 400.1 | B | 3.94 |
| 238 | 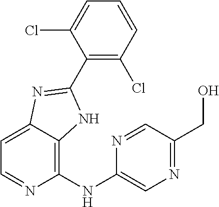 [SMILES: OCc1cnc(Nc2nccc3nc(-c4c(Cl)cccc4Cl)[nH]c23)cn1] | (5-(2-(2,6- dichlorophenyl)- 3H-imidazo[4,5- c]pyridin-4- ylamino)pyrazin- 2-yl)methanol | B | 387.0 | A | 3.25 |
| 239 | 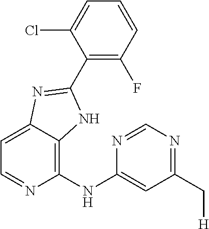 [SMILES: Cc1cc(Nc2nccc3nc(-c4c(F)cccc4Cl)[nH]c23)ncn1] | (6-(2-(2-chloro-6- fluorophenyl)-3H- imidazo[4,5- c]pyridin-4- ylamino)pyrimidin- 4-yl)methanol | B | 371.1 | C | 3.66 |
| 240 | 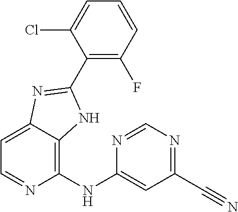 [SMILES: N#Cc1cc(Nc2nccc3nc(-c4c(F)cccc4Cl)[nH]c23)ncn1] | 6-(2-(2-chloro-6- fluorophenyl)- 3H-imidazo[4,5- c]pyridin-4- ylamino)pyrimidine- 4-carbonitrile | B | 366.1 | A | 3.38 |
| 241 | 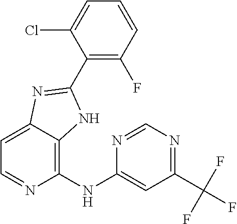 [SMILES: Fc1cccc(Cl)c1-c1nc2ccnc(Nc3cc(C(F)(F)F)ncn3)c2[nH]1] | 2-(2-chloro-6- fluorophenyl)- N-(6-(trifluoromethyl)- pyrimidin-4-yl)- 3H-imidazo[4,5- c]pyridin-4- amine | B | 409.1 | A | 4.37 |
| 242 | 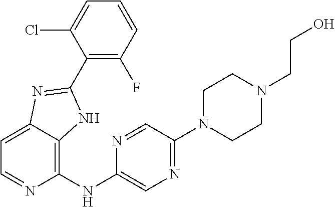 [SMILES: OCCN1CCN(c2cnc(Nc3nccc4nc(-c5c(F)cccc5Cl)[nH]c34)cn2)CC1] | 2-(4-(5-(2-(2-chloro-6- fluorophenyl)-3H- imidazo[4,5- c]pyridin-4- ylamino)pyrazin- 2-yl)piperazin-1- yl)ethanol | B | 469.3 | C | 4.16 |
| 243 | 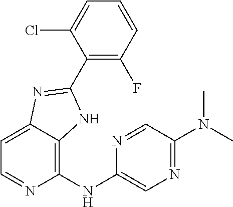 [SMILES: CN(C)c1cnc(Nc2nccc3nc(-c4c(F)cccc4Cl)[nH]c23)cn1] | N2-(2-(2-chloro-6- fluorophenyl)- 3H-imidazo[4,5- c]pyridin-4-yl)- N5,N5- dimethylpyrazine- 2,5-diamine | B | 384.1 | B | 3.92 |
| 244 | 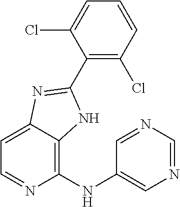 [SMILES: Clc1cccc(Cl)c1-c1nc2ccnc(Nc3cncnc3)c2[nH]1] | 2-(2,6- dichlorophenyl)- N-(pyrimidin-5- yl)-3H-imidazo[4,5- c]pyridin-4- amine | B | 357.1 | C | 4.31 |
| 245 | 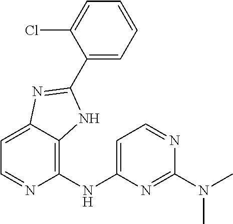 [SMILES: CN(C)c1nccc(Nc2nccc3nc(-c4ccccc4Cl)[nH]c23)n1] | N4-(2-(2- chlorophenyl)- 3H-imidazo[4,5- c]pyridin-4-yl)- N2,N2- dimethylpyrimidine- 2,4-diamine | B | 366.1 | A | 5.16 |
| 246 | 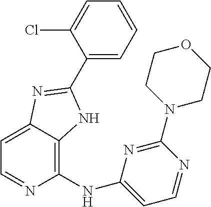 [SMILES: Clc1ccccc1-c1nc2ccnc(Nc3ccnc(N4CCOCC4)n3)c2[nH]1] | 2-(2-chlorophenyl)- N-(2-morpholino- pyrimidin-4-yl)- 3H-imidazo[4,5- c]pyridin-4- amine | B | 408.1 | A | 5.03 |
| 247 | 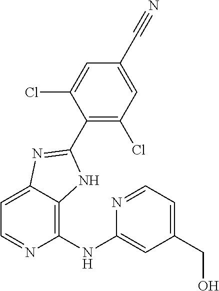 [SMILES: N#Cc1cc(Cl)c(-c2nc3ccnc(Nc4cc(CO)ccn4)c3[nH]2)c(Cl)c1] | 3,5-dichloro-4- (4-(4-(hydroxymethyl- ylamino)-3H- imidazo[4,5- c]pyridin-2- yl)benzonitrile | B | 411.0 | A | 3.56 |
| 248 | 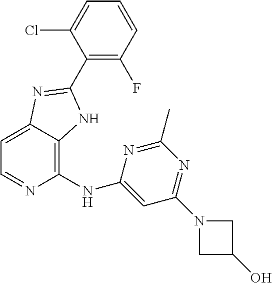 [SMILES: Cc1nc(Nc2nccc3nc(-c4c(F)cccc4Cl)[nH]c23)cc(N2CC(O)C2)n1] | 1-(6-(2-(2-chloro-6- fluorophenyl)- 3H-imidazo[4,5- c]pyridin-4- methylpyrimidin- 4-yl)azetidin-3-ol | B | 426.1 | C | 4.03 |
| 249 | 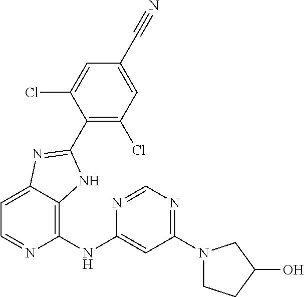 [SMILES: N#Cc1cc(Cl)c(-c2nc3ccnc(Nc4cc(N5CCC(O)C5)ncn4)c3[nH]2)c(Cl)c1] | 3,5-dichloro-4- (4-(6-(3- hydroxypyrrolidin- 1-yl)pyrimidin-4- ylamino)-3H- imidazo[4,5- c]pyridin-2- yl)benzonitrile | B | 467.1 | A | 3.10 |
| 250 | 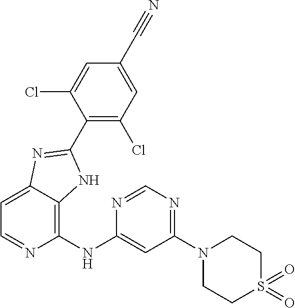 [SMILES: N#Cc1cc(Cl)c(-c2nc3ccnc(Nc4cc(N5CCS(=O)(=O)CC5)ncn4)c3[nH]2)c(Cl)c1] | 3,5-dichloro- 4-(4-(6- thiomorpholino- sulfonylpyrimidin-4- ylamino)-3H- imidazo[4,5- c]pyridin-2- yl)benzonitrile | B | 515.0 | A | 3.13 |
| 251 | 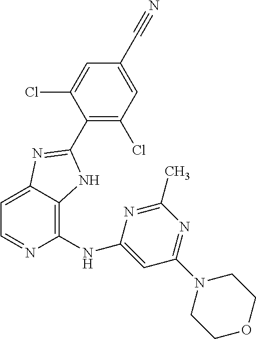 [SMILES: Cc1nc(Nc2nccc3nc(-c4c(Cl)cc(C#N)cc4Cl)[nH]c23)cc(N2CCOCC2)n1] | 3,5-dichloro-4- (4-(2-methyl-6- morpholino- pyrimidin-4- ylamino)-3H- imidazo[4,5- c]pyridin-2- yl)benzonitrile | B | 481.1 | A | 3.88 |
| 252 | 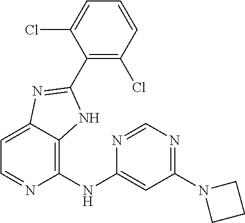 [SMILES: Clc1cccc(Cl)c1-c1nc2ccnc(Nc3cc(N4CCC4)ncn3)c2[nH]1] | N-(6-(azetidin- 1-yl)pyrimidin- 4-yl)-2-(2,6- dichlorophenyl)- 3H-imidazo[4,5- c]pyridin-4- amine | B | 412.1 | A | 4.13 |
| 253 | 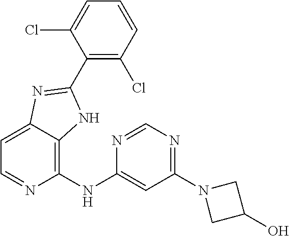 [SMILES: OC1CN(c2cc(Nc3nccc4nc(-c5c(Cl)cccc5Cl)[nH]c34)ncn2)C1] | 1-(6-(2-(2,6- dichlorophenyl)- 3H-imidazo[4,5- c]pyridin-4- ylamino)pyrimidin- 4-yl)azetidin-3-ol | B | 428.0 | A | 3.32 |
| 254 | 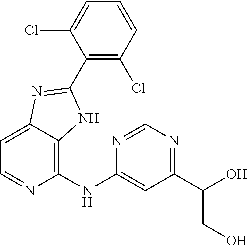 [SMILES: OCC(O)c1cc(Nc2nccc3nc(-c4c(Cl)cccc4Cl)[nH]c23)ncn1] | 1-(6-(2-(2,6- dichlorophenyl)- 3H-imidazo[4,5- c]pyridin-4- ylamino)pyrimidin-4- yl)ethane-1,2-diol | B | 417.1 | A | 2.65 |
| 255 | 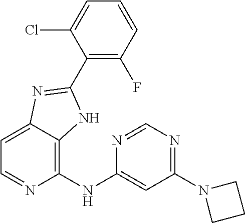 [SMILES: Fc1cccc(Cl)c1-c1nc2ccnc(Nc3cc(N4CCC4)ncn3)c2[nH]1] | N-(6-(azetidin- 1-yl)pyrimidin- 4-yl)-2-(2-chloro-6- fluorophenyl)-3H- imidazo[4,5-c]pyridin- 4-amine | B | 396.1 | C | 4.66 |
| 256 | 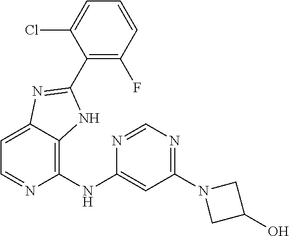 [SMILES: OC1CN(c2cc(Nc3nccc4nc(-c5c(F)cccc5Cl)[nH]c34)ncn2)C1] | 1-(6-(2-(2-chloro-6- fluorophenyl)- 3H-imidazo[4,5- c]pyridin-4- ylamino)pyrimidin- 4-yl)azetidin-3-ol | B | 412.1 | C | 3.87 |
| 257 | 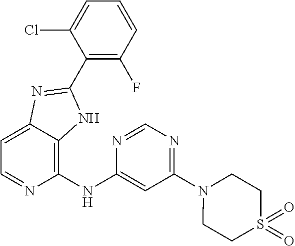 [SMILES: O=S1(=O)CCN(c2cc(Nc3nccc4nc(-c5c(F)cccc5Cl)[nH]c34)ncn2)CC1] | 2-(2-chloro-6- fluorophenyl)- N-(6-thiomorpholino- sulfonylpyrimidin- 4-yl)-3H-imidazo[4,5- c]pyridin-4-amine | B | 474.0 | A | 3.53 |
| 258 | 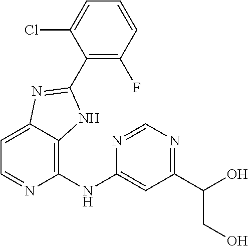 [SMILES: OCC(O)c1cc(Nc2nccc3nc(-c4c(F)cccc4Cl)[nH]c23)ncn1] | 1-(6-(2-(2-chloro-6- fluorophenyl)- 3H-imidazo[4,5- c]pyridin-4- ylamino)pyrimidin- 4-yl)ethane-1,2-diol | B | 401.0 | A | 2.56 |
| 259 | 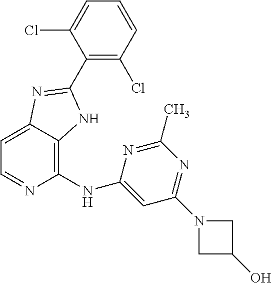 [SMILES: Cc1nc(Nc2nccc3nc(-c4c(Cl)cccc4Cl)[nH]c23)cc(N2CC(O)C2)n1] | 1-(6-(2-(2,6- dichlorophenyl)- 3H-imidazo[4,5- c]pyridin-4-ylamino)- 2-methylpyrimidin- 4-yl)azetidin-3-ol | B | 442.1 | A | 3.49 |
| 260 | 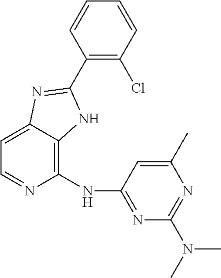 [SMILES: Cc1cc(Nc2nccc3nc(-c4ccccc4Cl)[nH]c23)nc(N(C)C)n1] | N-4-(2-(2- chlorophenyl)-3H- imidazo[4,5-c]pyridin- 4-yl)-N2,N2,6- trimethylpyrimidine- 2,4-diamine | B | 380.1 | A | 5.77 |
| 261 | 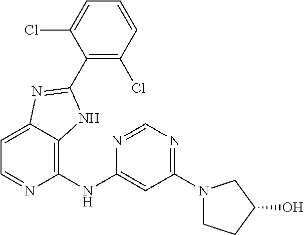 [SMILES: O[C@@H]1CCN(c2cc(Nc3nccc4nc(-c5c(Cl)cccc5Cl)[nH]c34)ncn2)C1] | (R)-1-(6-(2-(2,6- dichlorophenyl)- 3H-imidazo[4,5- c]pyridin-4- ylamino)pyrimidin- 4-yl)pyrrolidin-3-ol | B | 442.1 | C | 4.12 |
| 262 | 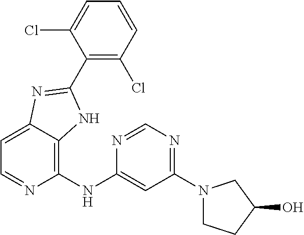 [SMILES: O[C@H]1CCN(c2cc(Nc3nccc4nc(-c5c(Cl)cccc5Cl)[nH]c34)ncn2)C1] | (S)-1-(6-(2-(2,6- dichlorophenyl)- 3H-imidazo[4,5- c]pyridin-4- ylamino)pyrimidin- 4-yl)pyrrolidin-3-ol | B | 442.1 | C | 4.13 |
| 263 | 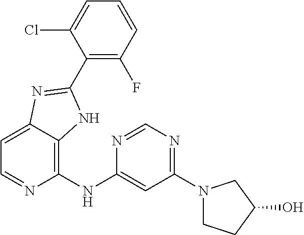 [SMILES: O[C@@H]1CCN(c2cc(Nc3nccc4nc(-c5c(F)cccc5Cl)[nH]c34)ncn2)C1] | (R)-1-(6-(2-(2- chloro-6- fluorophenyl)- 3H-imidazo[4,5- c]pyridin-4- ylamino)pyrimidin- 4-yl)pyrrolidin-3-ol | B | 426.1 | C | 4.00 |
| 264 | 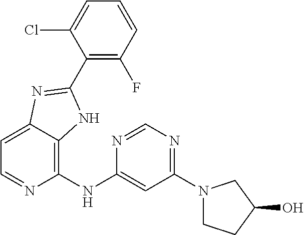 [SMILES: O[C@H]1CCN(c2cc(Nc3nccc4nc(-c5c(F)cccc5Cl)[nH]c34)ncn2)C1] | (S)-1-(6-(2-(2-chloro-6- fluorophenyl)-3H- imidazo[4,5- c]pyridin-4- ylamino)pyrimidin- 4-yl)pyrrolidin-3-ol | B | 426.2 | C | 4.00 |
| 265 | 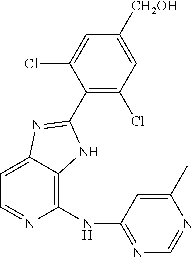 [SMILES: Cc1cc(Nc2nccc3nc(-c4c(Cl)cc(CO)cc4Cl)[nH]c23)ncn1] | (3,5-dichloro-4- (4-(6-methylpyrimidin- 4-ylamino)-3H- imidazo[4,5- c]pyridin-2- yl)phenyl)methanol | B | 401.1 | A | 3.12 |
| 266 | 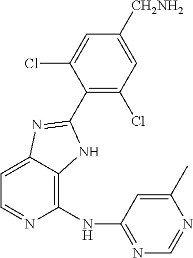 | 2-(4-(aminomethyl)- 2,6-dichlorophenyl)- N-(6-methylpyrimidin- 4-yl)-3H- imidazo[4,5- c]pyridin-4-amine | B | 400.1 | A | 3.68 |
| 267 | 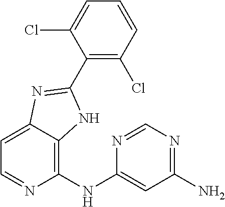 [SMILES: Nc1cc(Nc2nccc3nc(-c4c(Cl)cccc4Cl)[nH]c23)ncn1] | N4-(2-(2,6- dichlorophenyl)- 3H-imidazo[4,5- c]pyridin-4- yl)pyrimidine- 4,6-diamine | B | 372.0 | A | 3.10 |
| 268 | 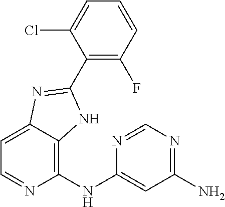 [SMILES: Nc1cc(Nc2nccc3nc(-c4c(F)cccc4Cl)[nH]c23)ncn1] | N4-(2-(2- chloro-6- fluorophenyl)- 3H-imidazo[4,5- c]pyridin-4- yl)pyrimidine- 4,6-diamine | B | 356.1 | C | 3.76 |
| 269 | 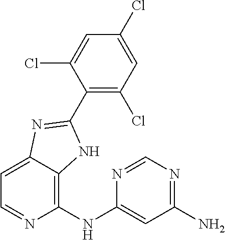 [SMILES: Nc1cc(Nc2nccc3nc(-c4c(Cl)cc(Cl)cc4Cl)[nH]c23)ncn1] | N4-(2-(2,4,6- trichlorophenyl)- 3H-imidazo[4,5- c]pyridin-4- yl)pyrimidine- 4,6-diamine | B | 408.0 | A | 3.49 |
| 270 | 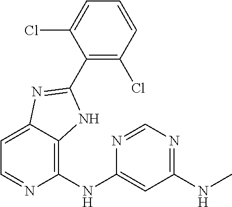 [SMILES: CNc1cc(Nc2nccc3nc(-c4c(Cl)cccc4Cl)[nH]c23)ncn1] | N4-(2-(2,6- dichlorophenyl)- 3H-imidazo[4,5- c]pyridin-4-yl)- N6-methylpyrimidine- 4,6-diamine | B | 386.1 | A | 3.50 |
| 271 | 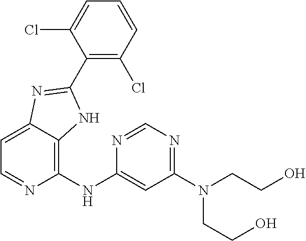 [SMILES: OCCN(CCO)c1cc(Nc2nccc3nc(-c4c(Cl)cccc4Cl)[nH]c23)ncn1] | 2,2′-(6-(2-(2,6- dichlorophenyl)- 3H-imidazo[4,5- c]pyridin-4- ylamino)pyrimidin- 4-ylazanediyl)diethanol | B | 460.1 | A | 3.86 |
| 272 | 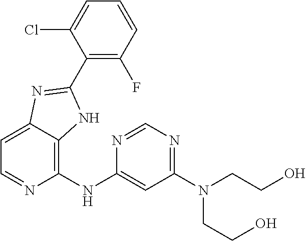 [SMILES: OCCN(CCO)c1cc(Nc2nccc3nc(-c4c(F)cccc4Cl)[nH]c23)ncn1] | 2,2′-(6-(2-(2-chloro-6- fluorophenyl)- 3H-imidazo[4,5- c]pyridin-4- ylamino)pyrimidin- 4-ylazanediyl)diethanol | B | 444.1 | A | 3.13 |
| 273 | 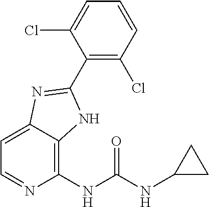 [SMILES: O=C(Nc1nccc2nc(-c3c(Cl)cccc3Cl)[nH]c12)NC1CC1] | 1-cyclopropyl- 3-(2-(2,6- dichlorophenyl)- 3H-imidazo[4,5- c]pyridin-4-yl)urea | B | 362.0 | A | 3.93 |
| 274 | 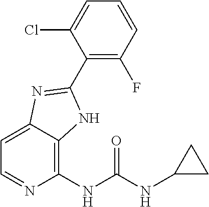 [SMILES: O=C(Nc1nccc2nc(-c3c(F)cccc3Cl)[nH]c12)NC1CC1] | 1-(2-(2-chloro-6- fluorophenyl)-3H- imidazo[4,5- c]pyridin-4-yl)-3- cyclopropylurea | B | 346.0 | B | 3.58 |
| 275 | 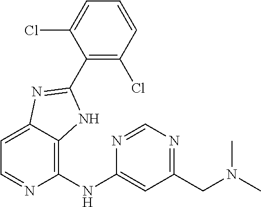 [SMILES: CN(C)Cc1cc(Nc2nccc3nc(-c4c(Cl)cccc4Cl)[nH]c23)ncn1] | 2-(2,6- dichlorophenyl)-N-(6- ((dimethylamino)- methyl)pyrimidin- 4-yl)-3H-imidazo[4,5- c]pyridin-4-amine | B | 414.2 | C | 4.36 |
| 276 | 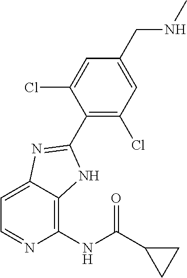 [SMILES: CNCc1cc(Cl)c(-c2nc3ccnc(NC(=O)C4CC4)c3[nH]2)c(Cl)c1] | N-(2-(2,6- dichloro-4- ((methylamino)- methyl)phenyl)- 3H-imidazo[4,5- c]pyridin-4- yl)cyclopropane- carboxamide | B | 390.1 | C | 3.78 |
| 277 | 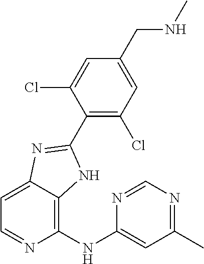 [SMILES: CNCc1cc(Cl)c(-c2nc3ccnc(Nc4cc(C)ncn4)c3[nH]2)c(Cl)c1] | 2-(2,6-dichloro- 4-((methylamino)- methyl)phenyl)- N-(6-methylpyrimidin- 4-yl)-3H- imidazo[4,5- c]pyridin-4- amine | B | 414.1 | C | 3.87 |
| 278 | 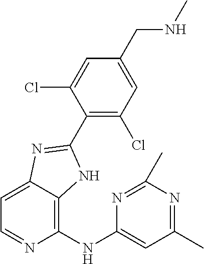 [SMILES: CNCc1cc(Cl)c(-c2nc3ccnc(Nc4cc(C)nc(C)n4)c3[nH]2)c(Cl)c1] | 2-(2,6-dichloro- 4-((methylamino)- methyl)phenyl)- N-(2,6- dimethylpyrimidin- 4-yl)-3H-imidazo[4,5- c]pyridin-4-amine | B | 428.1 | C | 4.03 |
| 279 | 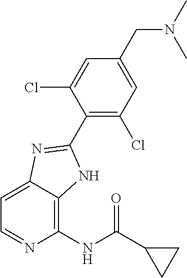 [SMILES: CN(C)Cc1cc(Cl)c(-c2nc3ccnc(NC(=O)C4CC4)c3[nH]2)c(Cl)c1] | N-(2-(2,6- dichloro-4- ((dimethylamino)- methyl)phenyl)- 3H-imidazo[4,5- c]pyridin-4- yl)cyclopropane- carboxamide | B | 404.1 | A | 3.98 |
| 280 | 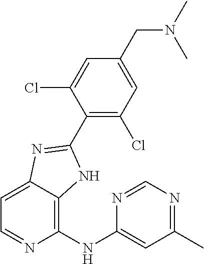 [SMILES: Cc1cc(Nc2nccc3nc(-c4c(Cl)cc(CN(C)C)cc4Cl)[nH]c23)ncn1] | 2-(2,6-dichloro-4- ((dimethylamino)- methyl)phenyl)-N-(6- methylpyrimidin- 4-yl)-3H-imidazo[4,5- c]pyridin-4-amine | B | 428.1 | A | 3.71 |
| 281 | 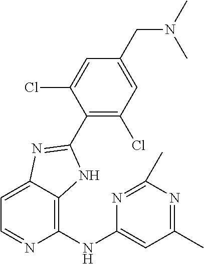 [SMILES: Cc1cc(Nc2nccc3nc(-c4c(Cl)cc(CN(C)C)cc4Cl)[nH]c23)nc(C)n1] | 2-(2,6-dichloro-4- ((dimethylamino)- methyl)phenyl)-N-(2,6- dimethylpyrimidin- 4-yl)-3H-imidazo[4,5- c]pyridin-4-amine | B | 442.1 | A | 3.88 |
| 282 | 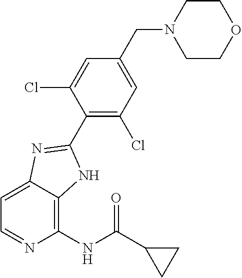 [SMILES: O=C(Nc1nccc2nc(-c3c(Cl)cc(CN4CCOCC4)cc3Cl)[nH]c12)C1CC1] | N-(2-(2,6- dichloro-4- (morpholinomethyl)- phenyl)-3H- imidazo[4,5- c]pyridin-4- yl)cyclopropane- carboxamide | B | 446.1 | A | 3.81 |
| 283 | 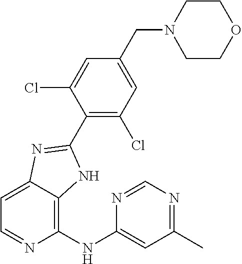 [SMILES: Cc1cc(Nc2nccc3nc(-c4c(Cl)cc(CN5CCOCC5)cc4Cl)[nH]c23)ncn1] | 2-(2,6-dichloro-4- (morpholinometh- yl)phenyl)-N- (6-methylpyrimidin- 4-yl)-3H-imidazo[4,5- c]pyridin-4-amine | B | 470.1 | A | 3.57 |
| 284 | 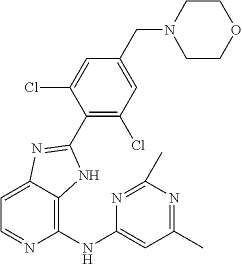 [SMILES: Cc1cc(Nc2nccc3nc(-c4c(Cl)cc(CN5CCOCC5)cc4Cl)[nH]c23)nc(C)n1] | 2-(2,6-dichloro-4- (morpholinomethyl)- phenyl)-N-(2,6- dimethylpyrimidin- 4-yl)-3H-imidazo[4,5- c]pyridin-4-amine | B | 484.1 | A | 3.75 |
| 285 | 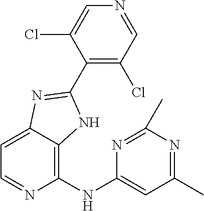 [SMILES: Cc1cc(Nc2nccc3nc(-c4c(Cl)cncc4Cl)[nH]c23)nc(C)n1] | 2-(3,5-dichloropyridin- 4-yl)-N-(2,6- dimethylpyrimidin- 4-yl)-3H-imidazo[4,5- c]pyridin-4-amine | C | 386.1 | A | 2.81 |
| 286 | 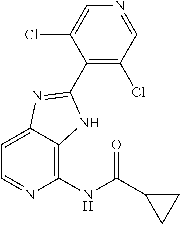 [SMILES: O=C(Nc1nccc2nc(-c3c(Cl)cncc3Cl)[nH]c12)C1CC1] | N-(2-(3,5- dichloropyridin- 4-yl)-3H-imidazo[4,5- c]pyridin-4- yl)cyclopropane- carboxamide | C | 348.0 | A | 2.43 |
| 287 | 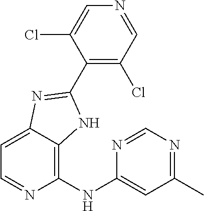 [SMILES: Cc1cc(Nc2nccc3nc(-c4c(Cl)cncc4Cl)[nH]c23)ncn1] | 2-(3,5-dichloropyridin- 4-yl)-N-(6- methylpyrimidin- 4-yl)-3H-imidazo[4,5- c]pyridin-4-amine | C | 372.1 | B | 3.85 |
| 288 | 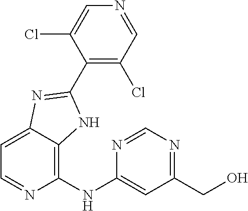 [SMILES: OCc1cc(Nc2nccc3nc(-c4c(Cl)cncc4Cl)[nH]c23)ncn1] | (6-(2-(3,5- dichloropyridin- 4-yl)-3H-imimidazo[4,5- c]pyridin-4-ylamino)- pyrimidin- 4-yl)methanol | C | 388.1 | B | 3.22 |
| 289 | 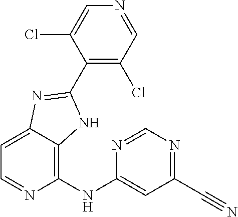 [SMILES: N#Cc1cc(Nc2nccc3nc(-c4c(Cl)cncc4Cl)[nH]c23)ncn1] | 6-(2-(3,5- dichloropyridin- 4-yl)-3H-imidazo[4,5- c]pyridin-4- ylamino)pyrimidine- 4-carbonitrile | C | 383.0 | A | 2.74 |
| 290 | 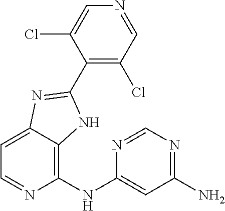 [SMILES: Nc1cc(Nc2nccc3nc(-c4c(Cl)cncc4Cl)[nH]c23)ncn1] | N4-(2-(3,5- dichloropyridin- 4-yl)-3H-imidazo[4,5- c]pyridin-4- yl)pyrimidine- 4,6-diamine | C | 373.1 | A | 2.41 |
| 291 | 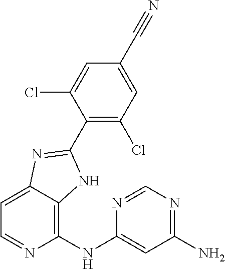 [SMILES: N#Cc1cc(Cl)c(-c2nc3ccnc(Nc4cc(N)ncn4)c3[nH]2)c(Cl)c1] | 4-(4-(6- aminopyrimidin- 4-ylamino)-3H- imidazo[4,5- c]pyridin-2-yl)-3,5- dichlorobenzonitrile | B | 397.0 | B | 3.41 |
| 292 | 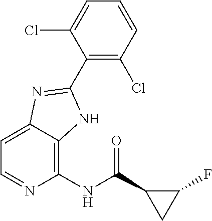 [SMILES: O=C(Nc1nccc2nc(-c3c(Cl)cccc3Cl)[nH]c12)[C@@H]1C[C@H]1F] | (1S,2R)-N-(2-(2,6- dichlorophenyl)-3H- imidazo[4,5- c]pyridin-4-yl)-2- fluorocyclopropane- carboxamide | B | 365.0 | A | 3.72 |
| 293 | 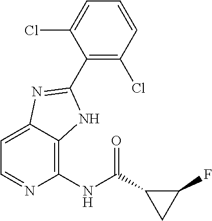 [SMILES: O=C(Nc1nccc2nc(-c3c(Cl)cccc3Cl)[nH]c12)[C@H]1C[C@@H]1F] | (1R,2S)-N-(2-(2,6- dichlorophenyl)- 3H-imidazo[4,5- c]pyridin-4-yl)-2- fluorocyclopropane- carboxamide | B | 365.0 | A | 3.63 |
| 294 | 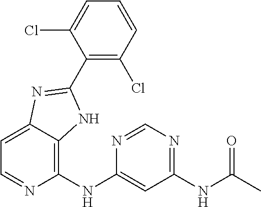 [SMILES: CC(=O)Nc1cc(Nc2nccc3nc(-c4c(Cl)cccc4Cl)[nH]c23)ncn1] | N-(6-(2-(2,6- dichlorophenyl)-3H- imidazo[4,5-c]pyridin- 4-ylamino)pyrimidin- 4-yl)acetamide | B | 414.0 | B | 3.34 |
| 295 | 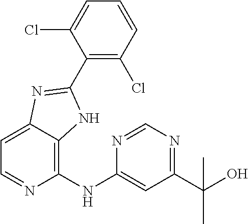 [SMILES: CC(C)(O)c1cc(Nc2nccc3nc(-c4c(Cl)cccc4Cl)[nH]c23)ncn1] | 2-(6-(2-(2,6- dichlorophenyl)-3H- imidazo[4,5-c]pyridin-4- ylamino)pyrimidin- 4-yl)propan-2-ol | B | 415.1 | C | 4.41 |
| 296 | 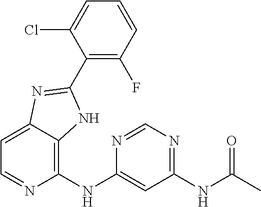 [SMILES: CC(=O)Nc1cc(Nc2nccc3nc(-c4c(F)cccc4Cl)[nH]c23)ncn1] | N-(6-(2-(2-chloro-6- fluorophenyl)-3H- imidazo[4,5-c]pyridin-4- ylamino)pyrimidin- 4-yl)acetamide | B | 398.2 | C | 4.07 |
| 297 | 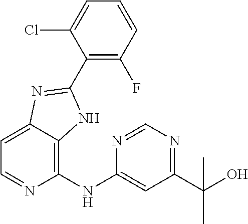 [SMILES: CC(C)(O)c1cc(Nc2nccc3nc(-c4c(F)cccc4Cl)[nH]c23)ncn1] | 2-(6-(2-(2-chloro-6- fluorophenyl)-3H- imidazo[4,5-c]pyridin-4- ylamino)pyrimidin- 4-yl)propan-2-ol | B | 399.2 | C | 4.27 |
| 298 | 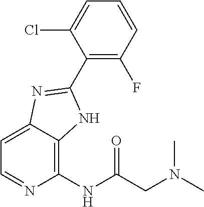 [SMILES: CN(C)CC(=O)Nc1nccc2nc(-c3c(F)cccc3Cl)[nH]c12] | N-(2-(2-chloro-6- fluorophenyl)-3H- imidazo[4,5-c]pyridin- 4-yl)-2-(dimethylamino)- acetamide | B | 348.1 | A | 3.44 |
| 299 | 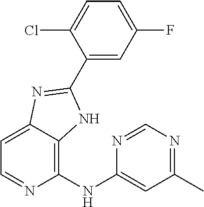 [SMILES: Cc1cc(Nc2nccc3nc(-c4cc(F)ccc4Cl)[nH]c23)ncn1] | 2-(2-chloro-5-fluorophenyl)- N-(6-methylpyrimidin- 4-yl)-3H-imidazo[4,5- c]pyridin-4-amine | B | 355.1 | A | 4.12 |
| 300 | 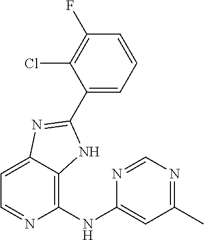 [SMILES: Cc1cc(Nc2nccc3nc(-c4cccc(F)c4Cl)[nH]c23)ncn1] | 2-(2-chloro-3-fluorophenyl)- N-(6-methylpyrimidin- 4-yl)-3H-imidazo[4,5- c]pyridin-4-amine | B | 355.1 | A | 4.02 |
| 301 | 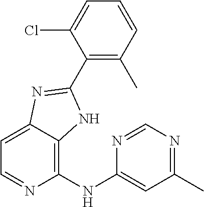 [SMILES: Cc1cc(Nc2nccc3nc(-c4c(C)cccc4Cl)[nH]c23)ncn1] | 2-(2-chloro-6-methylphenyl)- N-(6-methylpyrimidin- 4-yl)-3H-imidazo[4,5- c]pyridin-4-amine | B | 351.2 | C | 4.60 |
| 302 | 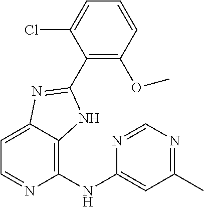 [SMILES: COc1cccc(Cl)c1-c1nc2ccnc(Nc3cc(C)ncn3)c2[nH]1] | 2-(2-chloro-6-methoxyphenyl)- N-(6-methylpyrimidin- 4-yl)-3H-imidazo[4,5- c]pyridin-4-amine | B | 367.2 | C | 4.42 |
| 303 | 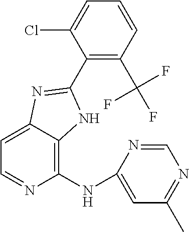 [SMILES: Cc1cc(Nc2nccc3nc(-c4c(Cl)cccc4C(F)(F)F)[nH]c23)ncn1] | 2-(2-chloro-6-(trifluoromethyl)- phenyl)-N-(6-methylpyrimidin- 4-yl)-3H-imidazo[4,5- c]pyridin-4-amine | B | 405.1 | A | 3.95 |
| 304 | 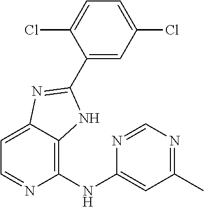 [SMILES: Cc1cc(Nc2nccc3nc(-c4cc(Cl)ccc4Cl)[nH]c23)ncn1] | 2-(2,5-dichlorophenyl)- N-(6-methylpyrimidin- 4-yl)-3H-imidazo[4,5- c]pyridin-4-amine | B | 371.1 | A | 4.24 |
| 305 | 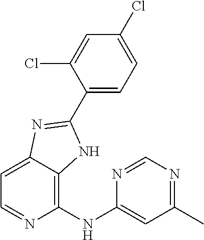 [SMILES: Cc1cc(Nc2nccc3nc(-c4ccc(Cl)cc4Cl)[nH]c23)ncn1] | 2-(2,4-dichlorophenyl)- N-(6-methylpyrimidin- 4-yl)-3H-imidazo[4,5- c]pyridin-4-amine | B | 371.1 | C | 5.32 |
| 306 | 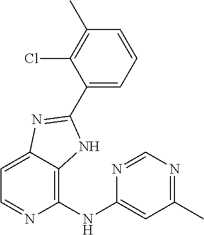 [SMILES: Cc1cc(Nc2nccc3nc(-c4cccc(C)c4Cl)[nH]c23)ncn1] | 2-(2-chloro-3-methylphenyl)- N-(6-methylpyrimidin- 4-yl)-3H-imidazo[4,5- c]pyridin-4-amine | B | 351.1 | A | 4.92 |
| 307 | 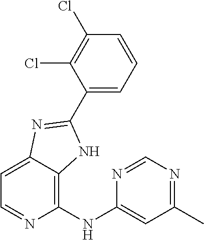 [SMILES: Cc1cc(Nc2nccc3nc(-c4cccc(Cl)c4Cl)[nH]c23)ncn1] | 2-(2,3-dichlorophenyl)- N-(6-methylpyrimidin- 4-yl)-3H-imidazo[4,5- c]pyridin-4-amine | B | 371.1 | C | 5.11 |
| 308 | 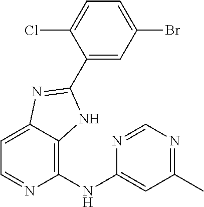 [SMILES: Cc1cc(Nc2nccc3nc(-c4cc(Br)ccc4Cl)[nH]c23)ncn1] | 2-(5-bromo-2-chlorophenyl)- N-(6-methylpyrimidin- 4-yl)-3H-imidazo[4,5- c]pyridin-4-amine | B | 415.0 | A | 4.56 |
| 309 | 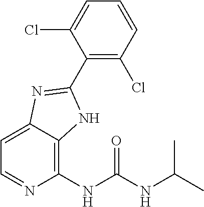 [SMILES: CC(C)NC(=O)Nc1nccc2nc(-c3c(Cl)cccc3Cl)[nH]c12] | 1-(2-(2,6-dichlorophenyl)- 3H-imidazo[4,5-c]pyridin- 4-yl)- 3-isopropylurea | B | 364.1 | A | 4.46 |
| 310 | 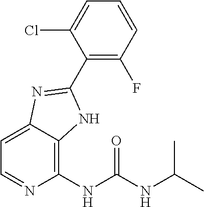 [SMILES: CC(C)NC(=O)Nc1nccc2nc(-c3c(F)cccc3Cl)[nH]c12] | 1-(2-(2-chloro- 6-fluorophenyl)- 3H-imidazo[4,5- c]pyridin-4-yl)- 3-isopropylurea | B | 348.1 | A | 4.29 |
| 311 | 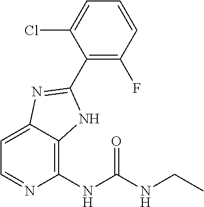 [SMILES: CCNC(=O)Nc1nccc2nc(-c3c(F)cccc3Cl)[nH]c12] | 1-(2-(2-chloro- 6-fluorophenyl)- 3H-imidazo[4,5- c]pyridin-4-yl)- 3-ethylurea | B | 334.1 | C | 4.61 |
| 312 | 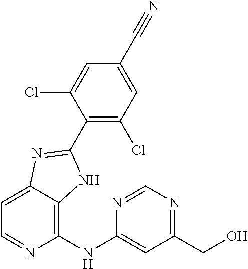 [SMILES: N#Cc1cc(Cl)c(-c2nc3ccnc(Nc4cc(CO)ncn4)c3[nH]2)c(Cl)c1] | 3,5-dichloro-4-(4-(6- (hydroxymethyl)pyrimidin- 4-ylamino)-3H-imidazo[4,5- c]pyridin-2-yl)benzonitrile | B | 412.1 | C | 3.81 |
| 313 | 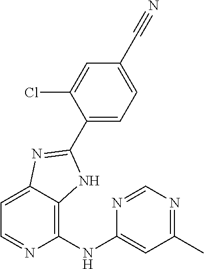 [SMILES: Cc1cc(Nc2nccc3nc(-c4ccc(C#N)cc4Cl)[nH]c23)ncn1] | 3-chloro-4-(4- (6-methylpyrimidin- 4-ylamino)-3H-imidazo[4,5- c]pyridin-2-yl)benzonitrile | B | 362.1 | A | 3.51 |
| 314 | 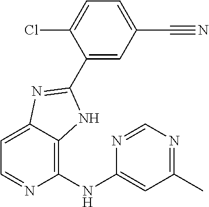 [SMILES: Cc1cc(Nc2nccc3nc(-c4cc(C#N)ccc4Cl)[nH]c23)ncn1] | 4-chloro-3-(4- (6-methylpyrimidin- 4-ylamino)-3H-imidazo[4,5- c]pyridin-2-yl)benzonitrile | B | 362.1 | A | 3.47 |
| 315 | 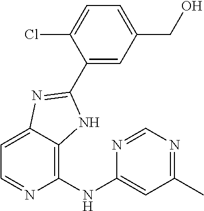 [SMILES: Cc1cc(Nc2nccc3nc(-c4cc(CO)ccc4Cl)[nH]c23)ncn1] | (4-chloro-3-(4-(6- methylpyrimidin- 4-ylamino)-3H-imidazo[4,5- c]pyridin-2-yl)phenyl)methanol | B | 367.2 | C | 4.01 |
| 316 | 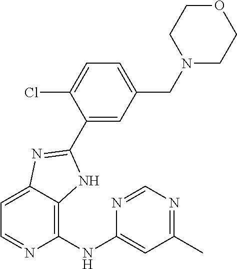 [SMILES: Cc1cc(Nc2nccc3nc(-c4cc(CN5CCOCC5)ccc4Cl)[nH]c23)ncn1] | 2-(2-chloro-5- (morpholinomethyl)phenyl)- N-(6-methylpyrimidin- 4-yl)-3H-imidazo[4,5- c]pyridin-4-amine | B | 436.1 | A | 4.14 |
| 317 | 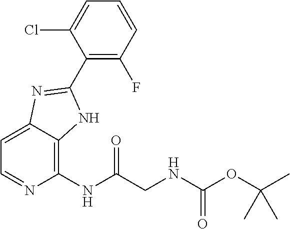 [SMILES: CC(C)(C)OC(=O)NCC(=O)Nc1nccc2nc(-c3c(F)cccc3Cl)[nH]c12] | tert-butyl 2-(2- (2-chloro-6-fluorophenyl)- 3H-imidazo[4,5-c]pyridin-4- ylamino)-2-oxoethylcarbamate | B | 420.1 | C | 4.85 |
| 318 | 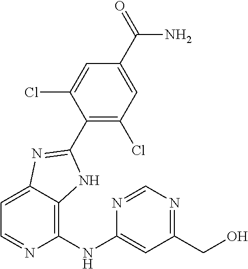 [SMILES: NC(=O)c1cc(Cl)c(-c2nc3ccnc(Nc4cc(CO)ncn4)c3[nH]2)c(Cl)c1] | 3,5-dichloro-4-(4-(6- (hydroxymethyl)- pyrimidin-4-ylamino)- 3H-imidazo[4,5- c]pyridin-2-yl)benzamide | B | 430.0 | C | 3.05 |
| 319 | 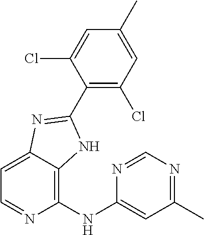 [SMILES: Cc1cc(Cl)c(-c2nc3ccnc(Nc4cc(C)ncn4)c3[nH]2)c(Cl)c1] | 2-(2,6-dichloro-4-methylphenyl)- N-(6-methylpyrimidin- 4-yl)-3H-imidazo[4,5- c]pyridin-4-amine | B | 385.1 | C | 4.97 |
| 320 | 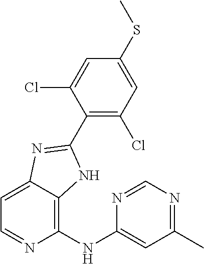 [SMILES: CSc1cc(Cl)c(-c2nc3ccnc(Nc4cc(C)ncn4)c3[nH]2)c(Cl)c1] | 2-(2,6-dichloro-4-(methylthio) phenyl)-N-(6-methylpyrimidin- 4-yl)-3H-imidazo[4,5- c]pyridin-4-amine | B | 417.0 | A | 4.40 |
| 321 | 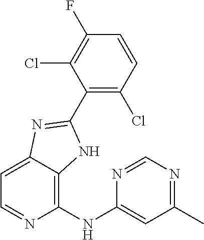 [SMILES: Cc1cc(Nc2nccc3nc(-c4c(Cl)ccc(F)c4Cl)[nH]c23)ncn1] | 2-(2,6-dichloro- 3-fluorophenyl)- N-(6-methylpyrimidin- 4-yl)-3H-imidazo[4,5- c]pyridin-4-amine | B | 389.1 | C | 4.65 |
| 322 | 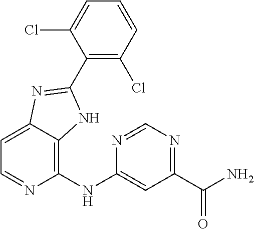 [SMILES: NC(=O)c1cc(Nc2nccc3nc(-c4c(Cl)cccc4Cl)[nH]c23)ncn1] | 6-(2-(2,6-dichlorophenyl)- 3H-imidazo[4,5-c]pyridin- 4-ylamino)pyrimidine- 4-carboxamide | B | 400.1 | C | 4.09 |
| 323 | 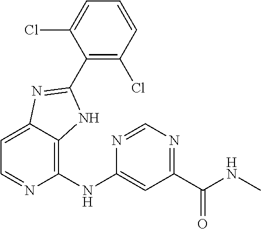 [SMILES: CNC(=O)c1cc(Nc2nccc3nc(-c4c(Cl)cccc4Cl)[nH]c23)ncn1] | 6-(2-(2,6-dichlorophenyl)- 3H-imidazo[4,5-c]pyridin- 4-ylamino)-N-methylpyrimidine- 4-carboxamide | B | 414.1 | A | 3.54 |
| 324 | 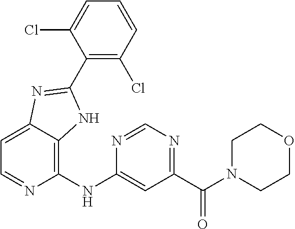 [SMILES: O=C(c1cc(Nc2nccc3nc(-c4c(Cl)cccc4Cl)[nH]c23)ncn1)N1CCOCC1] | (6-(2-(2,6-dichlorophenyl)- 3H-imidazo[4,5- c]pyridin-4-ylamino)pyrimidin- 4-yl)(morpholino)-methanone | B | 470.1 | C | 4.13 |
| 325 | 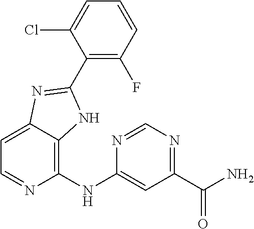 [SMILES: NC(=O)c1cc(Nc2nccc3nc(-c4c(F)cccc4Cl)[nH]c23)ncn1] | 6-(2-(2-chloro-6- fluorophenyl)-3H- imidazo[4,5-c]pyridin-4- ylamino)pyrimidine- 4-carboxamide | B | 384.1 | A | 3.07 |
| 326 | 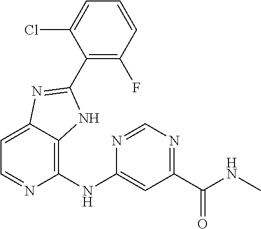 [SMILES: CNC(=O)c1cc(Nc2nccc3nc(-c4c(F)cccc4Cl)[nH]c23)ncn1] | 6-(2-(2-chloro- 6-fluorophenyl)-3H- imidazo[4,5-c]pyridin-4- ylamino)-N-methylpyrimidine- 4-carboxamide | B | 398.2 | C | 4.26 |
| 327 | 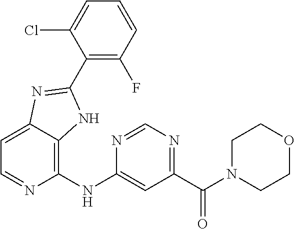 [SMILES: O=C(c1cc(Nc2nccc3nc(-c4c(F)cccc4Cl)[nH]c23)ncn1)N1CCOCC1] | (6-(2-(2-chloro-6- fluorophenyl)-3H-imidazo[4,5- c]pyridin-4-ylamino)pyrimidin- 4-yl)(morpholino)- methanone | B | 454.2 | C | 4.00 |
| 328 | 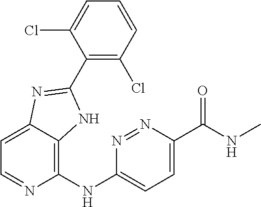 [SMILES: CNC(=O)c1ccc(Nc2nccc3nc(-c4c(Cl)cccc4Cl)[nH]c23)nn1] | 6-(2-(2,6-dichlorophenyl)- 3H-imidazo[4,5-c]pyridin-4- ylamino)-N-methylpyridazine- 3-carboxamide | B | 414.1 | C | 4.41 |
| 329 | 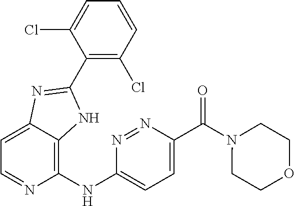 [SMILES: O=C(c1ccc(Nc2nccc3nc(-c4c(Cl)cccc4Cl)[nH]c23)nn1)N1CCOCC1] | (6-(2-(2,6-dichlorophenyl)- 3H-imidazo[4,5-c]pyridin-4- ylamino)pyridazin- 3-yl)(morpholino)- methanone | B | 470.1 | C | 4.28 |
| 330 | 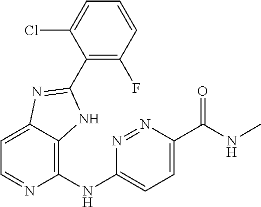 [SMILES: CNC(=O)c1ccc(Nc2nccc3nc(-c4c(F)cccc4Cl)[nH]c23)nn1] | 6-(2-(2-chloro-6-fluorophenyl)- 3H-imidazo[4,5-c]pyridin-4- ylamino)-N-methylpyridazine- 3-carboxamide | B | 398.2 | C | 4.69 |
| 331 | 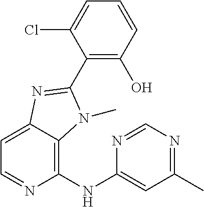 [SMILES: Cc1cc(Nc2nccc3nc(-c4c(O)cccc4Cl)n(C)c23)ncn1] | 3-chloro-2-(3-methyl-4-(6- methylpyrimidin- 4-ylamino)-3H-imidazo[4,5- c]pyridin-2-yl)phenol | B | 367.1 | A | 5.43 |
| 332 | 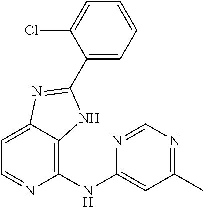 [SMILES: Cc1cc(Nc2nccc3nc(-c4ccccc4Cl)[nH]c23)ncn1] | 2-(2-chlorophenyl)- N-(6-methylpyrimidin- 4-yl)-3H-imidazo[4,5- c]pyridin-4-amine | B | 337.2 | C | 4.65 |
| 333 | 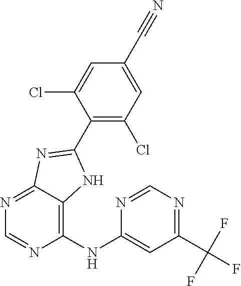 [SMILES: N#Cc1cc(Cl)c(-c2nc3ncnc(Nc4cc(C(F)(F)F)ncn4)c3[nH]2)c(Cl)c1] | 3,5-dichloro-4-(6-(6- (trifluoromethyl)- pyrimidin-4-ylamino)-7H- purin-8-yl)benzonitrile | E | 451.1 | A | 3.03 |
| 334 | 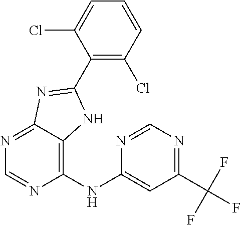 [SMILES: FC(F)(F)c1cc(Nc2ncnc3nc(-c4c(Cl)cccc4Cl)[nH]c23)ncn1] | 8-(2,6-dichlorophenyl)- N-(6-(trifluoromethyl)- pyrimidin-4-yl)- 7H-purin-6-amine | E | 426.1 | A | 3.03 |
| 335 | 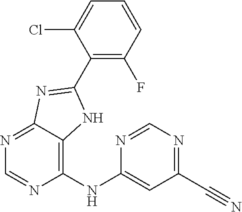 [SMILES: N#Cc1cc(Nc2ncnc3nc(-c4c(F)cccc4Cl)[nH]c23)ncn1] | 6-(8-(2-chloro-6-fluorophenyl)- 7H-purin-6-ylamino)pyrimidine- 4-carbonitrile | E | 367.1 | A | 2.52 |
| 336 | 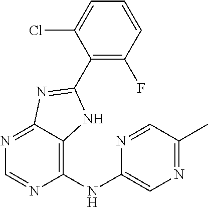 [SMILES: Cc1cnc(Nc2ncnc3nc(-c4c(F)cccc4Cl)[nH]c23)cn1] | 8-(2-chloro-6-fluorophenyl)- N-(5-methylpyrazin- 2-yl)-7H-purin-6-amine | E | 356.1 | A | 2.56 |
| 337 | 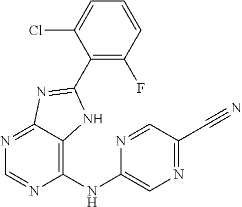 [SMILES: N#Cc1cnc(Nc2ncnc3nc(-c4c(F)cccc4Cl)[nH]c23)cn1] | 5-(8-(2-chloro-6-fluorophenyl)- 7H-purin-6-ylamino)pyrazine- 2-carbonitrile | E | 367.0 | A | 2.58 |
| 338 | 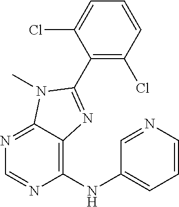 [SMILES: Cn1c(-c2c(Cl)cccc2Cl)nc2c(Nc3cccnc3)ncnc21] | 8-(2,6-dichlorophenyl)- 9-methyl-N-(pyridin-3-yl)- 9H-purin-6-amine | E | 371.0 | A | 4.97 |
| 339 | 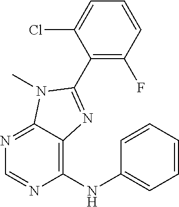 [SMILES: Cn1c(-c2c(F)cccc2Cl)nc2c(Nc3ccccc3)ncnc21] | 8-(2-chloro-6-fluorophenyl)- 9-methyl-N-phenyl-9H- purin-6-amine | E | 354.1 | B | 5.76 |
| 340 | 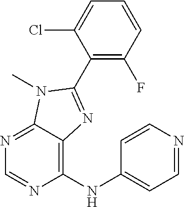 [SMILES: Cn1c(-c2c(F)cccc2Cl)nc2c(Nc3ccncc3)ncnc21] | 8-(2-chloro-6-fluorophenyl)- 9-methyl-N-(pyridin-4-yl)- 9H-purin-6-amine | E | 355.1 | A | 4.82 |
| 341 | 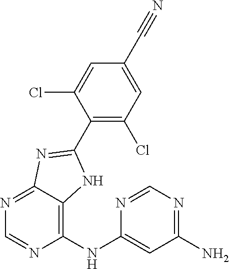 [SMILES: N#Cc1cc(Cl)c(-c2nc3ncnc(Nc4cc(N)ncn4)c3[nH]2)c(Cl)c1] | 4-(6-(6-aminopyrimidin- 4-ylamino)-7H-purin-8-yl)-3,5- dichlorobenzonitrile | E | 398.0 | B | 2.95 |
| 342 | 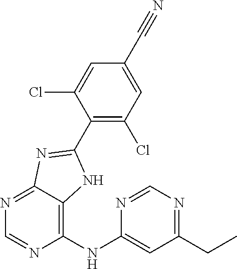 [SMILES: CCc1cc(Nc2ncnc3nc(-c4c(Cl)cc(C#N)cc4Cl)[nH]c23)ncn1] | 3,5-dichloro-4-(6-(6- ethylpyrimidin-4-ylamino)-7H- purin-8-yl)benzonitrile | E | 411.0 | A | 2.96 |
| 343 | 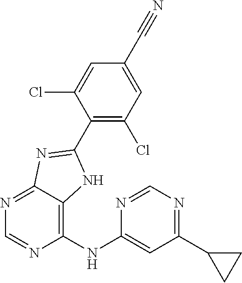 [SMILES: N#Cc1cc(Cl)c(-c2nc3ncnc(Nc4cc(C5CC5)ncn4)c3[nH]2)c(Cl)c1] | 3,5-dichloro-4-(6-(6- cyclopropyl-pyrimidin-4- ylamino)-7H-purin-8- yl)benzonitrile | E | 423.1 | C | 4.25 |
| 344 | 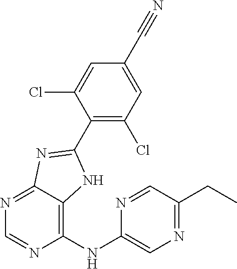 [SMILES: CCc1cnc(Nc2ncnc3nc(-c4c(Cl)cc(C#N)cc4Cl)[nH]c23)cn1] | 3,5-dichloro-4-(6-(5- ethylpyrazin-2-ylamino)-7H- purin-8-yl)benzonitrile | E | 411.1 | C | 4.42 |
| 345 | 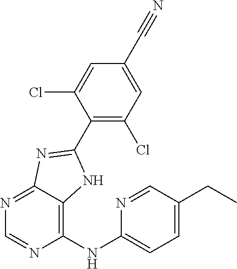 [SMILES: CCc1ccc(Nc2ncnc3nc(-c4c(Cl)cc(C#N)cc4Cl)[nH]c23)nc1] | 3,5-dichloro-4-(6-(5- ethylpyridin-2-ylamino)-7H- purin-8-yl)benzonitrile | E | 410.0 | A | 3.41 |
| 346 | 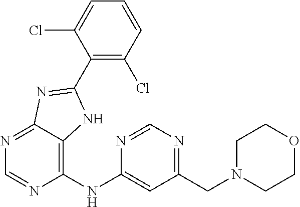 [SMILES: Clc1cccc(Cl)c1-c1nc2ncnc(Nc3cc(CN4CCOCC4)ncn3)c2[nH]1] | 8-(2,6-dichlorophenyl)- N-(6-(morpholinometh- yl)pyrimidin-4-yl)-7H-purin-6- amine | E | 457.1 | C | 3.83 |
| 347 | 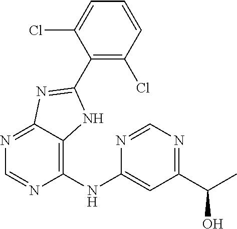 [SMILES: C[C@@H](O)c1cc(Nc2ncnc3nc(-c4c(Cl)cccc4Cl)[nH]c23)ncn1] | (R)-1-(6-(8-(2,6- dichlorophenyl)-7H-purin-6- ylamino)pyrimidin-4-yl)ethanol | E | 402.1 | A | 3.60 |
| 348 | 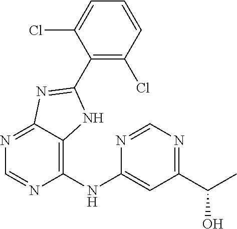 [SMILES: C[C@H](O)c1cc(Nc2ncnc3nc(-c4c(Cl)cccc4Cl)[nH]c23)ncn1] | (S)-1-(6-(8-(2,6- dichlorophenyl)-7H-purin-6- ylamino)pyrimidin-4-yl)ethanol | E | 402.1 | A | 3.60 |
| 349 | 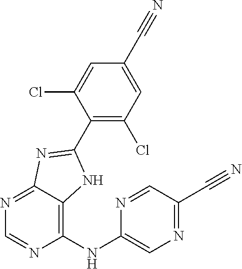 [SMILES: N#Cc1cc(Cl)c(-c2nc3ncnc(Nc4cnc(C#N)cn4)c3[nH]2)c(Cl)c1] | 5-(8-(2,6- dichloro-4-cyanophenyl)- 7H-purin-6-ylamino)pyrazine- 2-carbonitrile | E | 408.0 | A | 2.98 |
| 350 | 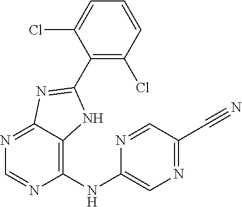 [SMILES: N#Cc1cnc(Nc2ncnc3nc(-c4c(Cl)cccc4Cl)[nH]c23)cn1] | 5-(8-(2,6-dichlorophenyl)- 7H-purin-6-ylamino)pyrazine- 2-carbonitrile | E | 383.0 | C | 4.39 |
| 351 | 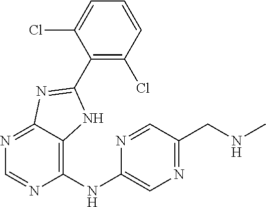 [SMILES: CNCc1cnc(Nc2ncnc3nc(-c4c(Cl)cccc4Cl)[nH]c23)cn1] | 8-(2,6-dichlorophenyl)- N-(5-((methylamino)- methyl)pyrazin-2-yl)-7H- purin-6-amine | E | 401.1 | A | 2.48 |
| 352 | 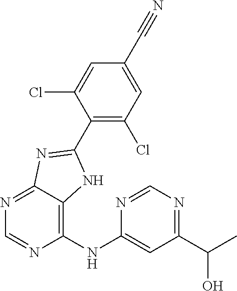 [SMILES: CC(O)c1cc(Nc2ncnc3nc(-c4c(Cl)cc(C#N)cc4Cl)[nH]c23)ncn1] | 3,5-dichloro-4-(6-(6-(1- hydroxyethyl)-pyrimidin-4- ylamino)-7H-purin-8- yl)benzonitrile | E | 427.1 | C | 3.39 |
| 353 | 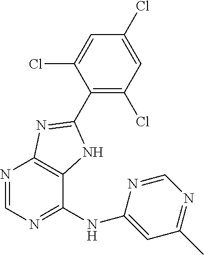 [SMILES: Cc1cc(Nc2ncnc3nc(-c4c(Cl)cc(Cl)cc4Cl)[nH]c23)ncn1] | N-(6-methylpyrimidin- 4-yl)-8-(2,4,6-trichlorophenyl)- 7H-purin-6-amine | E | 405.9 | B | 2.88 |
| 354 | 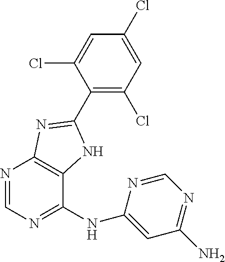 [SMILES: Nc1cc(Nc2ncnc3nc(-c4c(Cl)cc(Cl)cc4Cl)[nH]c23)ncn1] | N4-(8-(2,4,6-trichlorophenyl)- 7H-purin-6-yl)pyrimidine- 4,6-diamine | E | 406.9 | B | 2.75 |
| 355 | 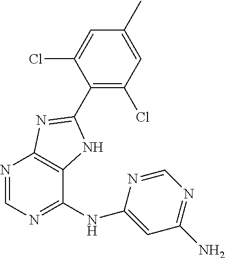 [SMILES: Cc1cc(Cl)c(-c2nc3ncnc(Nc4cc(N)ncn4)c3[nH]2)c(Cl)c1] | N4-(8-(2,6- dichloro-4-methylphenyl)- 7H-purin-6-yl)pyrimidine- 4,6-diaminel | E | 387.0 | B | 3.43 |
| 356 | 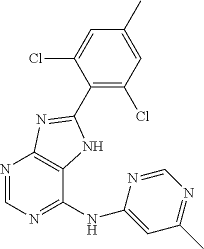 [SMILES: Cc1cc(Cl)c(-c2nc3ncnc(Nc4cc(C)ncn4)c3[nH]2)c(Cl)c1] | 8-(2,6-dichloro-4-methylphenyl)- N-(6-methylpyrimidin- 4-yl)-7H-purin-6-amine | E | 386.0 | B | 3.50 |
| 357 | 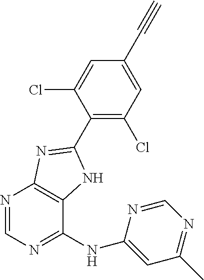 [SMILES: C#Cc1cc(Cl)c(-c2nc3ncnc(Nc4cc(C)ncn4)c3[nH]2)c(Cl)c1] | 8-(2,6-dichloro- 4-ethynylphenyl)-N-(6- methylpyrimidin-4-yl)-7H-amine | E | 397.0 | B | 3.47 |
| 358 | 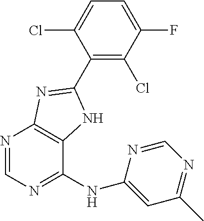 [SMILES: Cc1cc(Nc2ncnc3nc(-c4c(Cl)ccc(F)c4Cl)[nH]c23)ncn1] | 8-(2,6-dichloro-3-fluorophenyl)- N-(6-methylpyrimidin-4-yl)- 7H-purin-6-amine | E | 390.0 | B | 3.31 |
| 359 | 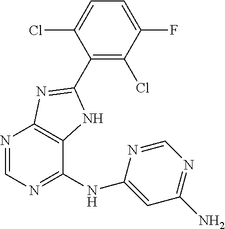 [SMILES: Nc1cc(Nc2ncnc3nc(-c4c(Cl)ccc(F)c4Cl)[nH]c23)ncn1] | N4-(8-(2,6-dichloro-3- fluorophenyl)-7H-purin-6- yl)pyrimidine-4,6-diamine | E | 391.0 | B | 3.20 |
| 360 | 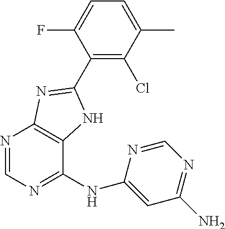 [SMILES: Cc1ccc(F)c(-c2nc3ncnc(Nc4cc(N)ncn4)c3[nH]2)c1Cl] | N4-(8-(2-chloro-6- fluoro-3-methylphenyl)- 7H-purin-6-yl)pyrimidine- 4,6-diamine | E | 371.1 | B | 3.29 |
| 361 | 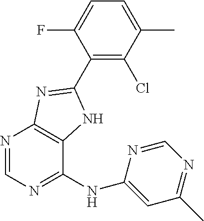 [SMILES: Cc1cc(Nc2ncnc3nc(-c4c(F)ccc(C)c4Cl)[nH]c23)ncn1] | 8-(2-chloro-6-fluoro-3- methylphenyl)-N-(6- methylpyrimidin-4-yl)-7H- purin-6-amine | E | 370.1 | B | 3.38 |
| 362 | 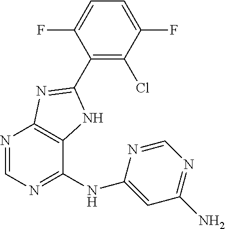 [SMILES: Nc1cc(Nc2ncnc3nc(-c4c(F)ccc(F)c4Cl)[nH]c23)ncn1] | N4-(8-(2- chloro-3,6-difluorophenyl)- 7H-purin-6-yl)pyrimidine- 4,6-diamine | E | 375.1 | B | 3.28 |
| 363 | 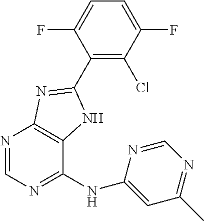 [SMILES: Cc1cc(Nc2ncnc3nc(-c4c(F)ccc(F)c4Cl)[nH]c23)ncn1] | 8-(2-chloro-3,6-difluorophenyl)- N-(6-methylpyrimidin- 4-yl)-7H-purin-6-amine | E | 374.1 | B | 3.35 |
| 364 | 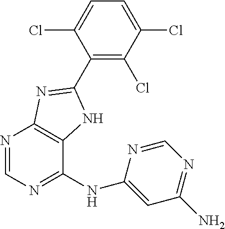 [SMILES: Nc1cc(Nc2ncnc3nc(-c4c(Cl)ccc(Cl)c4Cl)[nH]c23)ncn1] | N4-(8-(2,3,6-trichlorophenyl)- 7H-purin-6-yl)pyrimidine- 4,6-diamine | E | 407.0 | B | 3.54 |
| 365 | 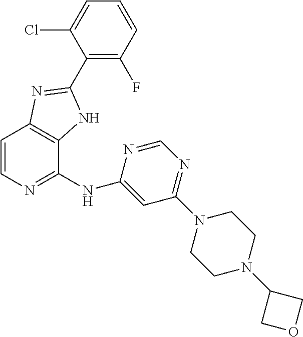 [SMILES: Fc1cccc(Cl)c1-c1nc2ccnc(Nc3cc(N4CCN(C5COC5)CC4)ncn3)c2[nH]1] | 2-(2-chloro-6-fluorophenyl)- N-(6-(4-(oxetan-3- yl)piperazin-1- yl)pyrimidin-4-yl)-3H- imidazo[4,5-c]pyridin-4-amine | A | 481.1 | B | 3.04 |
| 366 | 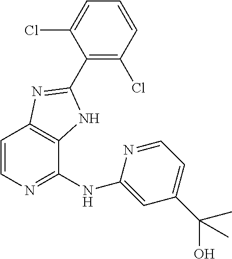 [SMILES: CC(C)(O)c1ccnc(Nc2nccc3nc(-c4c(Cl)cccc4Cl)[nH]c23)c1] | 2-(2-(2-(2,6-dichlorophenyl)- 3H-imidazo[4,5- c]pyridin-4-ylamino)pyridin- 4-yl)propan-2-ol | A | 414.0 | B | 3.78 |
| 367 | 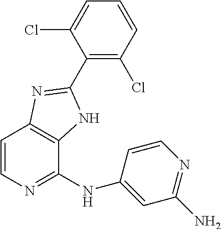 [SMILES: Nc1cc(Nc2nccc3nc(-c4c(Cl)cccc4Cl)[nH]c23)ccn1] | N4-(2-(2,6-dichlorophenyl)- 3H-imidazo[4,5-c]pyridin-4- yl)pyridine-2,4-diamine | A | 371.0 | B | 3.71 |
| 368 | 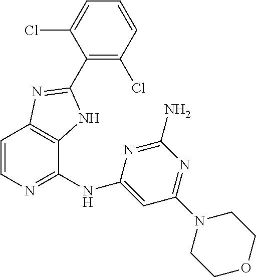 [SMILES: Nc1nc(Nc2nccc3nc(-c4c(Cl)cccc4Cl)[nH]c23)cc(N2CCOCC2)n1] | N4-(2-(2,6-dichlorophenyl)- 3H-imidazo[4,5-c]pyridin-4- yl)-6-morpholino-pyrimidine-2,4- diamine | A | 457.1 | B | 3.65 |
| 369 | 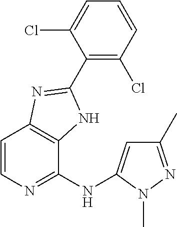 [SMILES: Cc1cc(Nc2nccc3nc(-c4c(Cl)cccc4Cl)[nH]c23)n(C)n1] | 2-(2,6-dichlorophenyl)- N-(1,3-dimethyl-1H- pyrazol-5-yl)-3H- imidazo[4,5-c]pyridin-4-amine | A | 373.0 | B | 3.41 |
| 370 | 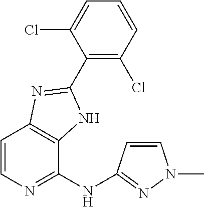 [SMILES: Cn1ccc(Nc2nccc3nc(-c4c(Cl)cccc4Cl)[nH]c23)n1] | 2-(2,6-dichlorophenyl)- N-(1-methyl-1H-pyrazol-3-yl)- 3H-imidazo[4,5-c]pyridin-4-amine | A | 359.0 | B | 3.66 |
| 371 | 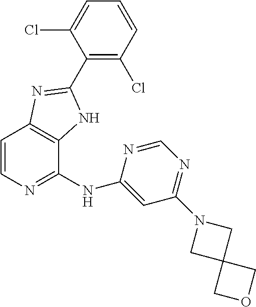 [SMILES: Clc1cccc(Cl)c1-c1nc2ccnc(Nc3cc(N4CC5(COC5)C4)ncn3)c2[nH]1] | N-(6-(2-oxa-6-azaspiro[3.3]- heptan-6-yl)pyrimidin-4- yl)-2-(2,6-dichlorophenyl)-3H- imidazo[4,5-c]pyridin-4-amine | A | 454.1 | B | 3.56 |
| 372 | 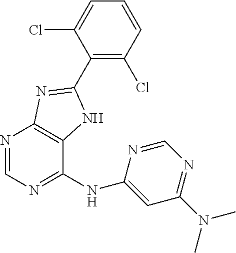 [SMILES: CN(C)c1cc(Nc2ncnc3nc(-c4c(Cl)cccc4Cl)[nH]c23)ncn1] | N4-(8-(2,6-dichlorophenyl)- 7H-purin-6-yl)-N6,N6- dimethylpyrimidine-4,6-diamine | E | 401.0 | B | 3.68 |
| 373 | 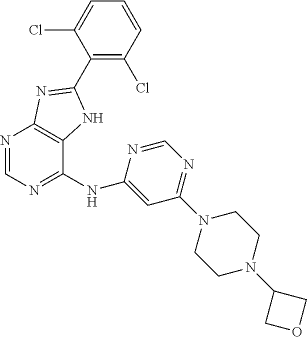 [SMILES: Clc1cccc(Cl)c1-c1nc2ncnc(Nc3cc(N4CCN(C5COC5)CC4)ncn3)c2[nH]1] | 8-(2,6-dichlorophenyl)- N-(6-(4-(oxetan-3-yl)piperazin- 1-yl)pyrimidin-4-yl)-7H- purin-6-amine | E | 498.1 | B | 3.02 |
| 374 | 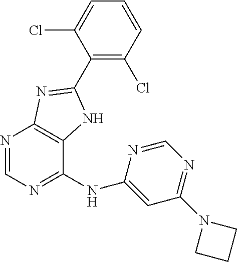 [SMILES: Clc1cccc(Cl)c1-c1nc2ncnc(Nc3cc(N4CCC4)ncn3)c2[nH]1] | N-(6- (azetidin-1-yl)pyrimidin- 4-yl)-8-(2,6-dichlorophenyl)- 7H-purin-6-amine | E | 413.0 | B | 3.61 |
| 375 | 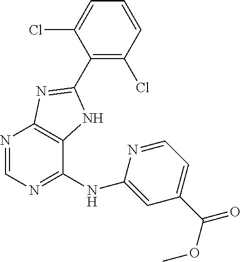 [SMILES: COC(=O)c1ccnc(Nc2ncnc3nc(-c4c(Cl)cccc4Cl)[nH]c23)c1] | methyl 2-(8-(2,6- dichlorophenyl)-7H-purin-6- ylamino)isonicotinate | E | 415.0 | B | 3.87 |
| 376 | 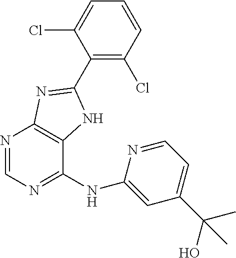 [SMILES: CC(C)(O)c1ccnc(Nc2ncnc3nc(-c4c(Cl)cccc4Cl)[nH]c23)c1] | 2-(2-(8-(2,6-dichlorophenyl)- 7H-purin-6-ylamino)pyridin-4- yl)propan-2-ol | E | 415.0 | B | 3.45 |
| 377 | 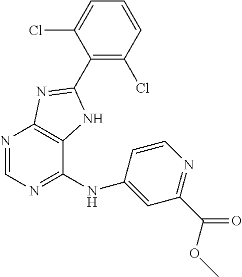 [SMILES: COC(=O)c1cc(Nc2ncnc3nc(-c4c(Cl)cccc4Cl)[nH]c23)ccn1] | methyl 4-(8-(2,6- dichlorophenyl)-7H- purin-6-ylamino)picolinate | E | 415.0 | B | 3.51 |
| 378 | 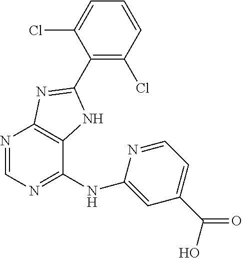 [SMILES: O=C(O)c1ccnc(Nc2ncnc3nc(-c4c(Cl)cccc4Cl)[nH]c23)c1] | 2-(8-(2,6-dichlorophenyl)- 7H-purin-6-ylamino)isonicotinic acid | E | 401.0 | B | 3.37 |
| 379 | 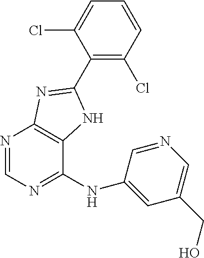 [SMILES: OCc1cncc(Nc2ncnc3nc(-c4c(Cl)cccc4Cl)[nH]c23)c1] | (5-(8-(2,6-dichlorophenyl)- 7H-purin-6-ylamino)pyridin- 3-yl)methanol | E | 387.0 | B | 3.04 |
| 380 | 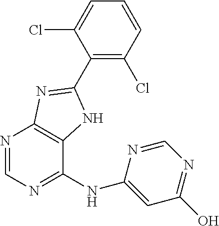 [SMILES: Oc1cc(Nc2ncnc3nc(-c4c(Cl)cccc4Cl)[nH]c23)ncn1] | 6-(8-(2,6-dichlorophenyl)- 7H-purin-6-ylamino)pyrimidin- 4-ol | E | 374.0 | B | 3.20 |
| 381 | 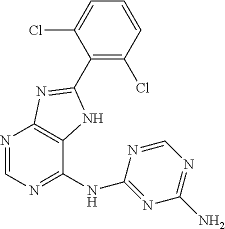 [SMILES: Nc1ncnc(Nc2ncnc3nc(-c4c(Cl)cccc4Cl)[nH]c23)n1] | (8-(2,6-dichlorophenyl)- 7H-purin-6-yl)-1,3,5-triazine- 2,4-diamine | E | 374.0 | B | 3.05 |
| 382 | 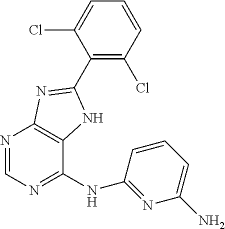 [SMILES: Nc1cccc(Nc2ncnc3nc(-c4c(Cl)cccc4Cl)[nH]c23)n1] | N2-(8-(2,6-dichlorophenyl)- 7H-purin-6-yl)pyridine- 2,6-diamine | E | 372.0 | B | 3.48 |
| 383 | 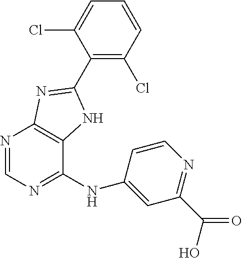 [SMILES: O=C(O)c1cc(Nc2ncnc3nc(-c4c(Cl)cccc4Cl)[nH]c23)ccn1] | 4-(8-(2,6-dichlorophenyl)- 7H-purin-6-ylamino)picolinic acid | E | 401.0 | B | 3.15 |
| 384 | 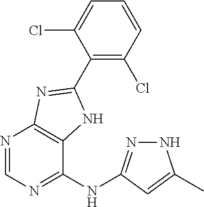 [SMILES: Cc1cc(Nc2ncnc3nc(-c4c(Cl)cccc4Cl)[nH]c23)n[nH]1] | 8-(2,6-dichlorophenyl)- N-(5-methyl-1H- pyrazol-3-yl)-7H-purin-6- amine | E | 360.0 | B | 4.29 |
| 385 | 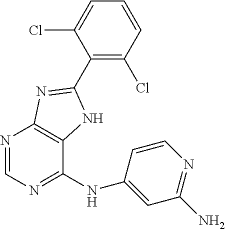 [SMILES: Nc1cc(Nc2ncnc3nc(-c4c(Cl)cccc4Cl)[nH]c23)ccn1] | N4-(8-(2,6-dichlorophenyl)- 7H-purin-6-yl)pyridine- 2,4-diamine | E | 372.0 | B | 3.39 |
| 386 | 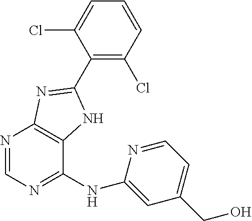 [SMILES: OCc1ccnc(Nc2ncnc3nc(-c4c(Cl)cccc4Cl)[nH]c23)c1] | (2-(8-(2,6-dichlorophenyl)- 7H-purin-6-ylamino)pyridin- 4-yl)methanol | E | 387.0 | B | 3.17 |
| 387 | 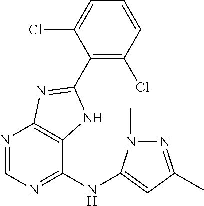 [SMILES: Cc1cc(Nc2ncnc3nc(-c4c(Cl)cccc4Cl)[nH]c23)n(C)n1] | 8-(2,6-dichlorophenyl)- N-(1,3-dimethyl-1H-pyrazol- 5-yl)-7H-purin-6-amine | E | 374.0 | B | 3.54 |
| 388 | 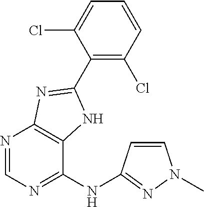 [SMILES: Cn1ccc(Nc2ncnc3nc(-c4c(Cl)cccc4Cl)[nH]c23)n1] | 8-(2,6-dichlorophenyl)- N-(1-methyl-1H-pyrazol- 3-yl)-7H-purin-6-amine | E | 360.0 | B | 3.39 |
| 389 | 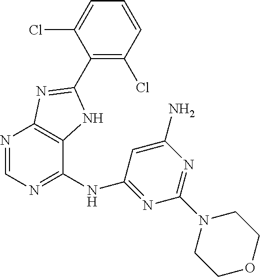 [SMILES: Nc1cc(Nc2ncnc3nc(-c4c(Cl)cccc4Cl)[nH]c23)nc(N2CCOCC2)n1] | N4-(8-(2,6-dichlorophenyl)- 7H-purin-6-yl)-2- morpholino-pyrimidine- 4,6-diamine | E | 458.0 | B | 3.58 |
| 390 | 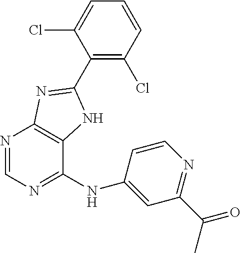 [SMILES: CC(=O)c1cc(Nc2ncnc3nc(-c4c(Cl)cccc4Cl)[nH]c23)ccn1] | 1-(4-(8-(2,6-dichlorophenyl)- 7H-purin-6-ylamino)pyridin- 2-yl)ethanone | E | 399.0 | B | 3.39 |
| 391 | 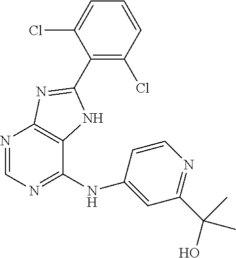 [SMILES: CC(C)(O)c1cc(Nc2ncnc3nc(-c4c(Cl)cccc4Cl)[nH]c23)ccn1] | 2-(4-(8-(2,6-dichlorophenyl)- 7H-purin-6-ylamino)pyridin- 2-yl)propan-2-ol | E | 415.0 | B | 3.29 |
| 392 | 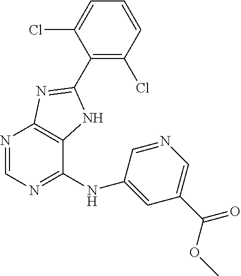 [SMILES: COC(=O)c1cncc(Nc2ncnc3nc(-c4c(Cl)cccc4Cl)[nH]c23)c1] | methyl 5-(8-(2,6- dichlorophenyl)- 7H-purin-6-ylamino)- nicotinate | E | 415.0 | B | 3.90 |
| 393 | 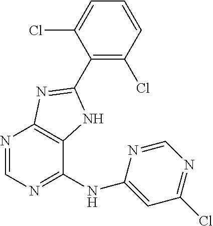 [SMILES: Clc1cc(Nc2ncnc3nc(-c4c(Cl)cccc4Cl)[nH]c23)ncn1] | N-(6-chloropyrimidin- 4-yl)-8-(2,6-dichlorophenyl)- 7H-purin-6-amine | E | 392.0 | B | 4.33 |
| 394 | 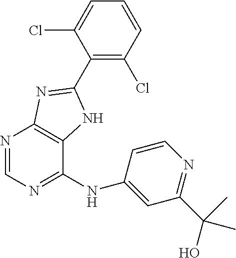 [SMILES: CC(C)(O)c1cc(Nc2ncnc3nc(-c4c(Cl)cccc4Cl)[nH]c23)ccn1] | 2-(4-(8-(2,6-dichlorophenyl)- 7H-purin-6-ylamino)pyridin- 2-yl)propan-2-ol | E | 415.0 | B | 3.41 |
| 395 | 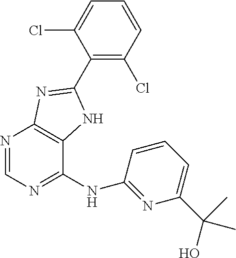 [SMILES: CC(C)(O)c1cccc(Nc2ncnc3nc(-c4c(Cl)cccc4Cl)[nH]c23)n1] | 2-(6-(8-(2,6-dichlorophenyl)- 7H-purin-6-ylamino)pyridin- 2-yl)propan-2-ol | E | 415.0 | B | 3.53 |
| 396 | 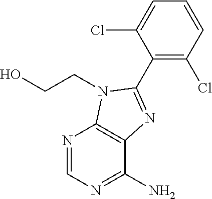 [SMILES: Nc1ncnc2c1nc(-c1c(Cl)cccc1Cl)n2CCO] | 2-(6-amino-8-(2,6- dichlorophenyl)-9H- purin-9-yl)ethanol | E | 324.0 | B | 2.83 |
| 397 | 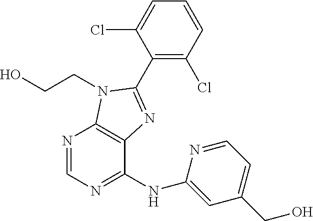 [SMILES: OCCn1c(-c2c(Cl)cccc2Cl)nc2c(Nc3cc(CO)ccn3)ncnc21] | 2-(8-(2,6-dichlorophenyl)- 6-(4-(hydroxymethyl)- pyridin-2-ylamino)-9H- purin-9-yl)ethanol | E | 431.0 | B | 3.19 |
| 398 | 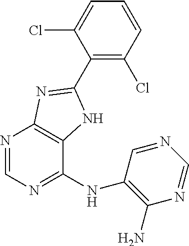 [SMILES: Nc1ncncc1Nc1ncnc2nc(-c3c(Cl)cccc3Cl)[nH]c12] | N5-(8-(2,6-dichlorophenyl)- 7H-purin-6-yl)pyrimidine- 4,5-diamine | E | 373.0 | B | 2.79 |
| 399 | 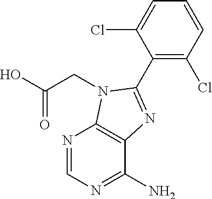 [SMILES: Nc1ncnc2c1nc(-c1c(Cl)cccc1Cl)n2CC(=O)O] | 2-(6-amino-8-(2,6-dichlorophenyl)- 9H-purin-9-yl)acetic acid | E | 338.0 | B | 2.83 |
| 400 | 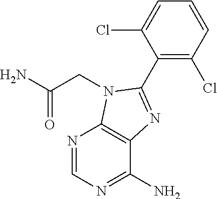 [SMILES: NC(=O)Cn1c(-c2c(Cl)cccc2Cl)nc2c(N)ncnc21] | 2-(6-amino-8-(2,6- dichlorophenyl)-9H-purin- 9-yl)acetamide | E | 337.0 | B | 2.54 |
| 401 | 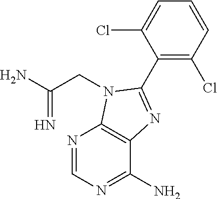 [SMILES: N=C(N)Cn1c(-c2c(Cl)cccc2Cl)nc2c(N)ncnc21] | 2-(6-amino-8-(2,6- dichlorophenyl)-9H-purin- 9-yl)acetimidamide | E | 336.0 | B | 2.27 |
| 402 | 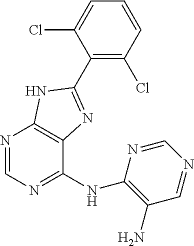 [SMILES: Nc1cncnc1Nc1ncnc2[nH]c(-c3c(Cl)cccc3Cl)nc12] | N4-(8-(2,6-dichlorophenyl)- 9H-purin-6-yl)pyrimidine- 4,5-diamine | E | 373.0 | B | 3.07 |
| 403 | 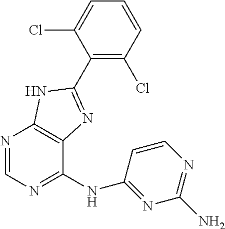 [SMILES: Nc1nccc(Nc2ncnc3[nH]c(-c4c(Cl)cccc4Cl)nc23)n1] | N4-(8-(2,6-dichlorophen- yl)-9H-purin-6-yl)pyrimidine- 2,4-diamine | E | 373.0 | B | 3.05 |
| 404 | 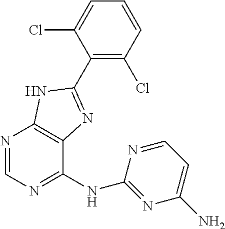 [SMILES: Nc1ccnc(Nc2ncnc3[nH]c(-c4c(Cl)cccc4Cl)nc23)n1] | N2-(8-(2,6-dichlorophenyl)- 9H-purin-6-yl)pyrimidine- 2,4-diamine | E | 373.0 | B | 3.20 |
| 405 | 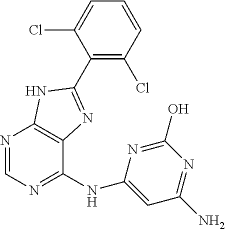 [SMILES: Nc1cc(Nc2ncnc3[nH]c(-c4c(Cl)cccc4Cl)nc23)nc(O)n1] | 4-amino-6-(8-(2,6- dichlorophenyl)-9H-purin- 6-ylamino)pyrimidin-2-ol | E | 389.0 | B | 3.32 |
| 406 | 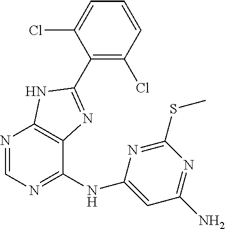 [SMILES: CSc1nc(N)cc(Nc2ncnc3[nH]c(-c4c(Cl)cccc4Cl)nc23)n1] | N-4-(8-(2,6-dichlorophenyl)- 9H-purin-6-yl)-2-(methylthio)- pyrimidine-4,6- diamine | E | 419.0 | B | 3.66 |
| 407 | 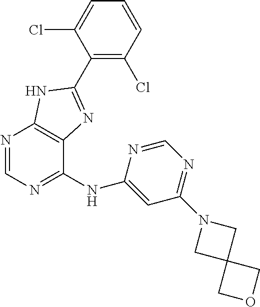 [SMILES: Clc1cccc(Cl)c1-c1nc2c(Nc3cc(N4CC5(COC5)C4)ncn3)ncnc2[nH]1] | N-(6-(2-oxa-6-azaspiro[3.3]- heptan-6-yl)pyrimidin- 4-yl)-8-(2,6-dichlorophenyl)- 9H-purin-6-amine | E | 455.0 | B | 3.37 |
| 408 | 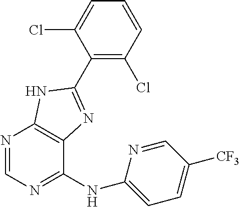 [SMILES: FC(F)(F)c1ccc(Nc2ncnc3[nH]c(-c4c(Cl)cccc4Cl)nc23)nc1] | 8-(2,6-dichlorophenyl)- N-(5-(trifluoromethyl) pyridin-2-yl)-9H-purin-6-amine | E | 424.9 | B | 3.84 |
| 409 | 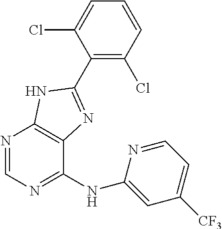 [SMILES: FC(F)(F)c1ccnc(Nc2ncnc3[nH]c(-c4c(Cl)cccc4Cl)nc23)c1] | 8-(2,6-dichlorophenyl)- N-(4-(trifluoromethyl)- pyridin-2-yl)-9H-purin- 6-amine | E | 424.9 | B | 3.83 |
| 410 | 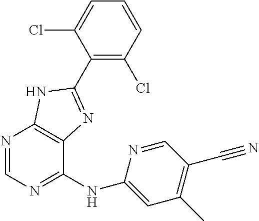 [SMILES: Cc1cc(Nc2ncnc3[nH]c(-c4c(Cl)cccc4Cl)nc23)ncc1C#N] | 6-(8-(2,6-dichlorophenyl)- 9H-purin-6-ylamino)-4- methylnicotinonitrile | E | 396.0 | B | 4.08 |
| 411 | 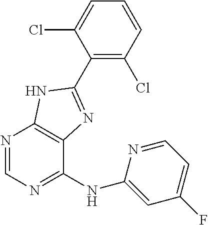 [SMILES: Fc1ccnc(Nc2ncnc3[nH]c(-c4c(Cl)cccc4Cl)nc23)c1] | 8-(2,6-dichlorophenyl)- N-(4-fluoropyridin- 2-yl)-9H-purin-6-amine | E | 374.9 | B | 2.97 |
| 412 | 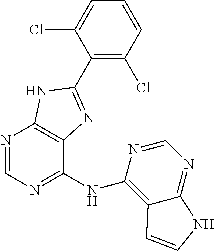 [SMILES: Clc1cccc(Cl)c1-c1nc2c(Nc3ncnc4[nH]ccc34)ncnc2[nH]1] | 8-(2,6-dichlorophenyl)-N-(7H- pyrrolo[2,3-d]-pyrimidin-4- yl)-9H-purin-6-amine | E | 396.9 | B | 2.73 |
| 413 | 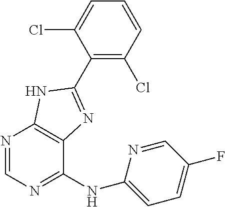 [SMILES: Fc1ccc(Nc2ncnc3[nH]c(-c4c(Cl)cccc4Cl)nc23)nc1] | 8-(2,6-dichlorophenyl)- N-(5-fluoropyridin- 2-yl)-9H-purin-6-amine | E | 375.0 | B | 3.70 |
| 414 | 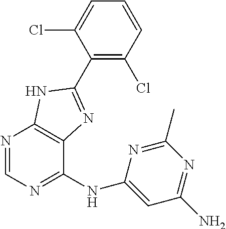 [SMILES: Cc1nc(N)cc(Nc2ncnc3[nH]c(-c4c(Cl)cccc4Cl)nc23)n1] | N4-(8-(2,6-dichlorophenyl)- 9H-purin-6-yl)-2-methylpyrimidine- 4,6-diamine | E | 387.1 | B | 3.24 |
| 415 | 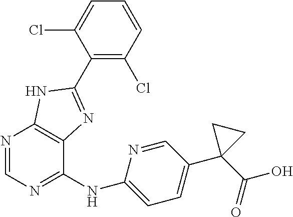 [SMILES: O=C(O)C1(c2ccc(Nc3ncnc4[nH]c(-c5c(Cl)cccc5Cl)nc34)nc2)CC1] | 1-(6-(8-(2,6-dichlorophenyl)- 9H-purin-6-ylamino)pyridin-3- yl)cyclopropane-carboxylic acid | E | 441.1 | B | 3.56 |
| 416 | 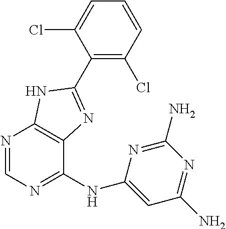 [SMILES: Nc1cc(Nc2ncnc3[nH]c(-c4c(Cl)cccc4Cl)nc23)nc(N)n1] | N4-(8-(2,6-dichlorophenyl)- 9H-purin-6-yl)pyrimidine- 2,4,6-triamine | E | 388.1 | B | 3.21 |
| 417 | 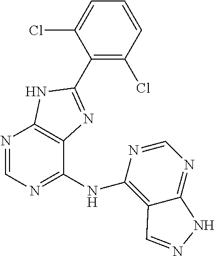 [SMILES: Clc1cccc(Cl)c1-c1nc2c(Nc3ncnc4[nH]ncc34)ncnc2[nH]1] | N-(8-(2,6-dichlorophenyl)- 9H-purin-6-yl)-1H-pyrazolo[3,4- d]pyrimidin-4-amine | E | 398.0 | B | 3.24 |
| 418 | 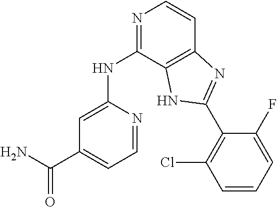 [SMILES: NC(=O)c1ccnc(Nc2nccc3nc(-c4c(F)cccc4Cl)[nH]c23)c1] | 2-(2-(2-chloro-6-fluorophenyl)- 3H-imidazo□[4,5-c]pyridin- 4-ylamino)-isonicotinamide | A | 383 | B | 3.24 |
| 419 | 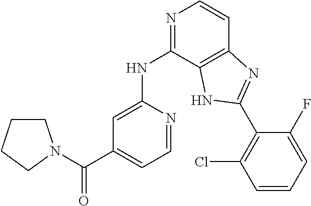 [SMILES: O=C(c1ccnc(Nc2nccc3nc(-c4c(F)cccc4Cl)[nH]c23)c1)N1CCCC1] | (2-(2-(2-chloro-6-fluorophenyl)- 3H-imidazo[4,5-c]pyridin-4- ylamino)pyridin-4-yl)(pyrrolidin- 1-yl)methanone | A | 437.1 | B | 3.71 |
| 420 | 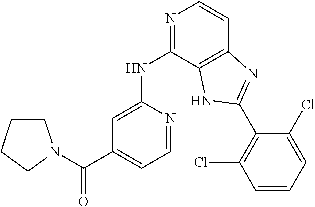 [SMILES: O=C(c1ccnc(Nc2nccc3nc(-c4c(Cl)cccc4Cl)[nH]c23)c1)N1CCCC1] | (2-(2-(2,6-dichlorophenyl)- 3H-imidazo[4,5-c]pyridin-4- ylamino)pyridin- 4-yl)(pyrrolidin-1-yl)methanone | A | 453.1 | B | 3.75 |
| 421 | 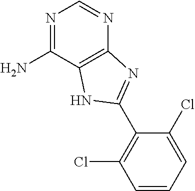 [SMILES: Nc1ncnc2nc(-c3c(Cl)cccc3Cl)[nH]c12] | 8-(2,6-dichlorophenyl)- 7H-purin-6-amine | E | 279.9 | B | 2.73 |
| 422 | 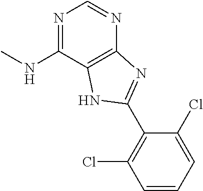 [SMILES: CNc1ncnc2nc(-c3c(Cl)cccc3Cl)[nH]c12] | 8-(2,6-dichlorophen-yl)- N-methyl-□7H-purin-6- amine | E | 293.9 | B | 2.95 |
| 423 | 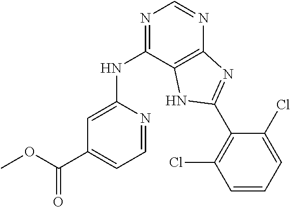 [SMILES: COC(=O)c1ccnc(Nc2ncnc3nc(-c4c(Cl)cccc4Cl)[nH]c23)c1] | methyl 2-(8-(2,6- dichlorophenyl)-7H-purin- 6-ylamino)isonicotinate | E | 415 | B | 3.87 |
| 424 | 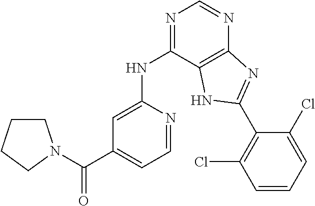 [SMILES: O=C(c1ccnc(Nc2ncnc3nc(-c4c(Cl)cccc4Cl)[nH]c23)c1)N1CCCC1] | (2-(8-(2,6-dichlorophenyl)- 7H-purin-6-ylamino)□pyridin- 4-yl)(pyrrolidin-1-yl)methanone | E | 454.1 | B | 3.61 |
| 425 | 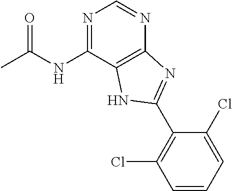 [SMILES: CC(=O)Nc1ncnc2nc(-c3c(Cl)cccc3Cl)[nH]c12] | N-(8-(2,6-dichlorophenyl)- 7H-purin-6-yl)acetamide | E | 322.0 | B | 3.30 |
| 426 | 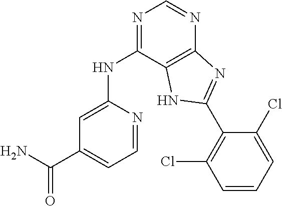 [SMILES: NC(=O)c1ccnc(Nc2ncnc3nc(-c4c(Cl)cccc4Cl)[nH]c23)c1] | 2-(8-(2,6-dichlorophenyl)- 7H-purin-6□-ylamino)- isonicotinamide | E | 400 | B | 3.16 |
| 427 | 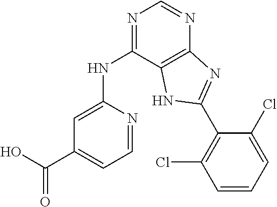 [SMILES: O=C(O)c1ccnc(Nc2ncnc3nc(-c4c(Cl)cccc4Cl)[nH]c23)c1] | 2-(8-(2,6-dichlorophenyl)- 7H-purin-6-ylamino)isonicotinic acid | E | 401 | B | 3.36 |
| 428 | 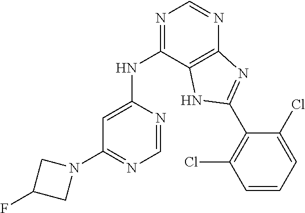 [SMILES: FC1CN(c2cc(Nc3ncnc4nc(-c5c(Cl)cccc5Cl)[nH]c34)ncn2)C1] | 2-(2,6-dichlorophenyl)- N-(6-(3-fluoroatzetidin-1- yl)-pyrimidin-4-yl)-3H- imidazo[4,5-c]pyridin-4- amine | A | 430 | B | 3.81 |
| 429 | 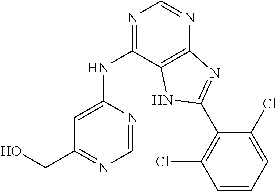 [SMILES: OCc1cc(Nc2ncnc3nc(-c4c(Cl)cccc4Cl)[nH]c23)ncn1] | (6-(8-(2,6-dichlorophenyl)- 7H-purin-6-ylamino)pyrimidin- 4-yl)methanol | E | 388 | B | 3.13 |
| 430 | 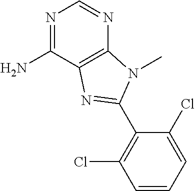 [SMILES: Cn1c(-c2c(Cl)cccc2Cl)nc2c(N)ncnc21] | 8-(2,6-dichlorophenyl)- 9-methyl-9H-purin-6-amine | E | 293.9 | B | 3.21 |
| 431 | 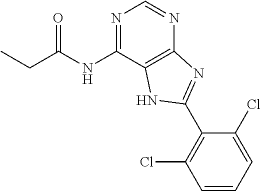 [SMILES: CCC(=O)Nc1ncnc2nc(-c3c(Cl)cccc3Cl)[nH]c12] | N-(8-(2,6-dichlorophenyl)- 7H-purin-6-yl)propionamide | E | 336 | B | 3.68 |
| 432 | 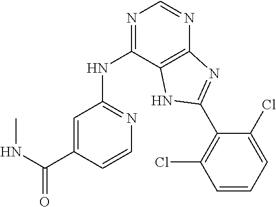 [SMILES: CNC(=O)c1ccnc(Nc2ncnc3nc(-c4c(Cl)cccc4Cl)[nH]c23)c1] | 2-(8-(2,6-dichlorophenyl)- 7H-purin-6-ylamino)-N-methyl- isonicotinamide | E | 414 | B | 3.33 |
| 433 | 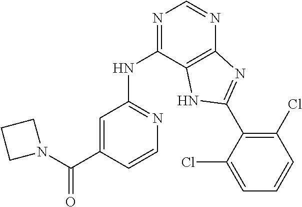 [SMILES: O=C(c1ccnc(Nc2ncnc3nc(-c4c(Cl)cccc4Cl)[nH]c23)c1)N1CCC1] | azetidin-1-yl(2-(8-(2,6- dichlorophenyl)-7H-purin-6- ylamino)pyridin- 4-yl)methanone | E | 440 | B | 3.50 |
| 434 | 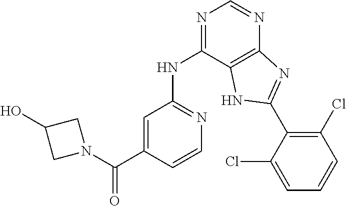 [SMILES: O=C(c1ccnc(Nc2ncnc3nc(-c4c(Cl)cccc4Cl)[nH]c23)c1)N1CC(O)C1] | (2-(8-(2,6-dichlorophenyl)- 7H-purin-6-ylamino)pyridin- 4-yl)(3-hydroxyazetidin- 1-yl)methanone | E | 456 | B | 3.18 |
| 435 | 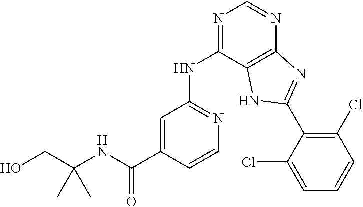 [SMILES: CC(C)(CO)NC(=O)c1ccnc(Nc2ncnc3nc(-c4c(Cl)cccc4Cl)[nH]c23)c1] | 2-(8-(2,6-dichlorophenyl)- 7H-purin-6-ylamino)-N- (1-hydroxy-2-methylpropan-2- yl)isonicotinamide | E | 472 | B | 3.47 |
| 436 | 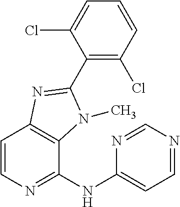 [SMILES: Cn1c(-c2c(Cl)cccc2Cl)nc2ccnc(Nc3ccncn3)c21] | [2-(2,6-Dichloro- phenyl)-3-methyl-3H- imidazo[4,5-c]pyridin-4-yl]- pyrimidin-4-yl-amine | C | 372.1 | B | 3.40 |
| 437 | 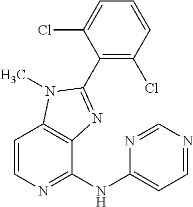 [SMILES: Cn1c(-c2c(Cl)cccc2Cl)nc2c(Nc3ccncn3)nccc21] | [2-(2,6-Dichloro- phenyl)-1-methyl-1H- imidazo-[4,5-c]pyridin- 4-yl]-pyrimidin-4-yl-amine | C | 372.6 | B | 3.31 |
| 438 | 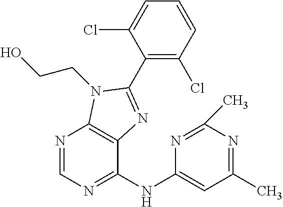 [SMILES: Cc1cc(Nc2ncnc3c2nc(-c2c(Cl)cccc2Cl)n3CCO)nc(C)n1] | 2-[8-(2,6-Dichloro- phenyl)-6-(2,6-dimethyl- pyrimidin-4-ylamino)- purin-9-yl]-ethanol | F | 430.1 | B | 3.19 |
| 439 | 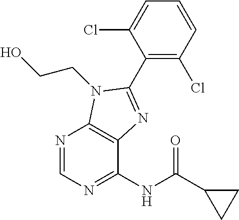 [SMILES: O=C(Nc1ncnc2c1nc(-c1c(Cl)cccc1Cl)n2CCO)C1CC1] | Cyclopropanecarboxylic acid [8-(2,6-dichloro-phenyl)- 9-(2-hydroxy-ethyl)-9H- purin-6-yl]-amide | F | 393.0 | B | 3.40 |
| 440 | 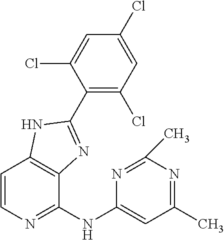 [SMILES: Cc1cc(Nc2nccc3[nH]c(-c4c(Cl)cc(Cl)cc4Cl)nc23)nc(C)n1] | (2,6-Dimethyl-pyrimidin-4- yl)-[2-(2,4,6-trichloro- phenyl)-1H-imidazo[4,5- c]pyridin-4-yl]-amine | C | 420.2 | B | 3.53 |
| 441 | 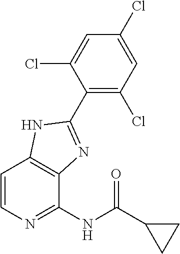 [SMILES: O=C(Nc1nccc2[nH]c(-c3c(Cl)cc(Cl)cc3Cl)nc12)C1CC1] | Cyclopropanecarboxylic acid [2-(2,4,6-trichloro- phenyl)-1H-imidazo[4,5- c]pyridin-4-yl]-amide | C | 382.3 | B | 3.80 |
| 442 | 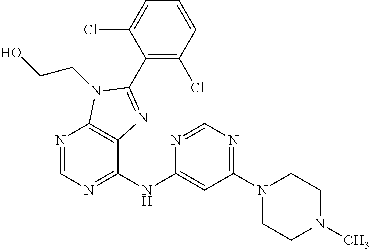 [SMILES: CN1CCN(c2cc(Nc3ncnc4c3nc(-c3c(Cl)cccc3Cl)n4CCO)ncn2)CC1] | 2-{8-(2,6-Dichloro- phenyl)-6-[6-(4-methyl- piperazin-1-yl)-pyrimidin- 4-ylamino]-purin-9-yl}- ethanol | F | 501.1 | B | 2.94 |
| 443 | 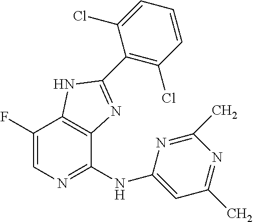 [SMILES: Cc1cc(Nc2ncc(F)c3[nH]c(-c4c(Cl)cccc4Cl)nc23)nc(C)n1] | [2-(2,6-Dichloro- phenyl)-7-fluoro-1H- imidazo[4,5-c]pyridin-4- yl]-(2,6-pyrimidin-4- yl)-amine | C | 404.0 | B | 3.44 |
| 444 | 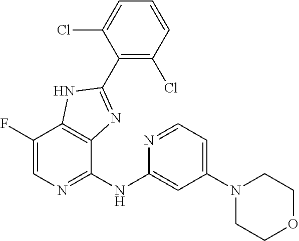 [SMILES: Fc1cnc(Nc2cc(N3CCOCC3)ccn2)c2nc(-c3c(Cl)cccc3Cl)[nH]c12] | [2-(2,6-Dichloro- phenyl)-7-fluoro-1H- imidazo[4,5-c]pyridin-4- yl]-(4-morpholin-4- yl-pyridin-2-yl)-amine | C | 460.1 | B | 4.11 |
| 445 | 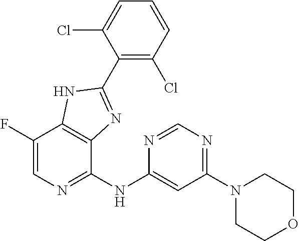 [SMILES: Fc1cnc(Nc2cc(N3CCOCC3)ncn2)c2nc(-c3c(Cl)cccc3Cl)[nH]c12] | [2-(2,6-Dichloro- phenyl)-7-fluoro-1H- imidazo[4,5-c]pyridin-4- yl]-(6-morpholin-4- yl-pyrimidin-4-yl)-amine | C | 461.5 | B | 3.97 |
| 446 | 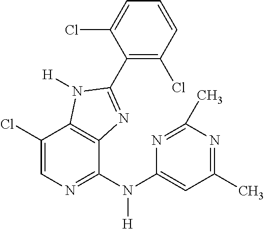 [SMILES: Cc1cc(Nc2ncc(Cl)c3[nH]c(-c4c(Cl)cccc4Cl)nc23)nc(C)n1] | [7-Chloro-2-(2,6-dichloro- phenyl)-1H-imidazo[4,5- c]pyridin-4-yl]-(2,6- dimethyl-pyrimidin-4- yl)-amine | C | 420.8 | B | 3.58 |
| 447 | 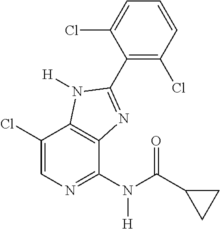 [SMILES: O=C(Nc1ncc(Cl)c2[nH]c(-c3c(Cl)cccc3Cl)nc12)C1CC1] | Cyclopropanecarboxylic acid [7-chloro-2-(2,6- dichloro-phenyl)-1H- imidazo[4,5-c]pyridin-4- yl]-amide | C | 382.3 | B | 3.99 |
| 448 | 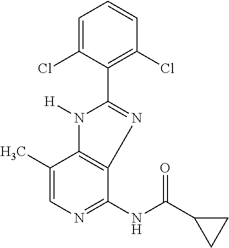 [SMILES: Cc1cnc(NC(=O)C2CC2)c2nc(-c3c(Cl)cccc3Cl)[nH]c12] | Cyclopropanecarboxylic acid [2-(2,6-dichloro- phenyl)-7-methyl-1H- imidazo[4,5-c]pyridin-4-yl]- amide | C | 362.7 | B | 3.55 |
| 449 | 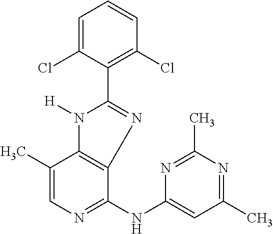 | [2-(2,6-Dichloro- phenyl)-7-methyl-1H- imidazo[4,5-c]pyridin-4- yl]-(2,6-dimethyl- pyrimidin-4-yl)-amine | C | 400.2 | B | 3.40 |
| 450 | 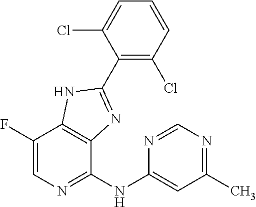 [SMILES: Cc1cc(Nc2ncc(F)c3[nH]c(-c4c(Cl)cccc4Cl)nc23)ncn1] | [2-(2,6-Dichloro- phenyl)-7-fluoro-1H- imidazo[4,5-c]pyridin-4- yl]-(6-methyl-pyrimidin-4- yl)-amine | C | 390.0 | B | 3.37 |
| 451 | 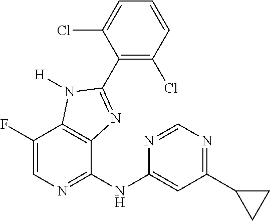 [SMILES: Fc1cnc(Nc2cc(C3CC3)ncn2)c2nc(-c3c(Cl)cccc3Cl)[nH]c12] | (6-Cyclopropyl- pyrimidin-4-yl)-[2-(2,6- dichloro-phenyl)-7- fluoro-1H-imidazo[4,5- c]pyridin-4-yl]-amine | C | 416.4 | B | 3.90 |
| 452 | 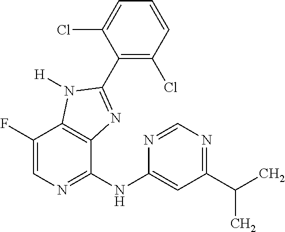 [SMILES: CC(C)c1cc(Nc2ncc(F)c3[nH]c(-c4c(Cl)cccc4Cl)nc23)ncn1] | [2-(2,6-Dichloro- phenyl)-7-fluoro-1H- imidazo[4,5-c]pyridin-4- yl]-(6-isopropyl- pyrimidin-4-yl)-amine | C | 418.3 | B | 3.86 |
| 453 | 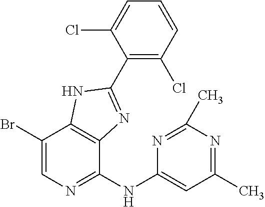 [SMILES: Cc1cc(Nc2ncc(Br)c3[nH]c(-c4c(Cl)cccc4Cl)nc23)nc(C)n1] | [7-Bromo-2-(2,6-dichloro- phenyl)-1H-imidazo[4,5- c]pyridin-4-yl]-(2,6- dimethyl-pyrimidin-4- yl)-amine | C | 465.9 | B | 3.67 |
| 454 | 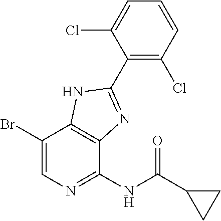 [SMILES: O=C(Nc1ncc(Br)c2[nH]c(-c3c(Cl)cccc3Cl)nc12)C1CC1] | Cyclopropanecarboxylic acid[7-bromo-2-(2,6- dichloro-phenyl)-1H- imidazo[4,5-c]pyridin-4- yl]-amide | C | 427.9 | B | 4.03 |
| 455 | 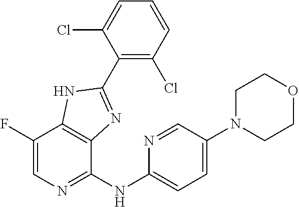 [SMILES: Fc1cnc(Nc2ccc(N3CCOCC3)cn2)c2nc(-c3c(Cl)cccc3Cl)[nH]c12] | [2-(2,6-Dichloro- phenyl)-7-fluoro-1H- imidazo[4,5-c]pyridin-4- yl]-(5-morpholin-4- yl-pyridin-2-yl)-amine | C | 460.0 | B | 4.04 |
| 456 | 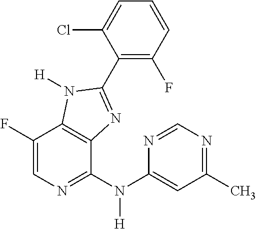 [SMILES: Cc1cc(Nc2ncc(F)c3[nH]c(-c4c(F)cccc4Cl)nc23)ncn1] | [2-(2-Chloro-6-fluoro- phenyl)-7-fluoro-1H- imidazo[4,5-c]pyridin-4- yl]-(6-methyl-pyrimidin-4- yl)-amine | C | 373.0 | B | 3.30 |
| 457 | 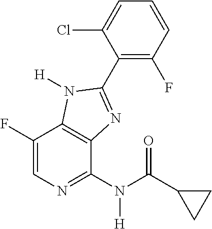 [SMILES: O=C(Nc1ncc(F)c2[nH]c(-c3c(F)cccc3Cl)nc12)C1CC1] | Cyclopropanecarboxylic acid[2-(2-chloro-6- fluoro-phenyl)-7- fluoro-1H-imidazo[4,5- c]pyridin-4-yl]-amide | C | 349.4 | B | 9.42 |
| 458 | 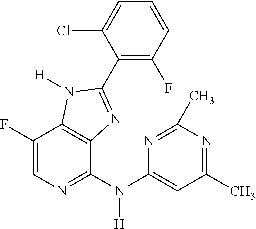 [SMILES: Cc1cc(Nc2ncc(F)c3[nH]c(-c4c(F)cccc4Cl)nc23)nc(C)n1] | [2-(2-Chloro-6-fluoro- phenyl)-7-fluoro-1H- imidazo[4,5-c]pyridin-4- yl]-(2,6-dimethyl- pyrimidin-4-yl)-amine | C | 387.0 | B | 8.18 |
| 459 | 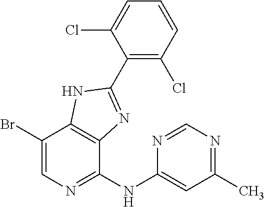 [SMILES: Cc1cc(Nc2ncc(Br)c3[nH]c(-c4c(Cl)cccc4Cl)nc23)ncn1] | [7-Bromo-2-(2,6-dichloro- phenyl)-1H-imidazo[4,5- c]pyridin-4-yl]-(6-methyl- pyrimidin-4-yl)-amine | C | 451.9 | B | 3.63 |
| 460 | 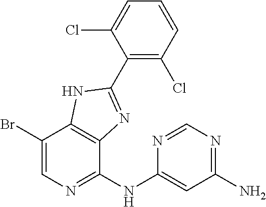 [SMILES: Nc1cc(Nc2ncc(Br)c3[nH]c(-c4c(Cl)cccc4Cl)nc23)ncn1] | N-[7-Bromo-2-(2,6-dichloro- phenyl)-1H-imidazo[4,5- c]pyridin-4-yl]-pyrimidine- 4,6-diamine | C | 452.9 | B | 3.71 |
| 461 | 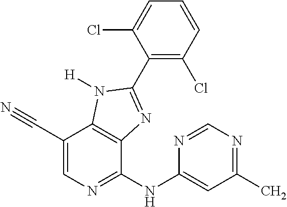 [SMILES: Cc1cc(Nc2ncc(C#N)c3[nH]c(-c4c(Cl)cccc4Cl)nc23)ncn1] | 2-(2,6-Dichloro- phenyl)-4-(6-methyl- pyrimidin-4-ylamino)-1H- imidazo[4,5-c]-pyridine-7- carbonitrile | C | 397.0 | B | 3.61 |
| 462 | 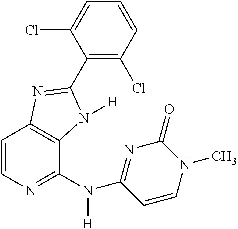 [SMILES: Cn1ccc(Nc2nccc3nc(-c4c(Cl)cccc4Cl)[nH]c23)nc1=O] | 4-[2-(2,6-Dichloro- phenyl)-3H-imidazo[4,5- c]pyridin-4-ylamino]-1- methyl-1H-pyrimidin-2-one | C | 388.7 | B | 3.22 |
| 463 | 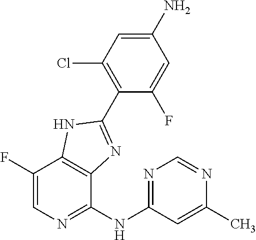 [SMILES: Cc1cc(Nc2ncc(F)c3[nH]c(-c4c(F)cc(N)cc4Cl)nc23)ncn1] | [2-(4-Amino-2-chloro-6- fluoro-phenyl)-7- fluoro-1H-imidazo[4,5-c]- pyridin-4-yl]-(6-methyl- pyrimidin-4-yl)-amine | C | 388.1 | B | 3.14 |
| 464 | 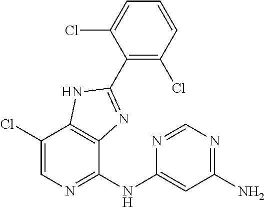 [SMILES: Nc1cc(Nc2ncc(Cl)c3[nH]c(-c4c(Cl)cccc4Cl)nc23)ncn1] | N-[7-Chloro-2-(2,6- dichloro-phenyl)-1H- imidazo[4,5-c]pyridin-4- yl]-pyrimidine-4,6-diamine | C | 407.6 | B | 3.63 |
| 465 | 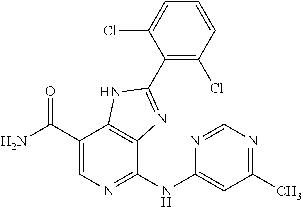 [SMILES: Cc1cc(Nc2ncc(C(N)=O)c3[nH]c(-c4c(Cl)cccc4Cl)nc23)ncn1] | 2-(2,6-Dichloro- phenyl)-4-(6-methyl- pyrimidin-4-ylamino)- 1H-imidazo[4,5-c]- pyridine-7-carboxylic acid amide | C | 415.0 | B | 3.23 |
| 466 | 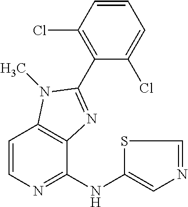 [SMILES: Cn1c(-c2c(Cl)cccc2Cl)nc2c(Nc3cncs3)nccc21] | [2-(2,6-Dichloro- phenyl)-1-methyl-1H- imidazo[4,5-c]pyridin-4- yl]-thiazol-5-yl-amine | C | 377.9 | B | 3.44 |
| 467 | 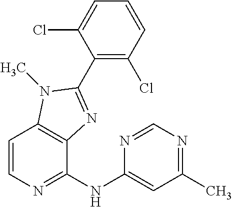 [SMILES: Cc1cc(Nc2nccc3c2nc(-c2c(Cl)cccc2Cl)n3C)ncn1] | [2-(2,6-Dichloro- phenyl)-1-methyl-1H- imidazo[4,5-c]pyridin-4- yl]-(6-methyl-pyrimidin-4- yl)-amine | C | 386.3 | B | 3.57 |
| 468 | 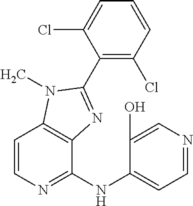 [SMILES: Cn1c(-c2c(Cl)cccc2Cl)nc2c(Nc3ccncc3O)nccc21] | 4-[2-(2,6-Dichloro- phenyl)-1-methyl-1H- imidazo[4,5-c]pyridin-4- ylamino]-pyridin-3-ol | C | 387.0 | B | 3.90 |
| 469 | 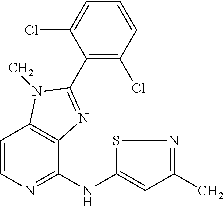 [SMILES: Cc1cc(Nc2nccc3c2nc(-c2c(Cl)cccc2Cl)n3C)sn1] | [2-(2,6-Dichloro- phenyl)-1-methyl-1H- imidazo[4,5-c]pyridin-4- yl]-(3-methyl-isothiazol-5- yl)-amine | C | 391.5 | B | 4.03 |
| 470 | 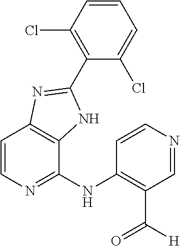 [SMILES: O=Cc1cnccc1Nc1nccc2nc(-c3c(Cl)cccc3Cl)[nH]c12] | 4-[2-(2,6-Dichloro- phenyl)-3H-imidazo[4,5- c]pyridin-4-ylamino]- pyridine-3-carbaldehyde | C | 385.9 | B | 3.26 |
| 471 | 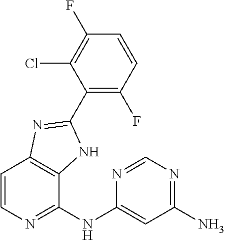 [SMILES: Nc1cc(Nc2nccc3nc(-c4c(F)ccc(F)c4Cl)[nH]c23)ncn1] | N-[2-(2-Chloro-3,6- difluoro-phenyl)-3H- imidazo[4,5-c]pyridin-4-yl]- pyrimidine-4,6-diamine | C | 374.0 | B | 3.27 |
| 472 | 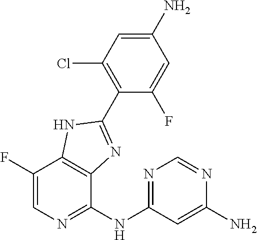 [SMILES: Nc1cc(F)c(-c2nc3c(Nc4cc(N)ncn4)ncc(F)c3[nH]2)c(Cl)c1] | N-[2-(4-Amino-2- chloro-6-fluoro-phenyl)-7- fluoro-1H-imidazo[4,5- c]pyridin-4-yl]-pyrimidine- 4,6-diamine | C | 389.2 | B | 3.30 |

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as defined by the claims.

What is claimed is:

1. A compound of Formulas Ia-Ib: 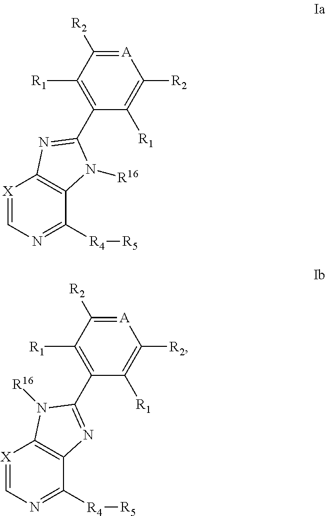
stereoisomers or pharmaceutically acceptable salts thereof, wherein:
A is CR³ or N;
X is CR¹⁵ or N;
R¹ is independently hydrogen, halogen, C₁-C₃ alkyl, C₃-C₄ cycloalkyl, —CF₃, —OR⁶, —SR⁶, —OCF₃, —CN, —NO₂, —NR⁶SO₂R⁷, —NR⁶C(O)R⁷ or —NR⁶R⁷, wherein both R¹ cannot be hydrogen at the same time, and wherein said alkyl and cycloalkyl are optionally substituted by halogen, OR⁶, —NR⁶R⁷ or phenyl;
R² and R³ are independently hydrogen, C₁-C₆ alkyl, C₂-C₆ alkenyl, C₂-C₆ alkynyl, halogen, —(C₀-C₃ alkyl)CN, —(C₀-C₃alkyl)OR⁸, —(C₀-C₃ alkyl)SR⁸, —(C₀-C₃ alkyl)NR⁸R⁹, —(C₀-C₃ alkyl)CF₃, —O(C₀-C₃alkyl)CF₃, —(C₀-C₃alkyl)NO₂, —(C₀-C₃alkyl)C(O)R⁸, —(C₀-C₃ alkyl)C(O)OR⁸, —(C₀-C₃alkyl)C(O)NR⁸R⁹, —(C₀-C₃ alkyl)NR⁸C(O)R⁹, —(C₀-C₃ alkyl)S(O)₁₋₂R⁸, —(C₀-C₃alkyl)NR⁸S(O)₁₋₂R⁹, —(C₀-C₃alkyl)S(O)₁₋₂NR⁸R⁹, —(C₀-C₃ alkyl)(C₃-C₆ cycloalkyl), —(C₀-C₃alkyl)(3-6-membered heterocyclyl), —(C₀-C₃ alkyl)(5-6-membered heteroaryl) or —(C₀-C₃ alkyl)phenyl, wherein R² and R³ are independently optionally substituted by R¹⁰;
R⁴ is —NH₂, —NH—, —NR⁶R⁷, —NR⁶C(O)—, —NR⁶C(O)O—, —NR⁶C(O)NR⁷—, —NR⁶S(O)₁₋₂— or —NR⁶S(O)₁₋₂NR⁷—;
R⁵ is absent, hydrogen, C₁-C₆ alkyl, C₂-C₆ alkenyl, C₂-C₆ alkynyl, C₃-C₈ cycloalkyl, phenyl or 3-10-membered heterocyclyl, wherein R⁵ is optionally substituted by R¹⁰;
R⁶ and R⁷ are each independently hydrogen, C₁-C₃ alkyl or C₃-C₄ cycloalkyl, wherein said alkyl and cycloalkyl are independently optionally substituted by halogen, oxo, —OR¹¹ or —NR¹¹R¹²; or
R⁶ and R⁷ are independently taken together with the atom to which they are attached to form a 3-6 membered heterocyclyl optionally substituted by halogen, oxo, —NR¹¹R¹² or C₁-C₃ alkyl;
R⁸ and R⁹ are each independently hydrogen, C₁-C₃ alkyl, C₃-C₆ cycloalkyl, phenyl, 3-6-membered heterocyclyl or 5-6-membered heteroaryl, wherein said alkyl, cycloalkyl, phenyl, heterocyclyl or heteroaryl are independently optionally substituted by R¹⁰; or
R⁸ and R⁹ are independently taken together with the atom to which they are attached to form a 3-6 membered heterocyclyl optionally substituted by halogen, oxo, —NR¹¹R¹² or C₁-C₃ alkyl;
R¹⁰ is independently hydrogen, oxo, C₁-C₆ alkyl, C₂-C₆ alkenyl, C₂-C₆ alkynyl, halogen, —(C₀-C₃ alkyl)CN, —(C₀-C₃alkyl)OR¹¹, —(C₀-C₃alkyl)SR¹¹, —(C₀-C₃ alkyl)NR¹¹R¹², —(C₀-C₃ alkyl)CF₃, —(C₀-C₃ alkyl)NO₂, —C═NH(OR¹¹), —(C₀-C₃ alkyl)C(O)R¹¹, —(C₀-C₃alkyl)C(O)OR¹¹, —(C₀-C₃ alkyl)OC(O)R¹¹, —(C₀-C₃ alkyl)OC(O)OR¹¹, —(C₀-C₃ alkyl)C(O)NR¹¹R¹², —(C₀-C₃alkyl)NR¹¹C(O)R¹², —(C₀-C₃ alkyl)NR¹¹C(O)OR¹², —(C₀-C₃alkyl)OC(O)NR¹¹R¹², —(C₀-C₃alkyl)S(O)₁₋₂R¹¹, —(C₀-C₃ alkyl)NR¹¹S(O)₁₋₂R¹², —(C₀-C₃ alkyl)S(O)₁₋₂NR¹¹R¹², —(C₀-C₃ alkyl)(C₃-C₈ cycloalkyl), —(C₀-C₃ alkyl)(3-10-membered heterocyclyl), —(C₀-C₃alkyl)C(O)(3-10-membered heterocyclyl) or —(C₀-C₃ alkyl)phenyl, wherein R¹⁰ is independently optionally substituted by halogen, oxo, —CF₃, —(C₀-C₃alkyl)OR¹³, —(C₀-C₃alkyl)NR¹³R¹⁴, —(C₀-C₃ alkyl)C(O)R¹³, —(C₀-C₃alkyl)S(O)₁₋₂R¹³, 3-10-membered heterocyclyl or C₁-C₃ alkyl optionally substituted by oxo, halogen, —NR¹³R¹⁴ or —OR¹³.
R¹¹ and R¹² are independently hydrogen, C₁-C₆ alkyl or —(C₀-C₃ alkyl)phenyl, wherein said alkyl and phenyl are independently optionally substituted by halogen, oxo, —OR¹³, —NR¹³R¹⁴, C₁-C₃ alkyl, —(C₀-C₃ alkyl)(C₃-C₆ cycloalkyl), —(C₀-C₃ alkyl)phenyl, —(C₀-C₃ alkyl)(3-6-membered heterocyclyl) or —(C₀-C₃ alkyl)(5-6-membered heteroaryl); or
R¹¹ and R¹² are taken together with the atom to which they attached to form a 3-6 membered heterocyclyl optionally substituted by halogen, oxo, —OR¹³, —NR¹³R¹⁴ or C₁-C₃ alkyl;
R¹³ and R¹⁴ are independently hydrogen, C₁-C₆ alkyl, OH or O(C₁-C₆ alkyl), wherein said alky is optionally substituted by halogen, —NH₂, —N(CH₃)₂ or oxo; or
R¹³ and R¹⁴ are taken together with the atom to which they attached to form a 3-6 membered heterocyclyl optionally substituted by halogen, oxo, —NH₂, —N(CH₃)₂ or C₁-C₃ alkyl;
R¹⁵ is hydrogen, halogen, C₁-C₆ alkyl, C₂-C₆ alkenyl, C₂-C₆ alkynyl, —(C₀-C₃ alkyl)CN, —(C₀-C₃ alkyl)OR¹⁸, —(C₀-C₃ alkyl)SR¹⁸, —(C₀-C₃ alkyl)NR¹⁸R¹⁹, —(C₀-C₃ alkyl)CF₃, —O(C₀-C₃ alkyl)CF₃, —(C₀-C₃ alkyl)NO₂, —(C₀-C₃ alkyl)C(O)R¹⁸, —(C₀-C₃ alkyl)C(O)OR¹⁸, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸—(C₀-C₃ alkyl)NR¹⁸S(O)₁₋₂R¹⁹, —(C₀-C₃ alkyl)S(O)₁₋₂NR¹⁸R¹⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R¹⁵ is optionally substituted by R¹⁰;
R¹⁶ is hydrogen, C₁-C₆ alkyl, C₂-C₆ alkenyl, C₂-C₆ alkynyl, —(C₀-C₃ alkyl)CN, —(C₁-C₃ alkyl)OR¹⁸, —(C₁-C₃ alkyl)SR¹⁸, —(C₁-C₃ alkyl)NR¹⁸R¹⁹, —(C₁-C₃ alkyl)CF₃, —O(C₁-C₃ alkyl)CF₃, —(C₂-C₃ alkyl)NO₂, —(C₀-C₃ alkyl)C(O)R¹⁸, —(C₀-C₃ alkyl)C(O)OR¹⁸, —(C₀-C₃ alkyl)C(═NR¹⁸)NR¹⁸R¹⁹, —(C₀-C₃ alkyl)C(O)NR¹⁸R¹⁹, —(C₀-C₃ alkyl)NR¹⁸C(O)R¹⁹, —(C₀-C₃ alkyl)S(O)₁₋₂R¹⁸, —(C₀-C₃ alkyl)NR¹⁸S(O)₁₋₂R¹⁹, —(C₀-C₃ alkyl)S(O)₁₋₂NR¹⁸R¹⁹, —(C₀-C₃ alkyl)(C₃-C₆ cycloalkyl), —(C₀-C₃ alkyl)(3-6-membered heterocyclyl), —(C₀-C₃ alkyl)(5-6-membered heteroaryl) or —(C₀-C₃ alkyl)phenyl, wherein R¹⁶ is optionally substituted by R¹⁰;
R¹⁸ and R¹⁹ are independently hydrogen or C₁-C₆ alkyl optionally substituted by halogen, oxo, CN, —OR²⁰, —SR²⁰ or —NR²⁰R²¹; or
R¹⁸ and R¹⁹ are taken together with the atom to which they attached to form a 3-6 membered heterocyclyl optionally substituted by halogen, oxo, C₁-C₃ alkyl, CN, —OR²⁰, —SR²⁰ or —NR²⁰R²¹; and
R²⁰ and R²¹ are independently hydrogen or C₁-C₆ alkyl optionally substituted by oxo, halogen, —OH or —NH₂,
with the proviso that Formulas Ia-Ib include compounds other than:
2-(6-amino-7H-purin-8-yl)phenol;
2-(2-fluoro-3-methoxyphenyl)-N-(pyridin-4-ylmethyl)-3H-imidazo[4,5-c]pyridin-4-amine;
8-(2,4-dichlorophenyl)-7H-purin-6-amine;
2-(2,5-dimethoxyphenyl)-N-methyl-3H-imidazo[4,5-c]pyridin-4-amine;
8-(2,3,5,6-tetrafluoro-4-(1H-imidazol-1-yl)phenyl)-7H-purin-6-amine; and
8-o-tolyl-7H-purin-6-amine

2. The compound of claim 1, wherein A is CR³ and X is CR¹⁵.

3. The compound of claim 1, wherein A is CR³ and X is N.

4. The compound of any one of claims 1-3, wherein R¹ is independently hydrogen, halogen, C₁-C₃ alkyl, —CF₃, —OR⁶, —SR⁶, —OCF₃, —NO₂ or —NR⁶R⁷, wherein both R¹ cannot be hydrogen at the same time, and wherein said alkyl is optionally substituted by halogen, OR⁶ or —NR⁶R⁷.

5. The compound of any one of claims 1-3, wherein one R¹ is halogen and the other R¹ is hydrogen, halogen, C₁-C₃ alkyl, C₃-C₄ cycloalkyl, —CF₃, —OH, —O(C₁-C₃ alkyl), —SH, —S(C₁-C₃ alkyl), —OCF₃, —CN, —NO₂, —NHSO₂CH₃, —NHC(O)R⁷ or —NR⁶R⁷, wherein said alkyl and cycloalkyl are optionally substituted by halogen, OR^{B}, —NR⁸R⁹ or phenyl.

6. The compound of any one of claims 1-5, wherein R² is independently hydrogen, halogen or C₁-C₆ alkyl optionally substituted by R¹⁰. In certain embodiments, R² is independently F, Cl, Br, —CH₂OH, —CH₂NH₂ or —CH₂morpholinyl.

7. The compound of any one of claims 1-6, wherein A is CR³ and R³ is hydrogen, C₁-C₆ alkyl, C₂-C₆ alkenyl, C₂-C₆ alkynyl, halogen, —(C₀-C₃ alkyl)CN, —(C₀-C₃ alkyl)OR⁸, —(C₀-C₃ alkyl)SR⁸, —(C₀-C₃ alkyl)NR⁸R⁹, —(C₀-C₃ alkyl)C(O)NR⁸R⁹, —(C₀-C₃ alkyl)S(O)₁₋₂R⁸ or —(C₀-C₃ alkyl)(3-6-membered heterocyclyl), wherein R³ is independently optionally substituted by R¹⁰.

8. The compound of any one of claims 1-7, wherein R³ is hydrogen, Cl, F, Br, —CH₃, acetylenyl, —NH₂, —CN, —S(O)₂CH₃, —C(O)NH₂, —CH₂NH₂, —CH₂OH, —CH₂NH(CH₃), —CH₂N(CH₃)₂ or —CH₂morpholinyl.

9. The compound of any one of claims 1-6, wherein the portion of Formula I having the structure: 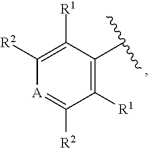
is selected from: 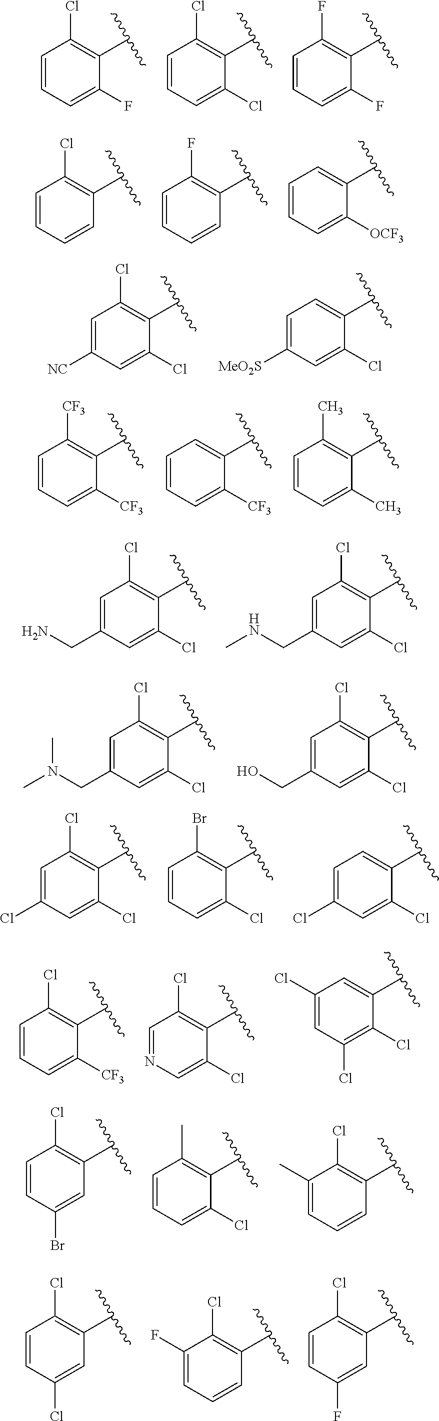 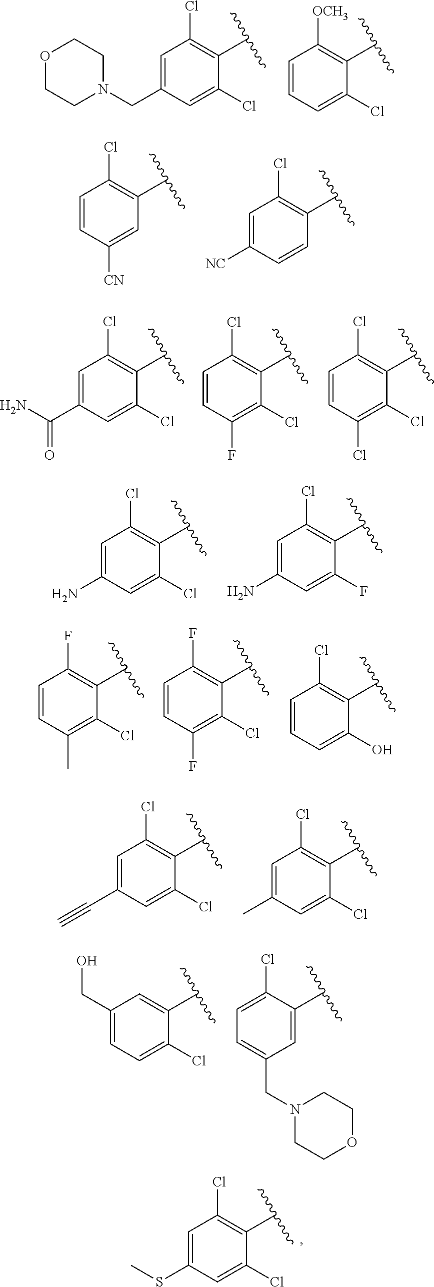
wherein the wavy lines represent the point of attachment in Formula I.

10. The compound of any one of claims 1-9, wherein R⁴ is —NH—, —NR⁶C(O)—, —NR⁶C(O)O— or —NR⁶C(O)NR⁷—.

11. The compound of any one of claims 1-9, wherein R⁵ is C₁-C₆ alkyl optionally substituted by halogen; C₃-C₆ cycloalkyl optionally substituted by halogen; phenyl optionally substituted by R¹⁰ or 3-10-membered heterocyclyl optionally substituted by R¹⁰.

12. The compound of any one of claims 1-10, wherein R⁵ is selected from hydrogen, methyl, ethyl, isopropyl, tert-butyl, —CH₂OH, —CH₂N(CH₃)₂, —CH₂NHC(O)OC(CH₃)₃, cyclopropyl, cyclobutyl,

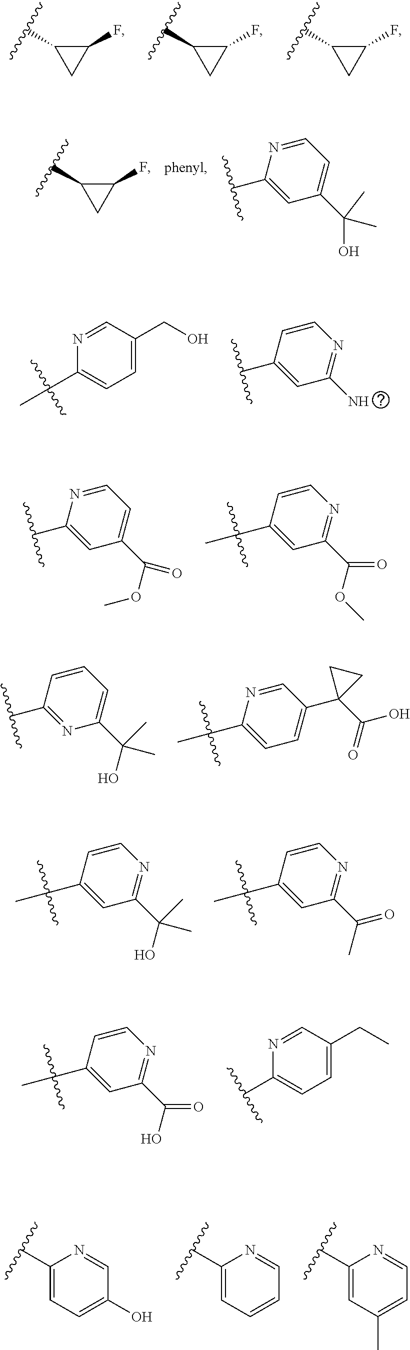

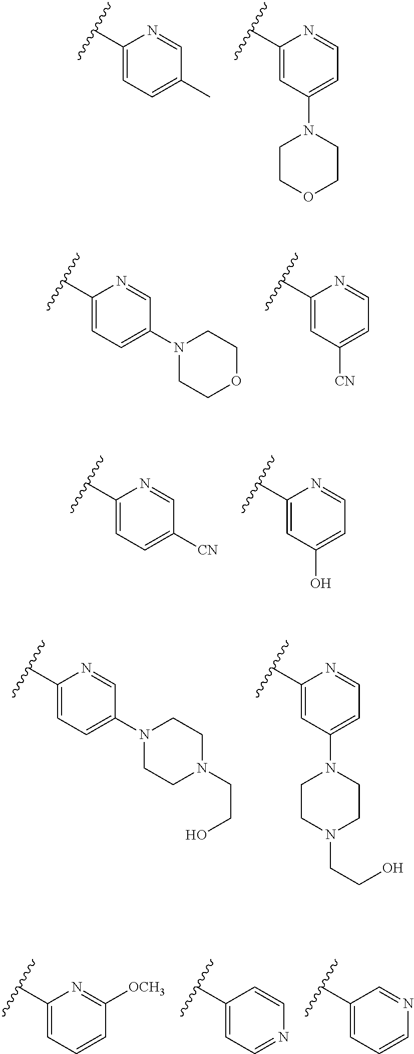

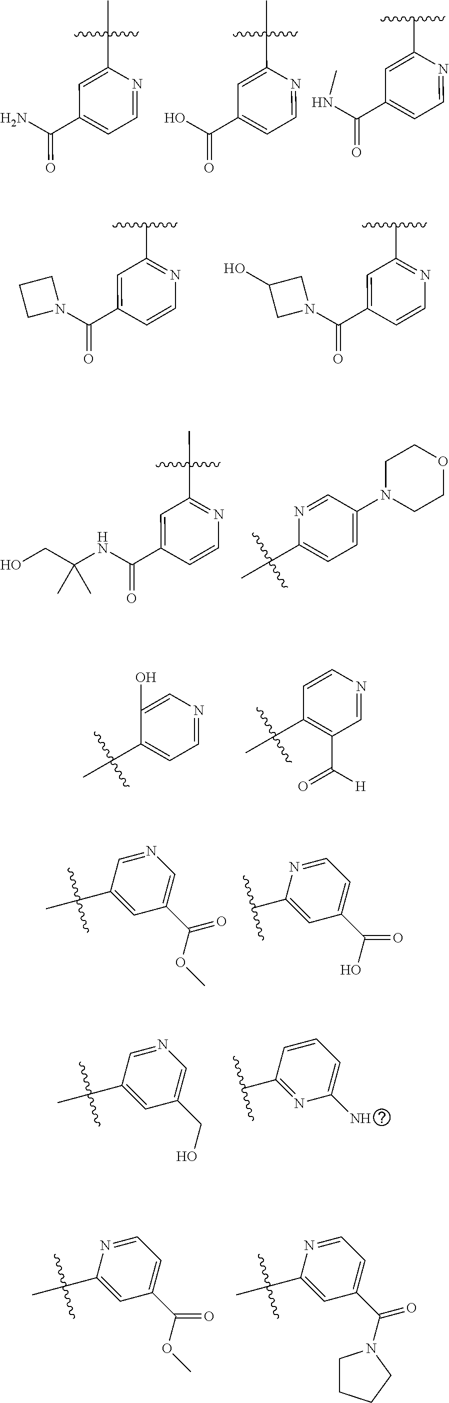

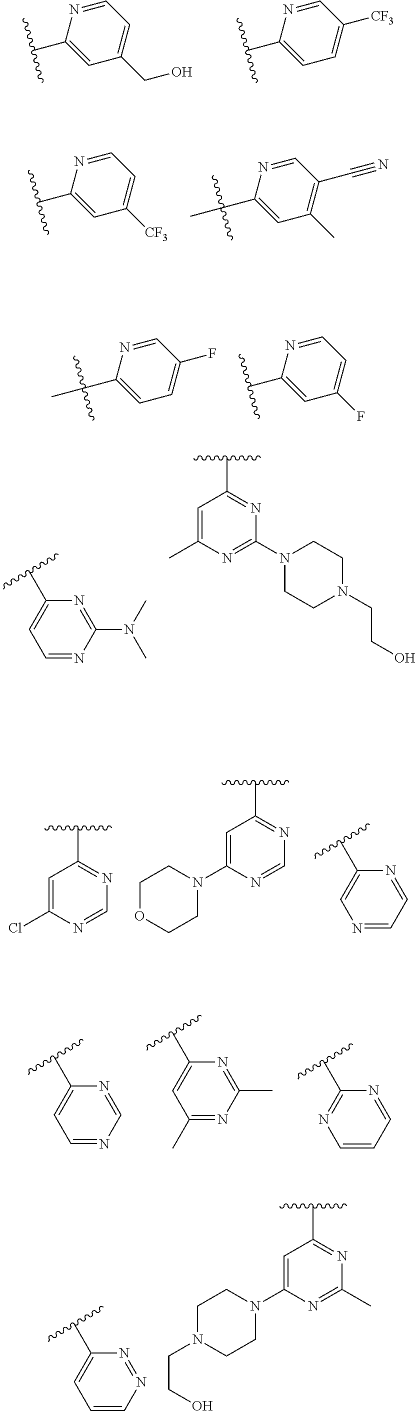

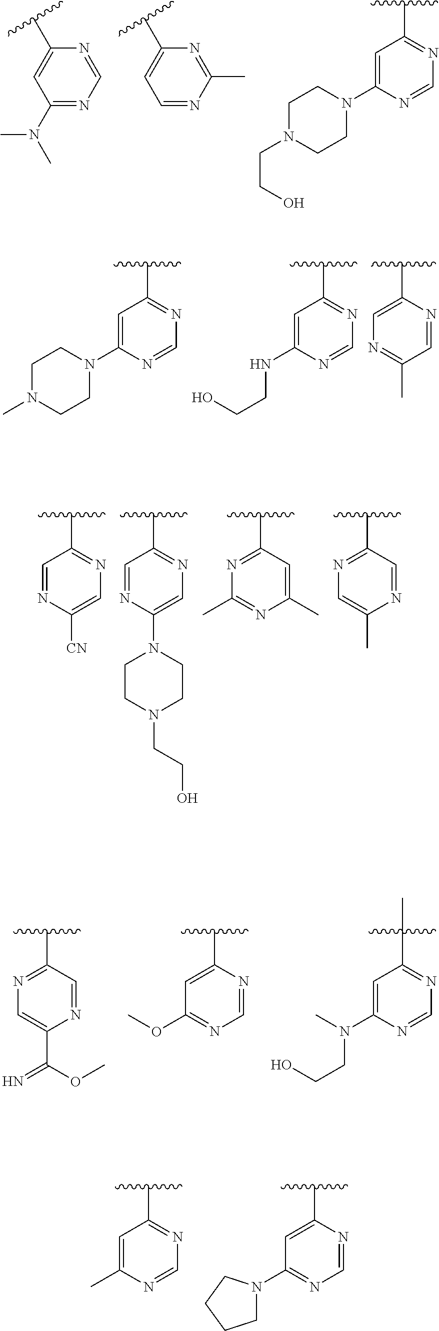

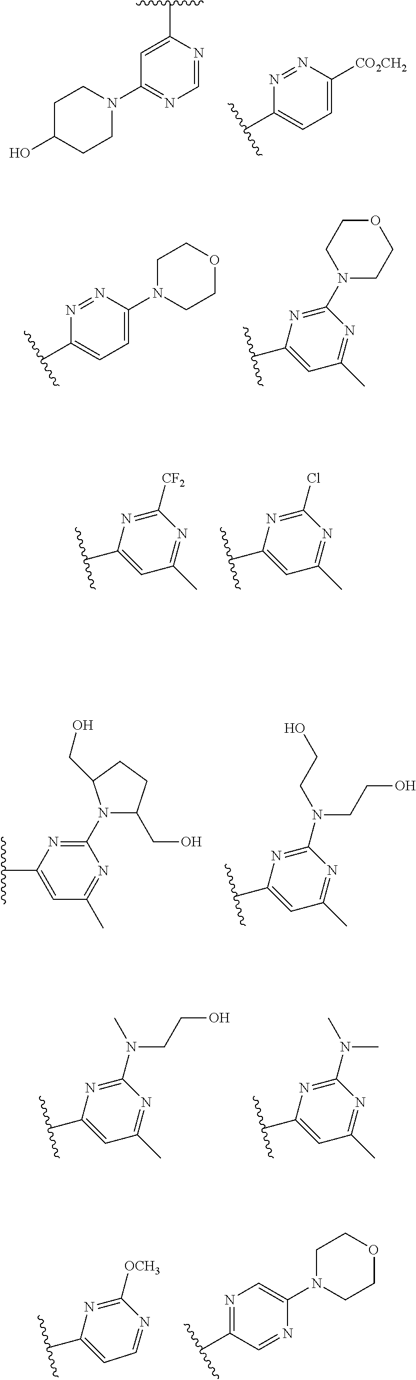

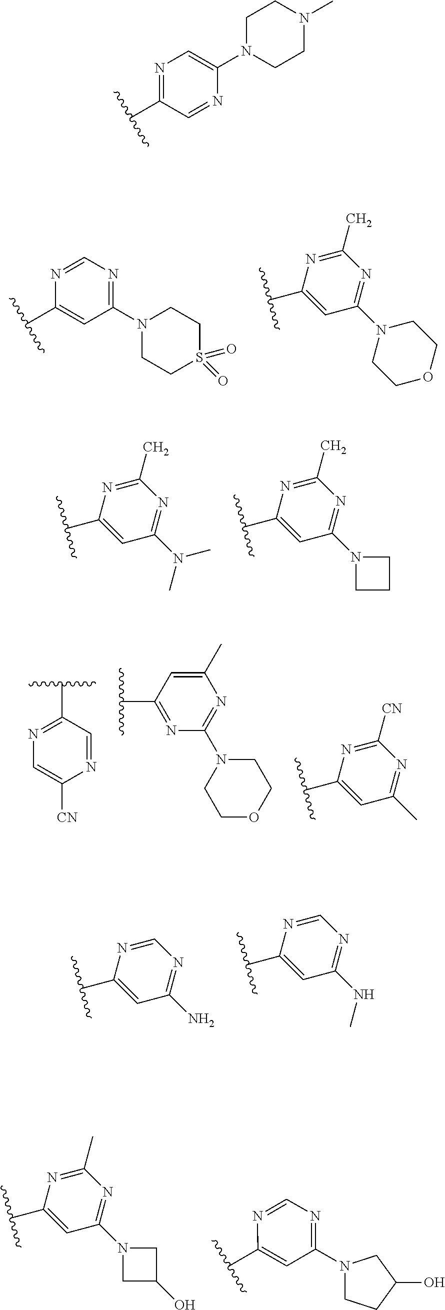

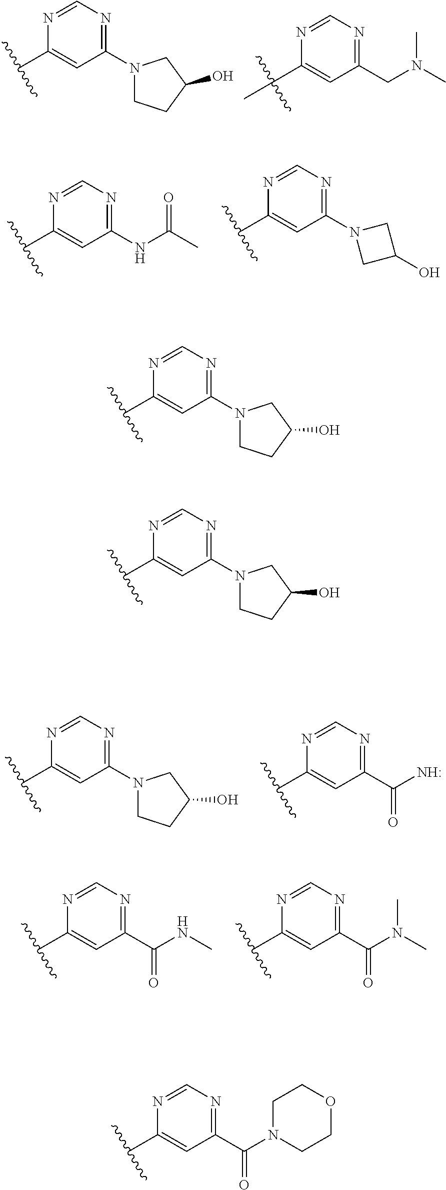

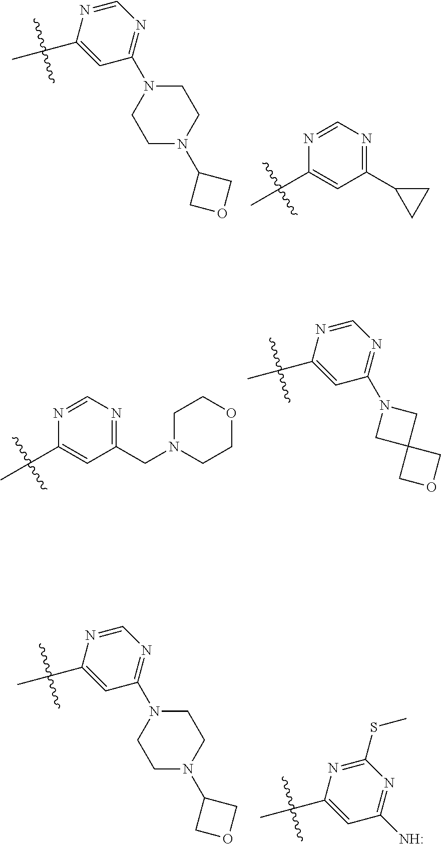

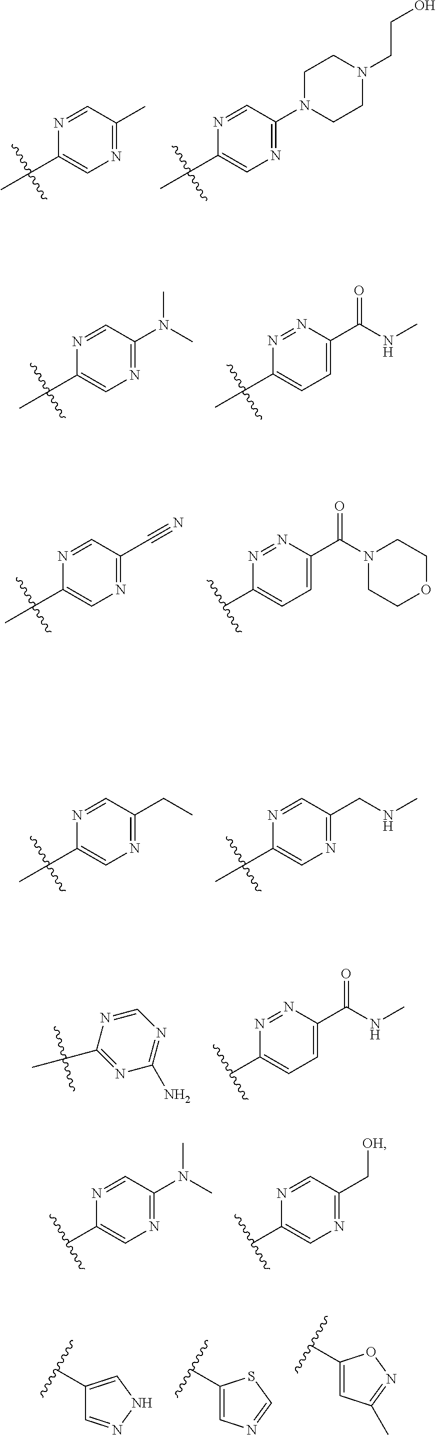

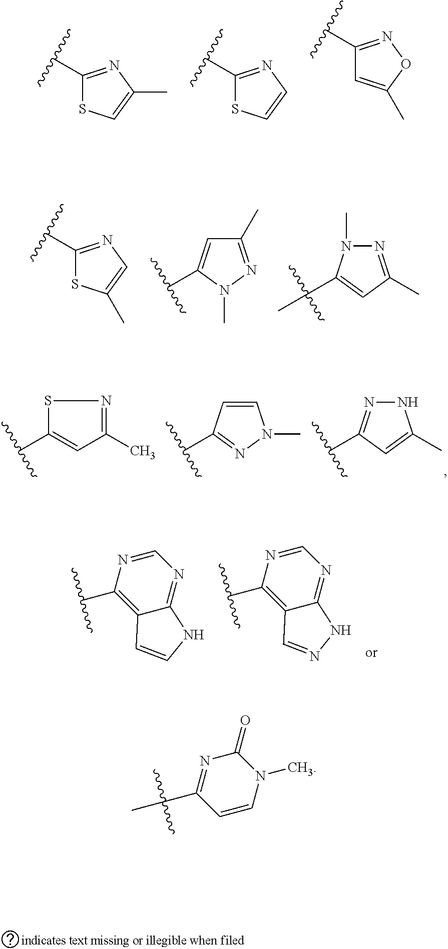

13. The compound of claim 1, wherein A is CR³; X is CH; R¹ is independently hydrogen, —OCH₃, —CF₃, —OCF₃, —CH₃, Cl, Br or F, wherein both R¹ cannot be hydrogen at the same time; R² is hydrogen, Cl or F; R³ is hydrogen or —CN; R⁴ is —NH—, NHC(O)—, NHC(O)NH— or NHC(O)O—; and R⁵ is cyclopropyl optionally substituted by halogen, or pyrimidinyl, pyridinyl, pyridazinyl or pyrazinyl optionally substituted by R¹⁰.

14. The compound of claim 1, selected from a compound of Examples 1-472.

15. A pharmaceutical composition comprising a compound of any one of claims 1-14 and a pharmaceutically acceptable carrier, adjuvant or vehicle.

16. A compound of any one of claims 1-14 for use in therapy.

17. A compound of any one of claims 1-14 for use in treating an inflammatory disease.

18. A compound of any one of claims 1-14 for use in treating psoriasis or inflammatory bowel disease.

19. A method of manufacturing a compound of any one of claims 1-14, comprising (a) reacting a compound of the formula: 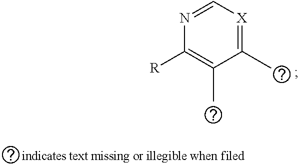
wherein R is halogen or leaving group, with a compound of the formula: 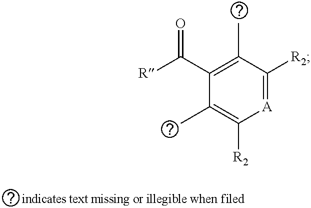
wherein R″ is halogen or leaving group to prepare a compound of formula iv:

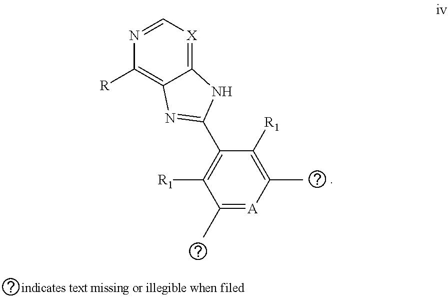

20. The method of claim 19, further comprising reacting a compound of formula iv with a compound of formula Lv-R¹⁶, wherein Lv is a leaving group, to form a compound of formulas va-vb:

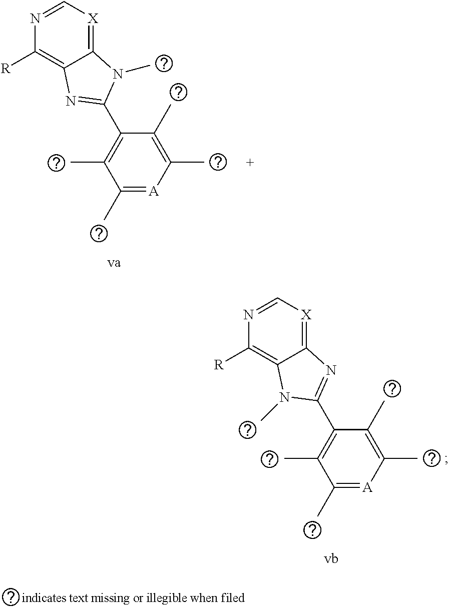

21. The method of claim 20, further comprising reacting a compound of formulas va-vb with a compound of the formula R⁴-R⁵ to form a compound of Formulas Ia-Ib.

22. The novel compounds, methods and uses substantially as described herein before.
